(19) United States
(12) Patent Application Publication
(10) Pub. No.: US 2026/0199535 A1
(43) Pub. Date: Jul. 16, 2026

(54) PCTA derivatives, conjugates thereof and uses thereof

(71) Applicant: NOVARTIS AG, Basel (CH)

(72) Inventors: Emmanuelle BRIARD, Huningue (FR); Claus EHRHARDT, Lörrach (DE); Philipp HOLZER, Sissach (CH); Pascal RIGOLLIER, Hagenthal-le-Bas (FR)

(21) Appl. No.: 19/132,568

(22) PCT Filed: Nov. 27, 2023
(86) PCT No.: PCT/IB2023/061928

§ 371 Date: May 23, 2025

Related U.S. Application Data
(60) Provisional application No. 63/385,071, filed on Nov. 28, 2022.

(51) Int. Cl. A61K 51/10; C07B 59/00; C07D 471/08; C07D 471/18; C07D 498/08

(52) U.S. Cl. CPC A61K 51/1093; A61K 51/1027; A61K 51/103; C07B 59/002; C07D 471/08; C07D 471/18; C07D 498/08

(57) ABSTRACT

The present disclosure relates to compounds comprising a macrocyclic chelator, and their conjugates with a target binding moiety. In particular, the present disclosure pertains to PCTA derivatives and conjugates thereof.

### TECHNICAL FIELD

The present disclosure relates to compounds comprising a macrocyclic chelator and a target binding moiety, as well as complexes thereof (e.g., with a radioisotope).

### BACKGROUND

Over the years, different macrocyclic chelators have been developed. The ability of these chelators to complex a number of different metal ions have drawn considerable interest. The fact that these macrocyclic chelators can form complexes with a radionuclides allows them to be used in different biomedical applications like diagnostic and therapeutics.

Some of the most commonly used macrocyclic chelators are DOTA (2,2′,2″,2′″-(1,4,7,10-Tetraazacyclododecane-1,4,7,10-tetrayl)tetraacetic acid) and NOTA (1,4,7-triazacyclononane-1,4,7-triacetic acid). These chelators have been widely developed and they are used in different applications, including radioligand therapy and imaging.

Radioligand therapies and/or diagnostics (RLT, RLD), typically have two main moieties: a chelator which complexes a radionuclide (like DOTA) and a target binding moiety which binds selectively to specific markers on target cells (like cancer cells). RLTs and RLDs can further comprise linkers or spacers, e.g., molecular entities used to increase the distance of target binding moiety from the chelators in order to prevent steric influence and loss of activity on the cell receptors upon functionalization. The length and composition of the linker can influence the binding affinity of the radiopharmaceutical to the receptor, the accumulation of radionuclides in tumor cells and the pharmacokinetic properties.

This approach to RLTs and RLDs has been particularly successful in the past, like with (¹⁷⁷Lu) oxodotreotide (or [¹⁷⁷Lu]-DOTA-TATE) which is used in the treatment of cancers which express somatostatin receptors.

Recently, PCTA (3,6,9,15-Tetraazabicyclo[9.3.1]pentadeca-1(15),11,13-triene-3,6,9-triacetic acid) based chelators have been developed. However, they have been less studied than others. The development of PCTA-derivatives that can be conjugated to a target binding moiety, and therefore, which could potentially be used in diagnostics and/or therapeutics, could be advantageous. Moreover, a PCTA-derivative capable of chelating a radionuclide at a low temperature (for e.g. ≤60° C.) with a high yield (for e.g. ≥85%) could provide additional advantages.

### SUMMARY

The present disclosure relates to a compound of formula (I)

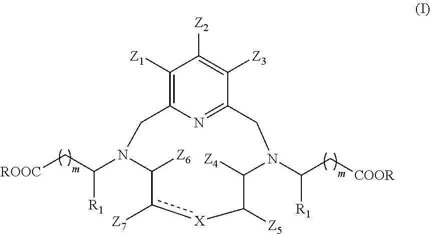

or a pharmaceutically acceptable salt thereof,
wherein
[symbol] is a single bond or a double bond,
when [symbol] is a single bond, X is —O— or

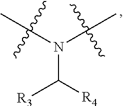

when [symbol] is a double bond, X is ═N— and Z₅ and Z₇ form, together with the N atom, a heteroaryl having 5 or 6 ring atoms, otherwise, Z₅ and Z₇ are each independently selected from the group consisting of H, an albumin-binder and —X₁-L₁-X₂-L₂-A;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

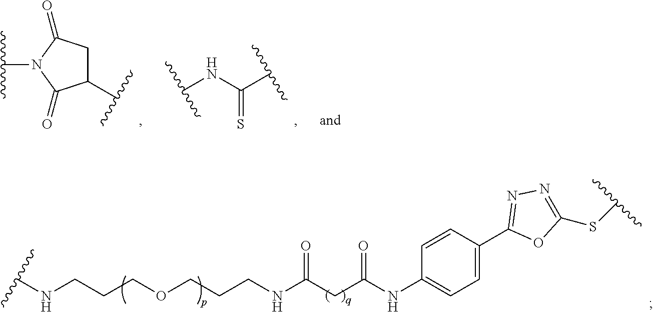

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
   n is 1, 2, 3, 4, 5, or 6;
   p is 1, 2, 3, 4, 5, or 6;
   q is 1, 2, 3, 4, 5, or 5; and
   A is a target binding moiety, e.g., selected from the group comprising a peptide, polypeptide, protein peptidomimetic, antibody and antigen-binding fragment, aptamer, DARPin, antisense oligonucleotide, siNA, small molecule, microparticle or nanoparticle.

The compounds of formula (I) have pharmacological properties that make them suitable for use in radioligand therapies. Indeed, the compounds of formula (I) can be easily labeled with a radionuclide, for example, ⁶⁸Ga and ¹⁷⁷Lu. The labeled compounds also have good stability and good biodistribution.

### DETAILED DESCRIPTION

### Definitions

Various embodiments of the disclosure are described herein. It will be recognized that features specified in each embodiment may be combined with other specified features to provide further embodiments.

The term “about” has herein the meaning that the following value may vary for ±20%, preferably ±10%, more preferably ±5%.

Unless otherwise defined, “%” has herein the meaning of weight percent (wt %), of yield percent, also referred to as weight by weight percent (w/w %).

The present disclosure encompasses the compounds of the disclosure, their stereoisomers, tautomers, enantiomers, diastereomers, racemates or mixtures thereof, and their hydrates, solvates or pharmaceutically acceptable salts.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The terms “pharmaceutically acceptable salts” refers to salts that retain the biological effectiveness and properties of the compounds of this disclosure and, which typically are not biologically or otherwise undesirable. In many cases, the compounds of the disclosure are capable of forming acid and/or base salts by virtue of the presence of amino and/or carboxyl groups or groups similar thereto. Pharmaceutically acceptable acid addition salts can be formed with organic acids and/or inorganic acids. Pharmaceutically acceptable base addition salts can be formed with organic bases and/or inorganic bases. Such salts are well-known from those skilled in the art. Examples of pharmaceutically acceptable salts include trifluoroacetic acid (TFA), acetate or hydrochloride salts.

As used herein, the terms “alkyl” and “C₁-Cₓ alkyl”, alone or as part of another substituent, refer to a linear or branched alkyl functional group having 1 to x carbon atoms, for examples 1 to 24, 1 to 20 carbon atoms, 1 to 12 carbon atoms, 1 to 6 carbon atoms, or 1 to 5 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g., n-hexyl, iso-hexyl).

Alkylene, used alone or as part of alkylene glycol for example, refers to a divalent saturated, straight-chained or branched alkyl group as defined herein.

As used herein, the terms “cycloalkyl”, and “carbocycle” refer to a saturated or partially unsaturated cyclic group. In an embodiment, cycloalkyl has 3 to 8 carbon atoms or 3 to 6 carbon atoms. The cycloalkyl can have a single ring or multiple rings fused together.

The cycloalkyl can also include spirocyclic rings. Suitable cycloalkyl groups include cyclopropyl, cyclobutyl, cyclopentyl and cyclohexyl.

As used herein, the terms “cycloalkylene” and “carbocyclo” refer to a divalent cycloalkyl as defined herein.

As used herein, the term “halogen” refers to a fluoro (—F), chloro (—Cl), bromo (—Br), or iodo (—I) group.

As used herein, the term “heteroalkyl”, refers to a straight or branched hydrocarbon chain consisting of 1 to 12 carbon atoms, e.g., 1 to 10 carbon atoms, or 1 to 6 carbon atoms, and from one to three heteroatoms selected from the group consisting of O, N, Si and S, and wherein the nitrogen and sulfur atoms may optionally be oxidized (for example: a sulfoxide or a sulfone) and the nitrogen heteroatom may optionally be quaternized. The heteroatom(s) O, N and S may be placed at any interior position of the heteroalkyl group or at the position at which the alkyl group is attached to the remainder of the molecule.

Heteroalkylene refers to a divalent heteroalkyl as defined above. For heteroalkylene groups, heteroatoms can also occupy either or both of the chain termini.

As used herein, the term “aryl” refers to a polyunsaturated, aromatic hydrocarbyl group having a single ring or multiple aromatic rings fused together, wherein at least one ring is aromatic. In an embodiment, aryl contains 5 to 10 ring atoms. The aromatic ring may optionally include one to two additional rings (cycloalkyl, heterocyclyl or heteroaryl as defined herein) fused thereto. Suitable aryl groups include phenyl, naphthyl and phenyl ring fused to a heterocyclyl, like benzopyranyl, benzodioxolyl, benzodioxanyl and the like.

As used herein, the term “heteroaryl” refers to a polyunsaturated, aromatic ring system having a single ring or multiple aromatic rings fused together or linked covalently. In an embodiment, heteroaryl contains 5 to 10 ring atoms, wherein at least one ring atom is a heteroatom selected from N, O, and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocyclyl” and “heterocycloalkyl” refer to a saturated or unsaturated cyclic group. In an embodiment, heterocyclyl comprises 3 to 10 ring atoms (or 3 to 8 ring atoms or 3 to 6 ring atoms), wherein at least one ring atom is a heteroatom selected from N, O, and S. The nitrogen and sulfur heteroatoms may optionally be oxidized and the nitrogen heteroatoms may optionally be quaternized. The heterocycle can include fused or bridged rings as well as spirocyclic rings. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

As used herein, the terms “heterocyclo” or “heterocycloalkylene” refer to a divalent heterocycle as defined herein.

Furthermore, alkyl, aryl, alkylene, arylene, heteroalkyl, heteroalkylene, C₃-C₈ carbocycle, C₃-C₈ carbocyclo, C₃-C₈ heterocycle, C₃-C₈ heterocyclo, and polyether can be optionally substituted with one or more of the substituents selected from: —X, —R′, —O—, —OR′, ═O, —SR′, —S—, —NR′₂, —NR′₃, ═NR′, —CX₃, —CN, —OCN, —SCN, —N═C═O, —NCS, —NO, —NO₂, ═N₂, —N₃, —NRC(═O)R′, —C(═O)R′, —C(═O)NR′₂, —SO₃⁻, —SO₃H, —S(═O)₂R′, —OS(═O)₂OR′, —S(═O)₂NR′, —S(═O)R′, —OP(═O)(OR′)₂, —P(═O)(OR′)₂, —PO₃—, —PO₃H₂, —C(═O)R′, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —C, —Br, or —I; and each R′ is independently —H, —C₁-C₂₀ alkyl, —C₆-C₁₀ aryl, or —C₃-C₁₀ heterocycle.

As used herein, the term “spacer” refers to a chemical structure covalently binding two components, like a chelator and A.

As used herein, the term “linker” refers to a chemical structure connecting moieties such as X₁ and X₂. The linker can increase the distance of the target binding moiety from the chelator in order to prevent steric influence and loss of activity on the cell receptors upon functionalization. The length and composition of the linker influences the binding affinity of the radiopharmaceutical to the receptor, the pharmacokinetics, the stability in the blood plasma and the accumulation of radionuclides in tumor cells. The linkers can be inert or functionalized. The linker(s) can also be categorized as cleavable or non-cleavable. Cleavable linkers can for example be split in the acidic environment of the lysosomes.

Non-exhaustive listing of linkers include: alkylene, heteroalkylene (so an alkylene interrupted by at least one heteroatom selected from Si, N, O and S); alkoxy; polyether such as polyalkylene glycol and typically polyethylene glycol; one or more natural or non-natural aminoacids such as glycine, alanine, proline, lysine, valine, N-methylglycine; C₃-C₈ heterocyclo; C₃—C carbocyclo; arylene, and any combination thereof. For example, a linker is a divalent linear alkylene group.

In some embodiments, the linker is selected from —C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁a alkylene-, —C₁-C₁a alkylene-C(═O)—, —C₁-C₁a heteroalkylene-C(═O)—, —C₃-C₈carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁a heteroalkylene-NH—, —C₃-C₈ carbocyclo-NH—, —O—(C₁-C₈ alkyl)-NH—, -arylene-NH—, —C₁-C₁a alkylene-arylene-NH—, -arylene-C₁-C₁₀ alkylene-NH—, —C₁-C₁₀ alkylene-(C₃-C₈carbocyclo)-NH—, —(C₃-C₈ carbocyclo)-C₁-C₁₀ alkylene-NH—, —C₃-C₈heterocyclo-NH—, —C₁—Cia alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—(C₁-C₈ alkyl)-)-S—, -arylene-S—, —C₁-C₁₀ alkylene-arylene-S—, -arylene-C₁-C₁₀ alkylene-S—, —C₁-C₁₀ alkylene-(C₃-C₈carbocyclo)-S—, —(C₃-C₈ carbocyclo)-C₁-C₁₀ alkylene-S—, —C₃-C₈ heterocyclo-S—, —C₁-C₁₀ alkylene-(C₃-C₃ heterocyclo)-S—, —(C₃-C₈ heterocyclo)-C₁-C₁₀ alkylene-S—, —C₁-C₁₀ alkylene-O—C(═O)—, —C₃-C₈ carbocyclo-O—C(═O)—, —O—(C₁-C₈ alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃—C carbocyclo)-O—C(═O)—, —(C₃-C₈ carbocyclo)-C₁-C₁₀ alkylene-O—C(═O)—, —C₃-C₈ heterocyclo-O—C(═O)—, —C₁-C₁₀ alkylene-(C₃-C₈ heterocyclo)-O—C(═O)—, —(C₃-C₈ heterocyclo)-C₁-C₁₀ alkylene-O—C(═O)—, and any combinations thereof.

Further, in an embodiment, any of the linker disclosed herein can be optionally substituted with one or more of the substituents selected from: albumin binder, —X, —R′, —O—, —OR′, ═O, —SR′, —S—, —NR′₂, —NR′₃+, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃⁻, —SO₃H, —S(═O)₂R′, —OS(═O)₂OR′, —S(═O)₂NR′, —S(═O)R′, —OP(═O)(OR′)₂, —P(═O)(OR′)₂, —PO₃⁻, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —Cl, —Br, or —I; and each R′ is independently —H, —C₁-C₂₀ alkyl, —C₆-C₁₀ aryl, or —C₃-C₁₀ heterocycle.

As used herein, the term “protecting group” refers to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007).

Protecting groups for protection of the carboxyl group, as described by Wutz et al. (pages 533-643), are used in certain embodiments. In some embodiments, the protecting group is removable by treatment with acid. Representative examples of carboxyl protecting groups include, but are not limited to, benzyl, p-methoxybenzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required.

Protecting group for protection of the amino group as described by Wutz et al. (pages 696-927), are used in certain embodiments. Representative examples of amino protecting groups include, but are not limited to, t-butyloxycarbonyl (Boc), 9-fluorenyl methoxycarbonyl (Fmoc), allyloxycarbonyl (alloc), N-(1-(4,4-dimethyl-2,6-dioxocyclohexylidene)ethyl) (Dde), 1-(1-Adamantyl)-1-Methylethoxycarbonyl (Adpoc), N-(1-(4,4-dimethyl-2,6-dioxocyclohex-1-ylidene)-3-methylbutyl) (ivDde), monomethoxytrityl (MMt) and 4-methyltrityl (Mtt). Persons skilled in the art will recognize appropriate situations in which protecting groups are required.

As used herein, the term “activated carboxylic acid” refers to a carboxylic acid group with the general formula —CO—X, wherein X is a leaving group. For example, activated forms of a carboxylic acid group may include, but are not limited to, acyl chlorides, symmetrical or unsymmetrical anhydrides, and esters. In some embodiments, the activated carboxylic acid group is an ester with pentafluorophenol, nitrophenol, benzotriazole, azabenzotriazole, thiophenol or N-hydroxysuccinimide (NHS) as leaving group

As used herein, the term cation refers to an ion having one or more positive charges. Examples of cation include H⁺, Na⁺, Li⁺, K⁺, Ca²⁺, Mg²⁺, and ammonium.

As used herein, the expression “target binding moiety” and “targeting ligand” are used interchangeably herein and refers to a part of a molecule which specifically binds with a target e.g., an antigen, a cell, cell type, tissue, organ, region of the body, or a compartment, typically a protein or a receptor, typically a receptor at the surface of a cell, in particular a cancerous cell.

Target binding moiety and targeting ligand include, but are not limited to peptides, polypeptide, proteins (such as antibodies, antibody fragments (including, but are not limited to, Fab, F(ab′)², a monospecific or bispecific Fab², a trispecific Fab³ scFv, dsFv, scFv-Fc, bispecific diabodies, trispecific triabodies, minibodies, a fragment of IgNAR (e.g., V-NAR), a fragment of hcIgG (e.g., VhH), bis-scFvs), affibodies, or fibronectin type III domains), peptidomimetics, fusion proteins/polypeptides, aptamers, DARPins, antisense oligonucleotides, siNA, small molecules, microparticle or nanoparticles.

In some embodiments, the target binding moiety in connection with a linker L is of the formula -L-R2-C(O)—R1; wherein
L is absent or present; R1 is an amino acid residue linked via an amino group thereof to the adjacent —C(O)— group (carbonyl group); and
R2 is an amino acid residue linked via an amino group thereof to the adjacent —C(O)— group.

In some embodiments, R1 is a glutamic acid residue; and R2 is a glutamic acid residue or a lysine residue;
As used herein, the term “amino acid” refers to naturally occurring and non-natural amino acids, as well as amino acid analogs and amino acid mimetics that function in a manner similar to the naturally occurring amino acids. Naturally occurring amino acids and those encoded by the genetic code, as well as those amino acids that are later modified, e.g., hydroxyproline, 2-carboxyglutamate, and 0-phosphoserine. Amino acid analogs refer to compounds that have the same basic chemical structure as a naturally occurring amino acid, i.e.: an α-carbon that is bound to a hydrogen, a carboxyl group, an amino group, and an R group, e.g., homoserine norleucine, methionine sulfoxide. methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “polypeptide” and “peptide” are used interchangeably herein to refer to polymers of amino acids of any length. The polymer may be linear or branched, it may comprise non-natural amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. In various embodiments, the polypeptide can be isolated from natural sources, can be a produced by recombinant techniques from a eukaryotic or prokaryotic host, or can be a product of synthetic procedures. The polypeptides or peptides described herein may be any polypeptides or peptides known in the art or their derivates or analogues, in particular the ones targeting receptors overexpressed in cancer. Nonlimiting examples of receptors as targets and nonlimiting examples of corresponding target binding said polypeptides or peptides are provided in Table 1, below

TABLE 1
Selected polypeptide or peptides

| Target binding | Target Receptors (including |
|---|---|
| polypeptides or peptides | subtypes nomenclature) |
| Somatostatin | sst₁, sst_{2A}, sst_{2B}, sst₃, sst₄, sst₅ |
| PSMA inhibitor | PSMA |
| FAP inhibitor | FAP |
| Folate | Folate |
| VIP | VPAC₁, VPAC₂ |
| PACAP | PAC₁ |
| α-M2 | α-M2 |
| Exendin | GLP-1 |
| RGD | ανβ3 |
| Substance P | Neurokinin-1 |
| Natriuretic peptides | NPR-A, NPR-C |
| CCK | CCK₁, CCK₂ |
| Gastrin | CCK₂ |
| Bombesin | BB₁ (NMB), BB₂ (GRP), BB₃, BB₄ |
| GRP | BB₂ (GRP) |
| Neurotensin | NT1, NT2, NT3 |
| NPY | Y₁, Y₂, Y₄, Y₅ |
| Substance P | NK₁, NK₂, NK₃ |
| Oxytocin | OT |
| LHRH | LHRH |
| GLP-1 | GLP-1 |
| Calcitonin | Calcitonin |
| Endothelin | ET_{A}, ET_{B} |
| Atrial natriuretic factor | ANP_{A}, ANP_{B} |
| α-MSH | α-MSH |

As used herein, the term “protein” refers to any organic compounds made of amino acids arranged in one or more linear chains and folded into a three-dimensional conformation. The amino acids in a polymer chain are joined together by the peptide bonds between the carboxyl and amino groups of adjacent amino acid residues. The term “protein” further includes, without limitation, peptides, single chain polypeptide or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, PEGylation and the like, incorporation of non-natural amino acids, and amino acid modification for chemical conjugation with another molecule.

As used herein, the term “peptidomimetics” refers to a synthetic chemical compound that has substantially the same functional characteristics of a naturally or non-naturally occurring polypeptide but different (though typically similar) structural characteristics. Such non-peptide compounds are termed “peptide mimetics” or “peptidomimetics.” Fauchere, J. Adv. Drug Res. 15:29 (1986); Veber and Freidinger TINS p. 392 (1985); Evans et al. J. Med. Chem. 30:1229 (1987). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as found in a polypeptide of interest, but have one or more peptide linkages optionally replaced by a linkage selected from the group consisting of, e.g., —CH₂NH—, —CH₂S—, —CH═CH— (cis and trans), —C(═O)CH₂—, —CH(OH)CH₂—, —CH₂SO— and —CH₂SO—. A mimetic can be either entirely composed synthetic, non-natural analogues of amino acids, or, is a chimeric molecule of partly natural peptide amino acids and partly non-natural analogs of amino acids. A mimetic can also incorporate any amount of natural amino acid conservative substitutions as long as such substitutions also do not substantially alter the mimetic's structure and/or activity.

As used herein, the term “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule catalysis of a reaction, etc. The two portions can be linked directly by a single peptide bond or through a peptide linker but are in reading frame with each other. Alternatively, a fusion protein refers to a protein which includes at least two distinct protein domains, wherein said two distinct protein domains are not naturally occurring in nature. Examples of fusion proteins includes Fc fusion proteins comprising a Fc fragment of an antibody fused to a non-antibody protein with target-binding properties, also called immunoadhesins.

As used herein, the term “antibody” refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region.

The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. In a wild-type antibody, at the N-terminus is a variable region and at the C-terminus is a constant region; the CH3 and CL domains actually comprise the carboxy-terminus of the heavy and light chain, respectively. The antibodies described herein may be any antibodies known in the art. Nonlimiting examples of antibodies are provided in Table 2, below.

TABLE 2
Selected antibodies

| Name (trademark) | Target | Cancer |
|---|---|---|
| Rituximab (Rituxan ®) | CD20 | B-cell non-Hodgkin lymphoma, chronic |
| | | lymphocytic leukemia |
| Trastuzumab (Herceptin ®) | HER2 | Breast, stomach |
| Ibritumomab | CD20 | B-cell non-Hodgkin lymphoma |
| tiuxetan (Zevalin ®) | | |
| Cetuximab (Erbitux ®) | EGFR | Colorectal, head & neck |
| Bevacizumab (Avastin ®) | VEGF | Colorectal, lung, brain, kidney, cervical, |
| | | ovarian, fallopian, peritoneal, liver |
| Panitumumab (Vectibix ®) | EGFR | Colorectal |
| Ofatumumab (Arzerra ®) | CD20 | Chronic lymphocytic leukemia |
| Ipilimumab (Yervoy ®) | CTLA4 | Melanoma, kidney, colorectal, liver, lung, |
| | | mesothelioma |
| Brentuximab | CD30 | Hodgkin lymphoma, T-cell non-hodgkin |
| vedotin (Adcetris ®) | | lymphoma |
| Pertuzumab (Perjeta ®) | HER2 | Breast |
| Ado-trastuzumab emtansine | HER2 | Breast |
| (Kadcyla ®) | | |
| Obinutuzumab (Gazyva ®) | CD20 | Chronic lymphocytic leukemia, B-cell non- |
| | | hodgkin lymphoma |
| Ramucirumab (Cyramza ®) | VEGFR2 | Stomach, colorectal, liver, lung |
| Pembrolizumab (Keytruda ®) | PD1 | Melanoma, lung, head & neck, hodgkin |
| | | lymphoma, Bladder, stomach, cervical, B- |
| | | cell non-hodgkin lymphoma, liver, kidney, |
| | | esophageal, endometrial, tumor |
| | | mutational burden-high, skin, breast |
| Blinatumomab (Blincyto ®) | CD19, CD3 | B-Cell acute lymphoblastic leukemia |
| Nivolumab (Opdivo ®) | PD1 | Melanoma, lung, kidney, hodgkin |
| | | lymphoma, head & neck, bladder, |
| | | colorectal, liver, esophageal, |
| | | mesothelioma |
| Dinutuximab (Unituxin ®) | GD2 | Neuroblastoma |
| Daratumumab (Darzalex ®) | CD38 | Multiple myeloma |
| Necitumumab (Portrazza ®) | EGFR | Lung |
| Elotuzumab (Empliciti ®) | SLAMF7 | Multiple myeloma |
| Atezolizumab (Tecentriq ®) | PDL1 | Bladder, lung, breast, liver, melanoma |
| Olaratumab (Lartruvo ®) | PDGFRA | Sarcoma |
| Avelumab (Bavencio ®) | PDL1 | Merkel cell carcinoma, bladder, kidney |
| Durvalumab (Imfinzi ®) | PDL1 | Lung |
| Inotuzumab | CD22 | B-Cell acute lymphoblastic leukemia |
| ozogamicin (Besponsa ®) | | |
| Tisagenlecleucel (Kymriah ®) | CD19 | B-Cell acute lymphoblastic leukemia, B- |
| | | cell non-hodgkin lymphoma |
| Gemtuzumab | CD33 | B-Cell acute lymphoblastic leukemia |
| ozogamicin (Mylotarg ®) | | |
| Axicabtagene | CD19 | B-cell non-hodgkin lymphoma |
| ciloleucel (Yescarta ®) | | |
| Mogamulizumab (Poteligeo ®) | CCR4 | T-Cell non-hodgkin lymphoma |
| Moxetumomab | CD22 | B-cell non-hodgkin lymphoma |
| pasudotox (Lumoxiti ®) | | |
| Cemiplimab (Libtayo ®) | PD1 | Cutaneous squamous cell carcinoma, |
| | | basal cell carcinoma, lung |
| Polatuzumab vedotin | CD79B | B-cell non-hodgkin lymphoma |
| (Polivy ®) | | |
| Enfortumab vedotin | NECTIN4 | Bladder |
| (Padcev ®) | | |
| Fam-trastuzumab | HER2 | Breast, stomach |
| deruxtecan (Enhertu ®) | | |
| Isatuximab (Sarclisa ®) | CD38 | Multiple myeloma |
| Sacituzumab govitecan | TROP2 | Breast, bladder |
| (Trodelvy ®) | | |
| Brexucabtagene | CD19 | B-cell non-hodgkin lymphoma |
| autoleucel (Tecartus ®) | | |
| Tafasitamab-cxix (Monjuvi ®) | CD19 | B-cell non-Hodgkin lymphoma |
| Belantamab mafodotin- | BCMA | Multiple myeloma |
| bimf (Blenrep ®) | | |
| Naxitamab-gqgk (Danyelza ®) | GD2 | Neuroblastoma |
| Margetuximab (Margenza ®) | HER2 | Breast |
| Lisocabtagene | CD19 | B-cell non-hodgkin lymphoma |
| maraleucel (Breyanzi ® | | |
| Idecabtagene | BCMA | Multiple myeloma |
| vicleucel (Abecma ®) | | |
| Ado-trastuzumab emtansine | HER2 | Breast |
| (Kadcyla ®) | | |
| Obinutuzumab (Gazyva ®) | CD20 | Chronic lymphocytic leukemia, B-cell |
| | | non-hodgkin lymphoma |
| Ramucirumab (Cyramza ® | VEGFR2 | Stomach, colorectal, liver, lung |
| Pembrolizumab (Keytruda ®) | PD1 | melanoma, lung, head & neck, hodgkin |
| | | lymphoma, bladder, stomach, cervical, B- |
| | | cell non-Hodgkin lymphoma, liver, kidney, |
| | | esophageal, endometrial, skin, breast |
| Blinatumomab (Blincyto ®) | CD19 × CD3 | B-Cell acute lymphoblastic leukemia |
| Nivolumab (Opdivo ®) | PD1 | Melanoma, lung, kidney, hodgkin |
| | | lymphoma, head & neck, bladder, |
| | | colorectal, liver, esophageal, |
| | | mesothelioma |
| Dinutuximab (Unituxin ®) | GD2 | Neuroblastoma |
| Daratumumab (Darzalex ®) | CD38 | Multiple myeloma |
| Necitumumab (Portrazza ® | EGFR | lung |
| Elotuzumab (Empliciti ®) | SLAMF7 | Multiple myeloma |
| Atezolizumab (Tecentriq ®) | PDL1 | Bladder, lung, breast, liver, melanoma |
| Olaratumab (Lartruvo ®) | PDGFRA | Sarcoma |
| Avelumab (Bavencio ® | PDL1 | Merkel cell carcinoma, bladder, kidney |

As used herein, the terms “Antibody fragment” or “antigen-binding fragment” of an antibody refer to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

As used herein, the term “Affibody” refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

As used herein, the term “bispecific antibody” refers to an antibody that binds to two or more different epitopes. In some embodiments, a bispecific antibody binds to two different targets. In some embodiments, a bispecific antibody binds to two different epitopes on a single target molecule. An antibody that binds to two different epitopes on a single target molecule is also known as a “biparatopic antibody.”

As used herein, the term “Fibronectin type III domain” refers to an evolutionarily conserved protein domain found in a wide-variety of extracellular proteins. The Fibronectin type III domain has been used as a molecular scaffold to produce molecules capable of selectively binding a specific antigen. Variants of the Fibronectin type III domains (FN3) that have been engineered for selective-binding may also be referred to as monobodies. FN3 domains may be biologically engineered by site-directed mutagenesis or mutagenic screening (e.g., CIS-display, phage display, yeast display, bacterial display, mRNA display, ribosome display).

As used herein, the term “DARPin” refers to an artificial polypeptide having high specificity and high binding affinity to a target protein, which is prepared via genetic engineering. DARPins are originated from natural ankyrin protein, and have a structure where at least 2 or at least 3 ankyrin repeat motifs, for example, 3, 4 or 5 ankyrin repeat motifs are repeated. For example, the DARPins comprising 3, 4 or 5 ankyrin repeat motifs may have a molecular weight of about 10 kDa, about 14 kDa, and about 18 kDa, respectively. DARPin includes a core part which carries out structural function and a target binding part outside of the core which binds to a target. The core part includes conserved amino acid sequence and the target binding part includes different amino acid sequence depending on the target

As used herein, the term “Aptamer” refers to a single-stranded oligonucleotide (single stranded DNA or RNA molecule) that can bind specifically to its target with high affinity. The aptamer described herein may be any aptamer known in the art. Nonlimiting examples of Aptamers and their targets are provided in Table 3, below.

TABLE 3
Selected Aptamers and their targets

| Aptamer | Target |
|---|---|
| Pegaptanib | Vascular Endothelial Growth Factor |
| E-10030 | Platelet-derived growth factor |
| ARC1905 | Complement component 5 |
| REG1/RB006 | Factor IXa |
| NU172 | Thrombin |
| ARC1779 | von Willebrand factor |
| AGRO001/AS1411 | Nucleolin |
| NOX-E36 | C-C Chemokine ligand 2 |
| NOX-A12 | Stromal cell-derived factor-1 |
| NOX-H94 | Hepcidin peptide hormone |
| EYE1 | Vascular endothelial growth factor |
| BAX499/ARC19499 | Tissue factor pathway inhibitor |
| ARC1905 | C5 |
| E-10030 | PDGF |
| REG1 | Coagulation factor IXa |
| ARC1779 | A1 domain of von Willebrand factor |
| NU172 | Thrombin |
| ARC19499 (BAX499) | TFPI |
| AS1411 (AGRO001) | Nucleolin |
| NOX-A12 | CXCL12 |
| NOX-E36 | CCL2 |
| NX21909 | Neutrophil elastase |
| TBA, NU172 and HB22 | Thrombin |
| TTA1 | Tenascin C |
| AptA and AptB | Mucin 1 glycoprotein |
| A9 and A10) | Prostate specific membrane antigen |
| F3 | Human matrix metalloprotease-9 |
| U2 | EGFRvIII |
| E07 | EGFR |
| mini 15-8 | HER2 |
| Apt3 and Apt3-amine | Carcinoembryonic antige |
| AS1411 | Nucleolin |

As used herein, the term “Antisense oligonucleotide” refers to a single-stranded nucleic acid molecule having a nucleobase sequence that permits hybridization to a corresponding segment of a target nucleic acid. Nonlimiting examples of Antisense oligonucleotides and their targets are provided in Table 4, below.

TABLE 4
Selected Antisense oligonucleotides and their targets

| Antisense oligonucleotide | Target |
|---|---|
| Custirsen (OGX-011) | Clusterin |
| EGFR antisense DNA | EGFR |
| Apatorsen (OGX-427 | Hsp27 |
| ISIS-STAT3Rx (ISIS 481464/AZD9150) | STAT3 |
| ISIS-ARRx (AZD5312) | Androgen Receptor |
| Trabedersen (AP 12009) | TGFβ2 |
| EZN-2968 | HIF-1α |
| LErafAON-ETU | c-raf |

As used herein, the term “short interfering nucleic acid” (siNA) refers to any nucleic acid molecule capable of inhibiting or down regulating gene expression or viral replication by mediating RNA interference (RNAi) or gene silencing in a sequence-specific manner. It includes short interfering RNA (siRNA), microRNA (miRNA), short interfering oligonucleotides and chemically-modified short interfering nucleic acid molecules. SiNAs are responsible for RNA interference, the process of sequence-specific post-transcriptional gene silencing in animals and plants. siNAs are generated by ribonuclease Ill cleavage from longer double-stranded RNA (dsRNA) which are homologous to, or specific to, the silenced gene target. The siNA described herein may be any siNA known in the art, in particular the ones targeting tumoral target. Nonlimiting examples of Gene-protein target of siNA and their cellular functions are provided in Table 5, below.

TABLE 5
Gene-protein target of siNA and their cellular functions

| Gene-protein target | Cellular function |
|---|---|
| B-raf | Serine/threonine kinase |
| Nox1 | Superoxide-generating oxidase |
| FAS/Her2 | Fatty acid synthase |
| Cyclin E | Cell-cycle control |
| Hec1 | Chromosomal segregation |
| Gp210 | Nuclear pore assembly |
| c-Kit | Signal transduction |
| MDR | Multidrug resistance |
| bcl-2 | Antiapoptotic |
| Livin | Antiapoptotic |
| survivin | Antiapoptotic |
| Ribonucleotide reductase | Gemcitabine resistance |
| Rho C | Cell motility |

As used herein, the term “small molecule” refers to a substance or compound that has a relatively low molecular weight (e.g. less than 4,000 Daltons, particularly less than 2000 Daltons). Typically, small molecules are organic, but are not proteins, polypeptides, or nucleic acids, though they may be amino acids or dipeptides. The small molecules described herein may be any small molecule known in the art, in particular the ones targeting tumoral target. Nonlimiting examples of small molecules and their targets are provided in Table 6, below

TABLE 6
Selected small molecules and their targets

| Compound name | Target |
|---|---|
| Erdafitinib | FGFR |
| Everolimus | mTOR |
| Abemaciclib | CDK4/6 |
| Alpelisib | PI3Ka |
| Anastrozole | Aromatase |
| Exemestane | Aromatase |
| Toremifene | Estrogen receptor |
| Fulvestrant | Estrogen receptor |
| Letrozole | Estrogen receptor |
| Palbociclib | CDK4/6 |
| Ribociclib | CDK4/6 |
| Lapatinib ditosylate | EGFR, ErbB2, ErbB4 |
| Olaparib | PARP1/2 |
| Megestrol acetate | Progestogen Receptor, Androgen Receptor |
| Neratinib maleate | HER2, EGFR |
| Tamoxifen citrate | Estrogen receptor |
| Talazoparib tosylate | PARP1 |
| Tucatinib | HER2, ErbB2 |
| Talazoparib tosylate | PARP1 |
| Goserelin acetate | LHRH agonist |
| Topotecan Hydrochloride | Topoisomerase I |
| Irinotecan Hydrochloride | Topoisomerase I |
| Regorafenib | Ret, Raf-1, VEGFR2 |
| Ziv-Aflibercept | KIT, PDGFRb, RAF, RET, VEGFR1/2/3 |
| Lenvatinib mesylate | VEGFR1/2/3, c-RET |
| Avapritinib | PDGFRa, c-Ki |
| Imatinib mesylate | v-Abl, c-Kit, PDGFR |
| Ripretinib | c-Kit, PDGFRa |
| Sunitinib malate | VEGFR2, PDGFRb, c-kit |
| Tivozanib Hydrochloride | VEGFR1/2/3 |
| Sorafenib tosylate | Raf-1, VEGFR2, B-Raf |
| Pazopanib Hydrochloride | VEGFR1/2/3 |
| Temsirolimus | mTOR |
| Dasatinib | Abl, Src, c-Kit |
| Ponatinib Hydrochloride | Abl, PDGFRa, VEGFR2, FGFR1, Src |
| Enasidenib mesylate | IDH2 |
| Gilteritinib fumarate | FLT3/AXL |
| Glasdegib maleate | Smoothened (Smo) |
| Ivosidenib | IDH |
| Midostaurin | PKCa/b/g, Syk, Flk-1 |
| Mitoxantrone Hydrochloride | Topoisomerase II, PKC |
| Venetoclax | Bcl-2 |
| Acalabrutinib | BTK |
| Duvelisib | PI3K d/g |
| Ibrutinib | BTK, Bmx, CSK, FGR, BRK |
| Idelalisib | p110d |
| Bosutinib | Src/Abl |
| Nilotinib | BCR-ABL |
| Cabozantinib-S-Malate | VEGFR2, c-Met, c-RET, c-Kit |
| Pemigatinib | FGFR |
| Afatinib dimaleate | EGFR, HER2 |
| Alectinib | ALK |
| Brigatinib | ALK, IGF-1R, FLT3, FLT3, EGFR |
| Capmatinib Hydrochloride | c-Met |
| Ceritinib | ALK |
| Crizotinib | ROS1, c-Met, ALK |
| Dabrafenib mesylate | B-Raf |
| Dacomitinib | EGFR, ErbB2, ErbB4 |
| Entrectinib | TrkA/B/C, ROS1, ALK |
| Erlotinib Hydrochloride | EGFR |
| Pralsetinib | RET |
| Gefitinib | EGFR |
| Lorlatinib | ALK, ROS1 |
| Trametinib dimethyl sulfoxide | MEK |
| Osimertinib mesylate | EGFR |
| Selpercatinib | c-RET |
| Tepotinib Hydrochloride | c-Met |
| Binimetinib | MEK |
| Encorafenib | RAF |
| Vemurafenib | B-RafV600E |
| Plerixafor | CXCR4, CXCL12 |
| Panobinostat lactate | HDAC |
| Selinexor | CRM1 |
| Fedratinib Hydrochloride | JAK2 |
| Ruxolitinib phosphate | Vera JAK1/2 |
| Copanlisib Hydrochloride | PI3Ka/b/g/d |
| Belinostat | HDAC |
| Zanubrutinib | BTK |
| Romidepsin | HDAC1, HDAC2 |
| Tazemetostat Hydrobromide | EZH2 |
| Umbralisib tosylate | PI3Kd |
| Vorinostat | HDAC |
| Rucaparib camsylate | PARP1 |
| Niraparib tosylate monohydrate | PARP1/PARP2 |
| Propranolol hydrochloride | b-Adrenoceptor |
| Apalutamide | Androgen receptor |
| Bicalutamide | Androgen Receptor |
| Darolutamide | Androgen receptor |
| Degarelix | GnRH receptor |
| Leuprolide acetate | GnRHR agonist |
| Enzalutamide | Androgen-receptor |
| Flutamide | Androgen-receptor |
| Nilutamide | Androgen-receptor |
| Relugolix | GnRHR |
| Vismodegib | Hedgehog |
| Sonidegib | Smo |
| Cobimetinib | MEK |
| Larotrectinib sulfate | TRK |
| Imiquimod | TLR7 |
| Vandetanib | VEGFR2 |
| Abiraterone acetate | CYP17 |
| Tretinoin | Retinoic acid receptor, Retinoid X receptor |
| Sotorasib | K-Ras (G12C) |

As used herein, the term “nanoparticle” refers to a material structure whose size in any dimension (e.g., x, y, and z Cartesian dimensions) is less than about 1 μm, e.g., less than about 500 nm or less than about 200 nm or less than about 100 nm, and greater than about 5 nm. A nanoparticle can have a variety of geometrical shapes, e.g., spherical, ellipsoidal.

As used herein, the term “microparticle” refers to a material structure whose size in any dimension (e.g., x, y, and z Cartesian dimensions) is about 10 μm, e.g., less than about 150 μm or less than about 100 μm or less than about 20 μm, and greater than about 10 μm. A microparticle can have a variety of geometrical shapes, e.g., spherical, ellipsoidal, etc.

As used herein, the term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

As used herein, the term “therapeutically effective amount” refers to an amount effective, at dosages and for periods of time necessary, to achieve a desired therapeutic result.

As used herein, the terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more antigen-binding molecules. In some embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

As used herein, the term “target” refers but non-limiting to an antigen, a cell, a cell type, a tissue, an organ, a region of the body or a compartment. The antigen described herein may be any antigen known in the art, in particular cancer antigen. Nonlimiting examples of targets and nonlimiting examples of cancers expressing said targets are provided in Table 7, below.

TABLE 7
Selected targets expressed by cancer

| Target | Exemplary Cancer(s) |
|---|---|
| CA6 | Ovarian, Breast, cervical, and pancreatic |
| CD19 | Leukemias and Lymphomas |
| CD20 | Lymphorns Leukemia |
| CD22 | Leukemias and Lymphomas |
| CD27L | Leukemias and Lymphomas |
| CD30 | Leukemias and Lymphomas |
| CD33 | Leukemias and Lymphomas |
| CD37 | Leukemias and Lymphomas |
| CD56 | Myeloma, Myeloid leukemia, Neuroendocrine tumors, |
| | Wilm's tumor, adult neuroblastoma, NK/T cell |
| | lymphomas, Pancreatic acinar-cell carcinoma, |
| | Pheochromocytoma, Small-cell lung carcinoma, and |
| | some mesodermally-derived tumors (e.g., myosarcoma) |
| CD66e | Colon |
| CD70 | Leukemias and Lymphomas |
| CD72 | Lymphorns Leukemia |
| CD74 | Glioma, Thyroid, Lymphoma, Lung, Liver, Pancreatic, |
| | Stomach, Colorectal, Head & neck, Renal, Urothelia, |
| | Prostate, Endometrial, Breast, Cervical, Ovarian, Skin |
| | and Melanoma |
| CD79a | Lymphoma, Non-small-cell lung and Myeloma |
| CD79b | Leukemias and Lymphomas |
| CD98 | Triple negative breast |
| CD138 | Leukemias and Lymphomas |
| CD174 | Colon and Gastric |
| CD180 | Colon and Lymphomas |
| CD227 | Breast and Bladder |
| (also known | |
| as MUC-1) | |
| CD326 (also | Colon |
| known as | |
| Epcam) | |
| HER2 | Breast, Colon, Esophageal, Gastric, Osteosarcoma, |
| | Desmoplastic small round cell, Squamous cell carcinoma |
| | of head and neck, Ovarian, Prostate, Pancreatic, |
| | Glioblastoma multiforme, Gastric junction |
| | adenocarcinoma, Non-small cell lung, Gastroesophageal |
| | junction adenocarcinoma, Cervical, Salivary gland, Soft |
| | tissue sarcoma, Thyroid, Leukemia, Melanoma, Ewing's |
| | sarcoma, Rhabdomyosarcoma and Neuroblastoma |
| EGFR | Lung, Colon, Head and neck |
| EGFRvIII | Glioma |
| FRα | Breast and Head and neck |
| GCC | Hepatocellular carcinoma |
| GPNMB | Melanoma |
| Mesothelin | Mesothelioma |
| MUC16 | Ovarian |
| NaPi2b | Ovarian |
| Nectin 4 | Breast, Lung and Ovarian |
| PSMA | Prostate and Brain |
| STEAP1 | Prostate |
| Trop-2 | Colon, Ovarian, Endometrial |
| ST4 (also | Colon, Ovarian, Endometrial, Esophageal, Breast, |
| known | Cervical, Non-small-cell-lung, Prostate, Renal, Gastric, |
| as TPBG) | and Bladder |
| AGS-16 | Tumors associated with Papilloma virus, Head and neck |
| | and Cervical |
| alpha v beta6 | Gliomas and Elanomas |
| CA19.9 | Colon |
| CAIX | Renal |
| 17 1A | Colon |
| CEACAMS | Colorectal |
| CRIPTO | Breast, Pancreatic, Ovarian, and Colon |
| DLL3 | Neuroendocrine tumors |
| DS6 | Gynecologic Malignancies |
| Endothelin B | Prostate |
| FAP alpha | Colorectal and Pancreatic |
| GD2 | Neuroblastoma, Small cell lung, sarcomas and Gliomas |
| PMEL 17 | Melanoma |
| SLC44A4 | Breast and Prostate |
| TENB2 | Prostate |
| TIM-1 | Ovarian, Lung and Renal |
| Endosialin | Sarcoma and Neuroblastoma |
| (also known | |
| as CD248 | |
| and Tem1) | |
| Fibronectin | Wide variety of cancers as a marker of tumor associated |
| Extra-domain | angiogenesis |
| B (also | |
| known as | |
| ED-B) | |
| LIV-1 (also | Breast and Epatocellular |
| known | |
| as ZIP6) | |
| Mucin 1 | Epithelial |
| ρ-cadherin | Gastric and Bladder |
| Fyn | Colon and Breast |
| SLTRK6 | Urothelial |
| Tenascin c | Glioblastoma multiforme |
| PRLR | Breast |
| Fibronectin | Tumor associated vasculature |
| GPA33 | Colon, Gastric and Pancreatic |
| splice | Glioblastoma |
| isoform of | |
| tenascin-C | |
| TAG-72 | Colon, Breast, Lung |
| B7-H3 | Prostate, Gliomas |
| LICAM | Ovarian, Glioma, Colon, Endometrial, other cancer stem |
| | cells and invasive epithelial tumors |
| Lewis Y | Breast, Gastrointestinal; Prostate |
| FGFR1-4 | Urothelial carcinoma, Acute lymphoblastic leukemia, |
| | Chronic myelogenous leukemia, Cholangiocarcinoma, |
| | Head and neck, Non small cell lung, Intrahepatic |
| | cholangiocarcinoma, Gastric, Small cell lung, Breast, |
| | Endometrial, Cervical and Rhabdomyosarcoma |
| mTOR | Breast, Pancreatic, Gastrointestinal, Lung, |
| | Subependymal giant cell astrocytoma and Renal |
| CDK4/6 | Breast |
| PI3Ka | Breast |
| Aromatase | Breast |
| ErbB2, | Breast |
| ErbB4 | |
| EGFR | Breast, Non-small cell lung and Pancreatic |
| PARP1/2 | Breast, Ovarian, Ovarian epithelial, fallopian tube, or |
| | primary peritoneal, Pancreatic and Prostate |
| Progestogen | Breast and Endometrial |
| Androgen | Breast, Endometrial and Prostate |
| Estrogen | Breast and Ductal carcinoma |
| LHRH | Breast and Prostate |
| agonist | |
| Topoiso- | Cervical, Ovarian, Colorectal, Rectal, Acute non- |
| merase I | lymphocytic leukemia and Acute non-lymphocytic |
| | leukemia |
| Raf-1 | Colorectal and Gastrointestinal stromal, Hepatocellular |
| | carcinoma and Rectal |
| VEGFR1/2/3 | Colorectal, Rectal, Renal cell and Medullary thyroid |
| PDGFRb, | Colorectal , Rectal, Gastrointestinal stromal, Acute |
| PDGFRa, | lymphoblastic leukemia and Chronic myelogenous |
| | leukemia |
| C-RET | Endometrial carcinoma, Hepatocellular carcinoma, |
| | Renal cell carcinoma, Thyroid, Medullary thyroid and |
| | Non-small cell lung |
| c-Kit | Gastrointestinal stromal |
| V-Abl | Acute lymphoblastic leukemia, Chronic eosinophilic |
| | leukemia or hypereosinophilic syndrome, Chronic |
| | myelogenous leukemia, Dermatofibrosarcoma |
| | protuberans, Gastrointestinal stromal, |
| | Myelodysplastic/myeloproliferative neoplasms, |
| | Systemic mastocytosis |
| B-Raf | Renal Cell, Hepatocellular carcinoma, Thyroid, |
| | Anaplastic thyroid, Melanoma and Non-small cell lug |
| Src | Acute lymphoblastic leukemia and Chronic |
| | myelogenous leukemia |
| IDH2 | Acute myeloid leukemia |
| FLT3/AXL | Acute myeloid leukemia |
| Smoothened | Acute myeloid leukemia |
| IDH | Acute myeloid leukemia |
| Syk | Acute myeloid leukemia and mast cell leukemia |
| Flk-1 | Acute myeloid leukemia and mast cell leukemia |
| PKCa/b/g | Acute non-lymphocytic leukemia, Acute non- |
| | lymphocytic leukemia and Prostate |
| Bcl-2 | Acute myeloid leukemia and Chronic lymphocytic |
| | leukemia and Small lymphocytic lymphoma |
| BTK | Chronic lymphocytic leukemia, Small lymphocytic |
| | lymphoma and Mantle cell lymphoma |
| PI3K d/g | Chronic lymphocytic leukemia, Small lymphocytic |
| | lymphoma and Follicular lymphoma |
| Bmx | Chronic lymphocytic leukemia, Small lymphocytic |
| | lymphoma, Mantle cell lymphoma, Mantle cell |
| | lymphoma and Waldenström macroglobulinemia |
| CSK | Chronic lymphocytic leukemia, Small lymphocytic |
| | lymphoma, Mantle cell lymphoma, Mantle cell |
| | lymphoma and Waldenström macroglobulinemia |
| BRK | Chronic lymphocytic leukemia, Small lymphocytic |
| | lymphoma, Mantle cell lymphoma, Mantle cell |
| | lymphoma and Waldenström macroglobulinemia |
| p110d | Chronic lymphocytic leukemia, Non-Hodgkin |
| | lymphoma, Follicular B-cell non-Hodgkin |
| | lymphoma and Small lymphocytic lymphoma |
| BCR | Chronic myelogenous leukemia |
| c-Met | Hepatocellular carcinoma, Medullary thyroid cancer, |
| | Renal cell, Anaplastic large cell lymphoma Carcinoma |
| | and Non-small cell lung |
| ALK | Non-small cell lung |
| IGF-1R | Non-small cell lung |
| FLT3 | Non-small cell lung |
| ROS1 | Anaplastic large cell lymphoma and Non-small cell lung |
| ErbB2, | Non-small cell lung |
| ErbB4 | |
| TrkA/B/C | Non-small cell lung |
| MEK | Anaplastic thyroid and Melanoma, Non-small cell lung |
| CXCR4 | Multiple myeloma and Non-Hodgkin lymphoma |
| HDAC | Multiple myeloma and Cutaneous T-cell lymphoma |
| CXCL12 | Multiple myeloma and Non-Hodgkin lymphoma |
| CRM1 | Diffuse large B-cell lymphoma and Multiple myeloma |
| JAK1/2 | Myelofibrosis |
| vera | Myelofibrosis and Polycythemia |
| PI3Ka/b/g/d | Marginal zone lymphoma and Follicular lymphoma |
| HDAC1, | Cutaneous T-cell lymphoma and Peripheral T-cell |
| HDAC2 | lymphoma |
| EZH2 | Epithelioid sarcoma and Follicular lymphoma |
| GnRHR | Prostate |
| Hedgehog | Basal cell carcinoma |
| Smo | Basal cell carcinoma |
| MEK | Melanoma |
| TLR7 | Basal cell carcinoma |
| CYP17 | Prostate |
| Retinoic acid | Acute promyelocytic leukemia |
| receptor, | |
| K-Ras | Non-small cell lung |
| Polysialic | Small cell lung and Wilms tumors |
| acid | |

As used herein, the term “Albumin-Binder” refers to a group, which binds noncovalently to human serum albumin (typically with a binding affinity less than about 10 MicroM). Albumin binding properties can be measured by surface plasmon resonance as described in J. Biol. Chem. 277(38), 35035-35042, (2002). Typical albumin binders suitable for use of the present invention include linear and branched lipophilic groups having 12-40 carbon atoms and a distal acidic group. Suitable albumin binders for use in the compounds of the present invention are selected from examples provided in Table 8, below.

TABLE 8
Selected Albumin-Binder

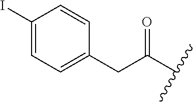 [SMILES: *C(=O)Cc1ccc(I)cc1]

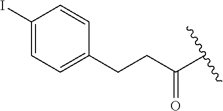 [SMILES: *C(=O)CCc1ccc(I)cc1]

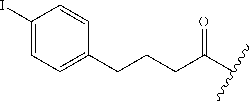 [SMILES: *C(=O)CCCc1ccc(I)cc1]

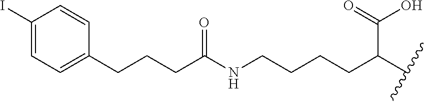 [SMILES: *C(CCCCNC(=O)CCCc1ccc(I)cc1)C(=O)O]

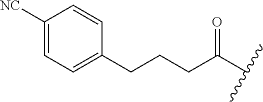 [SMILES: *C(=O)CCCc1ccc(C#N)cc1]

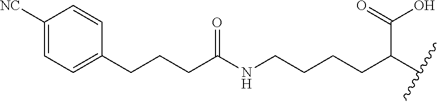 [SMILES: *C(CCCCNC(=O)CCCc1ccc(C#N)cc1)C(=O)O]

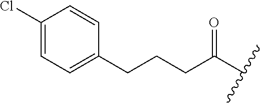 [SMILES: *C(=O)CCCc1ccc(Cl)cc1]

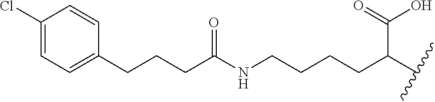 [SMILES: *C(CCCCNC(=O)CCCc1ccc(Cl)cc1)C(=O)O]

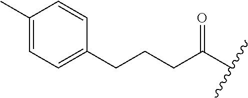 [SMILES: *C(=O)CCCc1ccc(C)cc1]

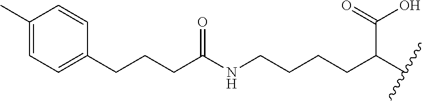 [SMILES: *C(CCCCNC(=O)CCCc1ccc(C)cc1)C(=O)O]

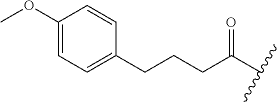 [SMILES: *C(=O)CCCc1ccc(OC)cc1]

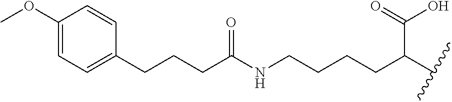 [SMILES: *C(CCCCNC(=O)CCCc1ccc(OC)cc1)C(=O)O]

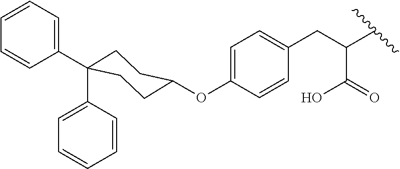 [SMILES: *C(Cc1ccc(OC2CCC(c3ccccc3)(c3ccccc3)CC2)cc1)C(=O)O]

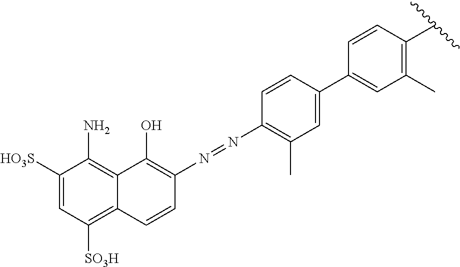 [SMILES: *c1ccc(-c2ccc(N=Nc3ccc4c(S(=O)(=O)O)cc(S(=O)(=O)O)c(N)c4c3O)c(C)c2)cc1C]

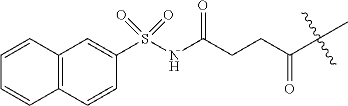 [SMILES: *C(=O)CCC(=O)NS(=O)(=O)c1ccc2ccccc2c1]

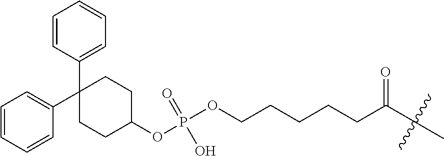 [SMILES: *C(=O)CCCCCOP(=O)(O)OC1CCC(c2ccccc2)(c2ccccc2)CC1]

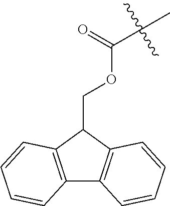 [SMILES: *C(=O)OCC1c2ccccc2-c2ccccc21]

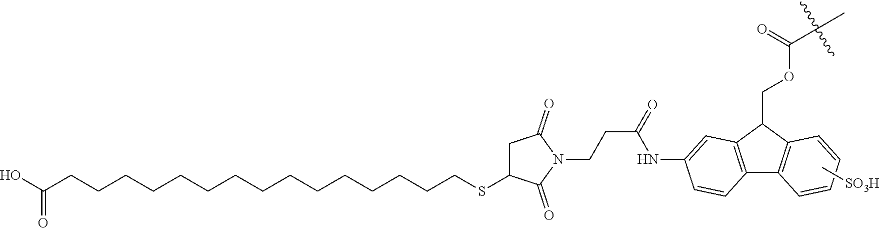

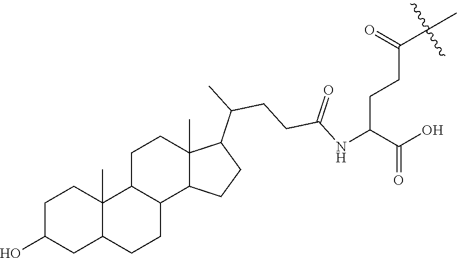 [SMILES: *C(=O)CCC(NC(=O)CCC(C)C1CCC2C3CCC4CC(O)CCC4(C)C3CCC12C)C(=O)O]

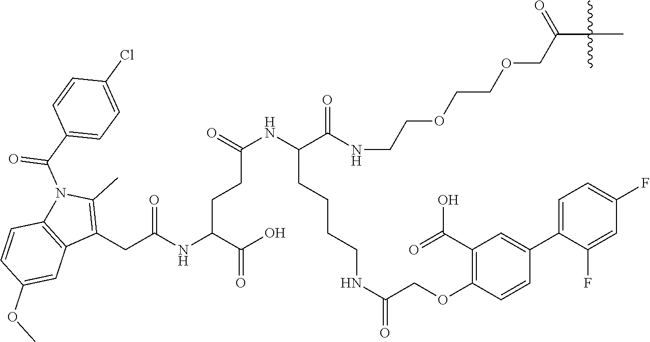 [SMILES: *C(=O)COCCOCCNC(=O)C(CCCCNC(=O)COc1ccc(-c2ccc(F)cc2F)cc1C(=O)O)NC(=O)CCC(NC(=O)Cc1c(C)n(C(=O)c2ccc(Cl)cc2)c2ccc(OC)cc12)C(=O)O]

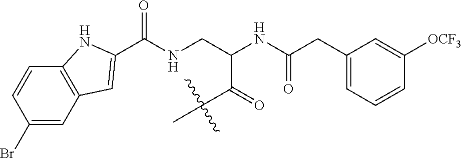 [SMILES: *C(=O)C(CNC(=O)c1cc2cc(Br)ccc2[nH]1)NC(=O)Cc1cccc(OC(F)(F)F)c1]

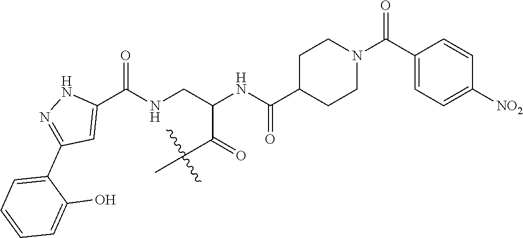 [SMILES: *C(=O)C(CNC(=O)c1cc(-c2ccccc2O)n[nH]1)NC(=O)C1CCN(C(=O)c2ccc([N+](=O)[O-])cc2)CC1]

As used herein, the terms “disease” or “disorder” refer to a condition where treatment is needed and/or desired.

As used herein, the terms “cancer” and “tumor” encompass solid and haematological/lymphatic cancers and also encompass malignant, pre-malignant, and benign growth, such as dysplasia. Also, included in this definition are cells having abnormal proliferation that is not impeded (e.g. immune evasion and immune escape mechanisms) by the immune system. Exemplary cancers include, but are mot limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; oesophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukoma; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (e.g., tongue, and pharynx); ovarian cancer; pancreatic cancer; prostate cancer; retinoblastoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; hulky discase NHL; mantle cell lymphoma AIDS-related lymphoma; and Waldenstrom's Macroglobulinemia; chronic lymphocytic leukemia (CLL); acute lymphoblastic leukemia (ALLH Hairy cell leukemia; chronic myeloblastic leukemia; as well as other carcinomas and sarcomas; and post-transplant lymphoproliferative disorder (PTLD), as well as abnormal vascular proliferation associated with phakomatoses.

The compounds of formula (I) are conjugates between a compound of formula (II) and a target binding moiety A. The compounds of formula (I) can be synthesized from compounds of formula (II) by reacting them with a target binding moiety A, using techniques well known from the person skilled in the art.

Typically, the compounds of formula (II) can be prepared according to the Schemes provided infra.

The generic schemes below provide guidance to the synthetic chemist of ordinary skill in the art, who will readily appreciate that the solvent, concentration, reagent, protecting group, order of synthetic steps, time, temperature, and the like can be modified as necessary.

The schemes provided infra are intended to represent single diastereomers/enantiomers as well as their isomeric mixtures. Separation of diastereomers/enantiomers may be performed according to techniques described herein. If not defined otherwise, in the general schemes described below, the substituents Z₄, Z₅ are as defined herein.

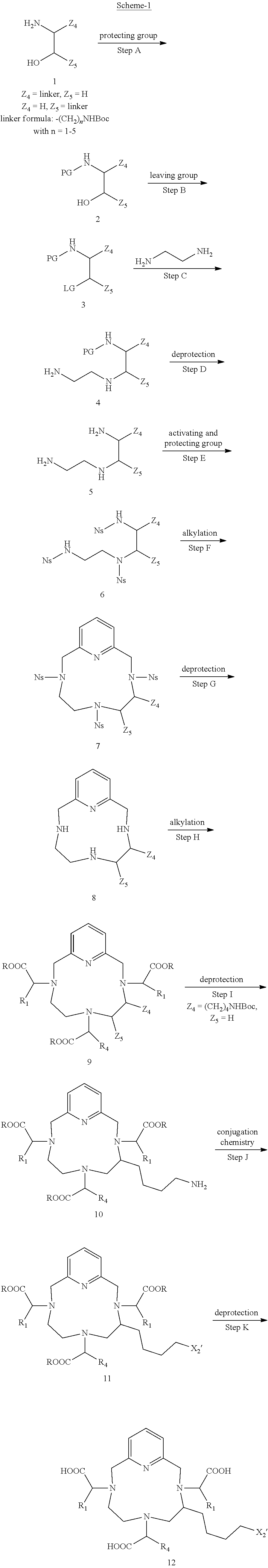

Scheme-1 provides a synthesis route for preparation of compounds from Formula Ila as disclosed herein. An appropriate amino-alcohol (1) is treated with a protecting group such as benzyl chloroformate. In the step B, the alcohol is converted into a leaving group, for example halo (such as chloro, bromo) methanesulfonate, p-toluenesulfonate, in the presence of a suitable base. In step C, the ethane-1,2-diamine is introduced and used as solvent. In step D, the protecting group is removed under appropriate conditions depending on the protecting group used. For example the carbamate of compound (4) is removed using conditions known in the art, in presence of hydrogen gas and palladium on charcoal to provide compound (5). In step E, compound (5) is treated in presence of 2-nitrobenzenesulfonylchloride and an appropriate base, such as sodium bicarbonate. The macrocyclisation step F is carried out by condensation of compound (6) and 2,6-bis(bromomethyl)pyridine in a solvent such as acetonitrile at reflux and in the presence of an excess of sodium carbonate as a base. In step G, the removal of the nosylate group is performed by treatment with thiophenol in the presence of a base such as potassium carbonate. In step H, the alkylation is achieved using the appropriate reagent such as the tert-butylbromoacetate, di-tert-butyl (S)-2-bromosuccinate, di-tert-butyl (R)-2-bromosuccinate, in a solvent such as DMF and in the presence of a base such as potassium carbonate. In step I, the protecting group is removed under appropriate conditions depending upon the protecting group used. For example, the Boc group of compound (9) is removed using an organic acid such as trifluoroacetic acid in a solvent such as dichloromethane or with a mineral acid such as hydrochloric acid in a solvent such as 1,4-dioxane to provide compound (10). In step J, the introduction of the maleimide is done stepwise. Treatment of compound (10) with maleic anhydride in the presence of a base such as triethylamine leads to an intermediate which cyclize in the presence of an activating group such as pentafluorophenol and base such as diisopropylmethanediimine to provide compound (11). Alternatively treatment of compound (10) with maleic anhydride in acetic anhydride in the presence of sodium acetate gives compound (11). In step K, the protecting groups are removed using trifluoroacetic acid in water.

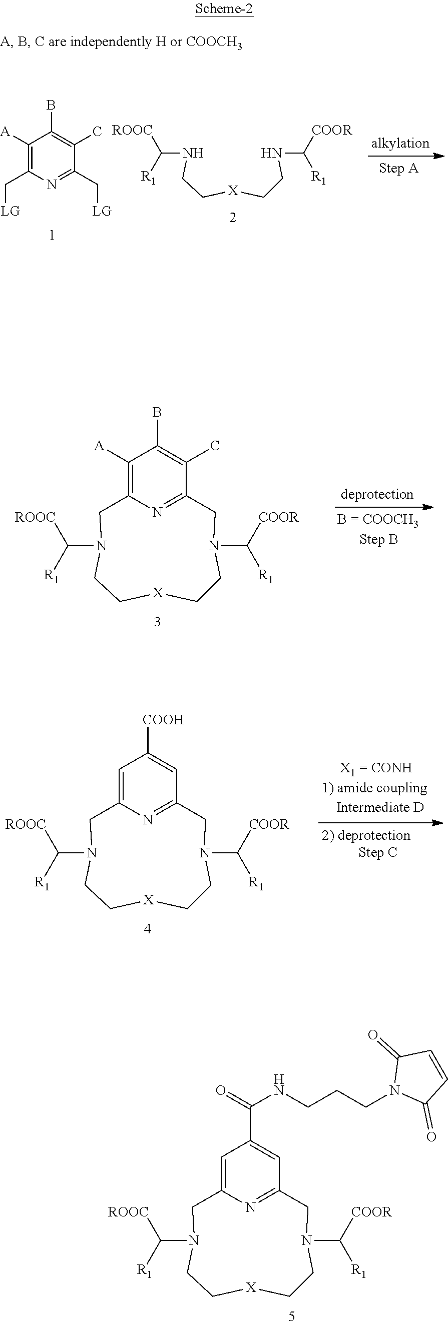

Scheme-2 provides a synthesis route for preparation of compounds from Formula IIb, IIc as disclosed herein. An appropriate heteroaromatic compound (1) such as methyl 2,6-bis(bromomethyl)isonicotinate is reacted with an appropriate diamine (2) such as tetra-tert-butyl 2,2′-((oxybis(ethane-2,1-diyl))bis(azanediyl))(2R,2′R)-disuccinate in the presence of a base such as sodium carbonate to provide after hydrolysis compound (4). In step C, the linker is introduced by reaction with a suitable maleimide functionalized linker for example the 4-(2,5-dioxo-2,5-dihydro-1H-pyrrol-1-yl)butan-1-aminium chloride followed by deprotection of the tert-butyl esters using for example trifluoroacetic acid.

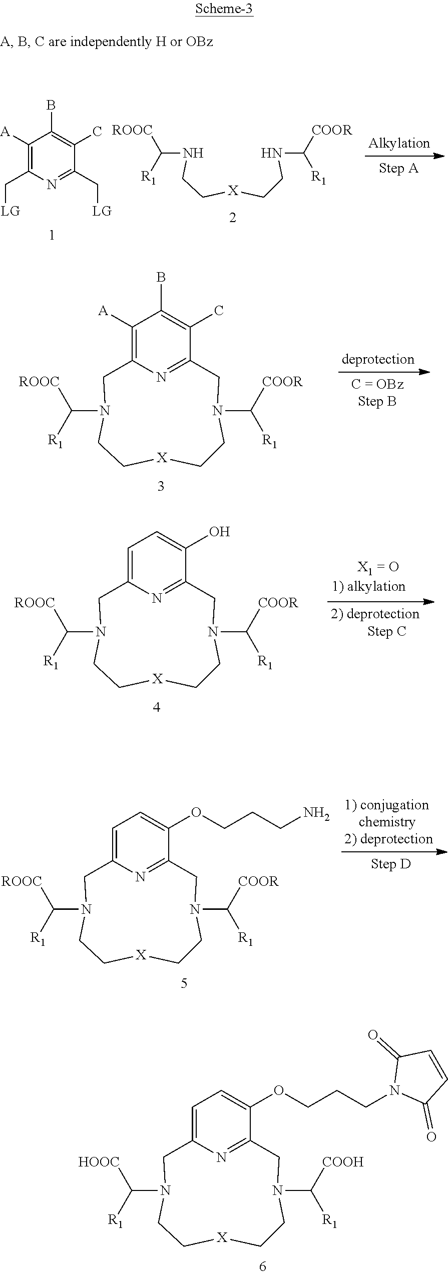

Scheme-3 provides a synthesis route for preparation of compounds from Formula IIc as disclosed herein. An appropriate heteroaromatic compound (1) such as 3-(benzyloxy)-2,6-bis(bromomethyl)pyridine is reacted with an appropriate diamine (2) such as tetra-tert-butyl 2,2′-((oxybis(ethane-2,1-diyl))bis(azanediyl))(2R,2′R)-disuccinate in the presence of a base such as sodium carbonate to provide after reduction compound (4). In step C, the linker is introduced by alkylation with a suitable N-Boc linker for example the tert-butyl (3-bromopropyl)carbamate, followed by deprotection of amine provides compound (5). In step D, the introduction of the phthalimide is done stepwise. Treatment of compound (5) with maleic anhydride in the presence of a base such as triethylamine leads to an intermediate which cyclize in the presence of an activating group such as pentafluorophenol and base such as diisopropylmethanediimine to give, after deprotection of the tert-butyl esters, compound (6).

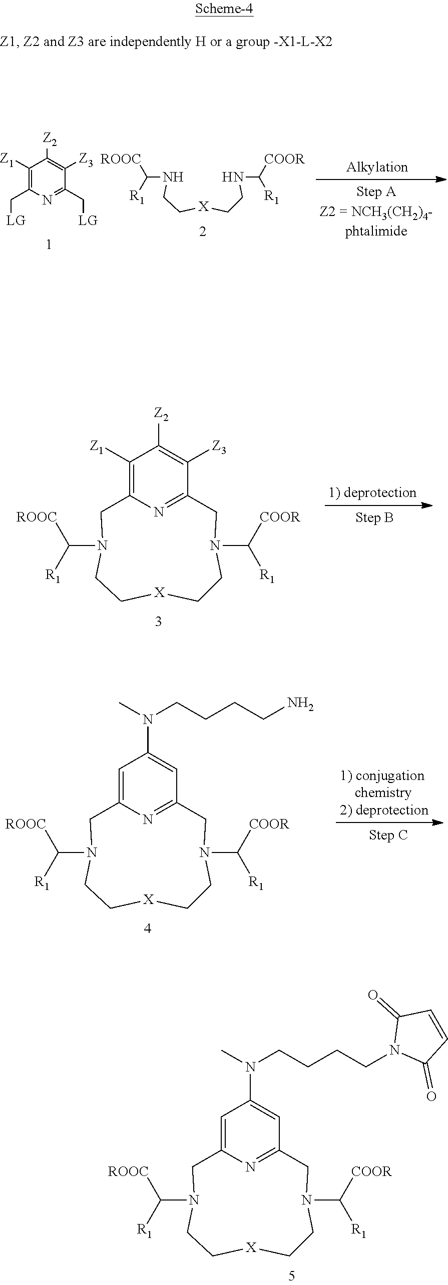

Scheme-4 provides a synthesis route for preparation of compounds from Formula IIb as disclosed herein. An appropriate heteroaromatic compound (1) such as 3-(benzyloxy)-2,6-bis(bromomethyl)pyridine is reacted with an appropriate diamine (2) such as di-tert-butyl 2,2′-((((2-(tert-butoxy)-2-oxoethyl)azanediyl)bis(ethane-2,1-diyl))bis(azanediyl))diacetate in the presence of a base such as sodium carbonate to provide after deprotection compound (4). In step C, the maleimide is introduced stepwise. Treatment of compound (5) with maleic anhydride in the presence of a base such as triethylamine leads to an intermediate which cyclize in the presence of an activating group such as pentafluorophenol and base such as diisopropylmethanediimine to provide, after deprotection of the tert-butyl esters, compound (5).

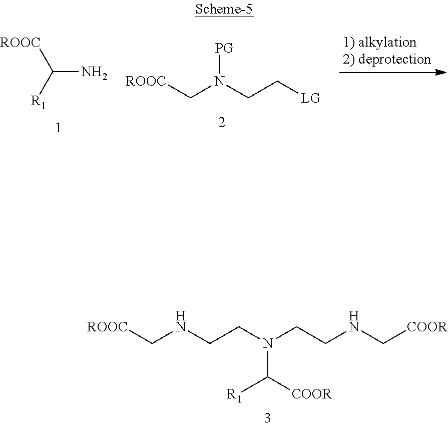

Compound (2) as shown and described above for Scheme-4 is a useful intermediate for preparing compounds from Formula (IIb, IIc). Compound 3 may be prepared by reaction of an amine 1 for example the tert-butyl glycinate hydrochloride salt with compound 2, for example the tert-butyl N-benzyl-N-(2-bromoethyl)glycinate, where LG is a leaving group, for example halo (such as bromo) in the presence of a suitable base, such as potassium carbonate. In a second step, the protecting group is removed under appropriate conditions. For example the benzyl group can be removed under reductive conditions, using palladium over charcoal and hydrogen gas.

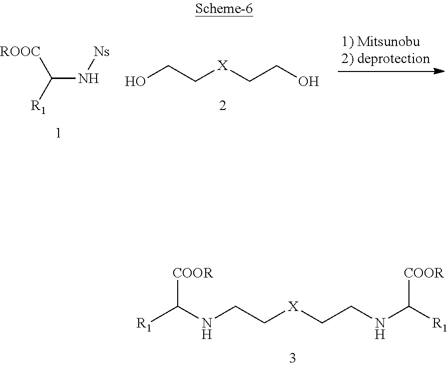

Compound (1) as shown and described above for Scheme-6 is a useful intermediate for preparing compounds from Formula (IIb, IIc). The N-alkylation of compound (1) can be performed under Mitsunobu conditions by treating nitrobenzenesulfonamides (1) such as di-tert-butyl ((2-nitrophenyl)sulfonyl)-D-aspartate and the alcohol (2) such as 2,2′-oxybis(ethan-1-ol) in the presence of triphenylphosphine and di-tert-butyl azodicarboxylate. The nosyl groups can be removed by thiolate nucleophiles, for example thiophenol or thioglycolic acid in the presence of a base to provide compound (3).

### Pharmaceutical Composition

The disclosure also relates to a pharmaceutical composition comprising a compound of formula (I) or (II) as disclosed herein and at least one pharmaceutically acceptable carrier.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for an intravenous, intramuscular or subcutaneous administration and the like.

The pharmaceutical compositions can take the form of an aqueous solution, for example an injectable formulation comprising at least one compound according to this disclosure.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above.

For parenteral administration in an aqueous solution, for example, the solution may be suitably buffered, and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In a particular, embodiment the pharmaceutical composition comprises one or more excipient(s) which is selected from stabilizers against radiolytic degradation, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

The doses used for the administration can be adapted as a function of various parameters, and in particular as a function of the mode of administration used, of the relevant pathology, or alternatively of the desired duration of treatment. It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein.

### Compound of Formula (I) or (II) for Use as a Medicament

The disclosure also relates to a compound of formula (I) or (II) as disclosed herein for use as a medicament. The compounds of formula (I) or (II) exhibit valuable pharmaceutical properties as indicated in the tests provided in the examples and are therefore indicated for therapy.

The disclosure also relates to a compound of formula (I) or (II) for use in treating cancer.

As used herein, the term “cancer” has its general meaning in the art and includes an abnormal state or condition characterized by rapidly proliferating cell growth. The term is meant to include all types of cancerous growths or oncogenic processes, metastatic tissues or malignantly transformed cells, tissues or organs, irrespective of histopathologic type or stage of invasiveness. The term cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the oesophages.

Examples of cancer include, but are not limited, to hematological malignancies such as B-cell lymphoid neoplasm, T-cell lymphoid neoplasm, non-hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm, and myeloid cell lineage neoplasm. Examples of non-hematological cancers include, but are not limited to, skin cancer, colon cancer, breast cancer, lung cancer, brain cancer, prostate cancer, head and neck cancer, pancreatic cancer, bladder cancer, colorectal cancer, bone cancer, cervical cancer, liver cancer, oral cancer, esophageal cancer, thyroid cancer, kidney cancer, stomach cancer and testicular cancer.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors and benign cancers. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Hence, the disclosure also relates to a method for treating cancer, the method comprising contacting cancer cells with a therapeutically efficient amount of the compound of formula (I) or (II) as described herein.

As used herein, the term “contacting” means any action which results in at least one compound comprising the therapeutic agent of the presently disclosed subject matter physically contacting at least one cancer cell. Contacting can include exposing the cell(s) or tumor(s) to the compound in an amount sufficient to result in contact of at least one compound with at least one cell or tumor. The method can be practiced in vitro or ex vivo by introducing, and preferably mixing, the compound and cell(s) or tumor(s) in a controlled environment, such as a culture dish or tube. The method can be practiced in vivo, in which case contacting means exposing at least one cell or tumor in a subject to at least one compound of the presently disclosed subject matter, such as administering the compound to a subject via any suitable route.

The disclosure also relates to a method for treating cancer, the method comprising administering to a subject in need thereof, preferably a human, a therapeutically efficient amount of the compound of formula (I) or (II) as described herein.

As used herein, the term “treating” includes reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the terms “therapeutically efficient amount” of a compound refer to an amount of the compound that will elicit the biological or medical response of a subject, for example, ameliorate the symptoms, alleviate conditions, slow or delay disease progression, or prevent a disease.

The disclosure also relates to the use of a compound of formula (I) or (II) for the manufacture of a medicament.

The disclosure also relates to the use of a compound of formula (I) or (II) for the manufacture of a medicament for the treatment of cancer.

### Compound of Formula (I) or (II) for Use in Imaging and Methods Thereof

The disclosure also relates to a compound of formula (I) or (II) for use in imaging, preferably in vivo imaging.

In a specific embodiment, the imaging method in which the compound of formula (I) or (II) is used is PET (positron emission tomography) or SPECT (Single photon emission computed tomography).

Hence, the disclosure also relates to a method for imaging, the method comprising contacting cancer cells with an efficient amount of the compound of formula (I) or (II).

The method can further comprise a step of detecting the signal derived from the decay of the radionuclide present in said compound.

In a specific embodiment, the present disclosure provides a method for detecting the presence or absence of tumors in a subject, comprising:
(i) administering a compound of formula (I) or (II), e.g. as an intravenous injection in said subject;
(ii) acquiring an image, typically by PET or SPECT imaging; and,
(iii) detecting the presence or absence of tumors in said subject.

The disclosure also relates to a compound of formula (I) or (II) for use in diagnostic, typically for use in diagnosing cancer disorders.

The disclosure also relates to a method for diagnosing and/or detecting cancer cells in a subject, the method comprising administering to said subject, preferably a human, an efficient amount of the compound of formula (I) or (II), and detecting the signal derived from the decay of the radionuclide present in said compound.

The present disclosure provides the following exemplary embodiments:
1. A compound, or a pharmaceutically acceptable salt thereof, comprising
   a) at least a chelator of formula (C):

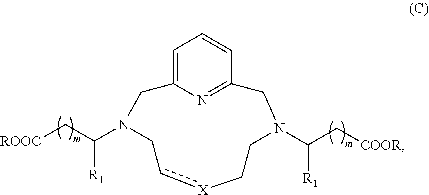

wherein
[symbol] is a single bond or a double bond,
when [symbol] is a single bond, X is —O— or

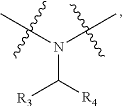

when [symbol] is a double bond, X is ═N—
each m is 0 to 5;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, heteroaryl having 5 to 10 ring atoms, or any combination thereof, and
b) target binding moiety; and,
c) optionally, a spacer covalently linking C to the target binding moiety A, said spacer preferably comprises or consists of optionally substituted alky, heteroalkyl, cycloalkyl, cycloheteroalkyl, aryl, heteroaryl, aralkyl, heteroaralkyl, alkenyl. heteroalkenyl, cycloalkenyl, cycloheteroalkenyl, alkynyl, sulfonyl, amines, ethers, thioethers, phosphines, phosphoramidates, carboxamides, esters. imidoesters, amidines. thioesters, sulfonamides, carbamates, ureas, guanidines. thioureas one or more natural or non-natural aminoacids such as glycine, alanine, proline, valine,
   wherein the chelator of formula (C) is optionally chelated to a radionuclide,
2. The compound according to embodiment 1, or a pharmaceutically acceptable salt thereof, wherein the chelator of formula (C) is capable of chelating a radionuclide at a temperature ≤60° C. more preferably ≤55° C., even more preferably ≤50° C. with a yield ≥85%, more preferably ≥90%, even more preferably ≥95%.
3. The compound according to embodiments 1 or 2, or a pharmaceutically acceptable salt thereof, wherein the chelator of formula (C) is capable of chelating a radionuclide in ≤60 min.
4. The compound according to any one of embodiments 1 to 3, or a pharmaceutically acceptable salt thereof, which is a compound of formula (Ca), (Cb), or (Cc)

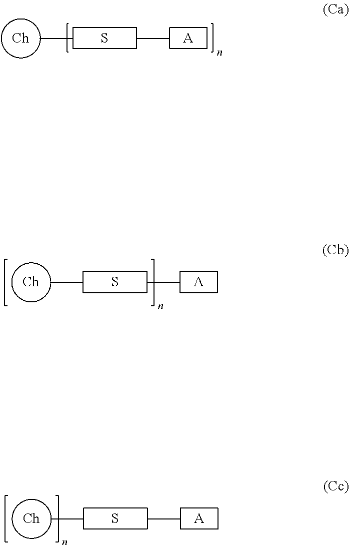

wherein Ch is the chelator of formula (C);
S is, independently at each occurrence, a bond or the spacer;
A is, independently at each occurrence, the target binding moiety; and
n is 1, 2, 3, 4, 5, 6 or 7.
5. The compound according to any one of embodiments 1 to 4, which is a compound of formula (I)

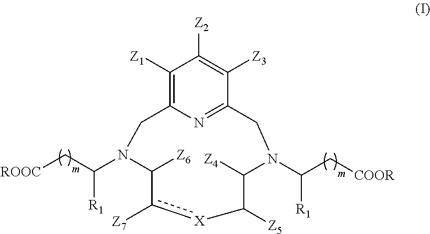

or a pharmaceutically acceptable salt thereof,
wherein
[symbol] is a single bond or a double bond,
when [symbol] is a single bond, X is —O— or

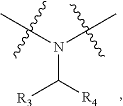

when [symbol] is a double bond, X is ═N— and Z₅ and Z₇ form, together with the N atom, a heteroaryl having 5 or 6 ring atoms, otherwise, Z₅ and Z₇ are each independently selected from the group consisting of H, an albumin-binder and —X₁-L₁-X₂-L₂-A;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

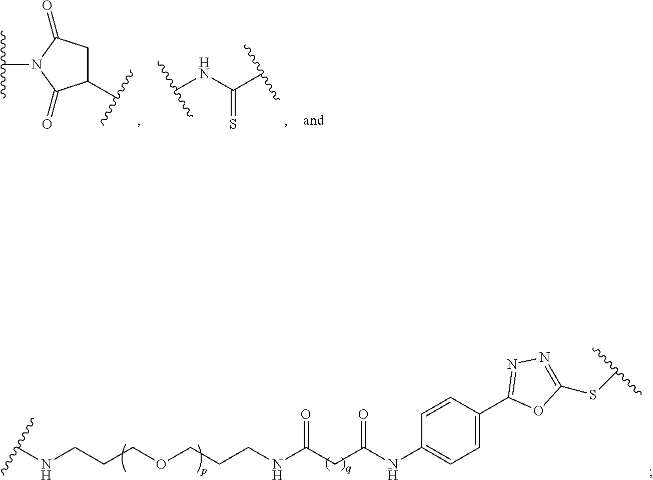

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
   n is 1, 2, 3, 4, 5, or 6;
   p is 1, 2, 3, 4, 5, or 6;
   q is 1, 2, 3, 4, 5, or 5; and
   A is a target binding moiety.
6. The compound of formula (I) according to embodiment 5, or a pharmaceutically acceptable salt thereof, wherein R is H.
7. The compound of formula (I) according to embodiments 5 or 6, or a pharmaceutically acceptable salt thereof, wherein each R₁ is independently selected from the group consisting of H, C₁-C₆ alkyl, C(═O)OR₂, (C₁-C₆ alkyl)-C(═O)OR₂, and heteroaryl having 5 to 10 ring atoms.
8. The compound of formula (I) according to any one of embodiments 5 to 7, or a pharmaceutically acceptable salt thereof, wherein each R₁ is independently selected from the group consisting of H, CH₃, C(═O)OH, CH₂C(═O)OH and pyridyl.
9. The compound of formula (I) according to any one of embodiments 5 to 8, or a pharmaceutically acceptable salt thereof, wherein R₃ is selected from the group consisting of H, C₁-C₃ alkyl, and C(═O)OR.
10. The compound of formula (I) according to any one of embodiments 5 to 9, or a pharmaceutically acceptable salt thereof, wherein R₃ is selected from the group consisting of H, CH₃ and C(═O)OH.
11. The compound of formula (I) according to any one of embodiments 5 to 10, or a pharmaceutically acceptable salt thereof, wherein R₄ is selected from the group consisting of H, C(═O)OR, (C₁-C₆ alkyl)-C(═O)OR and heteroaryl having 5 to 10 ring atoms.
12. The compound of formula (I) according to any one of embodiments 5 to 11, or a pharmaceutically acceptable salt thereof, wherein R₄ is selected from the group consisting of H, C(═O)OH, CH₂C(═O)OH and pyridyl.
13. The compound of formula (I) according to any one of embodiments 5 to 12, or a pharmaceutically acceptable salt thereof, wherein only one of Z₁, Z₂, Z₃, Z₄, Z₅, Z₆, and Z₇ is —X₁-L₁-X₂-L₂-A, and the other Z groups are H.
14. The compound of formula (I) according to any one of embodiments 5 to 13, or a pharmaceutically acceptable salt thereof, wherein X₁ is selected from the group consisting of —O—, —N(CH₃)— and —C(═O)NH— or absent.
15. A compound of formula (Ia):

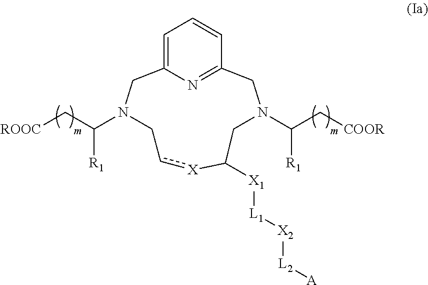

or a pharmaceutically acceptable salt thereof,
wherein
[symbol] is a single bond or a double bond,
when [symbol] is a single bond, X is —O— or

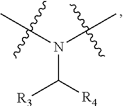

when [symbol] is a double bond, X is ═N— and the N atom and the 2 carbon atoms to
which is it bonded, form a heteroaryl having 5 or 6 ring atoms, each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms, or any combination thereof;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

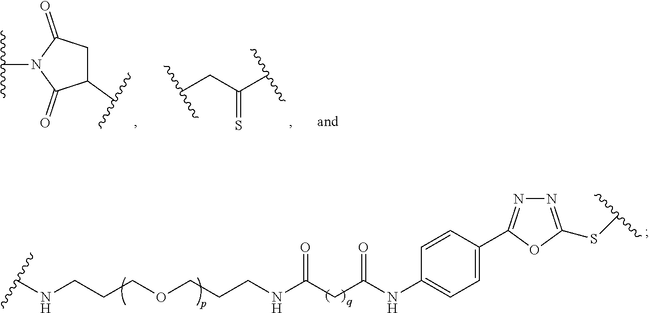

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
   n is 1, 2, 3, 4, 5, or 6;
   p is 1, 2, 3, 4, 5, or 6;
   q is 1, 2, 3, 4, 5, or 5; and
   A is a target binding moiety.
16. A compound of formula (Ib):

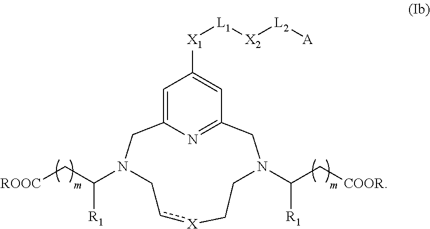

or a pharmaceutically acceptable salt thereof,
wherein
[symbol] is a single bond or a double bond,
when [symbol] is a single bond, X is —O— or

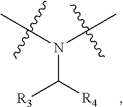

when [symbol] is a double bond, X is ═N— and the N atom and the 2 carbon atoms to which is it bonded, form a heteroaryl having 5 or 6 ring atoms,
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

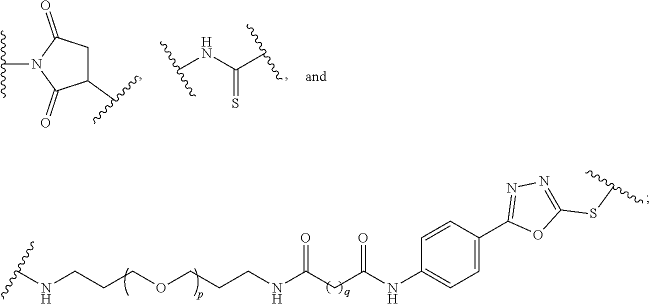

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
17. A compound of formula (Ic):

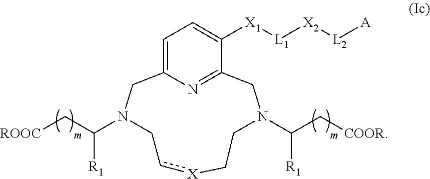

or a pharmaceutically acceptable salt thereof,
wherein
[symbol] is a single bond or a double bond,
when [symbol] is a single bond, X is —O— or

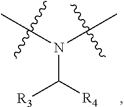

when [symbol] is a double bond, X is ═N— and the N atom and the 2 carbon atoms to which is it bonded, form a heteroaryl having 5 or 6 ring atoms,
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

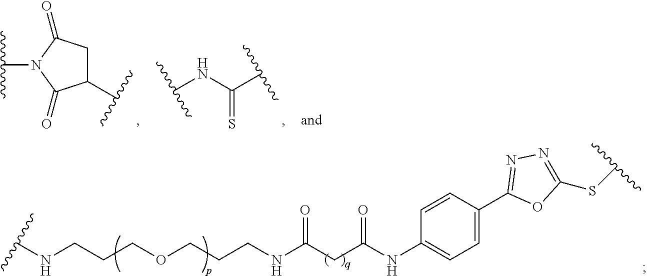

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
18. A compound of formula (Id):

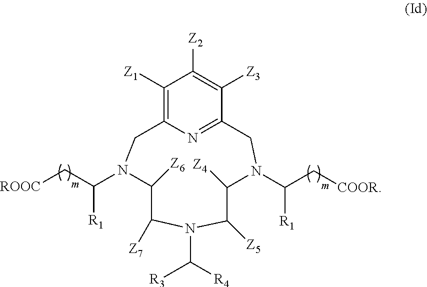

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

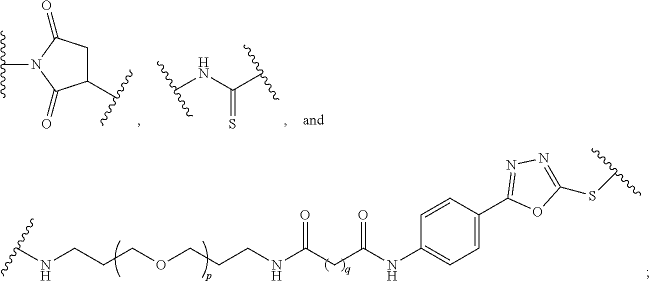

L₂ is a on or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
19. A compound of formula (Ie):

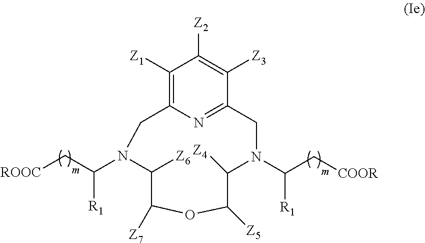

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

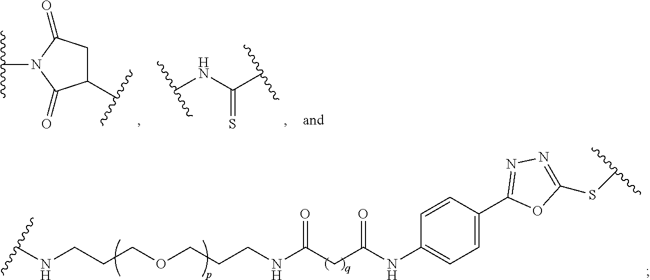

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
20. A compound of formula (If):

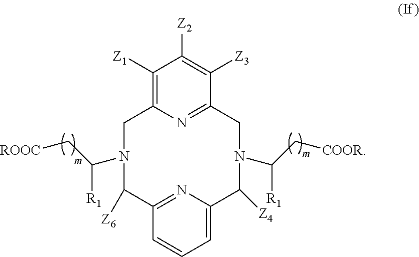

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

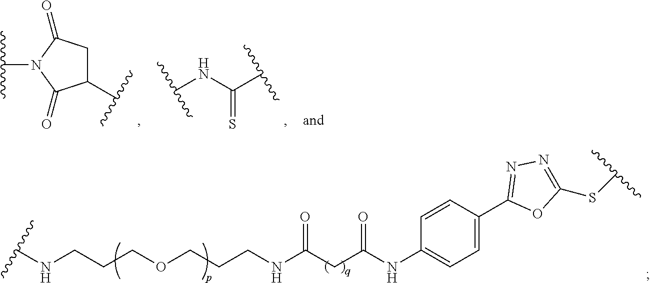

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
21. A compound of formula (Ig):

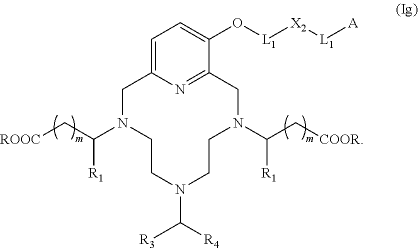

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

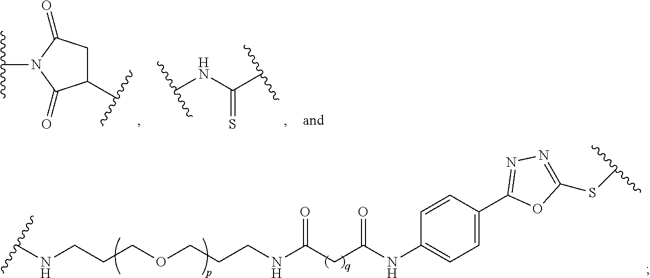

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
22. A compound of formula (Ih):

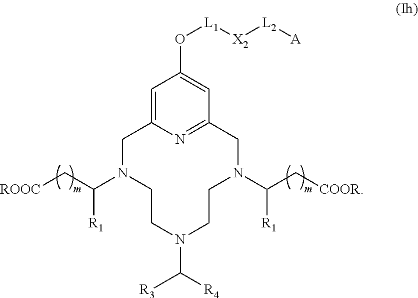

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

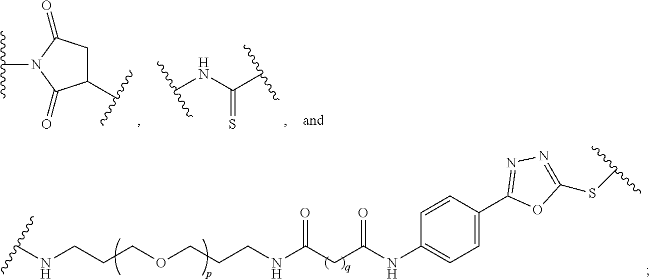

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
23. A compound of formula (Ii):

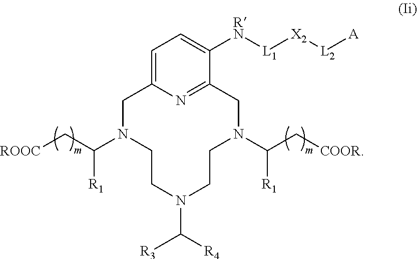

or a pharmaceutically acceptable salt thereof,
wherein
each R is independently selected from the group consisting of H and C₁-C₆ alkyl; each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

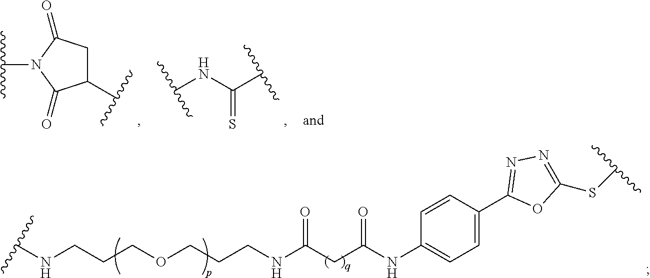

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
24. A compound of formula (Ij):

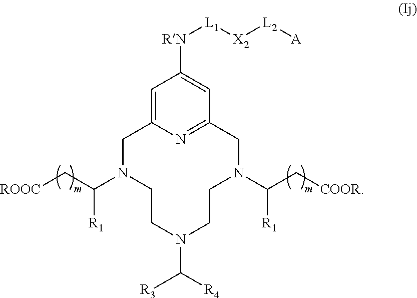

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

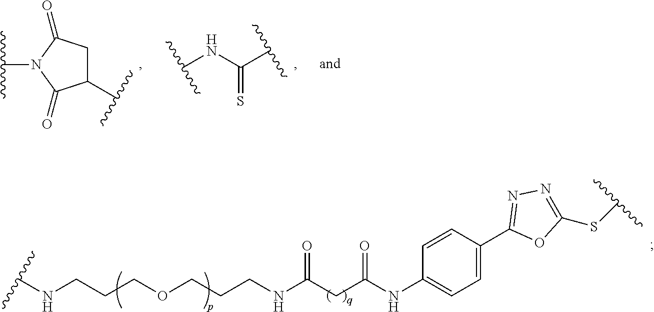

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
25. A compound of formula (Ik):

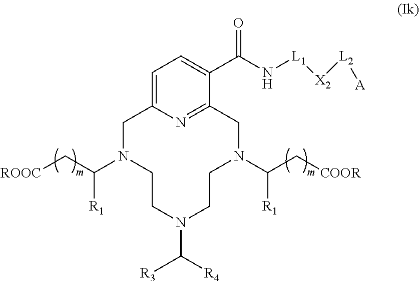

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

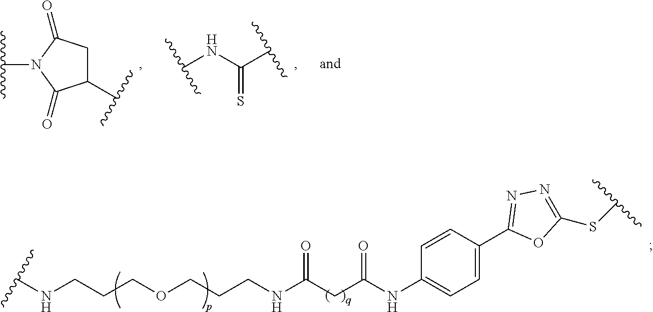

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
26. A compound of formula (Im):

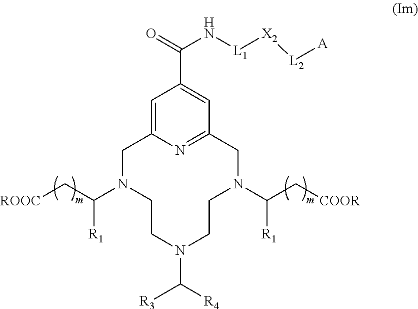

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R_{z} is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

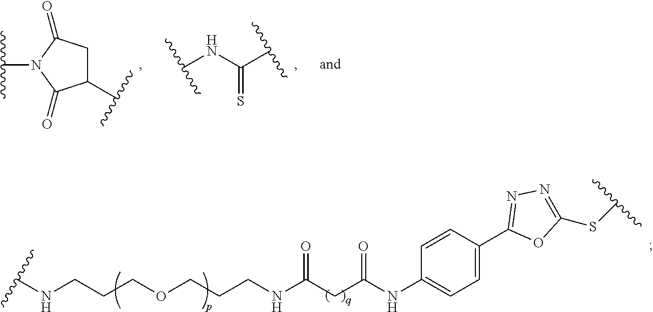

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₈ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6:
p is 1, 2, 3, 4, 5, or 6;
q is 1, 2, 3, 4, 5, or 5; and
A is a target binding moiety.
27. A compound of formula (In):

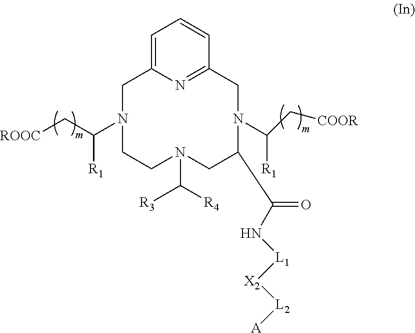

or a pharmaceutically acceptable salt thereof,
Wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

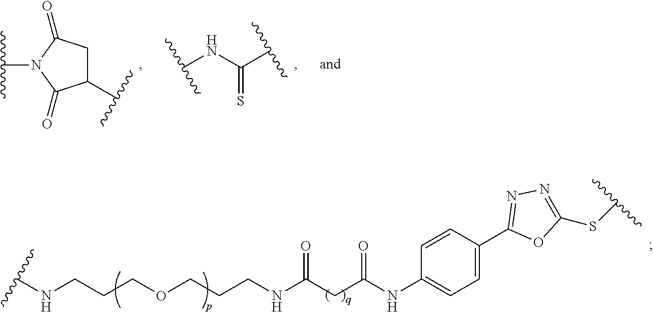

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
28. A compound of formula (Io):

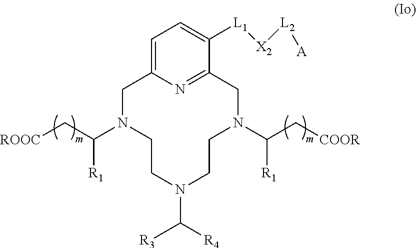

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

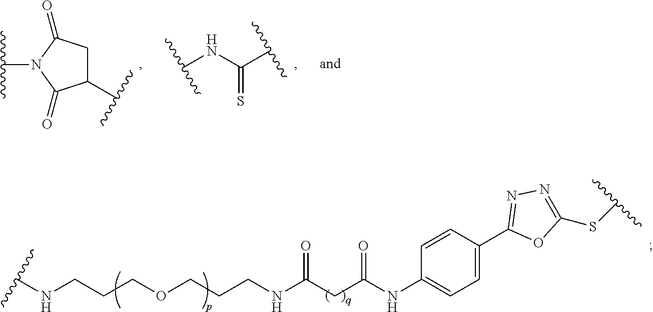

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
29. A compound of formula (Ip):

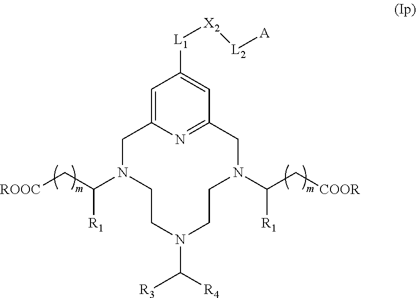

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

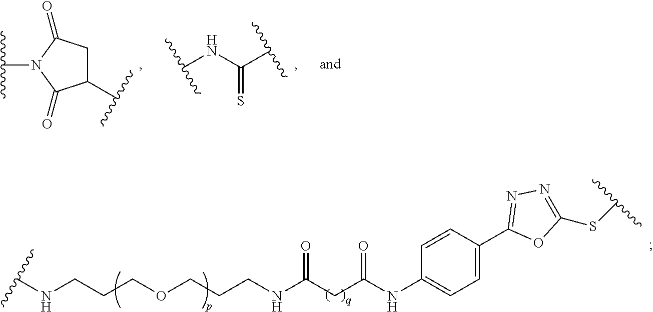

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
30. A compound of formula (Iq):

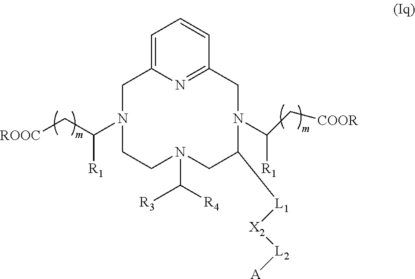

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

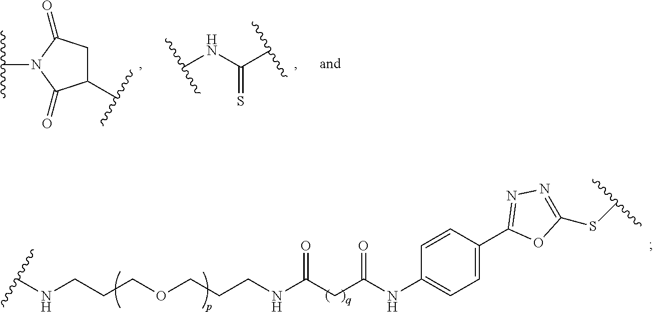

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
31. A compound of formula (Ig′):

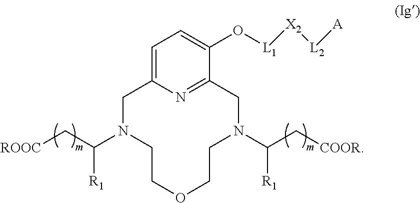

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

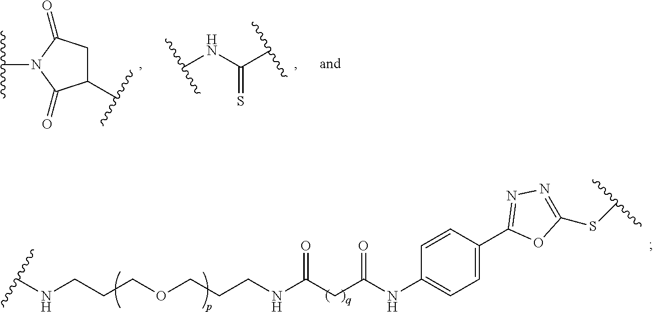

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
32. A compound of formula (Ih′):

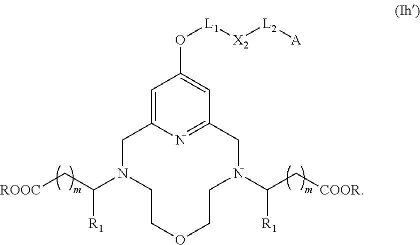

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

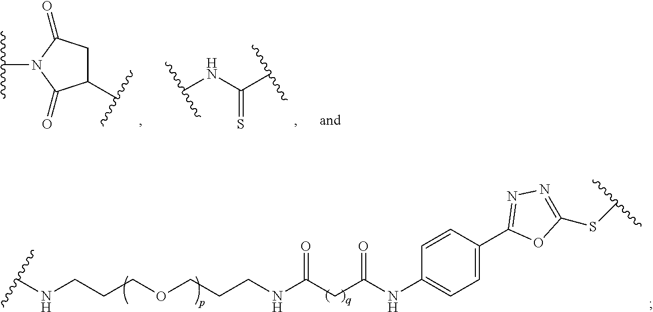

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
33. A compound of formula (Ii′):

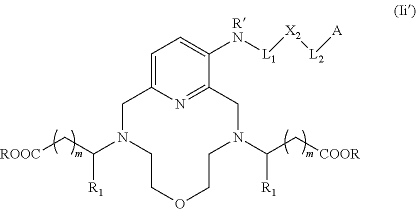

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

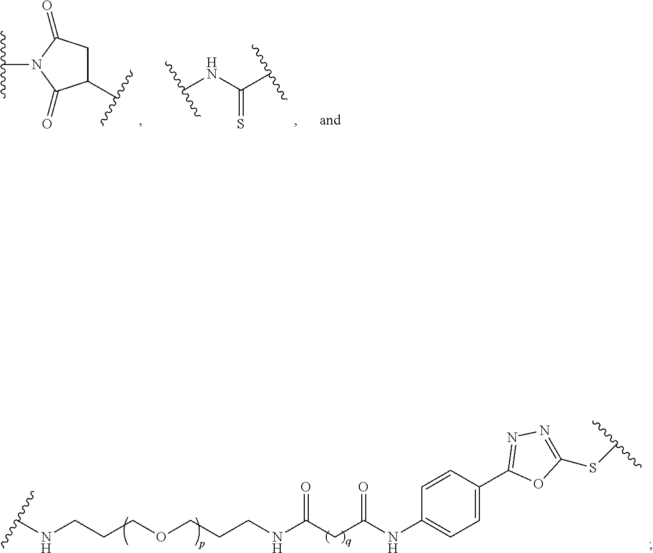

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
34. A compound of formula (Ij′):

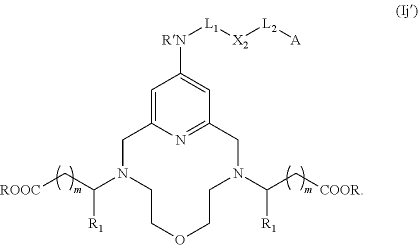

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

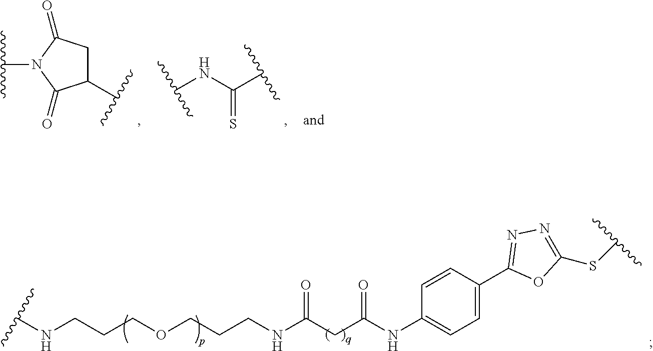

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
35. A compound of formula (Ik′):

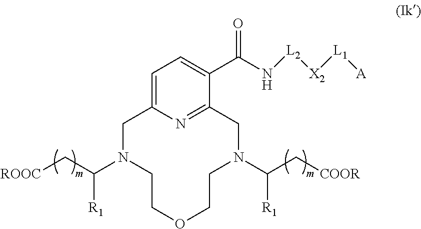

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

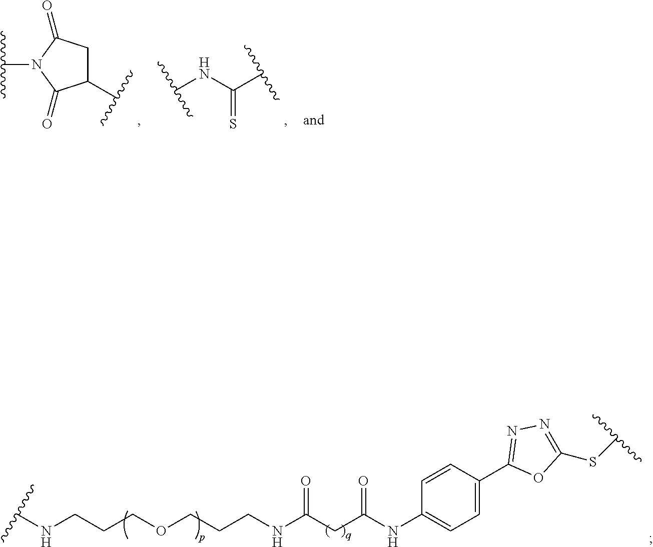

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
36. A compound of formula (Im′):

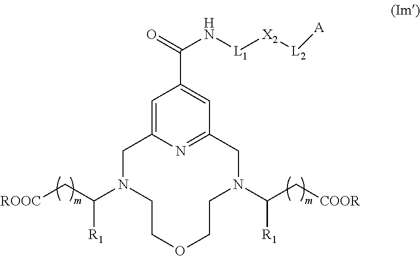

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

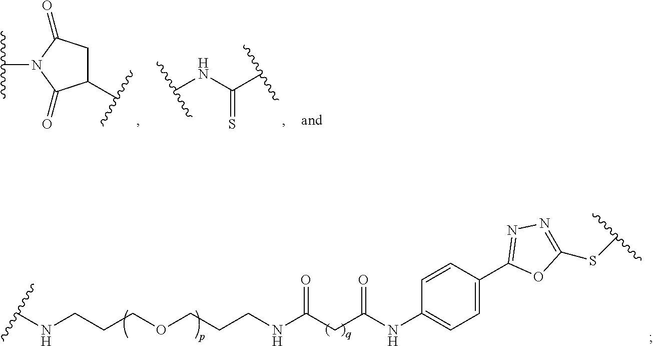

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
37. A compound of formula (In′)

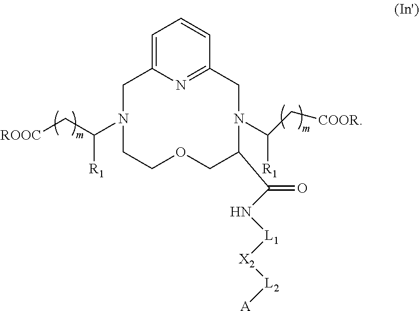

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

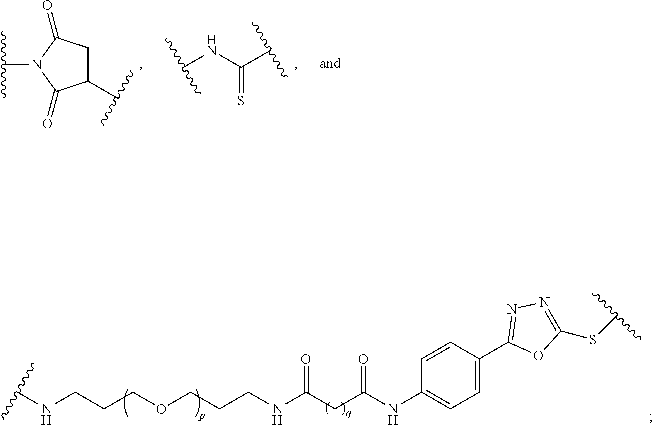

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
38. A compound of formula (Io′):

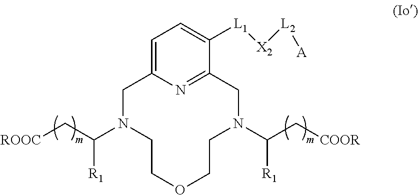

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

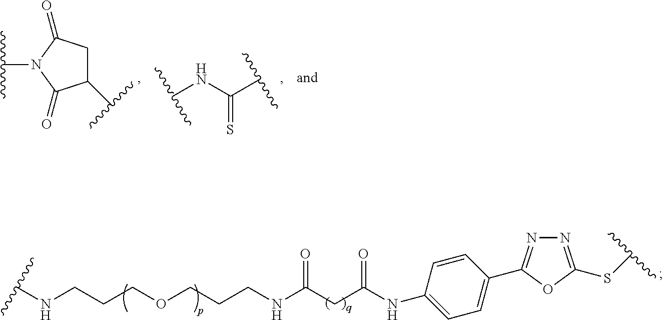

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
39. A compound of formula (Ip′):

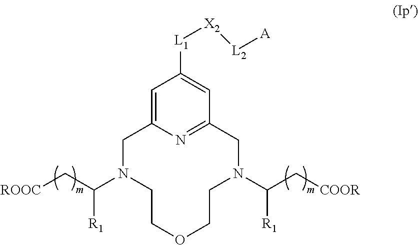

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

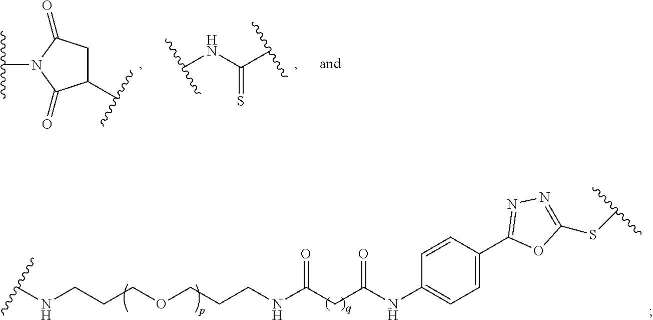

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
40. A compound of formula (Iq′)

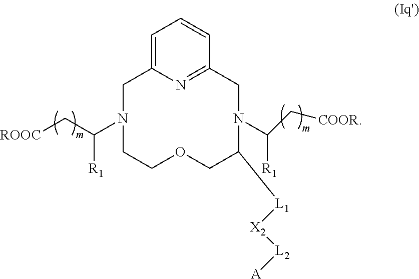

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

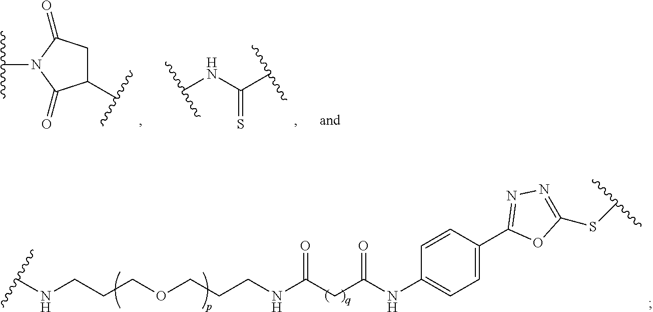

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
41. A compound of formula (Ig″):

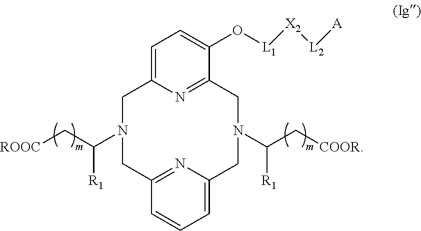

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

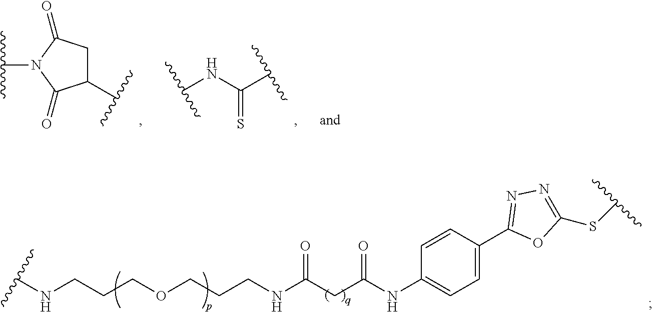

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
42. A compound of formula (Ih″):

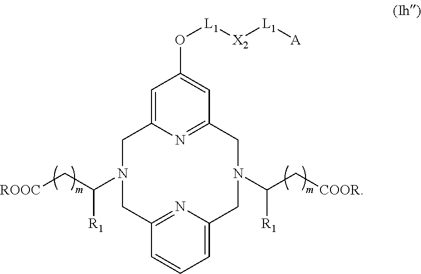

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

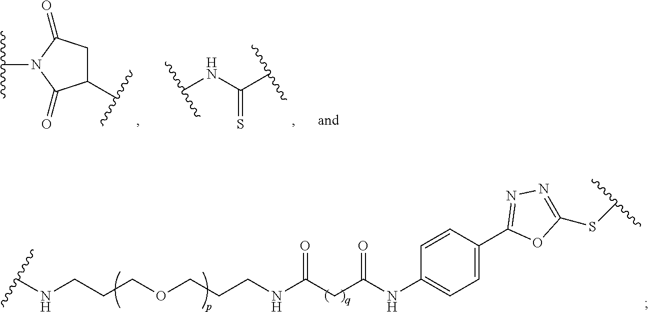

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
43. A compound of formula (Ii″):

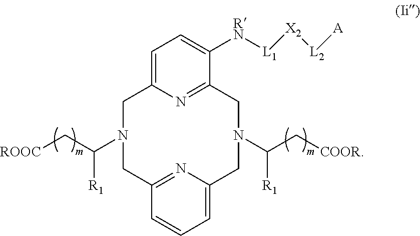

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

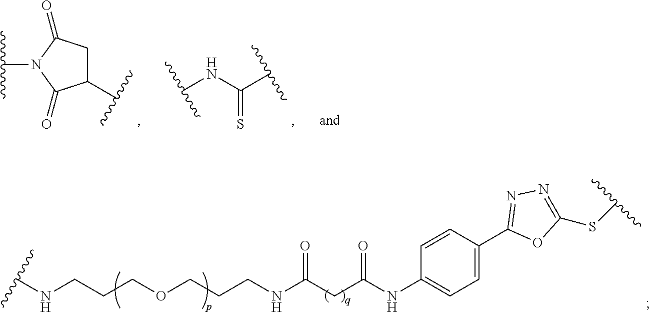

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
44. A compound of formula (Ij″):

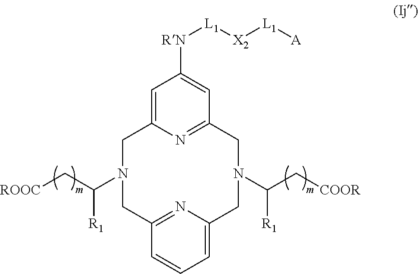

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

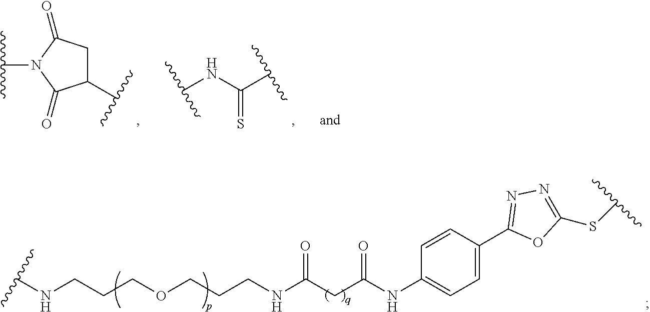

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
45. A compound of formula (Ik″):

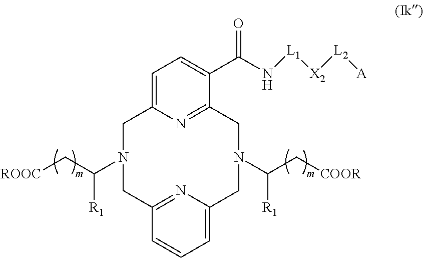

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

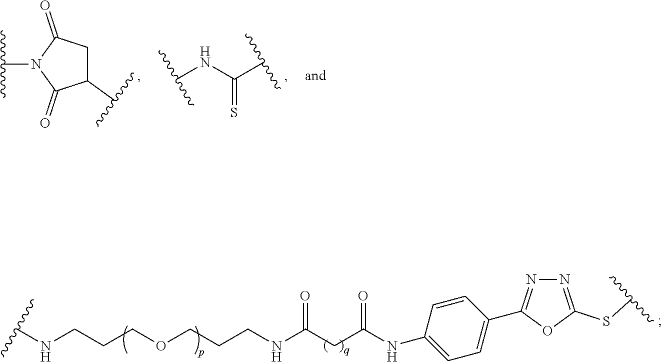

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
46. A compound of formula (Im″):

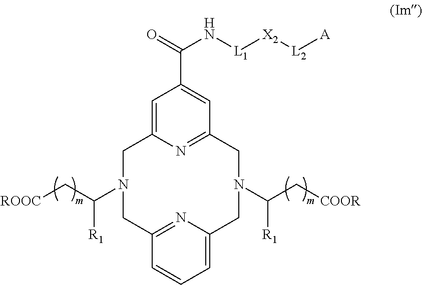

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

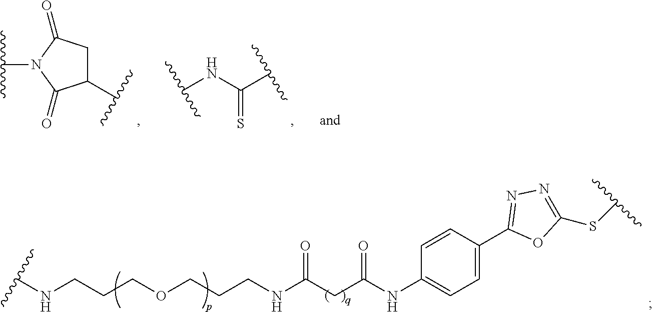

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
47. A compound of formula (In″):

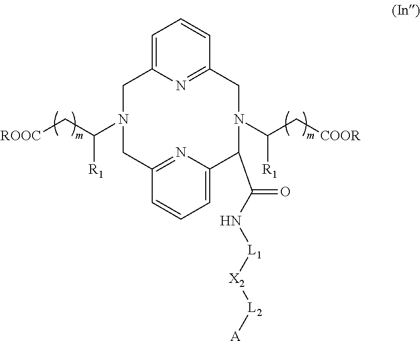

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂; R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

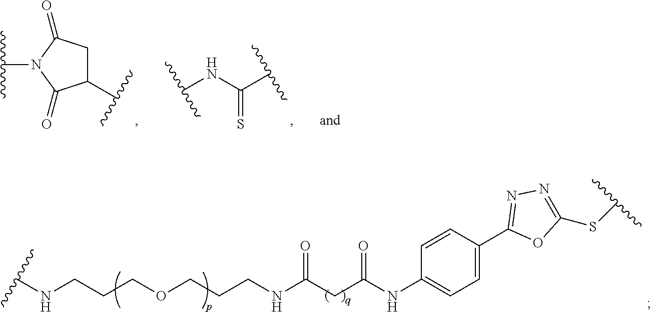

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
48. A compound of formula (Io″):

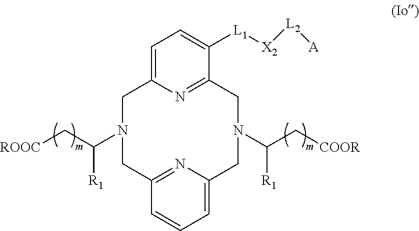

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

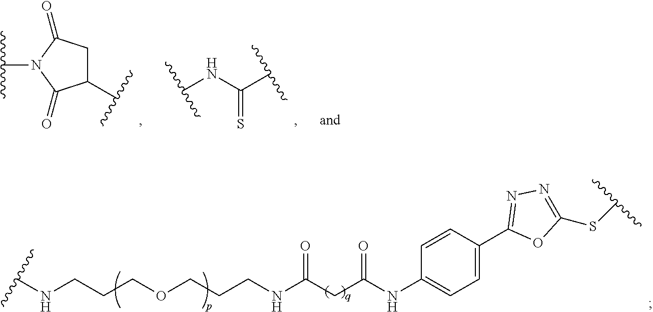

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
49. A compound of formula (Ip″):

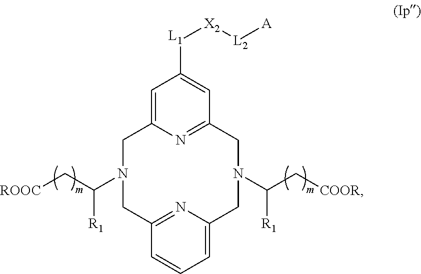

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁ aryl, and heteroaryl having 5 to 10 ring atoms;
X₂ is selected from the group consisting of:

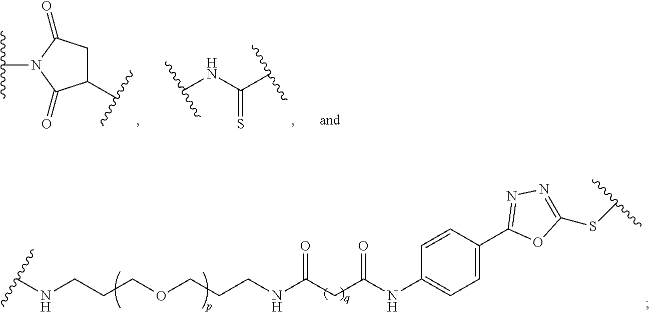

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
50. A compound of formula (Iq″):

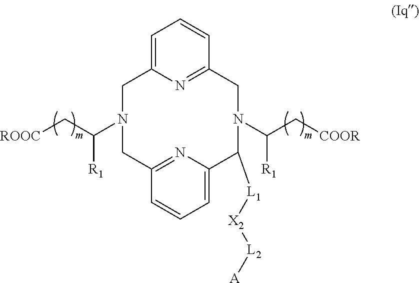

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of:

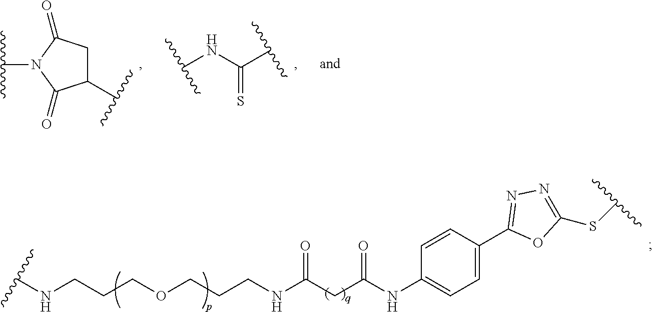

L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.
51. The compound of formula (I) according to any one of embodiments 6 to 56, or a pharmaceutically acceptable salt thereof, wherein R is H.
52. The compound of formula (I) according to any one of embodiments 6 to 57, or a pharmaceutically acceptable salt thereof, wherein each R₁ is independently selected from the group consisting of H, C₁-C₆ alkyl, C(═O)OR₂, (C₁-C₆ alkyl)-C(═O)OR₂, and heteroaryl having 5 to 10 ring atoms.
53. The compound of formula (I) according to any one of embodiments 6 to 58, or a pharmaceutically acceptable salt thereof, wherein each R₁ is independently selected from the group consisting of H, CH₃, C(═O)OH, CH₂C(═O)OH and pyridyl.
54. The compound of formula (I) according to any one of embodiments 6 or 59, or a pharmaceutically acceptable salt thereof, wherein L₁ is C₁-C₅ alkylene.
55. The compound of formula (I) according to any one of embodiments 5 to 60, or a pharmaceutically acceptable salt thereof, wherein L₂ is bond.
56. The compound of formula (I) according to any one of embodiments 5 to 61, or a pharmaceutically acceptable salt thereof, wherein X₂ is

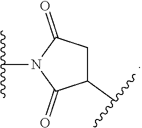 [SMILES: *C1CC(=O)N(*)C1=O]

57. The compound of formula (I) according to any one of embodiments 5 to 62, or a pharmaceutically acceptable salt thereof, wherein A is a target binding moiety comprising a peptide, polypeptide, protein, peptidomimetic, aptamer, DARPin, antisense oligonucleotide, siNA, small molecule, microparticle or nanoparticle.
58. The compound of formula (I) according to any one of embodiments 5 to 63, or a pharmaceutically acceptable salt thereof, wherein A is a target binding moiety comprising a peptide, polypeptide or protein.
59. The compound of formula (I) according to any one of embodiments 5 to 64, or a pharmaceutically acceptable salt thereof, wherein A is a target binding moiety comprising an antibody, a VhH antibody, a nanobody, a single domain antibody, a protein comprising an antigen-binding region of an antibody, or a fusion protein.
60. The compound of formula (I) according to any one of embodiments 5 to 65, wherein A is a target binding moiety comprising nimotuzumab, trastuzumab, sacituzumab, ramucirumab, cetuximab, enolituzumab, tusamitamab, amivantamab, or datopotamab.
61. The compound of formula (I) according to any of embodiments 4 to 66, wherein the compound is complexed with a radionuclide selected from ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, ⁶¹Cu, preferably selected from: ⁶⁸Ga, ⁶⁴Cu, ⁹⁰Y, ¹⁷⁷Lu, ²¹²Pb, ²²⁵Ac and ¹⁶¹Tb.
62. The compound of formula (I) according to any one of embodiments 5 to 14, which is selected from

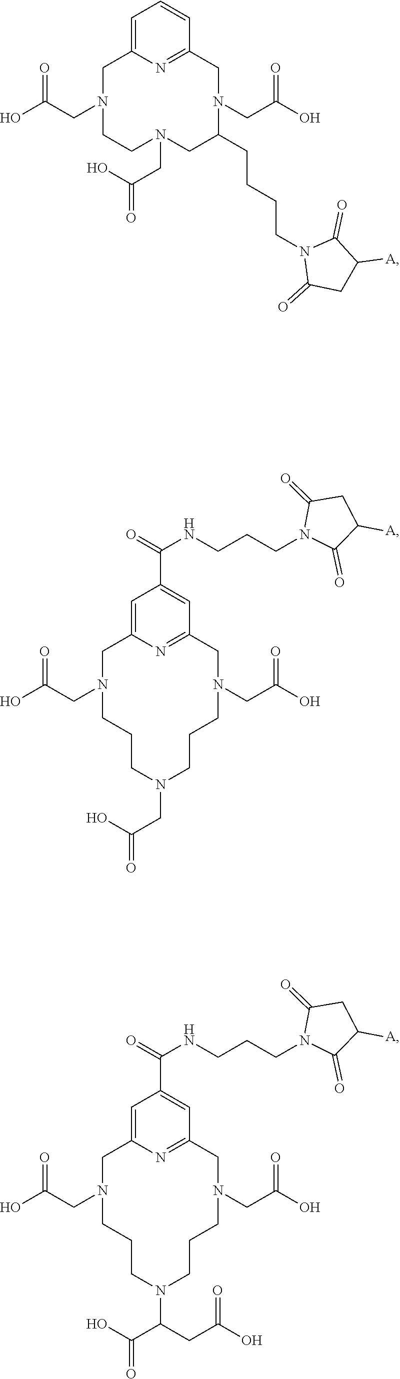

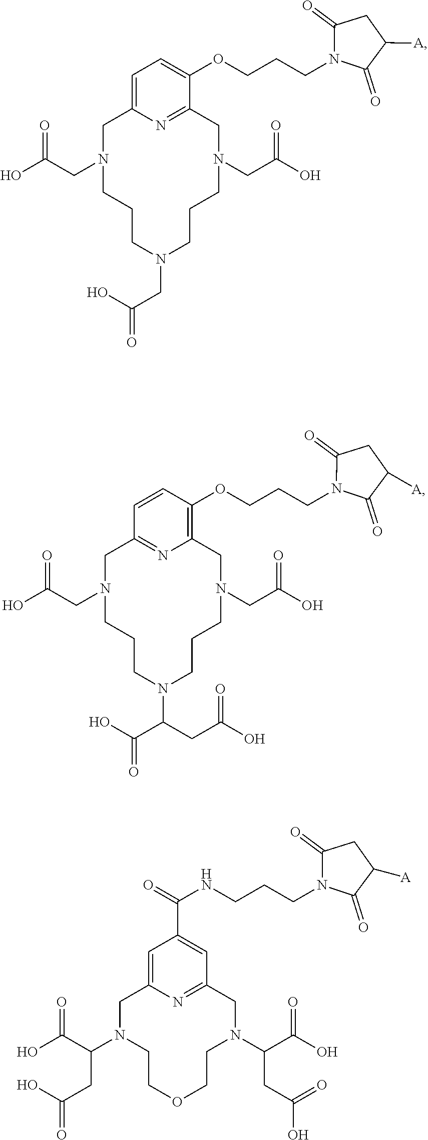

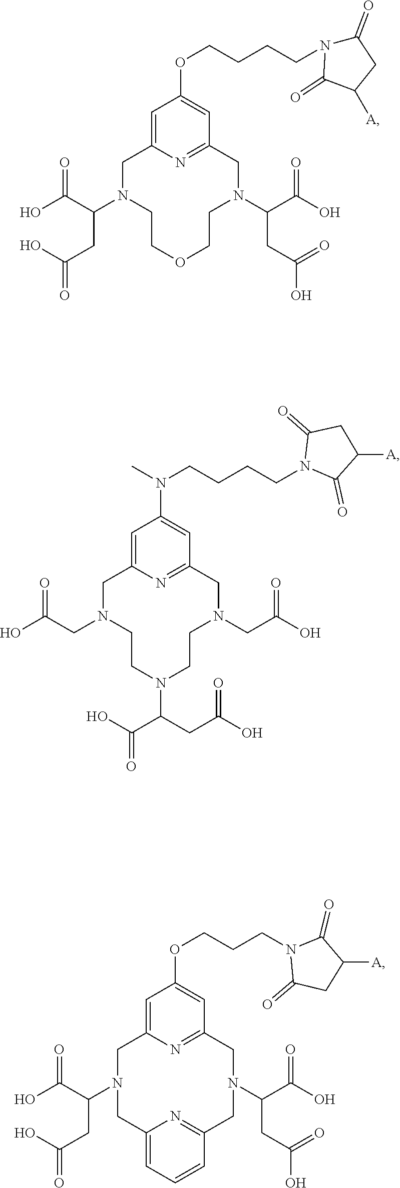

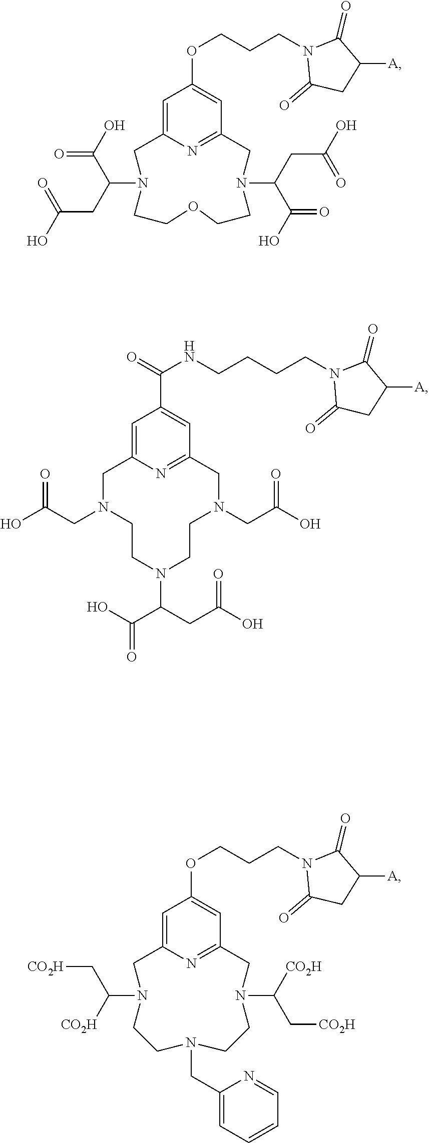

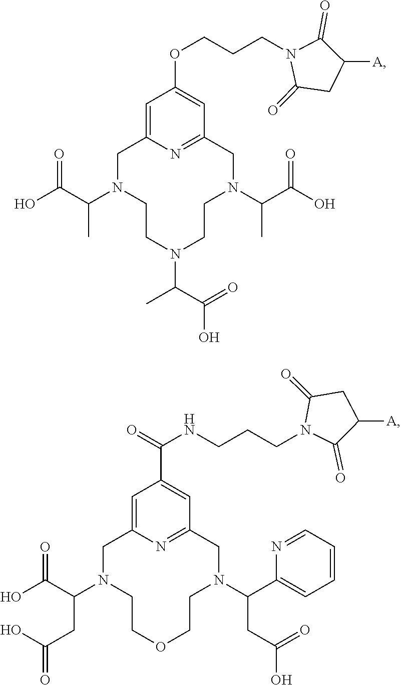

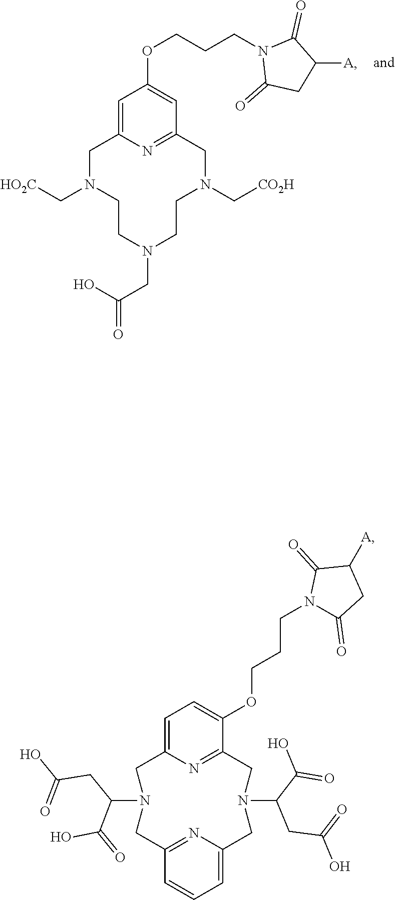

or a pharmaceutically acceptable salt thereof,
wherein A is a target-binding moiety as defined in any one of embodiments 63 to 66 and
wherein the compound is optionally complexed with a radionuclide selected from ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, ⁶¹Cu, preferably selected from: ⁶⁸Ga, ⁶⁴Cu, ⁹⁰Y, ¹⁷⁷Lu, ²¹²Pb, ²²⁵Ac and ¹⁶¹Tb.
63. The compound according to embodiment 68, which is selected from

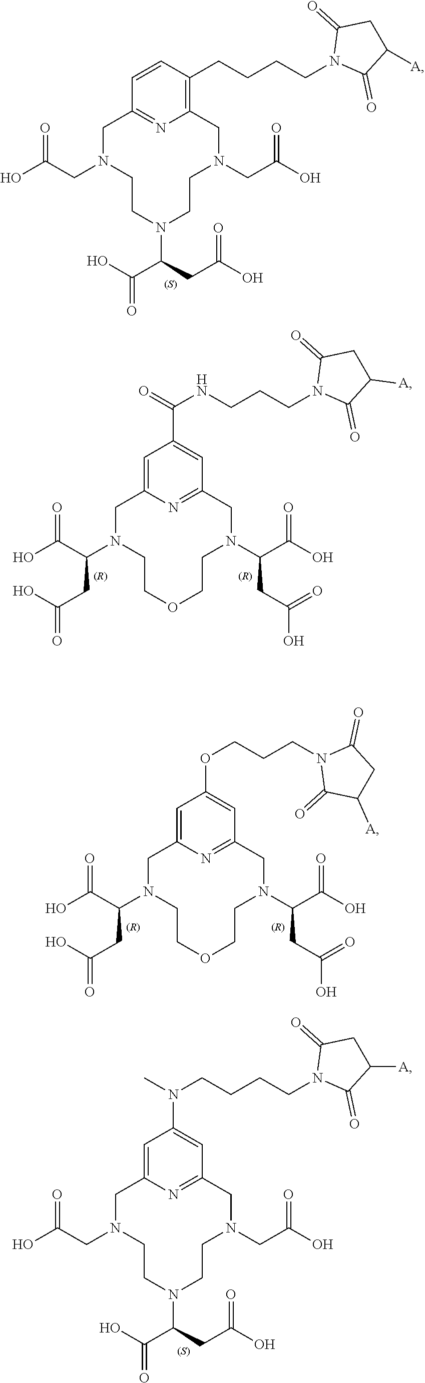

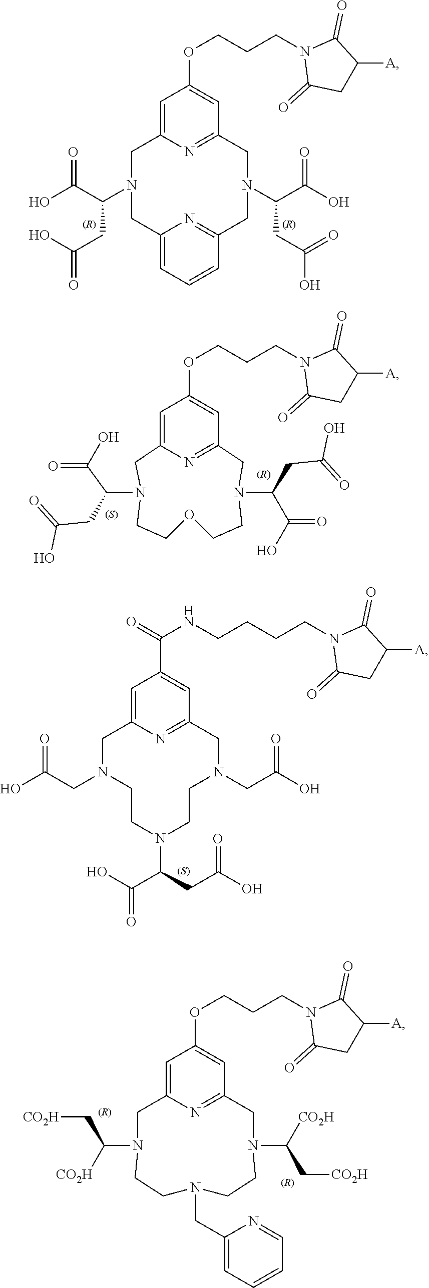

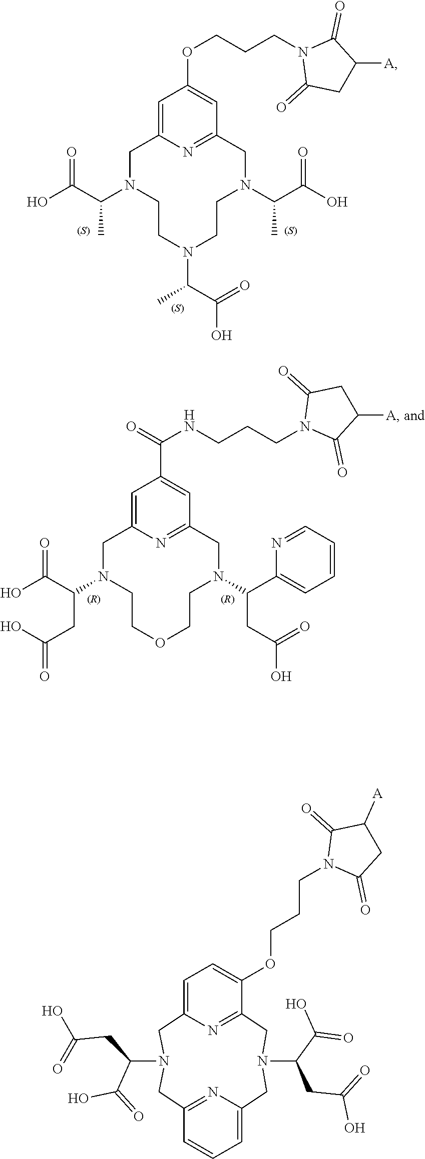

or a pharmaceutically acceptable salt thereof.
64. A compound of formula (Cd), or (Ce)

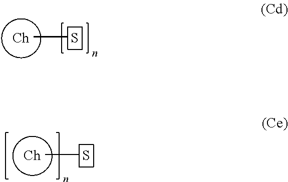

or pharmaceutically acceptable salts thereof,
wherein Ch is a chelator compound of formula (C) as defined in any one of embodiments 1 to 3, optionally chelated to a radionuclide;
S is, independently at each occurrence, a bond, an H or a spacer; for example, a spacer as defined in embodiment 1;
n is 1, 2, 3, 4, 5, 6 or 7.
65. The compound of embodiment 70, which is a compound of formula (II)

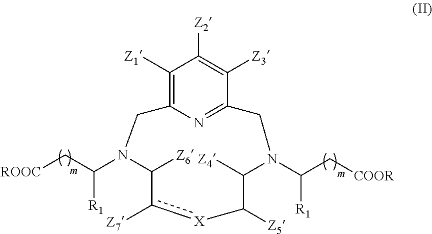

or a pharmaceutically acceptable salt thereof,
wherein
[symbol] is a single bond or a double bond, wherein
when [symbol] is a single bond, X is —O— or

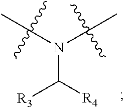

when [symbol] is a double bond, X is ═N— and Z₅′ and Z₇′ form, together with the N atom, a heteroaryl having 5 or 6 ring atoms, otherwise, Z₅′ and Z₇′ are each independently selected from the group consisting of H, an albumin-binder and —X₁-L₁-X₂′;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
Z₁′, Z₂′, Z₃′, Z₄′, and Z₆′ are each independently from the group consisting of H, an albumin-binder H or a group —X₁-L₁-X₂′, provided that at least one of Z₁′, Z₂′, Z₃′, Z₄′, Z₅′, Z₆′, of Z₇′ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

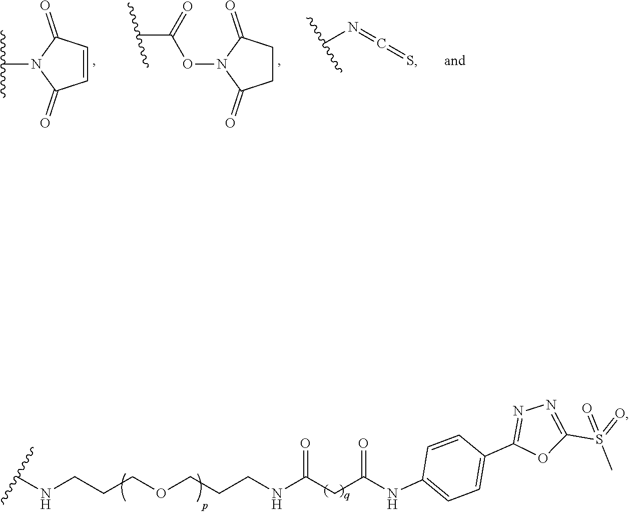

each m is independently 0 or 1;
n is 1, 2, 3, 4, 5, or 6;
p is 1, 2, 3, 4, 5, or 6;
q is 1, 2, 3, 4, 5, or 5.
66. The compound of formula (II) according to embodiment 71, or a pharmaceutically acceptable salt thereof, wherein R is H.
67. The compound of formula (II) according to embodiments 71 or 72, or a pharmaceutically acceptable salt thereof, wherein each R1 is independently selected from the group consisting of H, C₁-C₆ alkyl, C(═O)OR₂, (C₁-C₆ alkyl)-C(═O)OR₂, and heteroaryl having 5 to 10 ring atoms.
68. The compound of formula (II) according to any one of embodiments 71 to 73, or a pharmaceutically acceptable salt thereof, wherein each R₁ is independently selected from the group consisting of H, CH₃, C(═O)OH, CH₂C(═O)OH and pyridyl.
69. The compound of formula (II) according to any one of embodiments 71 to 74, or a pharmaceutically acceptable salt thereof, wherein R₃ is selected from the group consisting of H, C₁-C₃ alkyl, and C(═O)OR.
70. The compound of formula (II) according to any one of embodiments 71 to 75, or a pharmaceutically acceptable salt thereof, wherein R₃ is selected from the group consisting of H, CH₃ and C(═O)OH.
71. The compound of formula (II) according to any one of embodiments 71 to 76, or a pharmaceutically acceptable salt thereof, wherein R₄ is selected from the group consisting of H, C(═O)OR, (C₁-C₆ alkyl)-C(═O)OR and heteroaryl having 5 to 10 ring atoms.
72. The compound of formula (II) according to any one of embodiments 71 to 77, or a pharmaceutically acceptable salt thereof, wherein R₄ is selected from the group consisting of H, C(═O)OH, CH₂C(═O)OH and pyridyl.
73. The compound of formula (I) according to any one of embodiments 71 to 78, or a pharmaceutically acceptable salt thereof, wherein only one of Z′₁, Z′₂, Z′₃, Z′₄, Z′₅, Z′₆, and Z′₇ is —X₁-L₁-X′₂, and the other Z′ groups are H.
74. The compound of formula (I) according to any one of embodiments 71 to 79, or a pharmaceutically acceptable salt thereof, wherein X₁ is selected from the group consisting of —O—, —N(CH3)- and —C(═O)NH— or absent.
75. A compound of formula (IIa):

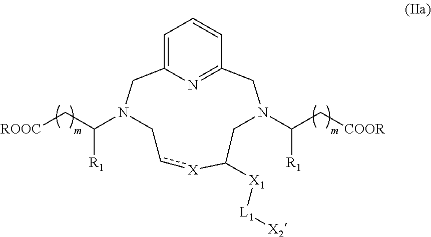

or a pharmaceutically acceptable salt thereof,
wherein
[symbol] is a single bond or a double bond, wherein
when [symbol] is a single bond, X is —O— or

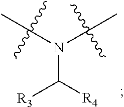

when [symbol] is a double bond, X is ═N—, the N atom together with the 2 carbon atoms to which it is linked, form a heteroaryl having 5 or 6 ring atoms;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

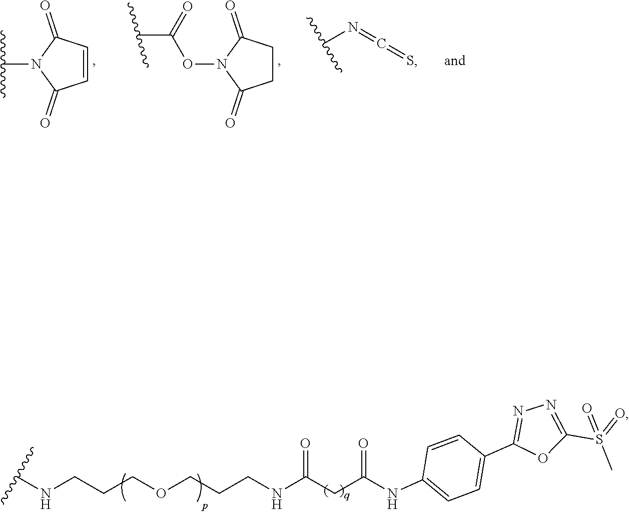

each m is independently 0 or 1;
n is 1, 2, 3, 4, 5, or 6;
p is 1, 2, 3, 4, 5, or 6;
q is 1, 2, 3, 4, 5, or 5.
76. A compound of formula (IIb):

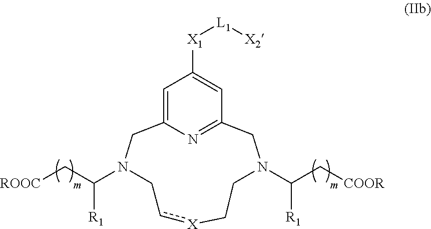

or a pharmaceutically acceptable salt thereof,
wherein
[symbol] is a single bond or a double bond, wherein
when [symbol] is a single bond, X is —O— or

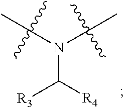

when [symbol] is a double bond, X is ═N—, the N atom together with the 2 carbon atoms to which it is linked, form a heteroaryl having 5 or 6 ring atoms;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁ aryl and heteroaryl having 5 to 10 ring atoms;
X₂′ is a group selected from the group consisting of:

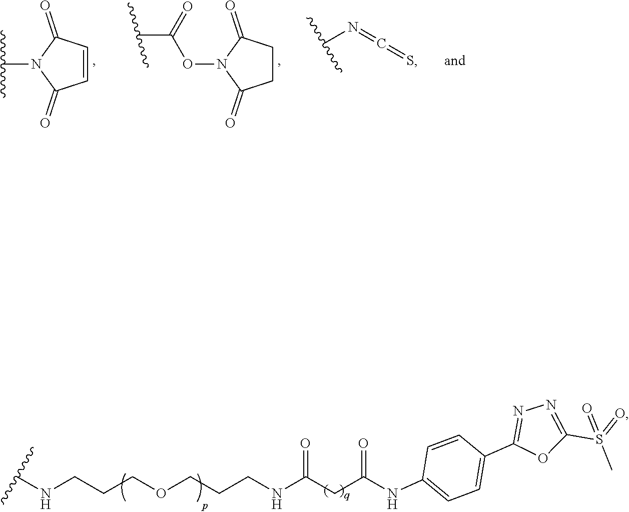

each m is independently 0 or 1;
n is 1, 2, 3, 4, 5, or 6;
p is 1, 2, 3, 4, 5, or 6;
q is 1, 2, 3, 4, 5, or 5.
77. A compound of formula (IIc):

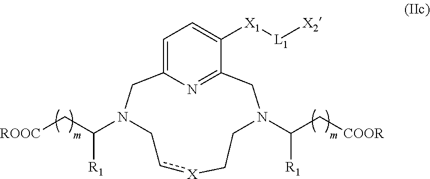

or a pharmaceutically acceptable salt thereof,
wherein
[symbol] is a single bond or a double bond, wherein
when [symbol] is a single bond, X is —O— or

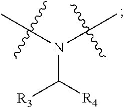

when [symbol] is a double bond, X is ═N—, the N atom together with the 2 carbon atoms to which it is linked, form a heteroaryl having 5 or 6 ring atoms;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

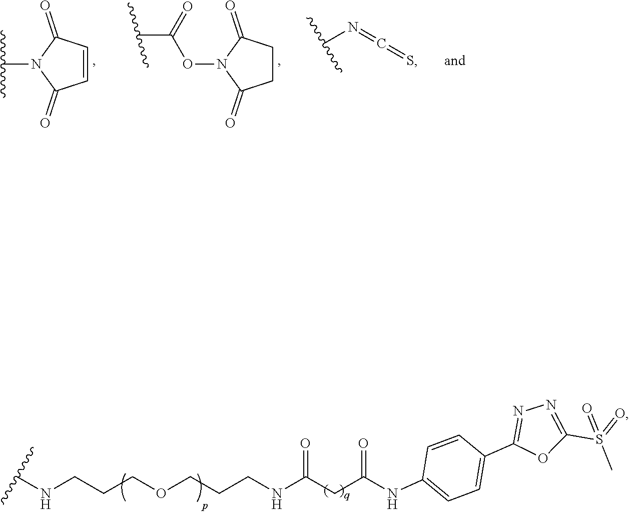

each m is independently 0 or 1;
n is 1, 2, 3, 4, 5, or 6;
p is 1, 2, 3, 4, 5, or 6;
q is 1, 2, 3, 4, 5, or 5.
78. A compound of formula (IId):

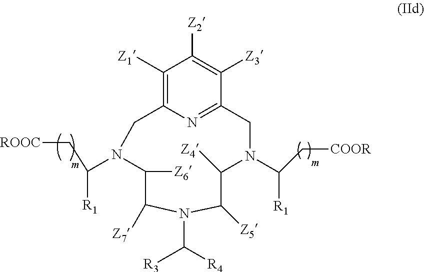

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
Z₁′, Z₂′, Z₃′, Z₄, Z₅′, Z₆′, and Z₇′ are each independently from the group consisting of H, an albumin-binder H or a group —X₁-L₁-X₂′, provided that at least one of Z₁′, Z₂′, Z₃′, Z₄, Z₅′, Z₆′, or Z₇′ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

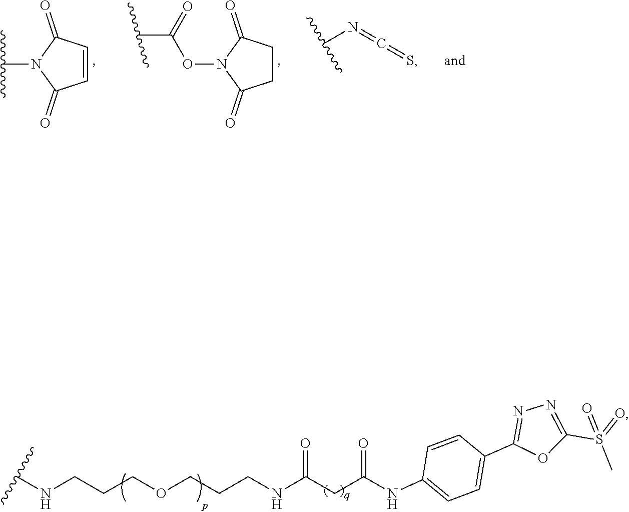

each m is independently 0 or 1;
n is 1, 2, 3, 4, 5, or 6;
p is 1, 2, 3, 4, 5, or 6;
q is 1, 2, 3, 4, 5, or 5.
79. A compound of formula (IIe):

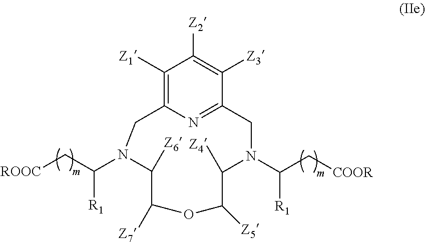

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
Z₁′, Z₂′, Z₃′, Z₄′, Z₅′, Z₆′, and Z₇′ are each independently from the group consisting of H, an albumin-binder H or a group —X₁-L₁-X₂′, provided that at least one of Z₁′, Z₂′, Z₃′, Z₄′, Z₅′, Z₆′, or Z₇′ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

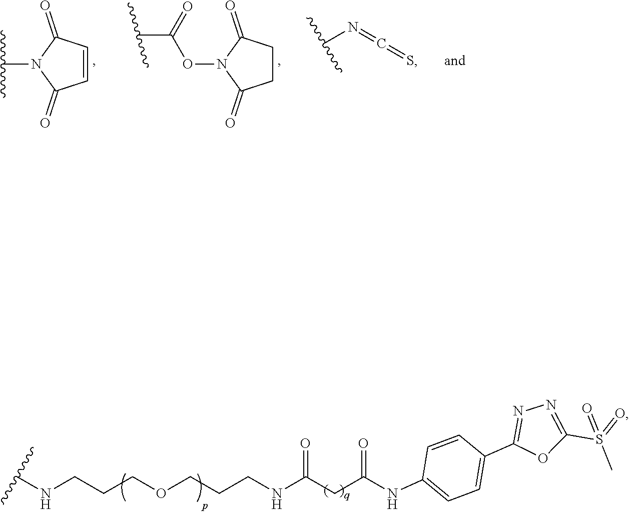

each m is independently 0 or 1;
n is 1, 2, 3, 4, 5, or 6;
p is 1, 2, 3, 4, 5, or 6;
q is 1, 2, 3, 4, 5, or 5.
80. A compound of formula (IId):

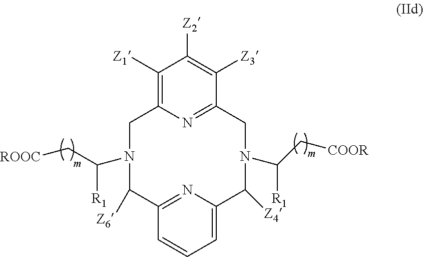

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl; Z₁′, Z₂′, Z₃′, Z₄, Z₅′, Z₆′, and Z₇′ are each independently from the group consisting of H, an albumin-binder H or a group —X₁-L₁-X₂′, provided that at least one of Z₁′, Z₂′, Z₃′, Z₄, Z₅′, Z₆′, or Z₇ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

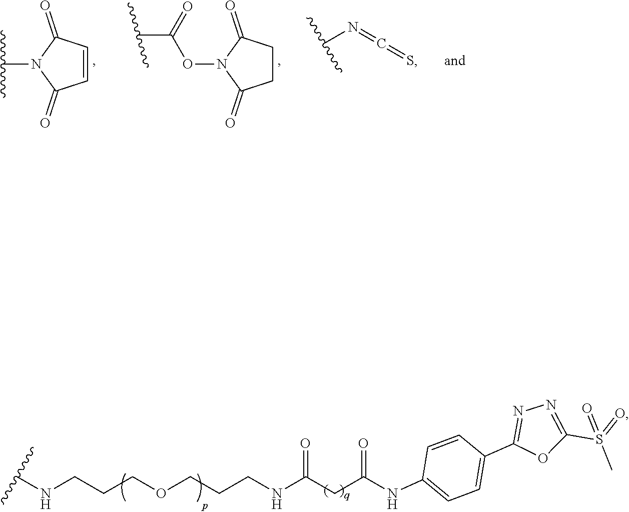

each m is independently 0 or 1;
n is 1, 2, 3, 4, 5, or 6;
p is 1, 2, 3, 4, 5, or 6; and
q is 1, 2, 3, 4, 5, or 5.
81. A compound of formula (IIg):

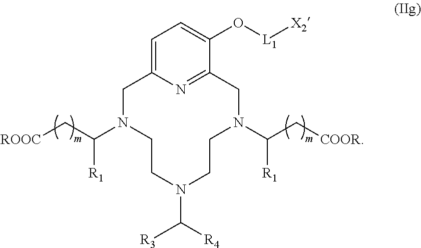

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

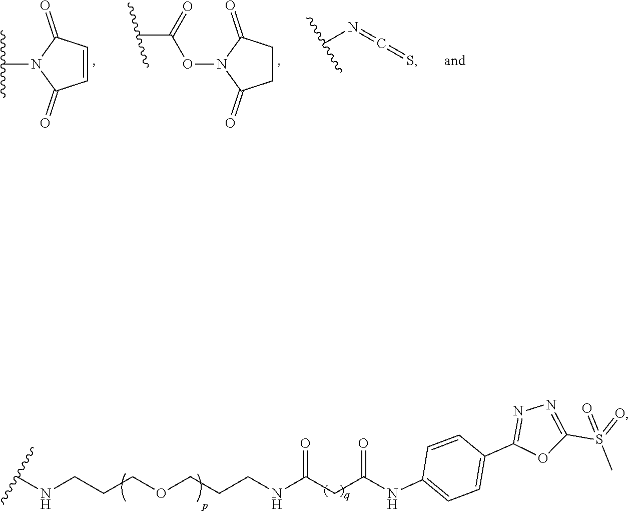

each m is independently 0 or 1;
n is 1, 2, 3, 4, 5, or 6;
p is 1, 2, 3, 4, 5, or 6; and
q is 1, 2, 3, 4, 5, or 5.
82. A compound of formula (IIh):

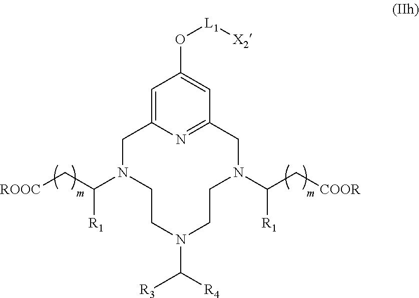

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

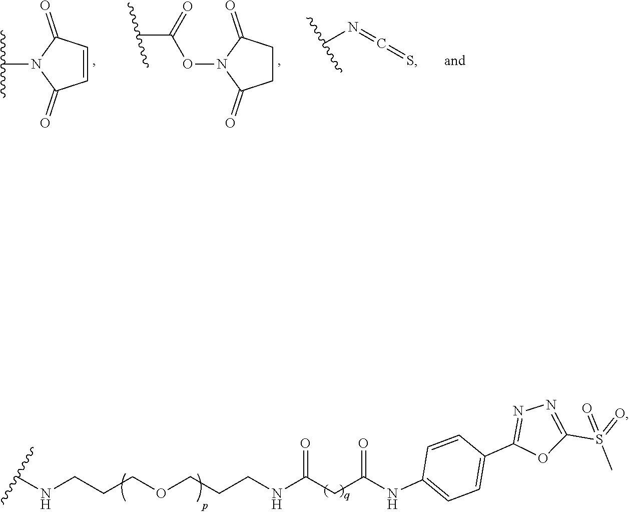

each m is independently 0 or 1;
n is 1, 2, 3, 4, 5, or 6;
p is 1, 2, 3, 4, 5, or 6; and
q is 1, 2, 3, 4, 5, or 5.
83. A compound of formula (IIi):

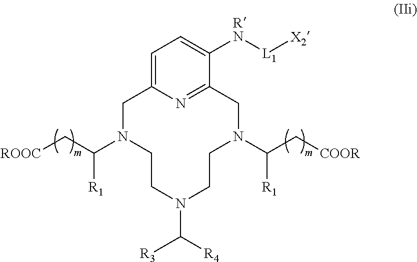

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

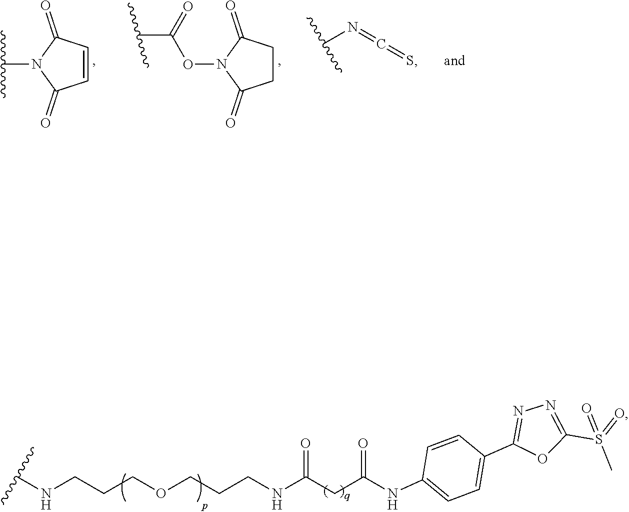

each m is independently 0 or 1;
n is 1, 2, 3, 4, 5, or 6;
p is 1, 2, 3, 4, 5, or 6; and
q is 1, 2, 3, 4, 5, or 5.
84. A compound of formula (IIj):

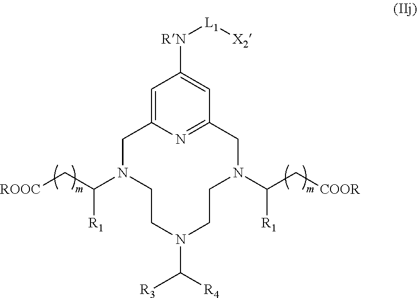

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

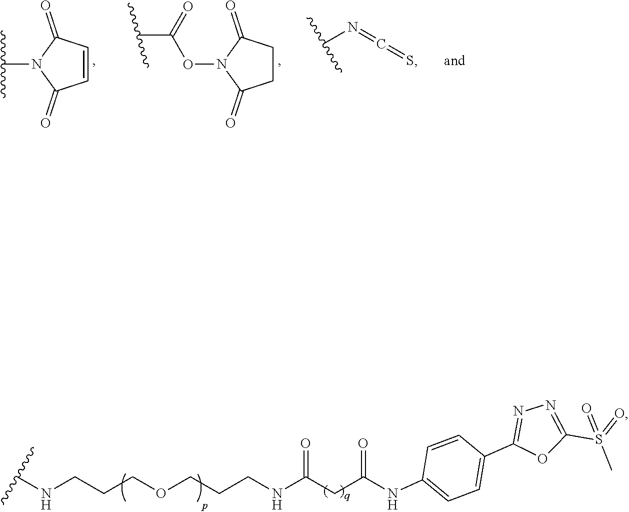

each m is independently 0 or 1;
n is 1, 2, 3, 4, 5, or 6;
p is 1, 2, 3, 4, 5, or 6; and
q is 1, 2, 3, 4, 5, or 5.
85. A compound of formula (IIk):

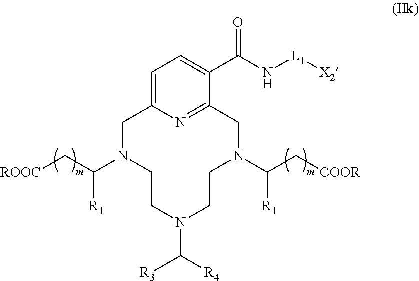

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁ aryl and heteroaryl having 5 to 10 ring atoms;
X₂′ is a group selected from the group consisting of:

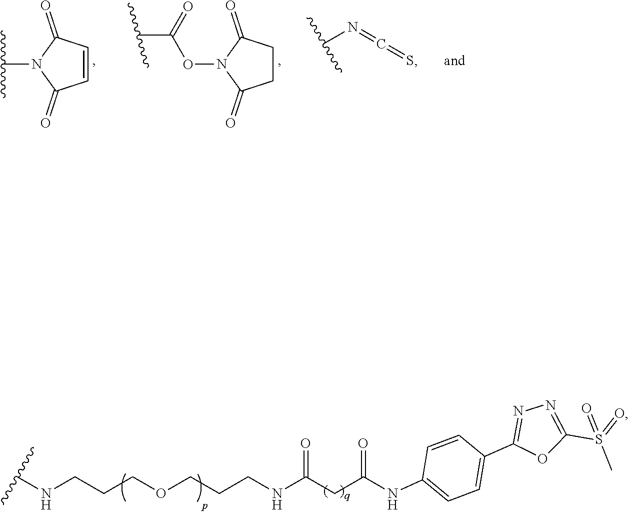

each m is independently 0 or 1;
n is 1, 2, 3, 4, 5, or 6;
p is 1, 2, 3, 4, 5, or 6; and
q is 1, 2, 3, 4, 5, or 5.
86. A compound of formula (IIm):

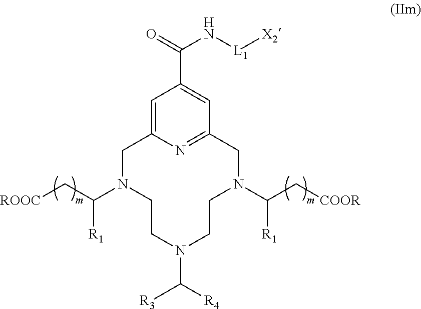

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

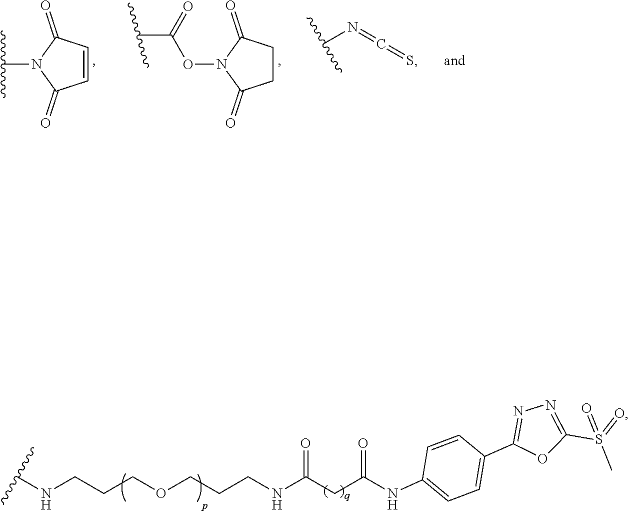

each m is independently 0 or 1;
n is 1, 2, 3, 4, 5, or 6;
p is 1, 2, 3, 4, 5, or 6; and
q is 1, 2, 3, 4, 5, or 5.
87. A compound of formula (IIn):

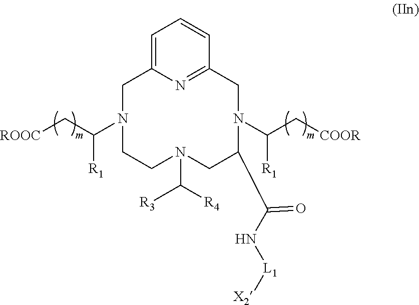

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

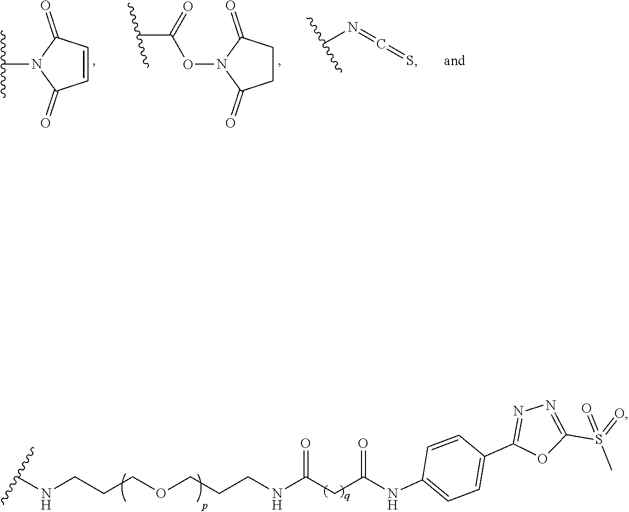

each m is independently 0 or 1;
n is 1, 2, 3, 4, 5, or 6;
p is 1, 2, 3, 4, 5, or 6; and
q is 1, 2, 3, 4, 5, or 5.
88. A compound of formula (IIo):

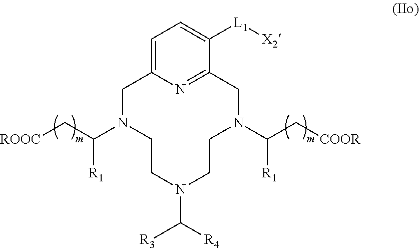

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

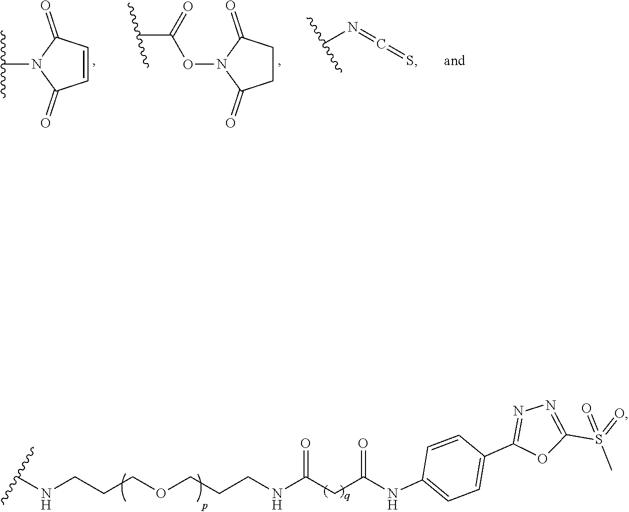

each m is independently 0 or 1;
n is 1, 2, 3, 4, 5, or 6;
p is 1, 2, 3, 4, 5, or 6; and
q is 1, 2, 3, 4, 5, or 5.
89. A compound of formula (IIp):

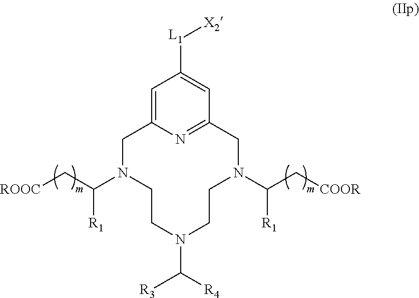

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃—C heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

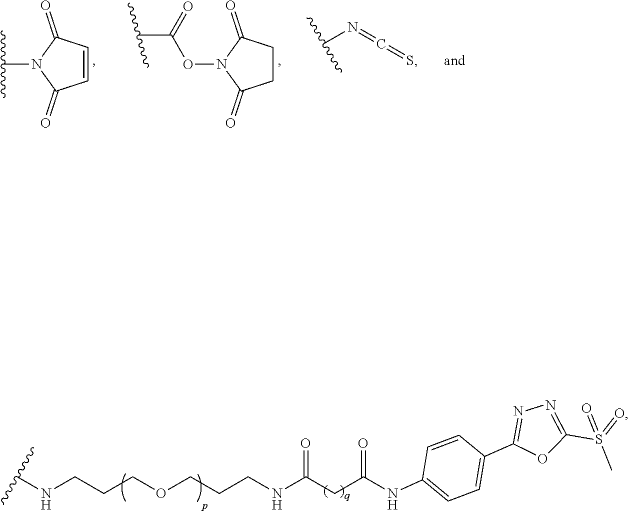

each m is independently 0 or 1;
n is 1, 2, 3, 4, 5, or 6;
p is 1, 2, 3, 4, 5, or 6; and
q is 1, 2, 3, 4, 5, or 5.
90. A compound of formula (IIq):

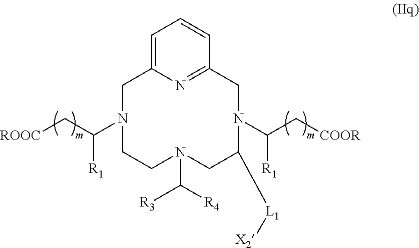

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

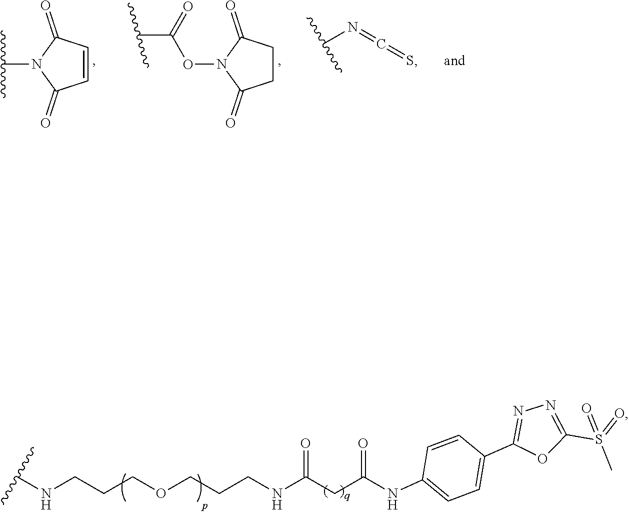

each m is independently 0 or 1;
n is 1, 2, 3, 4, 5, or 6;
p is 1, 2, 3, 4, 5, or 6; and
q is 1, 2, 3, 4, 5, or 5.
91. A compound of formula (IIg′):

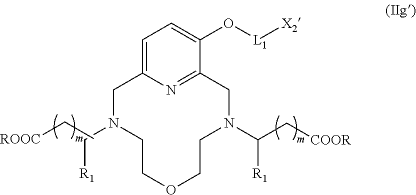

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

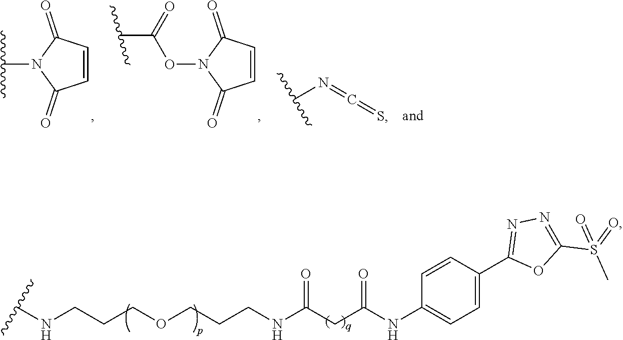

each m is independently 0 or 1;
n is 1, 2, 3, 4, 5, or 6;
p is 1, 2, 3, 4, 5, or 6; and
q is 1, 2, 3, 4, 5, or 5.
92. A compound of formula (IIh′):

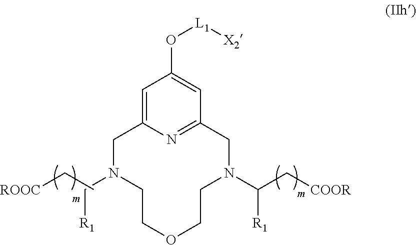

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

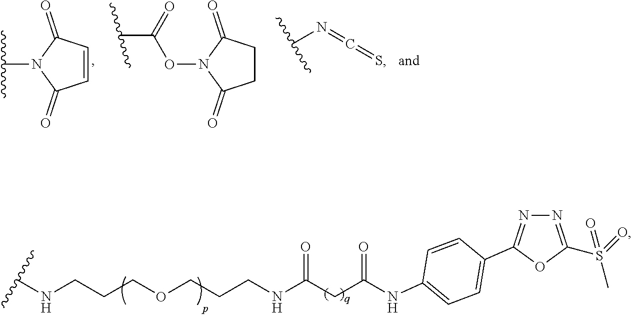

each m is independently 0 or 1;
n is 1, 2, 3, 4, 5, or 6;
p is 1, 2, 3, 4, 5, or 6; and
q is 1, 2, 3, 4, 5, or 5
93. A compound of formula (IIi′):

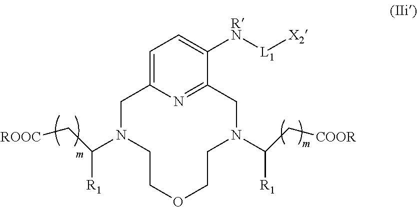

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

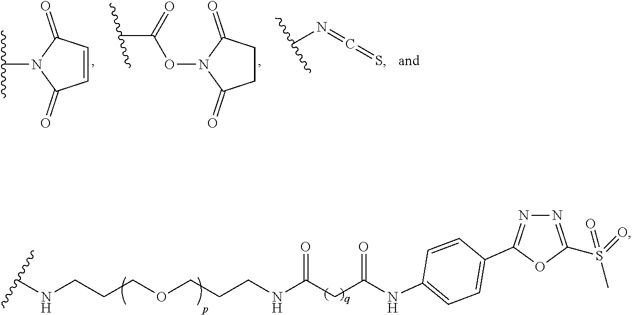

each m is independently 0 or 1;
n is 1, 2, 3, 4, 5, or 6;
p is 1, 2, 3, 4, 5, or 6; and
q is 1, 2, 3, 4, 5, or 5.
94. A compound of formula (IIj′):

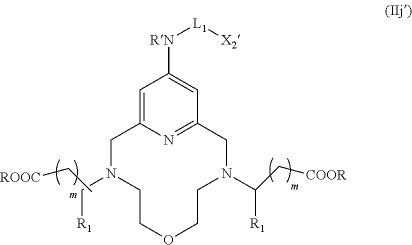

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

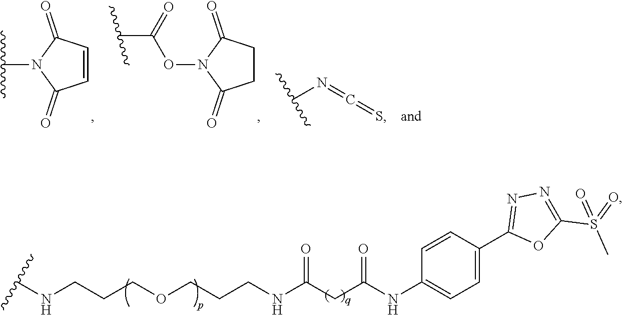

each m is independently 0 or 1;
n is 1, 2, 3, 4, 5, or 6;
p is 1, 2, 3, 4, 5, or 6; and
q is 1, 2, 3, 4, 5, or 5.
95. A compound of formula (IIk′):

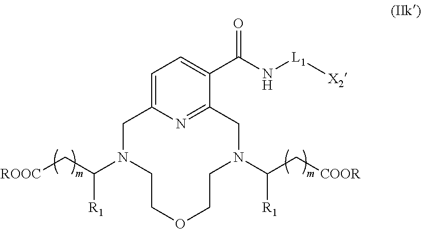

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

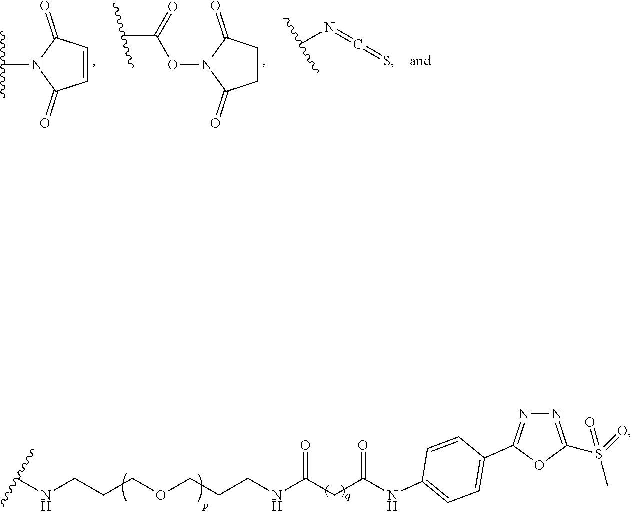

each m is independently 0 or 1;
n is 1, 2, 3, 4, 5, or 6;
p is 1, 2, 3, 4, 5, or 6; and
q is 1, 2, 3, 4, 5, or 5.
96. A compound of formula (IIm′):

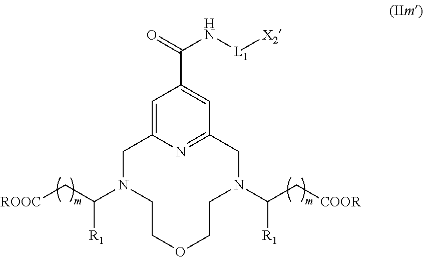

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

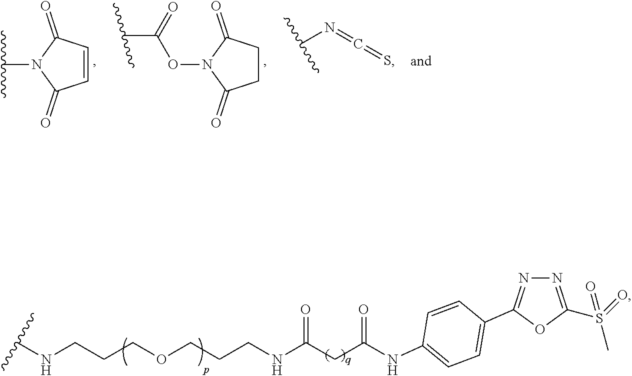

each m is independently 0 or 1;
n is 1, 2, 3, 4, 5, or 6;
p is 1, 2, 3, 4, 5, or 6; and
q is 1, 2, 3, 4, 5, or 5.
97. A compound of formula (IIn′):

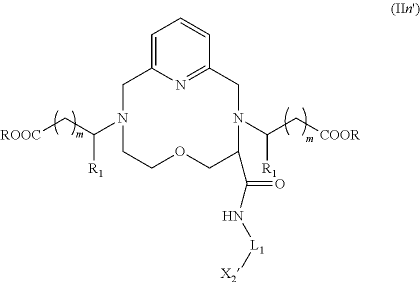

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

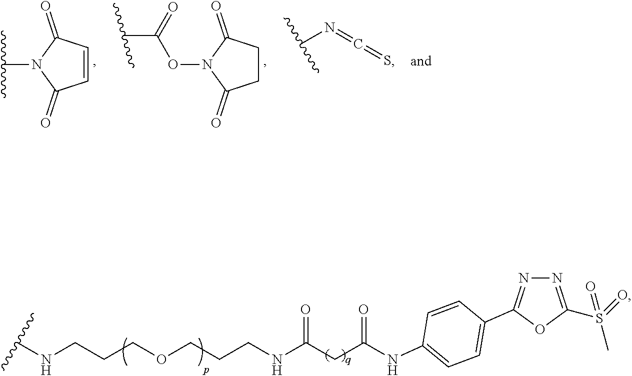

each m is independently 0 or 1;
n is 1, 2, 3, 4, 5, or 6;
p is 1, 2, 3, 4, 5, or 6; and
q is 1, 2, 3, 4, 5, or 5.
98. A compound of formula (IIo′):

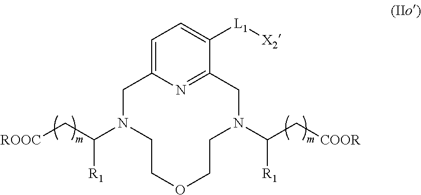

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

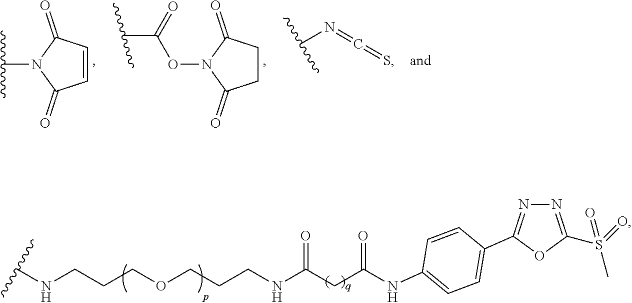

each m is independently 0 or 1;
n is 1, 2, 3, 4, 5, or 6;
p is 1, 2, 3, 4, 5, or 6; and
q is 1, 2, 3, 4, 5, or 5.
99. A compound of formula (IIp′):

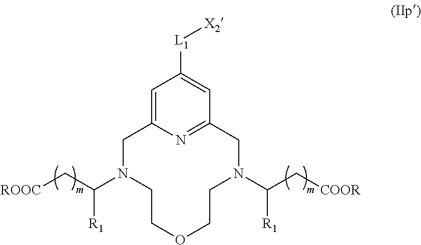

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

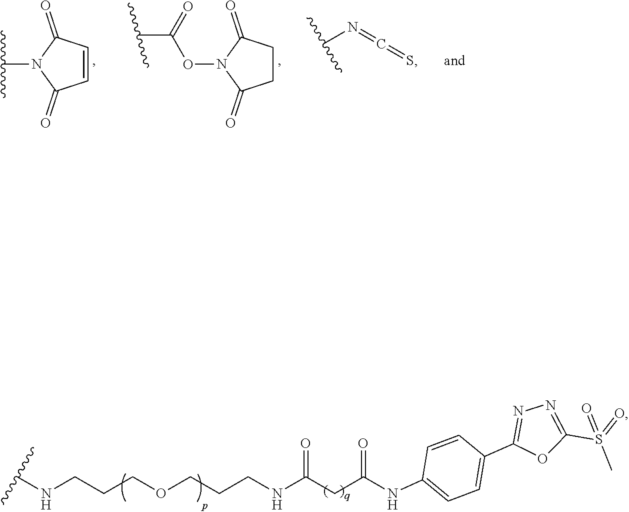

each m is independently 0 or 1;
n is 1, 2, 3, 4, 5, or 6;
p is 1, 2, 3, 4, 5, or 6; and
q is 1, 2, 3, 4, 5, or 5.
100. A compound of formula (IIq′)

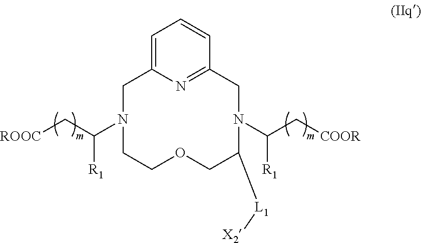

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

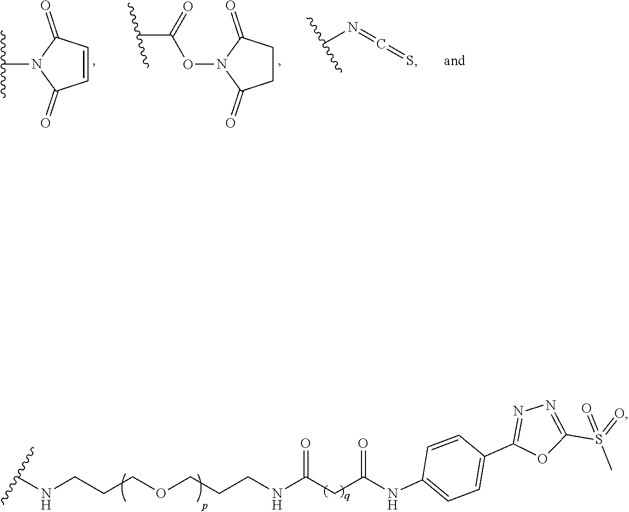

each m is independently 0 or 1;
n is 1, 2, 3, 4, 5, or 6;
p is 1, 2, 3, 4, 5, or 6; and
q is 1, 2, 3, 4, 5, or 5.
101. A compound of formula (IIg″):

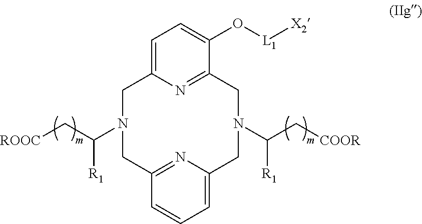

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

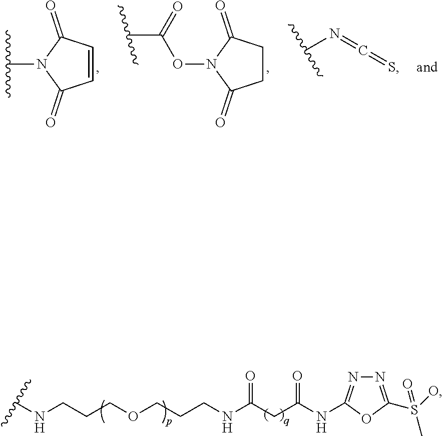

each m is independently 0 or 1;
n is 1, 2, 3, 4, 5, or 6;
p is 1, 2, 3, 4, 5, or 6; and
q is 1, 2, 3, 4, 5, or 5.
102. A compound of formula (IIh″):

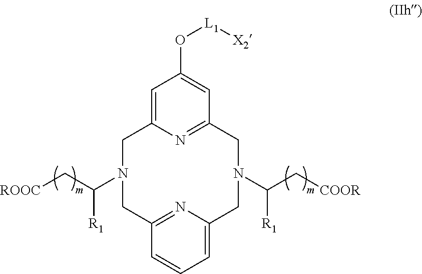

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

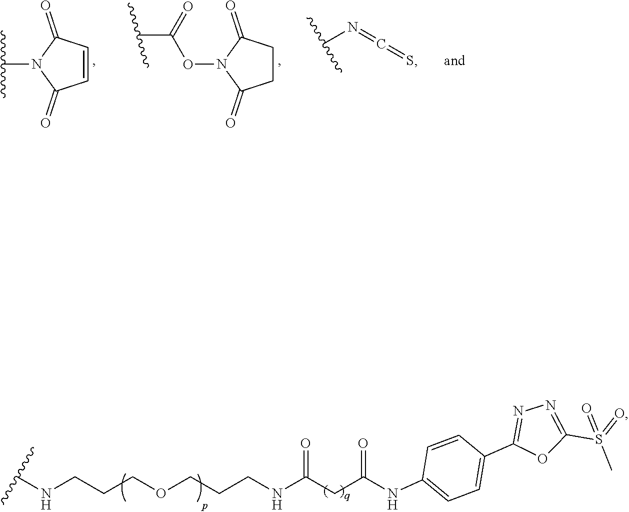

each m is independently 0 or 1;
n is 1, 2, 3, 4, 5, or 6;
p is 1, 2, 3, 4, 5, or 6; and
q is 1, 2, 3, 4, 5, or 5.
103. A compound of formula (IIi″):

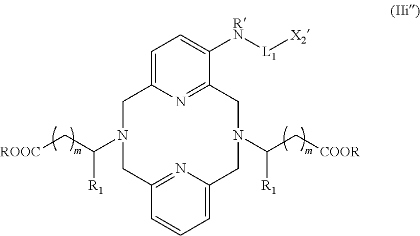

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

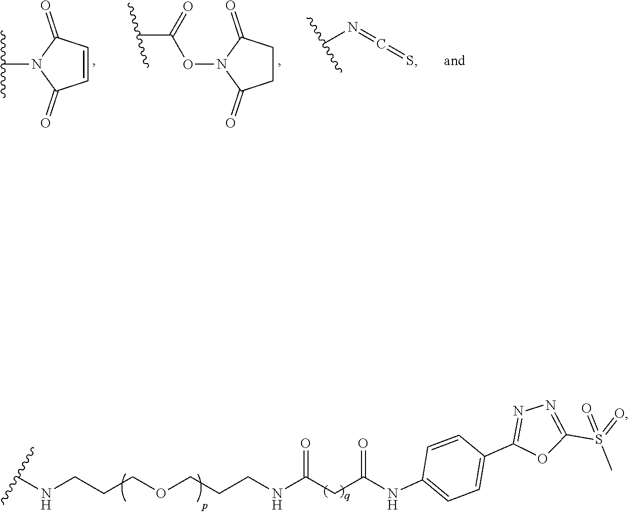

each m is independently 0 or 1;
n is 1, 2, 3, 4, 5, or 6;
p is 1, 2, 3, 4, 5, or 6; and
q is 1, 2, 3, 4, 5, or 5.
104. A compound of formula (IIj″):

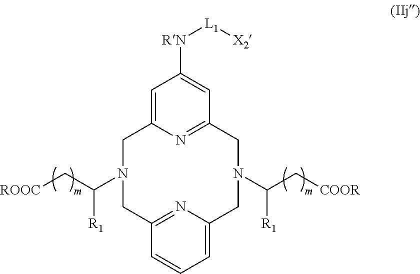

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

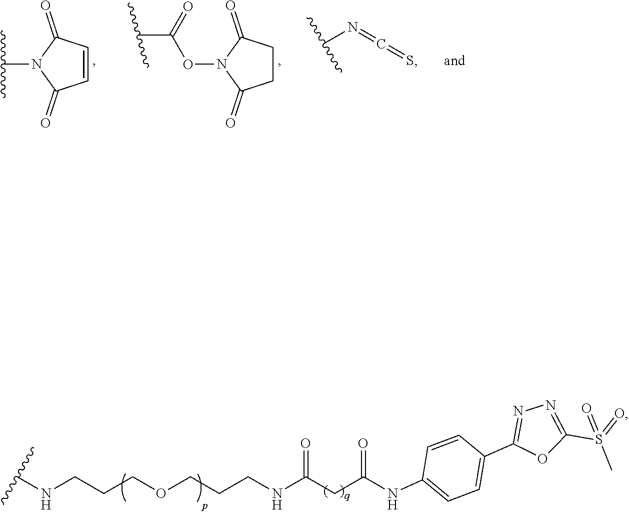

each m is independently 0 or 1;
n is 1, 2, 3, 4, 5, or 6;
p is 1, 2, 3, 4, 5, or 6; and
q is 1, 2, 3, 4, 5, or 5.
105. A compound of formula (IIk″):

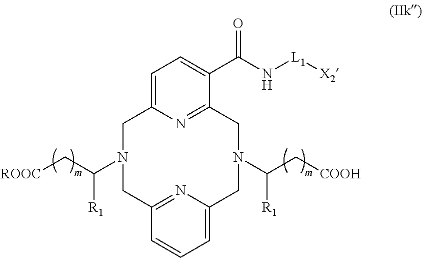

or a pharmaceutically acceptable salt thereof, wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

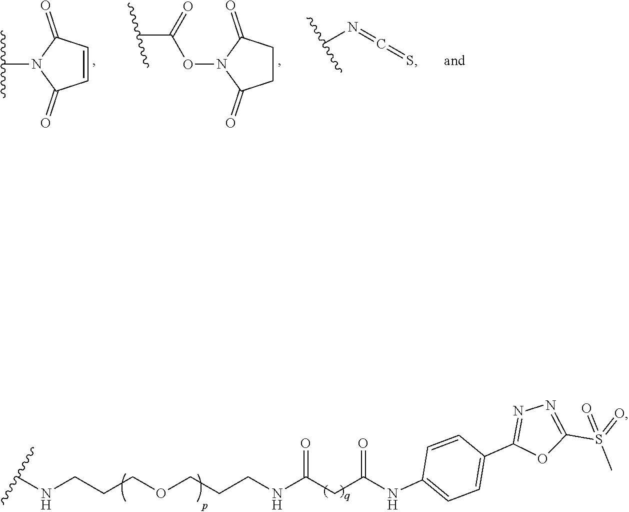

each m is independently 0 or 1;
n is 1, 2, 3, 4, 5, or 6;
p is 1, 2, 3, 4, 5, or 6; and
q is 1, 2, 3, 4, 5, or 5.
106. A compound of formula (IIm″):

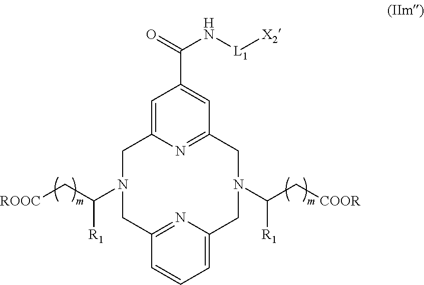

or a pharmaceutically acceptable salt thereof, wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

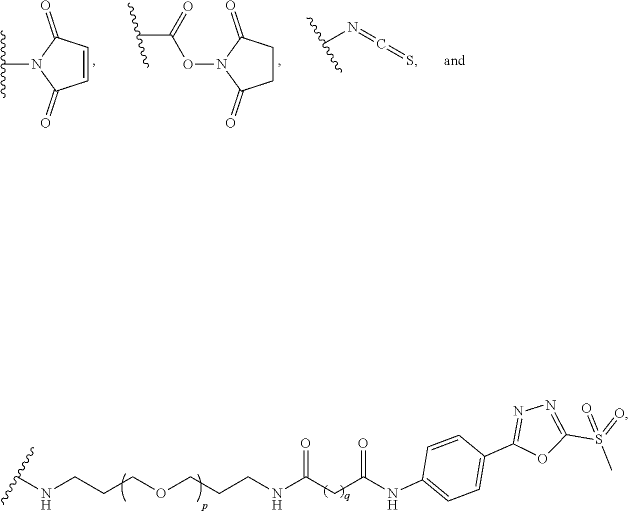

each m is independently 0 or 1;
n is 1, 2, 3, 4, 5, or 6;
p is 1, 2, 3, 4, 5, or 6; and
q is 1, 2, 3, 4, 5, or 5.
107. A compound of formula (IIn″):

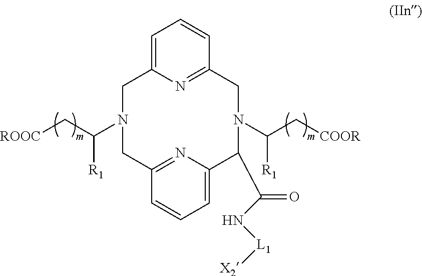

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

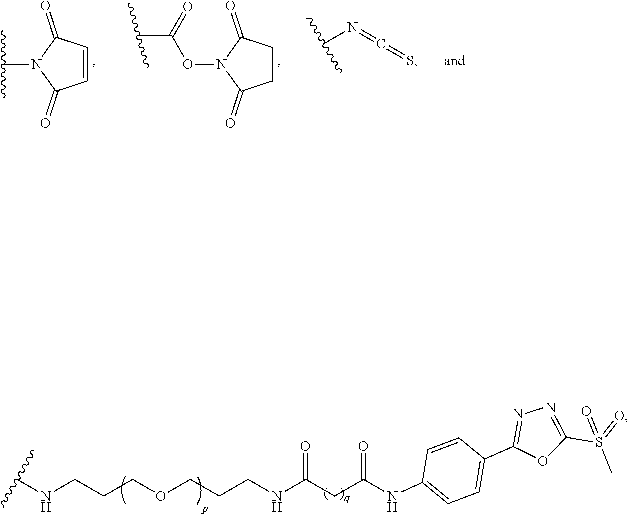

each m is independently 0 or 1;
n is 1, 2, 3, 4, 5, or 6;
p is 1, 2, 3, 4, 5, or 6; and
q is 1, 2, 3, 4, 5, or 5.
108. A compound of formula (IIo″):

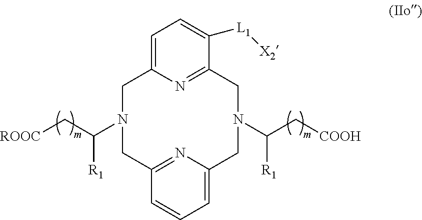

or a pharmaceutically acceptable salt thereof,
wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

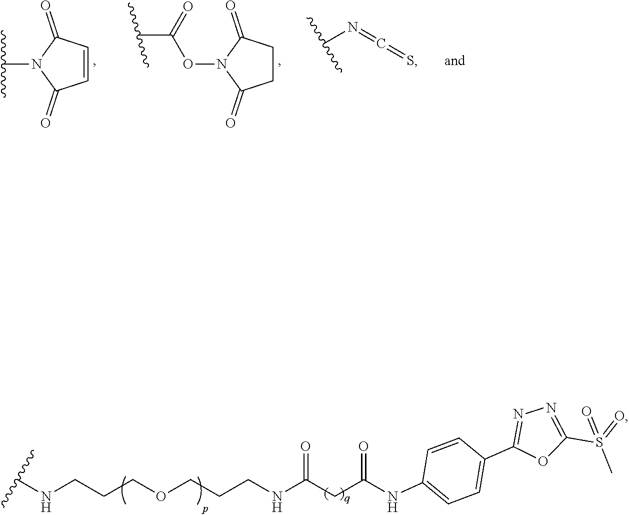

each m is independently 0 or 1;
n is 1, 2, 3, 4, 5, or 6;
p is 1, 2, 3, 4, 5, or 6; and
q is 1, 2, 3, 4, 5, or 5.
109. A compound of formula (IIp″):

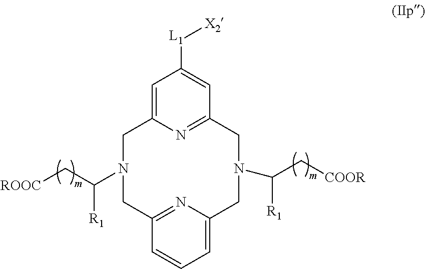

or a pharmaceutically acceptable salt thereof, wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

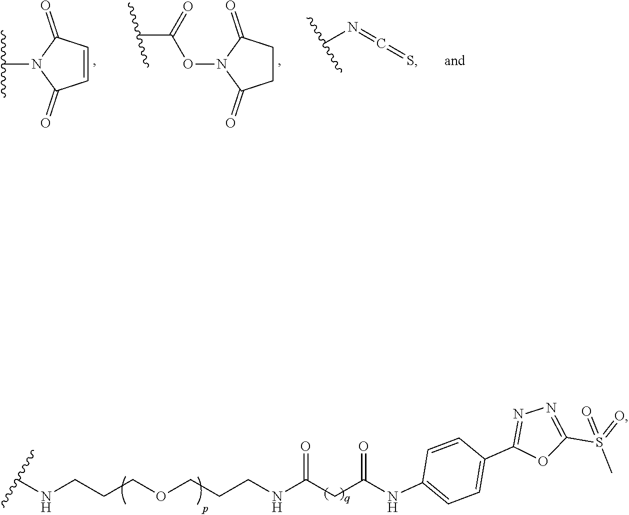

each m is independently 0 or 1;
n is 1, 2, 3, 4, 5, or 6;
p is 1, 2, 3, 4, 5, or 6; and
q is 1, 2, 3, 4, 5, or 5.
110. A compound of formula (IIq″):

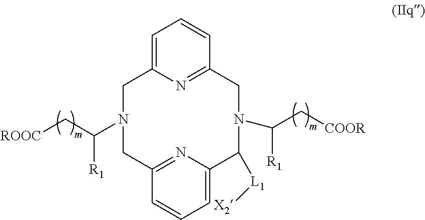

or a pharmaceutically acceptable salt thereof, wherein
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of:

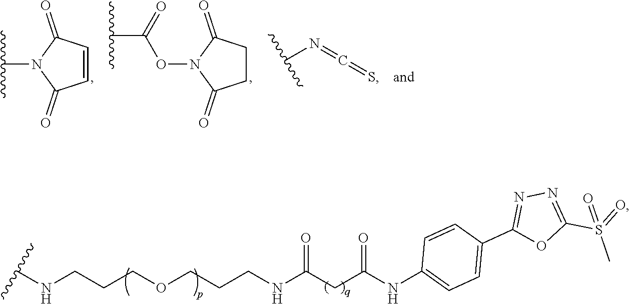

n is 1, 2, 3, 4, 5, or 6;
p is 1, 2, 3, 4, 5, or 6; and
q is 1, 2, 3, 4, 5, or 5
111. The compound of formula (II) according to any one of embodiments 71 to 119, or a pharmaceutically acceptable salt thereof, wherein R is H.
112. The compound of formula (II) according to any one of embodiments 71 to 120, or a pharmaceutically acceptable salt thereof, wherein each R₁ is independently selected from the group consisting of H, C₁-C₆ alkyl, C(═O)OR₂, (C₁-C₆ alkyl)-C(═O)OR₂, and heteroaryl having 5 to 10 ring atoms.
113. The compound of formula (II) according to any one of embodiments 71 to 121, or a pharmaceutically acceptable salt thereof, wherein each R₁ is independently selected from the group consisting of H, CH₃, C(═O)OH, CH₂C(═O)OH and pyridyl.
114. The compound of formula (II) according to any one of embodiments 71 or 122, or a pharmaceutically acceptable salt thereof, wherein L₁ is C₁-C₅ alkylene.
115. The compound of formula (II) according to any one of embodiments 71 to 123, or a pharmaceutically acceptable salt thereof, wherein X′₂ is

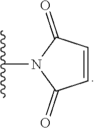 [SMILES: *N1C(=O)C=CC1=O]

116. The compound of formula (II) according to any of embodiments 71 to 124, wherein the compound is complexed with a radionuclide selected from ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, ⁶¹Cu, preferably selected from: ⁶⁸Ga, ⁶⁴Cu, ⁹⁰Y, ¹⁷⁷Lu, ²¹²Pb, ²²⁵Ac and ¹⁶¹Tb.
117. The compound of formula (II) according to any one of embodiments 71 to 80, which is selected from

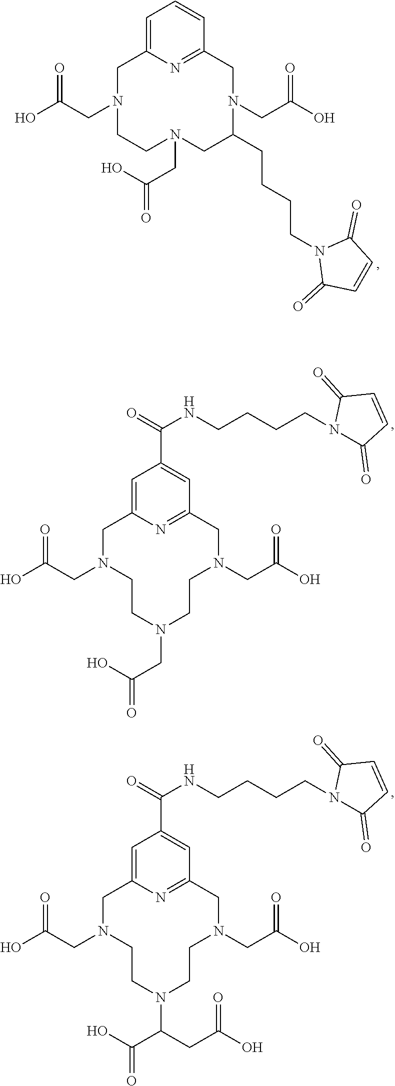 [SMILES: O=C(O)CN1CCN(CC(=O)O)CC(CCCCN2C(=O)C=CC2=O)N(CC(=O)O)Cc2cccc(n2)C1 ; O=C(O)CN1CCN(CC(=O)O)Cc2cc(C(=O)NCCCCN3C(=O)C=CC3=O)cc(n2)CN(CC(=O)O)CC1 ; O=C(O)CC(C(=O)O)N1CCN(CC(=O)O)Cc2cc(C(=O)NCCCCN3C(=O)C=CC3=O)cc(n2)CN(CC(=O)O)CC1]

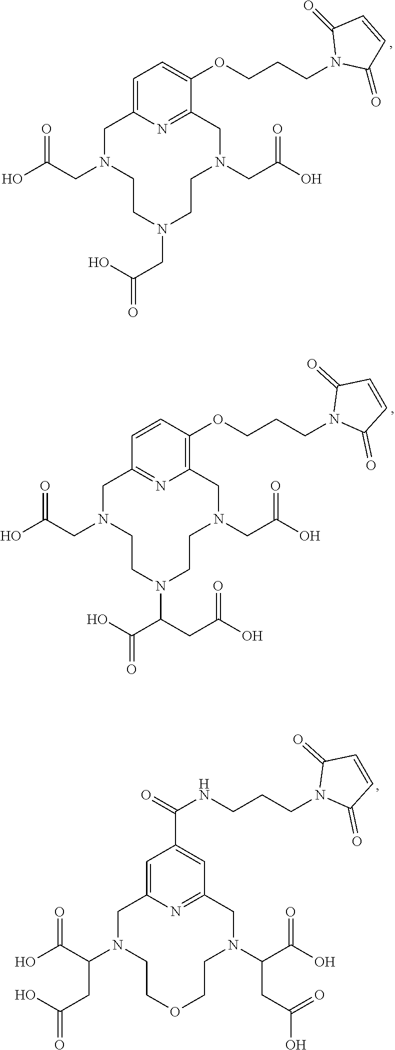 [SMILES: O=C(O)CN1CCN(CC(=O)O)Cc2ccc(OCCCN3C(=O)C=CC3=O)c(n2)CN(CC(=O)O)CC1 ; O=C(O)CC(C(=O)O)N1CCN(CC(=O)O)Cc2ccc(OCCCN3C(=O)C=CC3=O)c(n2)CN(CC(=O)O)CC1 ; O=C(O)CC(C(=O)O)N1CCOCCN(C(CC(=O)O)C(=O)O)Cc2cc(C(=O)NCCCN3C(=O)C=CC3=O)cc(n2)C1]

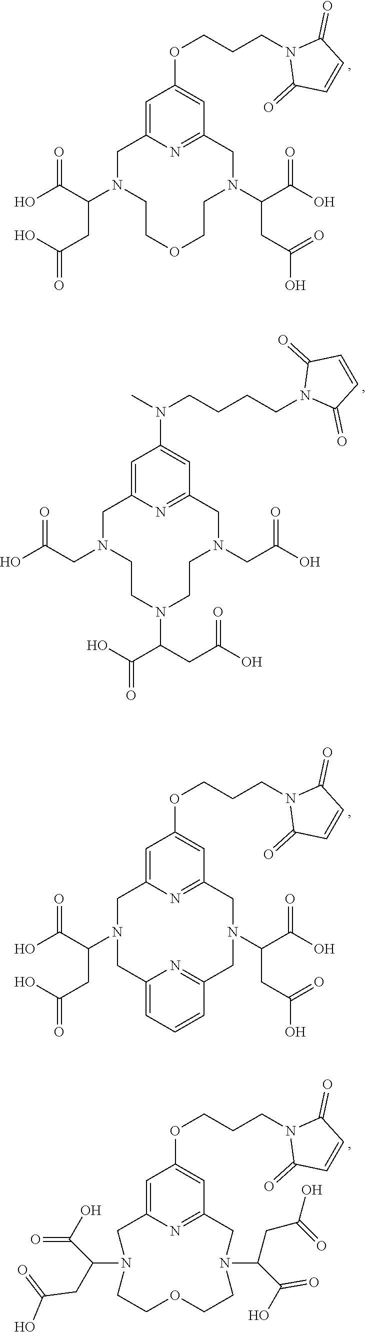 [SMILES: O=C(O)CC(C(=O)O)N1CCOCCN(C(CC(=O)O)C(=O)O)Cc2cc(OCCCN3C(=O)C=CC3=O)cc(n2)C1 ; CN(CCCCN1C(=O)C=CC1=O)c1cc2nc(c1)CN(CC(=O)O)CCN(C(CC(=O)O)C(=O)O)CCN(CC(=O)O)C2 ; O=C(O)CC(C(=O)O)N1Cc2cccc(n2)CN(C(CC(=O)O)C(=O)O)Cc2cc(OCCCN3C(=O)C=CC3=O)cc(n2)C1 ; O=C(O)CC(C(=O)O)N1CCOCCN(C(CC(=O)O)C(=O)O)Cc2cc(OCCCN3C(=O)C=CC3=O)cc(n2)C1]

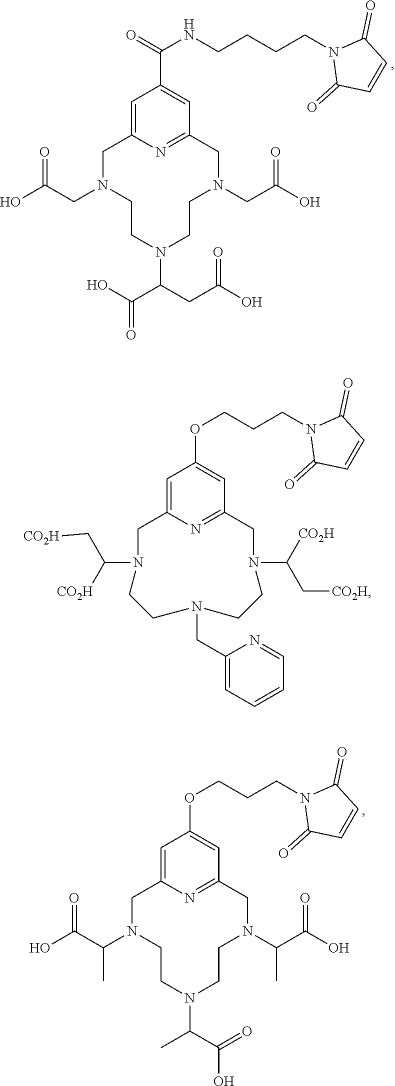 [SMILES: O=C(O)CC(C(=O)O)N1CCN(CC(=O)O)Cc2cc(C(=O)NCCCCN3C(=O)C=CC3=O)cc(n2)CN(CC(=O)O)CC1 ; O=C(O)CC(C(=O)O)N1CCN(Cc2ccccn2)CCN(C(CC(=O)O)C(=O)O)Cc2cc(OCCCN3C(=O)C=CC3=O)cc(n2)C1 ; CC(C(=O)O)N1CCN(C(C)C(=O)O)Cc2cc(OCCCN3C(=O)C=CC3=O)cc(n2)CN(C(C)C(=O)O)CC1]

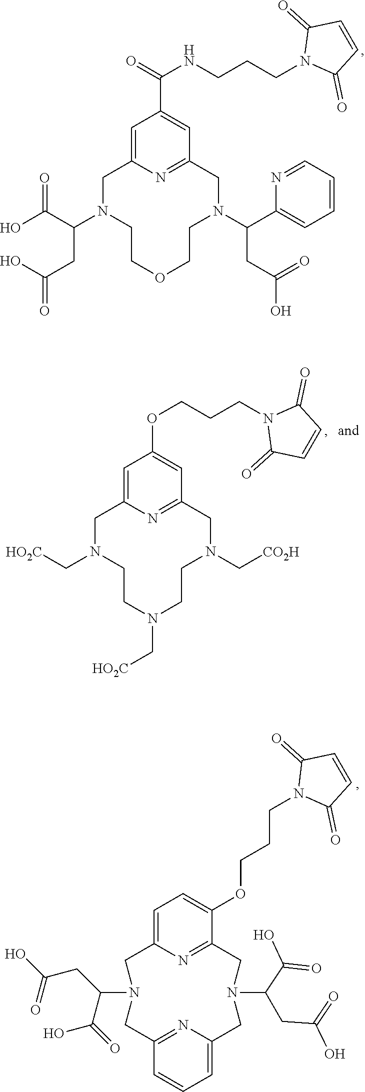 [SMILES: O=C(O)CC(C(=O)O)N1CCOCCN(C(CC(=O)O)c2ccccn2)Cc2cc(C(=O)NCCCN3C(=O)C=CC3=O)cc(n2)C1 ; O=C(O)CN1CCN(CC(=O)O)Cc2cc(OCCCN3C(=O)C=CC3=O)cc(n2)CN(CC(=O)O)CC1 ; O=C(O)CC(C(=O)O)N1Cc2cccc(n2)CN(C(CC(=O)O)C(=O)O)Cc2nc(ccc2OCCCN2C(=O)C=CC2=O)C1]

or a pharmaceutically acceptable salt thereof, and wherein the compound is optionally labeled with a radionuclide selected from ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, ⁶¹Cu, preferably selected from: ⁶⁸Ga, ⁶⁴Cu, ⁹⁰Y, ¹⁷⁷Lu, ²¹²Pb, ²²⁵Ac, and ¹⁶¹Tb.
118. The compound according to embodiment 68, which is selected from

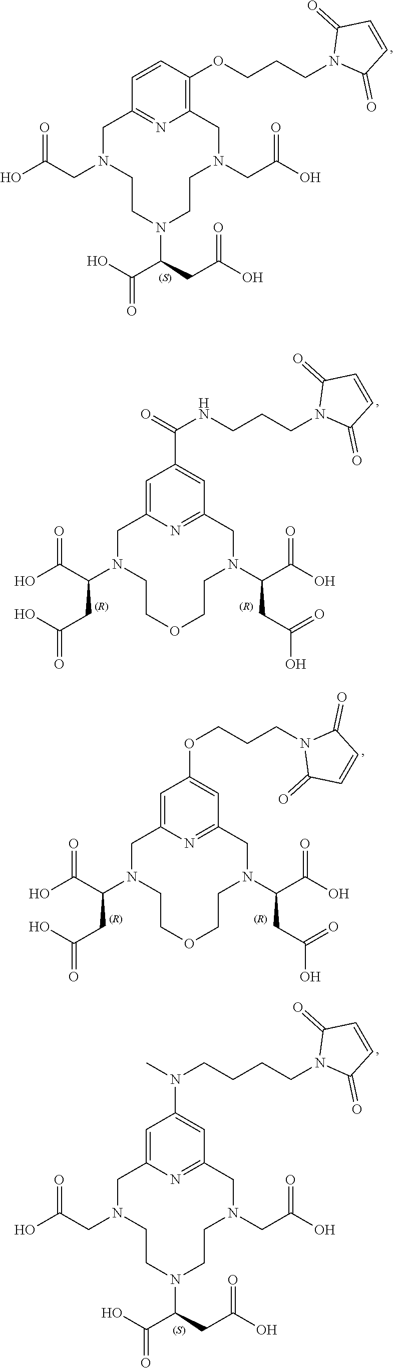

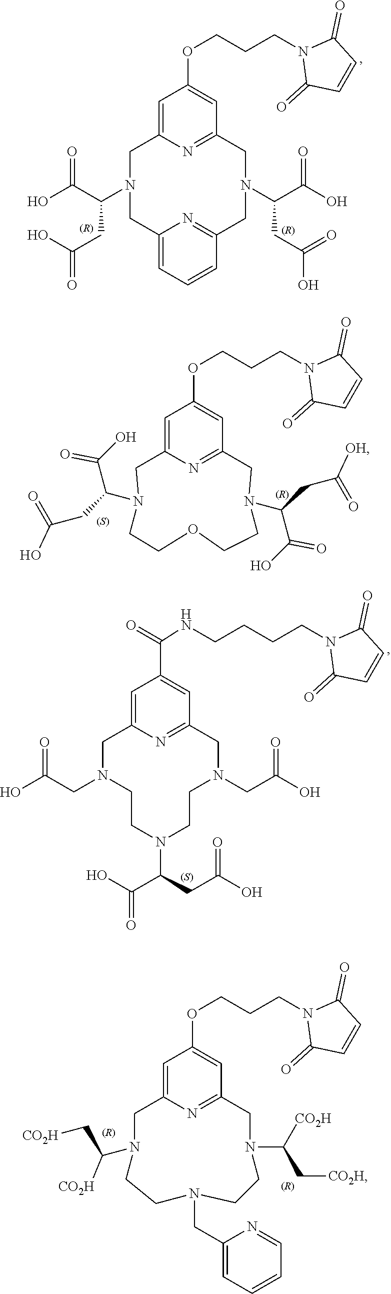

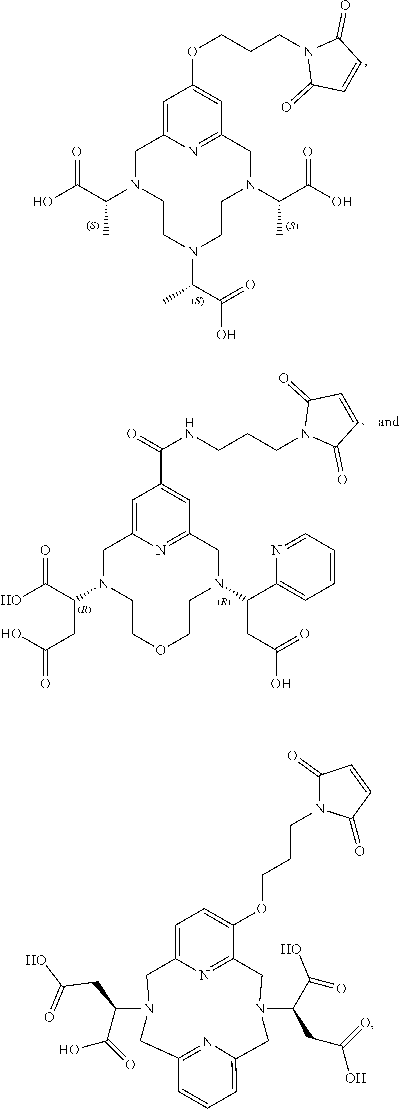

or a pharmaceutically acceptable salt thereof.
119. A pharmaceutical composition comprising a compound according to any of embodiments 1 to 127 and at least one pharmaceutically acceptable carrier
120. A compound, or a pharmaceutically acceptable salt thereof, according to any of embodiments 1 to 127, for use as a medicament.
121. The compound, or pharmaceutically acceptable salt thereof, for use according to embodiment 129, for use in treating cancer.
122. A method for treating cancer, wherein the method comprising contacting cancer cells with a therapeutically efficient amount of compound, or a pharmaceutically acceptable salt thereof, according to any of embodiments 1 to 127.
123. The compound, or a pharmaceutically acceptable salt thereof, according to any of embodiments 1 to 127, for use in imaging.
124. An imaging method, said method comprising contacting cancer cells with an efficient amount of a compound, or a pharmaceutically acceptable salt thereof, according to any of embodiments 1 to 127.
125. The compound, or a pharmaceutically acceptable salt thereof, according to any of embodiments 1 to 127 for use in diagnostic, typically for use in diagnosing cancer disorders.
126. A method for diagnosing and/or detecting cancer cells in a subject, the method comprising administering to said subject, preferably a human, an efficient amount of a compound, or a pharmaceutically acceptable salt thereof, according to any of embodiments 1-127.
127. The use of the compound of formula (I) according to claim 5, or a compound of formula (II) according to claim 71, for the manufacture of a pharmaceutical composition.
128. A method for synthesizing a compound of formula (I) as described in embodiment 5, said method comprising a step of reacting a targeting compound L₂-A, wherein L₂ is an optional linker and A is a target binding moiety, with a compound of formula (II):

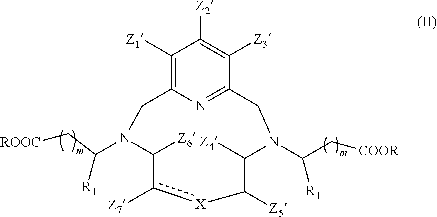

or a pharmaceutically acceptable salt thereof,
wherein
[symbol] is a single bond or a double bond, wherein
when [symbol] is a single bond, X is —O— or

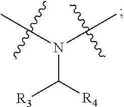

when [symbol] is a double bond, X is ═N— and Z₅′ and Z₇′ form, together with the N atom, a heteroaryl having 5 or 6 ring atoms, otherwise, Z₅′ and Z₇′ are each independently selected from the group consisting of H, an albumin-binder and —X₁-L₁-X₂′;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
Z₁′, Z₂′, Z₃′, Z₄′, and Z₆′ are each independently from the group consisting of H, an albumin-binder H or a group —X₁-L₁-X₂′, provided that at least one of Z₁′, Z₂′, Z₃′, Z₄′, Z₅′, Z₆′, of Z₇′ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁ aryl and heteroaryl having 5 to 10 ring atoms;
X₂′ is a group selected from the group consisting of:

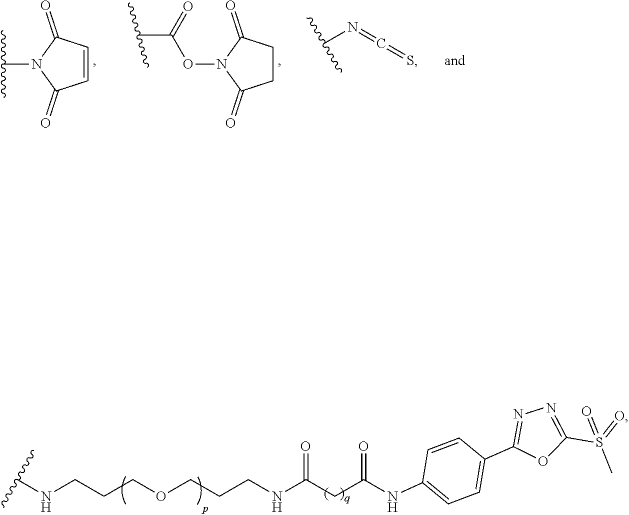

each m is independently 0 or 1;
n is 1, 2, 3, 4, 5, or 6;
p is 1, 2, 3, 4, 5, or 6; and
q is 1, 2, 3, 4, 5, or 5.
129. A compound capable of complexing ⁶⁸Ga and/or ¹⁷⁷Lu at a temperature of s 60° C., to achieve 85% complexation of the compound in a composition, said compound comprising formula (C)

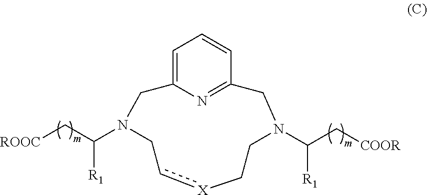

or a pharmaceutically acceptable salt thereof
wherein
[symbol] is a single bond or a double bond,
when [symbol] is a single bond, X is —O— or

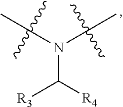

when [symbol] is a double bond, X is ═N—
each m is 0 to 5;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, heteroaryl having 5 to 10 ring atoms, or any combination thereof, and
the compound of formula (C) being optionally substituted.

### EXAMPLES

### General Conditions:

Mass spectra were acquired on LC-MS, SFC-MS, or GC-MS systems using electrospray, chemical and electron impact ionization methods from a range of instruments of the following configurations: Agilent 1100 HPLC systems with an Agilent 6110 Mass Spectrometer [M+H]+ refers to protonated molecular ion of the chemical species.

NMR spectra were run on BrukerAVANCE 400 MHz or 500 MHz NMR spectrometers using ICON-NMR, under TopSpin program control. Spectra were measured at 298K, unless indicated otherwise, and were referenced relative to the solvent resonance.

### Instrumentation

### LC/MS Methods:

LC-MS-1
System: Shimadzu (2020-single quad)
Column: synergi 2.5μ MAX-RP100 A Mercury
Column temperature: 40° C.
Gradient: 0.1/5, 0.5/5, 1.0/95, 1.5/95, 2.0/5, 3.0/5
Eluent A: 0.1% HCO₂H in water
Eluent B: CH₃CN
Flow: 2.0 mL/min
LC-MS-2
System: API 2000
Column: Kinetex EVO 2.6 μm, 50*4.6 mm, column temperature: 30° C.
Gradient: 0/30, 0.5/30, 1.5/95, 2.4/95, 2.5/30, 3.0/30
Eluent A: 0.1% HCO₂H in water
Eluent B: CH₃CN
Flow: 2.0 mL/min
LC-MS-3
System: EVO ESI 3200
Column: Kinetex EVO 2.6 μm, 50*4.6 mm, column temperature: 30° C.
Gradient: 0/30, 0.2/30, 0.7/95, 2.0/95, 2.5/30, 3.5/30
Eluent A: 0.1% HCO₂H in water
Eluent B: 0.1% HCO₂H in CH₃CN
Flow: 2.0 mL/min
LC-MS-4
System: EVO ESI 3200
Column: Kinetex EVO 2.6 μm, 50*4.6 mm, column temperature: 30° C.
Gradient: 0.0/20, 0.25/20, 1.0/95, 2.5/95, 3.0/20, 4.0/20, 5.0/20
Eluent A: 0.1% HCO₂H in water
Eluent B: 0.1% HCO₂H in CH₃CN
Flow: 2.0 mL/min LC-MS-5 (Using Shimadzu LCMA-2020): Kinetex EVO C18, particle size: 5 μm, column size: 2.1×30 mm, column temperature: 50° C.; flow rate: 1.5 mL/min; eluent A: 0.0375% TFA in water; eluent B: 0.01875% TFA in acetonitrile; gradient: from 5 to 95% B in 0.8 min then 95% B for 0.4 min acetonitrile then from 95 to 5% B in 0.35 min.
UPLC/MS Methods: Using Agilent 1100 HPLC systems with an Agilent 6110 Mass Spectrometer
LC-MS-6: Phenomenex Gemini C18; particle size: 3.0 μm; column size: 50×4.6 mm; column temperature: 50° C.; flow rate: 1 mL/min; eluent A: H₂O+0.1% TFA; eluent B: methanol+0.1% TFA; gradient: 5 to 95% B in 2.0 min, then 95% B for 0.2 min.
LC-MS-7: Waters BEH C18; particle size: 1.7 μm; column size: 50×2.1 mm; column temperature: 50° C.; flow rate: 0.8 mL/min; eluent A: H₂O+0.1% TFA; eluent B: acetonitrile+0.1% TFA; gradient: 0.20 min 5% B; 5% to 95% B in 1.30 min, 0.25 min 95% B.
LC-MS-8: CORTECS™ 018; particle size: 2.7 μm; column size: 50×2.1 mm; column temperature: 80° C.; flow rate: 1 mL/min; eluent A: H₂O+4.76% isopropanol+0.05% FA+3.75 mM AA; eluent B: isopropanol+0.05% FA; gradient: from 1 to 50% B in 1.4 min; 50 to 98% B in 0.3 min.
LC-MS-9: CORTECS™ 018; particle size: 2.7 μm; column size: 50×2.1 mm; column temperature: 80° C.; flow rate: 1 mL/min; eluent A: H₂O+0.05% FA+3.75 mM AA; eluent B: isopropanol+0.05% FA; gradient: curved from 1 to 98% B in 1.7 min.
LC-MS-10: CORTECS™ 018; particle size: 2.7 μm; column size: 50×2.1 mm; column temperature: 80° C.; flow rate: 1 mL/min; eluent A: water+4.76% isopropanol+0.05% FA+3.75 mM AA; eluent B: isopropanol+0.05% FA; gradient: from 1 to 50% B in 1.4 min; 50 to 98% B in 0.3 min.
LC-MS-11: ACQUITY UPLC® BEH C18; particle size: 1.7 μm; column size: 100×2.1 mm; column temperature: 80° C.; flow rate: 0.4 mL/min; eluent A: water+4.76% isopropanol+0.05% FA+3.75 mM AA; eluent B: isopropanol+0.05% FA; gradient: from 1 to 60% B in 8.4 min; 60 to 98% B in 1.0 min.
LC-MS-12: CORTECS™ 018; particle size: 2.7 μm; column size: 50×2.1 mm; column temperature: 80° C.; flow rate: 1 mL/min; eluent A: water+4.76% isopropanol+0.05% FA+3.75 mM AA; eluent B: isopropanol+0.05% FA; gradient: from 1 to 50% B in 1.4 min; 50 to 98% B in 0.3 min; detector: ELSD
LC-MS-13: CORTECS™ 018; particle size: 2.7 μm; column size: 50×2.1 mm; column temperature: 80° C.; flow rate: 1 mL/min; eluent A: H₂O+0.05% FA+3.75 mM AA; eluent B: isopropanol+0.05% FA; gradient: concave from 1 to 98% B in 1.4 min
LC-MS-14: CORTECS™ 018; particle size: 2.7 μm; column size: 50×2.1 mm; column temperature: 80° C.; flow rate: 1 mL/min; eluent A: water+0.05% FA+3.75 mM AA; eluent B: acetonitrile+0.04% FA; gradient: from 5 to 98% B in 1.4 min.
LC-MS-15: CORTECS™ 018; particle size: 2.7 μm; column size: 50×2.1 mm; column temperature: 80° C.; flow rate: 1 mL/min; eluent A: H2O+0.05% FA+3.75 mM AA; eluent B: isopropanol+0.05% FA; gradient: from 5 to 50% B in 1.4 min; 50 to 98% B in 0.3 min
LC-MS-16: ACQUITY UPLC® CSH™ 018; particle size: 1.7 μm, column size: 2.1×100 mm, column temperature: 80.0° C.; flow rate: 0.5 mL/min; eluent A: H2O+0.05% TFA, eluent B: CH₃CN+0.04% TFA; gradient: hold 5% B for 0.2 min; from 5% to 98% B in 9.2 min.

### Preparative Methods

Flash Chromatography: Normal phase chromatography was run on silica gel using prepacked columns (RediSep Rf cartridges, or SNAP cartridges onto Isolute, or on silica gel, or applied as solutions), or using glass columns following standard flash chromatography methodology, unless otherwise stated.
System: Teledyne ISCO, CombiFlash Rf, Biotage Isolera.

### Reverse Phase HPLC:

RP-HPLC-1: Column: Gemini (250 mm×21.2 mm, 5 μm); mobile phase: A=0.1% formic acid in water, B=acetonitrile; flow rate: 18 mL/min.
RP-HPLC-2: Waters; column: XBridge Prep C18; particle size 5 μm; column size: 30×100 mm; flow rate: 50 mL/min; mobile phase A: H₂O+0.1% TFA (1/1) and B: CH₃CN; gradient: 2 to 30% solvent A in 10 min
RP-HPLC-3: ACCQ prep; column: XBridge Prep C18; particle size 5 μm; column size: 30×100 mm; flow rate: 50 mL/min; mobile phase A: H₂O+0.1% TFA and B: CH₃CN; gradient: 2 to 20% solvent A in 10 min
RP-HPLC-4: ACCQ prep; column: XBridge Prep C18; particle size 5 μm; column size: 30×100 mm; flow rate: 50 mL/min; mobile phase A: H2O+0.1% TFA and B: CH3CN; gradient: 0 to 100% solvent B in 12 min
RP-HPLC-5: Sunfire prep; column: C18 OBD™; particle size 5 μm; column size: 30×100 mm; flow rate: 30 mL/min; mobile phase A: H2O+0.1% TFA and B: CH3CN; gradient: 0 to 30% solvent B in 30 min
RP-HPLC-6: ACCQ prep; column: XBridge Prep C18; particle size 5 μm; column size: 50×100 mm; flow rate: 100 mL/min; mobile phase A: H2O+0.1% TFA and B: CH3CN; gradient: 0 to 100% solvent B in 12 min

### ABBREVIATIONS

BOC tertiary butyl carboxy
br broad
d doublet
dd doublet of doublets
DCM dichloromethane
DMF N,N-dimethylformamide
DMSO dimethylsulfoxide
EDTA ethylenediamine tetraacetic acid
ESI electrospray ionization
EtOAc ethyl acetate
h hour(s)
HPLC high pressure liquid chromatography
LCMS liquid chromatography and mass spectrometry
MeOH methanol
MS mass spectrometry
m multiplet
mg milligram
min minutes
ml milliliter
mmol millimol
m/z mass to charge ratio
NMR nuclear magnetic resonance
ppm parts per million
rac racemic
Rt retention time
s singlet
t triplet
TFA trifluoroacetic acid
THF tetrahydrofuran

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### PREPARATION OF INTERMEDIATES

### Intermediate A: Di-tert-butyl 2,2′-((((2-(tert-butoxy)-2-oxoethyl)azanediyl)bis(ethane-2,1-diyl)bis(azanediyl))diacetate

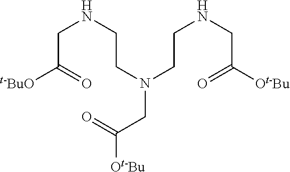 [SMILES: CC(C)(C)OC(=O)CNCCN(CCNCC(=O)OC(C)(C)C)CC(=O)OC(C)(C)C]

### Step 1: Di-tert-butyl 2,2′-((((2-(tert-butoxy)-2-oxoethyl)azanediyl)bis(ethane-2,1-diyl))bis(benzylazanediyl))diacetate

To a stirred suspension of tert-butyl glycinate hydrochloride salt (2.55 g, 15.29 mmol) in anhydrous CH₃CN (100 mL) was added anhydrous K₂CO₃ (21.09 g, 152.87 mmol) at RT. After stirring at 50° C. for 1 hour, the reaction mixture was cooled to RT and tert-butyl N-benzyl-N-(2-bromoethyl)glycinate, prepared according to Eur. J. Org. Chem. 2018, 1765-1773, supporting information, page 6, compound 5 (10.0 g, 30.57 mmol) in anhydrous CH₃CN (20 mL) was added at RT. The resulting mixture was heated to reflux for 24 hours and cooled to RT. The suspension was filtered through short celite plug and the filtrate was evaporated under reduced pressure. The residue was purified by flash chromatography using Redisep cartridge (80 g) eluting with hexane/EtOAc and afforded the title compound (12 g, crude) as a pale yellow viscous oil. LC-MS-4: Rt=1.551 min, m/z: 626.50 [M+H]; ¹H NMR (400 MHz, CDCl₃) δ=7.31-7.25 (m, 10H), 3.78 (s, 4H), 3.29 (s, 2H), 3.21 (s, 2H), 2.74 (s, 8H), 1.45 (s, 18H), 1.42 (s, 9H).

### Step 2: Di-tert-butyl 2,2′-((((2-(tert-butoxy)-2-oxoethyl)azanediyl)bis(ethane-2,1-diyl))bis(azanediyl))diacetate

A solution of di-tert-butyl 2,2′-((((2-(tert-butoxy)-2-oxoethyl)azanediyl)bis(ethane-2,1-diyl))bis(benzylazanediyl))diacetate (3.0 g, 31.9 mmol) in ethanol (30 mL) was treated with 30% w/w of Pd/C (900 mg) under argon atmosphere. The reaction mixture was placed under hydrogen gas at RT for 16 hours. The reaction mixture was filtered through a Celite bed and washed with methanol (2×50 mL). The combined organic layers were evaporated to obtain the title compound (6.0 g, crude for 2×3 g batches) as colorless oil. LC-MS-2: Rt=0.14 min, m/z: 446.20 [M+H]; ¹H NMR (400 MHz, DMSO-d₆) δ=3.15-3.22 (m, 8H), 2.61-2.62 (m, 4H), 2.52-2.54 (m, 4H), 1.35-1.41 (m, 27H).

### Intermediate B: Di-tert-butyl N,N-bis(2-((2-(tert-butoxy)-2-oxoethyl)amino)ethyl)-L-aspartate

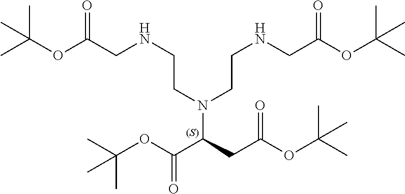

### Step 1: Di-tert-butyl L-aspartate

To a stirred solution of L-aspartic acid (5.0 g, 37.5 mmol) in tert-butylacetate (468 mL) was added dropwise 70% HClO₄ aq. at RT and the reaction mixture was stirred at RT for 16 hours. After completion, the reaction mixture was cooled to 0° C. and then extracted with cold aq. 0.5 N HCl solution. The aqueous layer was neutralized with saturated NaHCO₃ solution and extracted with EtOAc. The combined organic layers were dried using anhydrous Na₂SO₄, filtered and concentrated under reduced pressure to obtain the di-tert-butyl L-aspartate (3.0 g, 32.60%) as a colorless oil. LC-MS-2: Rt=0.13 min, m/z: 246.10 [M+H]; ¹H NMR (400 MHz, CDCl₃) δ=4.09 (t, J=5.0 Hz, 1H), 2.91 (d, J=5 Hz, 2H), 2.09 (s, 2H), 1.46-1.48 (m, 18H).

### Step 2: Di-tert-butyl N,N-bis(2-(benzyl(2-(tert-butoxy)-2-oxoethyl)amino)ethyl)-L-aspartate

Similarly to intermediate A, step 1, di-tert-butyl L-aspartate (1.14 g, 4.63 mmol), anhydrous K₂CO₃ (6.33 g, 45.86 mmol) and tert-butyl N-benzyl-N-(2-bromoethyl)glycinate (3.0 g, 9.17 mmol) in anhydrous CH₃CN (76.14 mL) were refluxed for 16 hours to obtain di-tert-butyl N,N-bis(2-(benzyl(2-(tert-butoxy)-2-oxoethyl)amino)ethyl)-L-aspartate (3.0 g, crude) as a colorless oil.
LC-MS-1: Rt=1.619 min, m/z: 741.10 [M+H]; ¹H NMR (300 MHz, CDCl₃) δ=7.21-7.31 (m, 10H), 3.73-3.75 (m, 5H), 3.20 (s, 4H), 2.63-2.71 (m, 8H), 1.41-1.45 (m, 36H).

### Step 3: Di-tert-butyl N,N-bis(2-((2-(tert-butoxy)-2-oxoethyl)amino)ethyl)-L-aspartate

Similarly to intermediate A, step 2, di-tert-butyl N,N-bis(2-(benzyl(2-(tert-butoxy)-2-oxoethyl)amino)ethyl)-L-aspartate (2.0 g, 3.99 mmol), 30% w/w Pd/C (600 mg) in EtOH (20 mL) was debenzylated to obtain di-tert-butyl N,N-bis(2-((2-(tert-butoxy)-2-oxoethyl)amino)ethyl)-L-aspartate (1.3 g, crude) as beige gum. ¹H NMR (400 MHz, DMSO-d₆) δ=3.61 (t, J=6.8 Hz, 1H), 3.56-3.64 (m, 1H), 3.21-3.43 (m, 4H), 3.16-3.21 (m, 4H), 2.53-2.63 (m, 8H), 1.34-1.44 (m, 36H).

### Intermediate C: 4-(2,5-Dioxo-2,5-dihydro-1H-pyrrol-1-yl)butan-1-aminium chloride

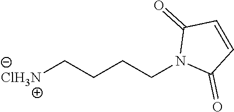

To a stirred solution of tert-butyl (4-(2,5-dioxo-2,5-dihydro-1H-pyrrol-1-yl)butyl)carbamate prepared according to the procedure of JACS, 2018, Vol. 140(20), p. 6278-6287, supporting information, page S5, compound 3-Bu (1.9 g, 7.09 mmol) in dry CH₂Cl₂ (30 mL) under argon was dropwise added 4M solution of HCl in dioxane (7 mL, 28.34 mmol) at 0° C. The reaction mixture was slowly warmed to RT and stirred for 3 hours. The resulting mixture was evaporated and the residue was triturated with CH₂Cl₂, followed by diethyl ether and n-pentane to remove the volatiles and DEAD impurities from previous step. The crude was dried in a rotary evaporator at 50° C. and the title compound was obtained as white solid (0.91 g, 62.93%). LC-MS-2: Rt=0.10 min, m/z: 169.30 [M+H−Cl]; ¹H NMR (300 MHz, DMSO-d₆) δ=7.95-8.19 (m, 3H), 7.08 (s, 2H), 3.33-3.47 (m, 2H), 2.67-2.80 (m, 2H), 1.44-1.63 (m, 4H).

### Intermediate D: 1-(3-aminopropyl)-1H-pyrrole-2,5-dione

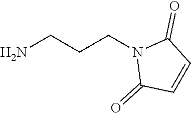 [SMILES: NCCCN1C(=O)C=CC1=O]

### Step 1: T-butyl (3-(2,5-dioxo-2,5-dihydro-1H-pyrrol-1-yl)propyl)carbamate

To a solution of 1H-pyrrole-2,5-dione (1.22 g, 12.56 mmol) and triphenylphosphine (3.23 g, 12.33 mmol) in THF (50 mL) was added tert-butyl (3-hydroxypropyl)carbamate (1.951 ml, 11.41 mmol), followed by DIAD (2.66 ml, 13.70 mmol). After stirring for 16 hours at 20° C., the reaction mixture was concentrated under reduced pressure and the residue was purified by flash chromatography eluting with AcOEt/Heptane 0:1 to 6:4 to give the title compound (4.5 g, 9.81 mmol, 86% yield). LC-MS-8: Rt=0.65 mins; MS m/z [M+H−Boc]⁺ 155.

### Step 2: 1-(3-Aminopropyl)-1H-pyrrole-2,5-dione

A solution of tert-butyl (3-(2,5-dioxo-2,5-dihydro-1H-pyrrol-1-yl)propyl)carbamate (4.5 g, 9.81 mmol) in HCl/Dioxane 4N (50 mL) was stirred for 11 hours at 20° C. The reaction mixture was concentrated under reduced pressure to give the title compound (3.74 g). It was used in the next step without further purification. LC-MS-8: Rt=0.14 mins; MS m/z [M+H]⁺ 155.

### Intermediate E: 3-(benzyloxy)-2,6-bis(bromomethyl)pyridine

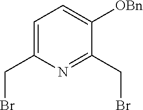 [SMILES: BrCc1ccc(OCc2ccccc2)c(CBr)n1]

Step 1: (3-(Benzyloxy)pyridine-2,6-diyl)dimethanol was prepared according to U.S. Pat. No. 8,268,810 B2, page 12, example 4. ¹H NMR (300 MHz, DMSO-d₆) δ=7.27-7.37 (m, 4H), 7.18-7.27 (m, 1H), 5.18 (t, J=1.0 Hz, 2H), 4.50 (s, 2H), 4.49 (s, 2H).

To a solution of (3-(benzyloxy)pyridine-2,6-diyl)dimethanol (1.5 g, 6.12 mmol) in CHCl₃ (15 mL) at 0° C. was added dropwise PBr₃ (3.48 g, 12.85 mmol) over a period of 15 mins at 0° C. The reaction mixture was slowly warmed to RT and stirred at RT for 6 hours. The solvent was evaporated and the residue was quenched with sat. NaHCO₃, extracted with CH₂Cl₂ and washed with brine solution. The combined organic layers were dried over anhydrous Na₂SO₄, filtered and evaporated. The crude compound was purified through flash chromatography on silicagel eluting with hexane and EtOAc (25%) afforded the title compound (240 mg, 15%) as a white solid. LC-MS-2: Rt=1.831 min, m/z: 371.80 [M+H]; ¹HNMR (300 MHz, CDCl₃) δ=7.18-7.49 (m, 5H), 5.18 (s, 2H), 4.66 (s, 2H), 4.52 (s, 2H).

### Intermediate F: Tetra-tert-butyl 2,2′-((oxybis(ethane-2,1-diyl))bis(azanediyl))(2R,2′R)-disuccinate

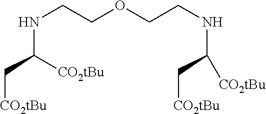 [SMILES: CC(C)(C)OC(=O)C[C@@H](NCCOCCN[C@H](CC(=O)OC(C)(C)C)C(=O)OC(C)(C)C)C(=O)OC(C)(C)C]

### Step 1: Di-tert-butyl ((2-nitrophenyl)sulfonyl)-D-aspartate

To a solution of di-tert-butyl D-aspartate (HCl) (10 g, 35.5 mmol) and NaHCO₃ (8.94 g, 106 mmol) in tetrahydrofuran (300 mL) was added a solution of 2-nitrobenzenesulfonyl chloride (9.44 g, 42.6 mmol) in tetrahydrofuran (300 mL) at 0° C. After stirring overnight at RT, the reaction mixture was concentrated under reduced pressure. The residue was dissolved in a saturated NaHCO₃ aqueous solution (200 mL) and extracted with EtOAc (3×100 mL). The combined organic layers were dried (Phase Separator) and concentrated under reduced pressure. The crude product was purified by flash chromatography on silica gel and eluted with cyclohexane/EtOAc 70:30 to afford the title compound as a yellow oil (14.5 g, 85%). LC-MS-14: Rt=1.03 mins; MS m/z [M−H]-429.4.

### Step 2: Tetra-tert-butyl 2,2′-((oxybis(ethane-2,1-diyl))bis(((2nitrophenyl)sulfonyl)azanediyl))(2R,2′R)-disuccinate

To a solution of 2,2′-oxybis(ethan-1-ol) (1.25 mL, 13.19 mmol) in THF (300 mL) was added at RT under argon di-tert-butyl ((2-nitrophenyl)sulfonyl)-D-aspartate (14.20 g, 33.0 mmol) and PPh₃ (10.38 g, 39.6 mmol). The reaction mixture was cooled to 0° C. and a solution of di-tert-butyl azodicarboxylate (9.11 g, 39.6 mmol) in THF (50 mL) was added dropwise over 10 min. After stirring at RT for 16 hours, the reaction mixture was filtered through a Celite bed and the filtrate was concentrated under reduced pressure. The crude product purified by flash chromatography and eluted with cyclohexane/EtOAc 60:40 to afford the title compound as a yellow oil (8.3 g, 60.8%)

### Step 3: Tetra-tert-butyl 2,2′-((oxybis(ethane-2,1-diyl))bis(azanediyl))(2R,2′R)-disuccinate

To a solution of tetra-tert-butyl 2,2′-((oxybis(ethane-2,1-diyl))bis(((2-nitrophenyl)sulfonyl)azanediyl))(2R,2′R)-disuccinate (8.3 g, 8.91 mmol) in DMF (dry) (107 mL) (12 ml/mmol SM), K₂CO₃ (9.86 g, 71.3 mmol) was added, followed by the dropwise addition of thiophenol (4.59 mL, 44.6 mmol) (1-2.5 equiv./Ns group). After stirring at RT for 16 hours, the reaction mixture was concentrated under reduced pressure. The residue was extracted with EtOAc/water, the combined organic layers washed with a saturated solution of NaHCO₃ and brine, dried (Phase Separator) and concentrated under reduced pressure. The crude product was purified by flash chromatography eluting with CH₂Cl₂/MeOH/NH₄OH 92:8:0.8 afforded the title compound as a yellow oil (3.9 g). LC-MS-8: Rt=1.02 mins; MS m/z [M+H]⁺ 561.7.

### Intermediate G: Tert-butyl (3-((2,6-bis(bromomethyl)pyridin-4-yl)oxy)propyl)carbamate

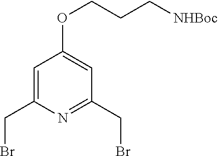 [SMILES: CC(C)(C)OC(=O)NCCCOc1cc(CBr)nc(CBr)c1]

### Step 1: Tert-butyl (3-((2,6-bis(hydroxymethyl)pyridin-4-yl)oxy)propyl)carbamate

Tert-butyl (3-((2,6-bis(hydroxymethyl)pyridin-4-yl)oxy)propyl)carbamate was prepared following the procedure described in Org. Biomol. Chem., 2012, 10, 9183-9190, page 9185, compound 4. LC-MS-8: Rt=0.31 mins; MS m/z [M+H]⁺ 313.4.

### Step 2: Tert-butyl (3-((2,6-bis(bromomethyl)pyridin-4-yl)oxy)propyl)carbamate

To a solution of tert-butyl (3-((2,6-bis(hydroxymethyl)pyridin-4-yl)oxy)propyl)carbamate (3.0 g, 9.60 mmol) in acetonitrile (150 mL) was added at 0° C. under argon PPh₃ (7.56 g, 28.8 mmol) and then CBr₄ (9.55 g, 28.8 mmol) dropwise. After stirring at 0° C. for 1 hour, the reaction mixture was concentrated under reduced pressure. The crude product was purified by flash chromatography eluting with cyclohexane/EtOAc 80:20 afforded the title compound as a colorless oil (3.1 g). LC-MS-8: Rt=1.09 mins; MS m/z [M+H]⁺ 439.3.

### Intermediate H: 2-(4-(Methylamino)butyl)isoindoline-1,3-dione

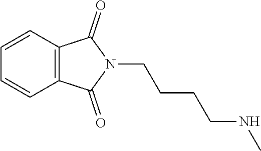 [SMILES: CNCCCCN1C(=O)c2ccccc2C1=O]

2-(4-(Methylamino)butyl)isoindoline-1,3-dione was prepared according to the procedure of WO 2017/060167, page 202, intermediate 17.2.
LC-MS-3: Rt=0.37 min, [M+H−HCl] 233.00; ¹H NMR (400 MHz, DMSO-d6) δ=8.75-8.91 (m, 2H), 7.79-7.93 (m, 4H), 3.54-3.62 (m, 17H), 3.28-3.42 (m, 2H), 2.75-2.95 (m, 2H), 1.62 (br d, J=3.6 Hz, 4H).

### Example 1: 2,2′,2″-(4-(4-(2,5-Dioxo-2,5-dihydro-1H-pyrrol-1-yl)butyl)-3,6,9-triaza-1(2,6)-pyridinacyclodecaphane-3,6,9-triyl)triacetic acid

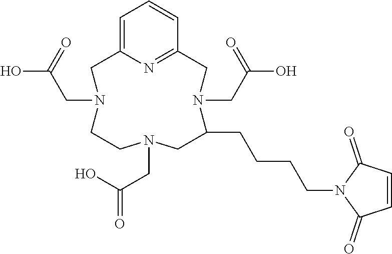 [SMILES: O=C(O)CN1CCN(CC(=O)O)CC(CCCCN2C(=O)C=CC2=O)N(CC(=O)O)Cc2cccc(n2)C1]

### Step 1: Benzyl tert-butyl (6-hydroxyhexane-1,5-diyl)dicarbamate

To a mixture of tert-butyl (5-amino-6-hydroxyhexyl)carbamate (28.0 g, 120 mmol, 1.00 eq) in THF (224 mL) and CH₃CN (224 mL) was added K₂CO₃ (33.3 g, 241 mmol, 2.00 eq), followed by CbzCl (41.1 g, 241 mmol, 34.2 mL, 2.00 eq) at 20° C. After stirring at 25° C. for 2 hours, the reaction mixture was diluted with water (1.00 L) and extracted with ethyl acetate (1.00 L×2). The combined organic layers were separated, washed with brine (0.80 L), dried over Na₂SO₄, filtered and concentrated under reduced pressure. The product was purified by flash chromatography on silica using a gradient petroleum ether/ethyl acetate=2/1 to 1/1 afforded the title compound as colorless oil (34.0 g, 76.9% yield). ¹H NMR (400 MHz, DMSO-d6) δ 7.44-7.26 (m, 5H), 6.93 (br d, J=8.40 Hz, 1H), 6.74 (br t, J=4.80 Hz, 1H), 5.00 (s, 2H), 4.59 (t, J=5.60 Hz, 1H), 3.39 (br d, J=4.00 Hz, 1H), 3.27-3.19 (m, 1H), 2.87 (q, J=6.40 Hz, 2H), 1.57-1.45 (m, 1H), 1.41-1.12 (m, 14H).

### Step 2: 2-(((Benzyloxy)carbonyl)amino)-6-((tert-butoxycarbonyl)amino)hexyl methanesulfonate

To a mixture of benzyl tert-butyl (6-hydroxyhexane-1,5-diyl)dicarbamate (34.0 g, 92.7 mmol, 1.00 eq), DMAP (283 mg, 2.32 mmol, 0.02 eq) and Et₃N (18.7 g, 185 mmol, 25.8 mL, 2.00 eq) in CH₂Cl₂ (680 mL) was added MsCl (11.6 g, 102 mmol, 7.90 mL, 1.10 eq) at 5-10° C. The mixture was warmed to 25° C. slowly and stirred for 2 hours. The reaction mixture was added to ice water (500 mL), and the aqueous layer was extracted with dichloromethane (500 mL×2). The combined organic layers were washed with saturated NH₄Cl (500 mL×2), brine (500 mL), dried over Na₂SO₄, filtered and concentrated under reduced pressure. The product was used in the next step without further purification. The title compound (40.0 g, 89.0 mmol, 96.9% yield) was obtained as white solid.

### Step 3: Benzyl tert-butyl (6-((2-aminoethyl)amino)hexane-1,5-diyl)dicarbamate

A solution of 2-(((benzyloxy)carbonyl)amino)-6-((tert-butoxycarbonyl)amino)hexyl methanesulfonate (40.0 g, 89.9 mmol, 1.00 eq) in ethane-1,2-diamine (215 g, 3.59 mol, 240 mL, 39.8 eq) was heated to 50° C. and stirred for 2 hours. The reaction mixture was diluted with ice water (2.00 L) and the aqueous layer was extracted with ethyl acetate (1.50 L). The combined organic layers were washed with brine (1.00 L), dried over Na₂SO₄, filtered and concentrated under reduced pressure. The product was used in the next step without further purification. The title compound (30.0 g, crude) was obtained as colorless oil.

### Step 4: Tert-butyl (5-amino-6-((2-aminoethyl)amino)hexyl)carbamate

To a solution of benzyl tert-butyl (6-((2-aminoethyl)amino)hexane-1,5-diyl)dicarbamate (30.0 g, 73.4 mmol, 1.00 eq) in MeOH (300 mL) was added Pd/C (3.00 g, 73.4 mmol, 10.0% purity, 1.00 eq) under N₂ atmosphere. The resulting mixture was purged and degassed with H₂ (50 Psi×3). After stirring at 25° C. for 3 hours, the reaction mixture was filtered, the cake was washed with ethyl acetate (200 mL×2), and the filtrate was concentrated under reduced pressure. The title compound (20.0 g, crude) was obtained as colorless oil and used in the next step without further purification.

### Step 5: Tert-butyl (6-((2-nitro-N-(2-((2-nitrophenyl)sulfonamido)ethyl)phenyl)sulfonamido)-5-((2-nitrophenyl)sulfonamido)hexyl)carbamate

To a solution of tert-butyl (5-amino-6-((2-aminoethyl)amino)hexyl)carbamate (20.0 g, 72.8 mmol, 1.00 eq) in THF (200 mL) was added NaHCO₃ (21.4 g, 255 mmol, 9.92 mL, 3.50 eq) under N₂ atmosphere, followed by a solution of 2-nitrobenzenesulfonyl chloride (56.5 g, 255 mmol, 3.50 eq) in THF (100 mL) at 0-5° C. After stirring at 50° C. for 60 hours, the reaction mixture was added to water (300 mL) and extracted with ethyl acetate (300 mL×2). The combined organic layers were separated, washed with brine (300 mL), dried over Na₂SO₄, filtered and concentrated under reduced pressure. The product was purified by flash chromatography on silica gel eluting with petroleum ether/ethyl acetate=1:1 to 1:2, afforded the title compound (32.0 g, 34.0 mmol, 46.7% yield, 88.3% purity) as a white solid. LC-MS-5: Rt=0.96 mins; MS m/z [M+H]⁺ 730.1; ¹H NMR (400 MHz, DMSO-d6) δ 8.21-8.11 (m, 1H), 8.06-7.92 (m, 6H), 7.91-7.74 (m, 6H), 6.60 (br t, J=5.20 Hz, 1H), 3.48-3.27 (m, 5H), 3.20 (br dd, J=6.40, 14.4 Hz, 1H), 3.06-2.90 (m, 2H), 2.69-2.56 (m, 2H), 1.35 (s, 9H), 1.13-0.95 (m, 3H), 0.82 (br s, 1H).

### Step 6: Tert-butyl (4-(3,6,9-tris((2-nitrophenyl)sulfonyl)-3,6,9-triaza-1(2,6)-pyridinacyclodecaphane-4-yl)butyl)carbamate

To a solution of tert-butyl (6-((2-nitro-N-(2-((2-nitrophenyl)sulfonamido)ethyl)phenyl)sulfonamido)-5-((2-nitrophenyl)sulfonamido)hexyl)carbamate (21.0 g, 25.3 mmol, 1.00 eq) in DMA (630 mL) was added K₂CO₃ (13.9 g, 101 mmol, 4.00 eq). After stirring at 100° C. for 10 mins, a solution of 2,6-bis(bromomethyl)pyridine (10.0 g, 37.9 mmol, 1.50 eq) in DMA (210 mL) was added drop-wise into the mixture over 20 mins. The resulting mixture was stirred at 100° C. for 4 hours. After completion, the reaction mixture was added to water (600 mL) and extracted with ethyl acetate (600 mL). The organic layers were separated, washed with brine (600 mL×3), dried over Na₂SO₄, filtered and concentrated under reduced pressure. The product was purified by column chromatography on silica gel eluting with petroleum ether/ethyl acetate=2:1 to 1:1. The title compound (20.0 g) was obtained as yellow solid. LC-MS-5: Rt=1.0 mins; MS m/z [M+H]⁺ 933.3; ¹H NMR (400 MHz, DMSO-d6) δ 8.08-7.79 (m, 14H), 7.38 (br s, 1H), 7.27 (d, J=7.60 Hz, 1H), 6.66 (br t, J=5.20 Hz, 1H), 4.74-4.50 (m, 2H), 4.43 (br s, 1H), 4.15 (br s, 1H), 3.96 (br s, 1H), 3.79 (br s, 1H), 3.57 (br d, J=11.6 Hz, 2H), 3.42 (br d, J=6.00 Hz, 1H), 2.73-2.61 (m, 2H), 1.52 (br s, 1H), 1.36 (s, 10H), 1.13 (br s, 2H), 0.89 (br s, 2H).

### Step 7: Tert-butyl (4-(3,6,9-triaza-1(2,6)-pyridinacyclodecaphane-4-yl)butyl)carbamate

To a mixture of tert-butyl (4-(3,6,9-tris((2-nitrophenyl)sulfonyl)-3,6,9-triaza-1(2,6)-pyridinacyclodecaphane-4-yl)butyl)carbamate (16.0 g, 17.1 mmol, 1.00 eq) in THF (192 mL) was added K₂CO₃ (23.7 g, 171 mmol, 10.0 eq) and thiophenol (7.56 g, 68.6 mmol, 7.00 mL, 4.00 eq). After stirring at 50° C. for 12 hours, the reaction mixture was engaged to the next step.

### Step 8: Tri-tert-butyl 2,2′,2″-(4-(4-((tert-butoxycarbonyl)amino)butyl)-3,6,9-triaza-1(2,6)-pyridinacyclodecaphane-3,6,9-triyl)triacetate

To a mixture of tert-butyl (4-(3,6,9-triaza-1(2,6)-pyridinacyclodecaphane-4-yl)butyl)carbamate (6.40 g, 16.9 mmol, 1.00 eq) was added THF (128 mL), K₂CO₃ (7.03 g, 50.8 mmol, 3.00 eq) and NaI (127 mg, 847 umol, 0.05 eq), followed by tert-butyl 2-bromoacetate (9.92 g, 50.8 mmol, 7.52 mL, 3.00 eq). After stirring at 50° C. for 6 hours, the reaction mixture was added to water (300 mL) and extracted with ethyl acetate (300 mL×2). The organic layers were separated, washed with brine (300 mL), dried over Na₂SO₄, filtered and concentrated under reduced pressure to afford a yellow oil residue (28.0 g). The residue was purified by preparative HPLC: Phenomenex luna C18 (250*70 mm, 10 um); mobile phase: [water (0.1% TFA)-ACN]; B %: 38%-68%, 19 mins. The title compound was obtained as yellow solid (7.00 g). It was dissolved in 30% ACN-H₂O and purified by preparative HPLC: Luna 25×200 mm, C18 10 mm, 110 A+Gemin 150×30 mm, C18 5 mm, 110 A. The title compound (3.54 g, 4.05 mmol, 23.8% yield, 95.4% purity, TFA) was obtained as yellow solid. LC-MS-5: Rt=0.80 mins; MS m/z [M+H]⁺ 720.4; ¹H NMR (400 MHz, CH₃OD-d) δ 7.85-7.66 (m, 1H), 7.21 (br d, J=7.60 Hz, 2H), 4.48-4.33 (m, 1H), 4.30-4.12 (m, 2H), 4.11-3.95 (m, 2H), 3.88-3.47 (m, 6H), 3.36 (br s, 1H), 3.35 (s, 1H), 3.28-2.98 (m, 5H), 1.77 (br d, J=8.00 Hz, 1H), 1.68-1.27 (m, 35H), 1.21 (br s, 7H).

### Step 9: Tri-tert-butyl 2,2′,2″-(4-(4-aminobutyl)-3,6,9-triaza-1(2,6)-pyridinacyclodecaphane-3,6,9-triyl)triacetate

A solution of tri-tert-butyl 2,2′,2″-(4-(4-((tert-butoxycarbonyl)amino)butyl)-3,6,9-triaza-1(2,6)-pyridinacyclodecaphane-3,6,9-triyl)triacetate (300 mg, 0.42 mmol) in CH₂Cl₂ (5 mL) was treated with TFA (0.32 mL, 4.17 mmol). After stirring at RT for 4 hours, the reaction mixture was cooled to 0° C. and extracted with CH₂Cl₂ (×3). The combined organic layers were washed with a saturated solution of NaHCO₃ and brine, dried and concentrated under reduced pressure to provide the title compound as a yellow oil (295 mg). The product was used in next step directly. LC-MS-8: Rt=0.72 mins; MS m/z [M+H]⁺ 620.9.

### Step 10: Tri-tert-butyl 2,2′,2″-(4-(4-(2,5-dioxo-2,5-dihydro-1H-pyrrol-1-yl)butyl)-3,6,9-triaza-1(2,6)-pyridinacyclodecaphane-3,6,9-triyl)triacetate

To a solution of tri-tert-butyl 2,2′,2″-(4-(4-aminobutyl)-3,6,9-triaza-1(2,6)-pyridinacyclodecaphane-3,6,9-triyl)triacetate (295 mg, 0.48 mmol) in CH₃CN (4 mL) was added at 0° C. NaHCO₃ (200 mg, 2.38 mmol) and methyl 2,5-dioxo-2,5-dihydro-1H-pyrrole-1-carboxylate (89 mg, 0.57 mmol). After stirring at RT for 2 hours, water (1 mL) was added and the resulting mixture was stirred at RT for 16 hours. The reaction mixture was extracted with EtOAc, and the combined organic layers were washed with H₂O, dried and concentrated under reduced pressure. The crude product was purified by flash chromatography on silica gel, eluting with CH₂Cl₂/MeOH/NH₄OH (90:10:1) and afforded the title compound as a colorless oil (147 mg, 43.2%). LC-MS-8: Rt=1.02 mins; MS m/z [M+H]⁺ 701.9.

### Step 11: 2,2′,2″-(4-(4-(2,5-Dioxo-2,5-dihydro-1H-pyrrol-1-yl)butyl)-3,6,9-triaza-1(2,6)-pyridinacyclodecaphane-3,6,9-triyl)triacetic acid

A solution of tri-tert-butyl 2,2′,2″-(4-(4-(2,5-dioxo-2,5-dihydro-1H-pyrrol-1-yl)butyl)-3,6,9-triaza-1(2,6)-pyridinacyclodecaphane-3,6,9-triyl)triacetate (147 mg, 0.210 mmol) in CH₂Cl₂ (2 mL) was treated with TFA (2 mL). After stirring at RT for 4 hours, the reaction mixture was concentrated under reduced pressure and purified by preparative HPLC (RP-HPLC-2) to provide 46 mg of title compound as a white powder. LC-MS-9: Rt=0.53 mins; MS m/z [M+H]⁺ 532.4; ¹H NMR (600 MHz, DMSO-d6) δ 12.49-12.59 (m, 1H), 7.73 (br s, 1H), 7.16-7.23 (m, 2H), 7.00 (br s, 2H), 4.34 (br d, J=18.06 Hz, 1H), 4.19 (br s, 1H), 3.94-4.06 (m, 3H), 3.64 (br d, J=8.30 Hz, 3H), 3.65 (br d, J=8.41 Hz, 3H), 3.58-3.59 (m, 3H), 3.22-3.33 (m, 5H), 3.18 (br d, J=13.58 Hz, 2H), 3.06 (br s, 1H), 1.71 (br s, 1H), 1.52 (br s, 2H), 1.45 (br s, 1H), 1.34 (br s, 1H), 1.23 (br s, 2H).

### Example 2: 2,2′,2″-(14-((4-(2,5-Dioxo-2,5-dihydro-1H-pyrrol-1-yl)butyl)carbamoyl)-3,6,9-triaza-1(2,6)-pyridinacyclodecaphane-3,6,9-triyl)triacetic acid

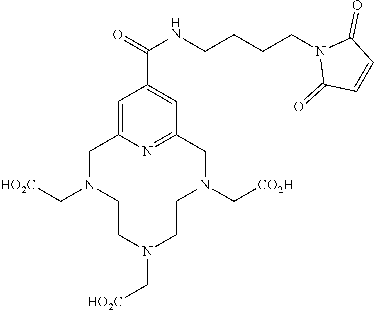 [SMILES: O=C(O)CN1CCN(CC(=O)O)Cc2cc(C(=O)NCCCCN3C(=O)C=CC3=O)cc(n2)CN(CC(=O)O)CC1]

### Step 1: Tri-tert-butyl 2,2′,2″-(14-(methoxycarbonyl)-3,6,9-triaza-1(2,6)-pyridinacyclodecaphane-3,6,9-triyl)triacetate

A stirred suspension of intermediate A (2.0 g, 4.49 mmol) and anhydrous Na₂CO₃ (2.38 g, 22.45 mmol) in dry CH₃CN (2176.46 mL, 0.002 M solution) was heated to reflux for 1.5 hours and then cooled to RT. Methyl 2,6-bis(bromomethyl)isonicotinate (1.441 g, 4.491 mmol), prepared according to the procedure from Bioorg. Med. Chem. Lett. 22 (2012) 2684-2688, page 2686, compound 9, was added in one portion and then heated to reflux for 48 hours. After completion, the reaction mixture was cooled to RT and filtered through a short celite bed. The filtrate was concentrated under reduced pressure. Another 1 g batch of intermediate A was taken for macrocyclization and the combined batches were purified through gravity column using neutral alumina eluting with CH₂Cl₂ followed by 2% MeOH in CH₂Cl₂ to afford the title compound as a mixture of sodium complex of the title compound and free ligand as white gum (2.6 g, 63.70%). LC-MS-4: Rt=2.32 min, m/z: 608.10 [M+H]⁺; ¹H NMR (400 MHz, CDCl₃) δ=7.66 (s, 1H), 7.63 (s, 1H), 4.12-4.20 (m, 2H), 3.81-3.95 (m, 6H), 3.41-3.55 (m, 6H), 3.19 (s, 2H), 2.51-2.57 (m, 2H), 2.20-2.30 (m, 1H), 1.80-1.90 (m, 2H), 1.45-1.49 (m, 27H).

### Removal of Na Complex:

To the sodium salt of tri-tert-butyl 2,2′,2″-(14-(methoxycarbonyl)-3,6,9-triaza-1(2,6)-pyridinacyclodecaphane-3,6,9-triyl)triacetate (2.6 g, 4.29 mmol) in CH₂Cl₂ (458.8 mL) was added a saturated solution of mono sodium salt of EDTA in water (152.92 mL) at RT and stirred at RT for 16 hours. The organic layer was separated, dried over anhydrous Na₂SO₄, filtered and evaporated to obtain tri-tert-butyl 2,2′,2″-(14-(methoxycarbonyl)-3,6,9-triaza-1(2,6)-pyridinacyclodecaphane-3,6,9-triyl)triacetate free of sodium complex (2.4 g, 88.93%). LC-MS-2: Rt=1.60 min. m/z: 607.00 [M+H]; ¹H NMR (400 MHz, CDCl₃) δ=(C-09189-023-45C) 7.65 (s, 2H), 4.17 (s, 4H), 3.87-3.98 (m, 3H), 3.30-3.49 (m, 5H), 1.37-1.54 (m, 36H)

### Step 2: 3,6,9-Tris(2-(tert-butoxy)-2-oxoethyl)-3,6,9-triaza-1(2,6)-pyridinacyclodecaphane-14-carboxylic acid

To a stirred solution of tri-tert-butyl 2,2′,2″-(1⁴-(methoxycarbonyl)-3,6,9-triaza-1(2,6)-pyridinacyclodecaphane-3,6,9-triyl)triacetate (0.50 g, 0.82 mmol) in methanol (21.4 mL) and water (10.7 mL), was added anhydrous Na₂CO₃ (86 mg, 0.81 mmol). After stirring at RT for 4 hours, a second equivalent of anhydrous Na₂CO₃ was added (86 mg, 0.825 mmol) and the resulting mixture was stirred for 18 hours at RT. After completion, the solvent was evaporated and the pH of the aqueous layer was adjusted the pH 6 using 0.1 N HCl. The mixture was lyophilized to obtain the crude 3,6,9-tris(2-(tert-butoxy)-2-oxoethyl)-3,6,9-triaza-1(2,6)-pyridinacyclodecaphane-14-carboxylic acid as an off white fluffy solid (450 mg, crude). LC-MS-1: Rt=1.406 min, m/z: 593.30 [M+H]; HPLC; ¹H NMR (400 MHz, CDCl₃) δ=7.44-7.50 (m, 2H), 3.98-4.08 (m, 4H), 3.60-3.72 (m, 4H), 3.32-3.41 (m, 4H), 3.10-3.21 (m, 6H), 1.51 (s, 9H), 1.44 (s, 18H).

### Step 3: Tri-tert-butyl 2,2′,2″-(14-((4-(2,5-dioxo-2,5-dihydro-1H-pyrrol-1-yl)butyl)carbamoyl)-3,6,9-triaza-1(2,6)-pyridinacyclodecaphane-3,6,9-triyl)triacetate

To a solution of 3,6,9-tris(2-(tert-butoxy)-2-oxoethyl)-3,6,9-triaza-1(2,6)-pyridinacyclodecaphane-14-carboxylic acid (0.450 g, 0.760 mmol) in CH₂Cl₂ (10.0 mL) was added HATU (0.37 g, 0.99 mmol) at 0° C. The reaction mixture was stirred for 10 min and the HCl salt of intermediate C (155 mg, 0.76 mmol) was added followed by NEt₃ (0.22 g, 2.17 mmol) at the same temperature and then slowly warmed to RT and stirred at RT for 16 hours. After completion, the reaction mixture was quenched with ice water and the aqueous layer was extracted with ethylacetate. The combined organic layers were dried with anhydrous Na₂SO₄, filtered and evaporated. The crude product was purified through flash chromatography on silica gel eluting with an ammonia solution (4%) in methanol and CH₂Cl₂ to obtain the title compound (90 mg, 17.73%) as an off white solid. LC-MS-2: Rt=1.50 min, m/z: 743.00 [M+H]; ¹H NMR (400 MHz, CDCl₃) δ=7.57 (s, 2H), 6.66 (s, 2H), 3.94-4.04 (m, 2H), 3.67-3.77 (m, 2H), 3.53-3.59 (m, 3H), 3.37-3.49 (m, 6H), 3.20-3.30 (m, 2H), 3.00 (s, 2H), 2.79 (s, 2H), 2.54-2.67 (m, 2H), 2.38-2.50 (m, 2H), 2.03-2.13 (m, 2H), 1.80-1.93 (m, 2H), 1.37-1.72 (m, 27H).

### Step 4: 2,2′,2″-(14-((4-(2,5-Dioxo-2,5-dihydro-1H-pyrrol-1-yl)butyl)carbamoyl)-3,6,9-triaza-1(2,6)-pyridinacyclodecaphane-3,6,9-triyl)triacetic acid

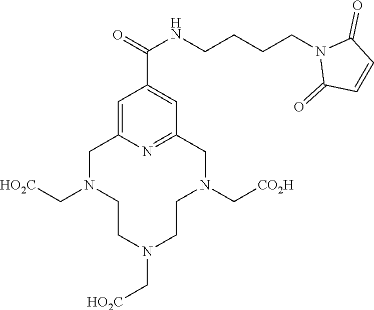 [SMILES: O=C(O)CN1CCN(CC(=O)O)Cc2cc(C(=O)NCCCCN3C(=O)C=CC3=O)cc(n2)CN(CC(=O)O)CC1]

A solution of tri-tert-butyl 2,2′,2″-(14-((4-(2,5-dioxo-2,5-dihydro-1H-pyrrol-1-yl)butyl)carbamoyl)-3,6,9-triaza-1(2,6)-pyridinacyclodecaphane-3,6,9-triyl)triacetate (88 mg, 0.12 mmol) in CH₂Cl₂ (2 mL) was treated with TFA (2 mL) at RT and stirred for 4 hours. The reaction mixture was concentrated under reduced pressure and the residue was purified by preparative HPLC (RP-HPLC-2) to provide the title compound as a white solid (41 mg, 54.8% yield). LC-MS-9: Rt=0.47 mins; MS m/z [M+H]⁺ 575.5. ¹H NMR (600 MHz, DMSO-d6) δ=11.86-13.01 (m, 1H), 8.68-8.71 (m, 1H), 7.55 (s, 3H), 7.01 (s, 2H), 4.12 (s, 3H), 4.03 (br s, 5H), 3.55 (br s, 11H), 3.42-3.49 (m, 18H), 3.21-3.27 (m, 12H), 1.77 (quin, J=7.15 Hz, 1H).

### Example 3: 2-(3,9-Bis(carboxymethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinic acid

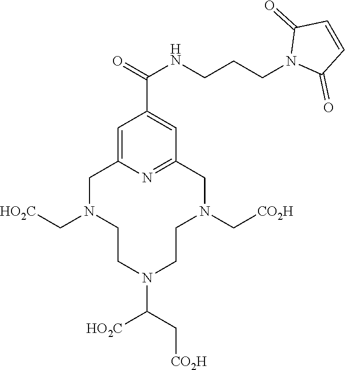 [SMILES: O=C(O)CC(C(=O)O)N1CCN(CC(=O)O)Cc2cc(C(=O)NCCCN3C(=O)C=CC3=O)cc(n2)CN(CC(=O)O)CC1]

### Step 1: Di-tert-butyl 2-(3,9-bis(2-(tert-butoxy)-2-oxoethyl)-1⁴-(methoxycarbonyl)-3,6,9-triaza-1(2,6)-pyridinacyclodecaphane-6-yl)succinate

A suspension of di-tert-butyl N,N-bis(2-((2-(tert-butoxy)-2-oxoethyl)amino)ethyl)aspartate (0.72 g, 1.29 mmol) and anhydrous Na₂CO₃ (0.682 g, 10.71 mmol) in dry CH₃CN (261.9 mL) was heated to reflux for 1.5 hours and then cooled to RT. Methyl 2,6-bis(bromomethyl)isonicotinate, prepared according to the procedure from Bioorg. Med. Chem. Lett. 22 (2012) 2684-2688, page 2686, compound 9 (0.413 g, 1.287 mmol) was added in one portion and then heated to reflux for 24 h. After completion, the reaction mixture was cooled to RT and filtered through a short celite plug. The solvents were evaporated under reduced pressure to obtain a mixture of sodium and free complex of the title compound as beige solid (0.75 g, crude). The residue was treated with saturated mono sodium EDTA solution in CH₂Cl₂ and stirred for 16 hours. The organic layer was separated, dried over anhydrous Na₂SO₄, filtered and evaporated under reduced pressure to obtain 650 mg of crude title compound as a free complex. LC-MS-1: Rt=1.467 min, m/z: 721.30 [M+H]; ¹H NMR (400 MHz, CDCl₃) δ=7.64 (s, 2H), 4.16-4.39 (m, 4H), 3.95 (s, 4H), 3.45-3.67 (m, 5H), 3.16-3.42 (m, 6H), 2.72-2.99 (m, 3H), 1.36-1.59 (m, 36H)

### Step 2: 3,9-Bis(2-(tert-butoxy)-2-oxoethyl)-6-(1,4-di-tert-butoxy-1,4-dioxobutan-2-yl)-3,6,9-triaza-1(2,6)-pyridinacyclodecaphane-1⁴-carboxylic acid

To a solution of di-tert-butyl 2-(3,9-bis(2-(tert-butoxy)-2-oxoethyl)-1⁴-(methoxycarbonyl)-3,6,9-triaza-1(2,6)-pyridinacyclodecaphane-6-yl)succinate (0.75 g, 1.13 mmol) in methanol (20 mL) and water (10 mL) was added anhydrous Na₂CO₃ (0.104 g, 0.98 mmol). After completion, the solvent was evaporated under reduced pressure, the aqueous layer was adjusted to pH 6 using 0.1 N HCl, and lyophilized. The residue was purified by preparative HPLC (RP-HPLC-1) eluting with 0.1% formic acid in water and MeCN to afford the title compound as a formate salt and white colour solid (315 mg, 41.8%). LC-MS-1: Rt=1.60 min, m/z: 707.20 [M+H]; ¹H NMR (400 MHz, CDCl₃) δ=7.87 (s, 2H), 4.26 (s, 2H), 4.04-4.15 (m, 1H), 3.37-3.54 (m, 3H), 2.94-3.20 (m, 5H), 2.66-2.86 (m, 2H), 2.07-2.46 (m, 7H), 1.39-1.46 (m, 36H).

### Step 3: Di-tert-butyl 2-(3,9-bis(2-(tert-butoxy)-2-oxoethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinate

To a solution of 3,9-bis(2-(tert-butoxy)-2-oxoethyl)-6-(1,4-di-tert-butoxy-1,4-dioxobutan-2-yl)-3,6,9-triaza-1(2,6)-pyridinacyclodecaphane-1⁴-carboxylic acid (141 mg, 0.20 mmol) in dry CH₂Cl₂ (5 mL), was added at RT HATU (99 mg, 0.26 mmol). The reaction mixture was stirred for 20 min and a solution of intermediate D (26 mg, 0.127 mmol) in CH₂Cl₂ (5 mL) was added, followed by dropwise addition of NEt₃ (0.061 mL, 0.44 mmol). After stirring at RT for 1 hour, the pH was adjusted to pH 8-9 using a saturated solution of NaHCO₃ and the resulting mixture extracted with CH₂Cl₂. The combined organic layers were dried and concentrated under reduced pressure. The crude product was purified by flash chromatography eluting with CH₂Cl₂/MeOH/NH₄OH (95:5:0.5) provided the title compound as a colorless oil. LC-MS-8: Rt=1.04 mins; MS m/z [M+H]⁺ 843.9.

### Step 4: 2-(3,9-Bis(carboxymethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinic acid

A solution of di-tert-butyl 2-(3,9-bis(2-(tert-butoxy)-2-oxoethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinate (115 mg, 0.136 mmol) in CH₂Cl₂ (2 mL) was treated with TFA (2 mL). After stirring at RT for 18 hours, the reaction mixture was concentrated under reduced pressure and the residue was purified using by HPLC (RP-HPLC-3) to give the title compound as a white solid (43 mg, 23.5%). LC-MS-9: Rt=0.14 mins; MS m/z [M+H]⁺ 619.6; ¹H NMR (400 MHz, DMSO-d6) δ 11.96-12.89 (m, 1H), 8.69 (t, J=5.39 Hz, 1H), 7.52 (s, 6H), 7.17 (s, 1H), 7.08 (s, 1H), 7.01 (s, 7H), 4.46-4.56 (m, 3H), 4.07-4.24 (m, 16H), 3.56-3.59 (m, 15H), 3.41-3.65 (m, 39H), 3.11-3.31 (m, 21H), 2.95-3.12 (m, 15H), 1.69-1.83 (m, 6H).

### Example 4: 2,2′,2″-(1³-(3-(2,5-Dioxo-2,5-dihydro-1H-pyrrol-1-yl)propoxy)-3,6,9-triaza-1(2,6)-pyridinacyclodecaphane-3,6,9-triyl)triacetic acid

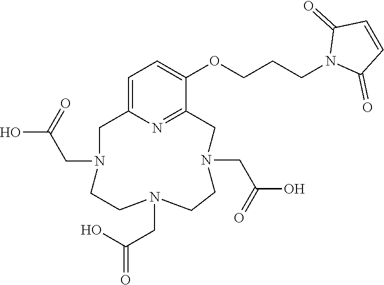 [SMILES: O=C(O)CN1CCN(CC(=O)O)Cc2ccc(OCCCN3C(=O)C=CC3=O)c(n2)CN(CC(=O)O)CC1]

### Step 1: Tri-tert-butyl 2,2′,2″-(1³-(benzyloxy)-3,6,9-triaza-1(2,6)-pyridinacyclodecaphane-3,6,9-triyl)triacetate

A suspension of di-tert-butyl 2,2′-((((2-(tert-butoxy)-2-oxoethyl)azanediyl)bis(ethane-2,1-diyl))bis(azanediyl))diacetate (0.25 g, 0.56 mmol) and anhydrous Na₂CO₃ (0.29 g, 2.81 mmol) in dry CH₃CN (272 mL) was heated to reflux for 1.5 hours and then cooled to RT. 3-(benzyloxy)-2,6-bis(bromomethyl)pyridine (0.21 g, 0.56 mmol) was added in one portion and then heated to reflux for 16 hours. After completion, the reaction mixture was cooled to RT, filtered through a short celite plug and the solvent was evaporated under reduced pressure. The resulting residue was absorbed onto isolute HMN and purified by chromatography eluting with DCM/MeOH:aqNH₃ (90:9:1). The solvents were evaporated under reduced pressure and the residue treated with saturated mono NaEDTA (14.75 mL, 58.82 w/v) in CH₂Cl₂ (44.11 mL, 176.47 w/v), and stirred at RT for 16 hours. The organic layers were separated, dried with anhydrous Na₂SO₄, filtered and the solvents evaporated to obtain the title compound as colorless gum (400 mg, 37%). LC-MS-2: Rt=1.66 min, m/z: 655.60 [M+H]

### Step 2: Tri-tert-butyl 2,2′,2″-(1³-hydroxy-3,6,9-triaza-1(2,6)-pyridinacyclodecaphane-3,6,9-triyl)triacetate

A solution of tri-tert-butyl 2,2′,2″-(13-(benzyloxy)-3,6,9-triaza-1(2,6)-pyridinacyclodecaphane-3,6,9-triyl)triacetate (0.3 g, 0.458 mmol) in ethanol (15 mL) was treated with wet Pd/C (0.150 g) at RT. The solution was placed under hydrogen using a hydrogen bladder and stirred at RT for 6 hours. After completion, the reaction mixture was filtered through a Celite bed and washed with methanol. The solvents were evaporated and the title compound (0.25 g, crude) was used to the next step without further purification. LC-MS-2: Rt=1.41 min, m/z: 565.50 [M+H].

### Step 3: Tri-tert-butyl 2,2′,2″-(1³-(3-((tert-butoxycarbonyl)amino)propoxy)-3,6,9-triaza-1(2,6)-pyridinacyclodecaphane-3,6,9-triyl)triacetate

To a solution of tri-tert-butyl 2,2′,2″-(13-hydroxy-3,6,9-triaza-1(2,6)-pyridinacyclodecaphane-3,6,9-triyl)triacetate (0.250 g, 0.443 mmol) in CH₃CN (2.5 mL) was added anhydrous K₂CO₃ (0.092 g, 0.664 mmol) followed by a solution of tert-butyl (3-bromopropyl)carbamate (0.126 g, 0.532 mmol) in CH₃CN (2.5 mL) at RT. The reaction mixture was heated to reflux for 16 hours and then cooled. The precipitate was filtered through a celite plug and the filtrate was evaporated. The crude mixture was absorbed onto isolute HMN and purified by chromatography eluting with CH₂Cl₂ and an ammonia solution in methanol as eluent (90:9:1, DCM:MeOH:7N NH₃ in MeOH) gave the title compound as a beige gum (170 mg, 53.19%). LC-MS-1: Rt=1.437 min, m/z: 722.25 [M+H]; ¹H NMR (400 MHz, CDCl₃) δ=7.21-7.30 (m, 3H), 7.16 (br d, J=8.3 Hz, 1H), 7.01-7.08 (m, 1H), 5.07 (t, J=1.0 Hz, 1H), 4.25-4.31 (m, 1H), 3.90-4.14 (m, 5H), 3.59-3.75 (m, 3H), 3.26-3.54 (m, 6H), 3.13 (s, 2H), 2.49-2.71 (m, 3H), 2.18-2.28 (m, 2H), 1.94-2.16 (m, 2H), 1.35-1.54 (m, 38H).

### Step 4: Tri-tert-butyl 2,2′,2″-(1³-(3-aminopropoxy)-3,6,9-triaza-1(2,6)-pyridinacyclodecaphane-3,6,9-triyl)triacetate

To a solution of tri-tert-butyl 2,2′,2″-(1³-(3-((tert-butoxycarbonyl)amino)propoxy)-3,6,9-triaza-1(2,6)-pyridinacyclodecaphane-3,6,9-triyl)triacetate (95 mg, 0.118 mmol) in CH₂Cl₂ (237 μl) was added TFA (45.6 μl, 0.59 mmol) at 0 to 5° C. After stirring at RT for 6 days, the reaction mixture was evaporated, dissolved with CH₂Cl₂ and evaporated again. The residue was purified by preparative HPLC (RP-HPLC-3). The solvent was evaporated and the aqueous layer was extracted with EtOAc and washed with Na₂CO₃ 0.1M. The combined organic layers were dried by passing through a phase separating cartridge and the solvents evaporated under reduced pressure to provide the title compound (25 mg white powder). LC-MS-10: Rt=0.68 mins; MS m/z [M+H]⁺ 622.5.

### Step 5: Tri-tert-butyl 2,2′,2″-(1³-(3-(2,5-dioxo-2,5-dihydro-1H-pyrrol-1-yl)propoxy)-3,6,9-triaza-1(2,6)-pyridinacyclodecaphane-3,6,9-triyl)triacetate

To a solution of tri-tert-butyl 2,2′,2″-(1³-(3-aminopropoxy)-3,6,9-triaza-1(2,6)-pyridinacyclodecaphane-3,6,9-triyl)triacetate (22 mg, 0.035 mmol) and Et₃N (9.81 μl, 0.071 mmol) in 0.9 mL of toluene was added dropwise a solution of maleic anhydride (3.85 mg, 0.039 mmol) in 0.6 mL of toluene at 0° C. The reaction mixture was stirred at 5-10° C. for 1 hour. The toluene was evaporated under reduced pressure. The residue was solved in CH₂Cl₂ (0.146 mL) and under argon was added pentafluorophenol (9.97 mg, 0.053 mmol) and DIC (0.017 mL, 0.106 mmol). The resulting solution was stirred at 30° C. for 20 hours. The reaction mixture was evaporated and the residue was partitioned between water and EtOAc. The organic layer was separated and water layer extracted with EtOAc (5 mL). The organic layers were combined, dried by passing through a phase separating cartridge and evaporated under reduced pressure to provide the title compound (29 mg) as a brown oil. LC-MS-11: Rt=4.59 mins; MS m/z [M+H]⁺ 702.4.

### Step 6: 2,2′,2″-(1³-(3-(2,5-Dioxo-2,5-dihydro-1H-pyrrol-1-yl)propoxy)-3,6,9-triaza-1(2,6)-pyridinacyclodecaphane-3,6,9-triyl)triacetic acid

To a solution of tri-tert-butyl 2,2′,2″-(1³-(3-(2,5-dioxo-2,5-dihydro-1H-pyrrol-1-yl)propoxy)-3,6,9-triaza-1(2,6)-pyridinacyclodecaphane-3,6,9-triyl)triacetate (26 mg, 0.028 mmol) in CH₂Cl₂ (93 μl) was added at 0-5° C. TFA (185 μl). After stirring at RT for 4 hours, the reaction mixture was evaporated. The residue was solved in CH₃CN-water 7:3 and purified by preparative HPLC (RP-HPLC-3) to provide the title compound as a white powder (4.5 mg). LC-MS-12: Rt=0.14 mins; MS m/z [M]⁺ 533.3. ¹H NMR (400 MHz, DMSO-d6) δ=8.11 (s, 1H), 7.58 (d, J=8.5 Hz, 1H), 7.46 (d, J=8.5 Hz, 1H), 6.97 (s, 2H), 4.63 (s, 4H), 4.19 (d, J=5.4 Hz, 4H), 4.07 (t, J=6.1 Hz, 2H), 3.78 (s, 2H), 3.61 (t, J=6.6 Hz, 2H), 3.52-3.44 (m, 4H), 3.26-3.04 (m, 4H), 2.00 (t, J=6.3 Hz, 2H).

### Example 5: (S)-2-(3,9-bis(carboxymethyl)-1³-(3-(2,5-dioxo-2,5-dihydro-1H-pyrrol-1-yl)propoxy)-3,6,9-triaza-1(2,6)-pyridinacyclodecaphane-6-yl)succinic acid

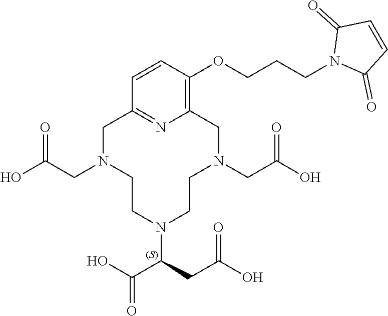

### Step 1: Di-tert-butyl (S)-2-(1³-(benzyloxy)-3,9-bis(2-(tert-butoxy)-2-oxoethyl)-3,6,9-triaza-1(2,6)-pyridinacyclodecaphane-6-yl)succinate

To a stirred suspension of di-tert-butyl N,N-bis(2-((2-(tert-butoxy)-2-oxoethyl)amino)ethyl)-L-aspartate (0.425 g, 0.760 mmol) and anhydrous Na₂CO₃ (0.402 g, 3.799 mmol) in dry CH₃CN (462 mL) was heated to reflux for 1.5 hours and then cooled to RT. To this solution 3-(benzyloxy)-2,6-bis(bromomethyl)pyridine (0.280 g, 0.760 mmol) was added in one portion and the resulting mixture was heated to reflux for 12 hours. After completion, the reaction mixture was cooled to RT, filtered through a short celite plug and the solvent was removed under reduced pressure. The crude was treated with a saturated mono NaEDTA (14.75 mL, 58.82 w/v) solution in CH₂Cl₂ (44.11 mL, 176.47 w/v) and stirred at RT for 16 hours. The organic layer was separated, dried with anhydrous Na₂SO₄, filtered and evaporated under reduced pressure. The residue was absorbed onto isolute HMN and purified by flash chromatography on silicagel eluting with CH₂Cl₂/MeOH:aqNH₃ (90:9:1) afforded the title compound as a colourless gum (400 mg, 59%). LC-MS-1: Rt=1.493 min, m/z: 769.25 [M+H]; ¹HNMR (CDCl₃, 300 MHz) δ=7.26-7.46 (m, 5H), 5.03-5.19 (m, 1H), 4.07-4.24 (m, 1H), 3.85-4.03 (m, 1H), 3.55-3.82 (m, 2H), 3.19-3.43 (m, 2H), 2.78-3.03 (m, 1H), 2.07-2.65 (m, 3H), 1.85-2.00 (m, 1H), 1.57-1.80 (m, 6H), 0.99-1.57 (m, 41H).

### Step 2: Di-tert-butyl (S)-2-(3,9-bis(2-(tert-butoxy)-2-oxoethyl)-1³-hydroxy-3,6,9-triaza-1(2,6)-pyridinacyclodecaphane-6-yl)succinate

Di-tert-butyl (S)-2-(3,9-bis(2-(tert-butoxy)-2-oxoethyl)-1³-hydroxy-3,6,9-triaza-1(2,6)-pyridinacyclodecaphane-6-yl)succinate was prepared by a method similar to that of Example 4, step 2, starting with di-tert-butyl (S)-2-(13-(benzyloxy)-3,9-bis(2-(tert-butoxy)-2-oxoethyl)-3,6,9-triaza-1(2,6)-pyridinacyclodecaphane-6-yl)succinate (0.38 g, 0.494 mmol) to afford the title compound (0.24 g, crude) as beige liquid. LC-MS-1: Rt=1.43 min, m/z: 677.25 [M−H].

### Step 3: Di-tert-butyl (S)-2-(3,9-bis(2-(tert-butoxy)-2-oxoethyl)-1³-(3-((tert-butoxycarbonyl)amino)propoxy)-3,6,9-triaza-1(2,6)-pyridinacyclodecaphane-6-yl)succinate

To a solution of di-tert-butyl (S)-2-(3,9-bis(2-(tert-butoxy)-2-oxoethyl)-1³-hydroxy-3,6,9-triaza-1(2,6)-pyridinacyclodecaphane-6-yl)succinate (0.240 g, 0.354 mmol) in CH₃CN (2.5 mL) was added anhydrous K₂CO₃ (0.073 g, 0.531 mmol) followed by a solution of tert-butyl (3-bromopropyl)carbamate (0.101 g, 0.425 mmol) in CH₃CN (2.5 mL) at RT. The reaction mixture was heated to reflux for 16 hours at 70° C. and then cooled. The precipitate was removed through a celite bed and the filtrate was evaporated. The residue was absorbed onto isolute HMN and purified by flash chromatography silicagel eluting with CH₂Cl₂ and ammonia solution (7N) in methanol as eluents (90:9:1, CH₂Cl₂:MeOH:7N NH₃ in MeOH) afforded the title compound as a beige gum (170 mg, 53.19%). LC-MS-2: Rt=1.66 min, m/z: 836.90 [M+H]; ¹HNMR (CDCl₃, 400 MHz) δ=7.35-7.33 (d, 1H), 7.22-7.18 (d, 1H), 5.18-5.12 (m, 2H), 4.31 (bs, 1H), 4.18-4.09 (m, 2H), 3.99-3.88 (m, 2H), 3.74-3.52 (m, 4H), 3.38-3.21 (m, 5H), 2.91-2.88 (m, 1H), 2.79-2.72 (m, 1H), 2.5-2.48 (m, 1H), 2.37-2.32 (m, 2H), 2.11-1.89 (m, 4H), 1.97-1.422 (m, 45).

### Step 4: Di-tert-butyl (S)-2-(1³-(3-aminopropoxy)-3,9-bis(2-(tert-butoxy)-2-oxoethyl)-3,6,9-triaza-1(2,6)-pyridinacyclodecaphane-6-yl)succinate

A solution of di-tert-butyl (S)-2-(3,9-bis(2-(tert-butoxy)-2-oxoethyl)-1³-(3-((tert-butoxycarbonyl)amino)propoxy)-3,6,9-triaza-1(2,6)-pyridinacyclodecaphane-6-yl)succinate (80 mg, 0.096 mmol) in CH₂Cl₂ (1 mL), was treated with TFA (0.037 mL, 0.478 mmol). The solution was stirred at 20° C. for 7 days. Two equivalents of TFA were added to the mixture and the solution was stirred for an additional 3 days at 20° C. The reaction mixture was treated with a saturated solution of NaHCO₃. The organic phase was separated, dried over MgSO₄ and concentrated under reduced pressure to give 87 mg of crude material. The crude was purified by ACCQ prep (Waters SunFire Prep C18 OBD 5 μm 19*50, Flow 20 mL/min, H₂O (0.1% TFA)/Acetonitrile 95/5=>50/50 during 10 min, RT 8.8-9.2 min). The product fractions were combined, evaporated under reduced pressure to remove the CH₃CN, then treated with a saturated solution of NaHCO₃, and extracted with AcOEt. The organic phase was dried over MgSO₄ and concentrated in vacuum to give the title compound (15 mg, 0.020 mmol, 21.09% yield). LC-MS-12: Rt=0.91 mins; MS m/z [M+H]⁺ 736.5.

### Step 5: Di-tert-butyl (S)-2-(3,9-bis(2-(tert-butoxy)-2-oxoethyl)-1³-(3-(2,5-dioxo-2,5-dihydro-1H-pyrrol-1-yl)propoxy)-3,6,9-triaza-1(2,6)-pyridinacyclodecaphane-6-yl)succinate

To a solution of di-tert-butyl (S)-2-(1³-(3-aminopropoxy)-3,9-bis(2-(tert-butoxy)-2-oxoethyl)-3,6,9-triaza-1(2,6)-pyridinacyclodecaphane-6-yl)succinate (15 mg, 0.020 mmol) in toluene (1 mL), was added NEt₃ (5.68 μl, 0.041 mmol), at 20° C. Then a solution of maleic anhydride (2.198 mg, 0.022 mmol) in toluene (0.5 mL) was added dropwise. After stirring for 1 hour, the reaction mixture was evaporated under reduced pressure. The residue was dissolved in CH₂Cl₂ (1 mL), and pentafluorophenol (5.63 mg, 0.031 mmol) and DIC (9.53 μl, 0.061 mmol) were added. The solution was stirred for 16 hours at 30° C. The reaction mixture was evaporated under reduced pressure. The residue was dissolved in AcOEt and washed with H₂O. The organic phase was isolated, dried over MgSO₄ and concentrated under reduced pressure to give the title compound (29 mg, 0.020 mmol, 96% yield). LC-MS-12: Rt=1.14 mins; MS m/z [M+H]⁺ 816.4.

### Step 6: (S)-2-(3,9-Bis(carboxymethyl)-1³-(3-(2,5-dioxo-2,5-dihydro-1H-pyrrol-1-yl)propoxy)-3,6,9-triaza-1(2,6)-pyridinacyclodecaphane-6-yl)succinic acid

To a solution of di-tert-butyl (S)-2-(3,9-bis(2-(tert-butoxy)-2-oxoethyl)-1³-(3-(2,5-dioxo-2,5-dihydro-1H-pyrrol-1-yl)propoxy)-3,6,9-triaza-1(2,6)-pyridinacyclodecaphane-6-yl)succinate (29 mg, 0.036 mmol) in CH₂Cl₂ (1 mL), was added TFA (2 mL). After stirring at 20° C. for 16 hours, the reaction mixture was concentrated in vacuum to give 87 mg of crude material. The crude was purified by HPLC (RP-HPLC-3). The product fractions were lyophilized to give the title compound (7 mg, 8.78 μmol, 24.71% yield). LC-MS-12: Rt=0.17 mins; MS m/z [M+H]⁺ 592.3. ¹HNMR (400 MHz, DMSO-d6) δ=7.38 (br d, J=8.22 Hz, 1H), 7.22 (br s, 1H), 6.99 (s, 2H), 4.38-4.50 (m, 1H), 4.05-4.14 (m, 2H), 4.01 (br s, 4H), 3.64 (br s, 2H), 3.59 (br t, J=6.05 Hz, 4H), 3.17 (br s, 4H), 2.93-3.09 (m, 4H), 1.92-2.01 (m, 2H), 1.00 (br d, J=6.09 Hz, 2H).

### Example 6: (2R,2′R)-2,2′-(14-((3-(2,5-Dioxo-2,5-dihydro-1H-pyrrol-1-yl)propyl)carbamoyl)-6-oxa-3,9-diaza-1(2,6)-pyridinacyclodecaphane-3,9-diyl)disuccinic acid

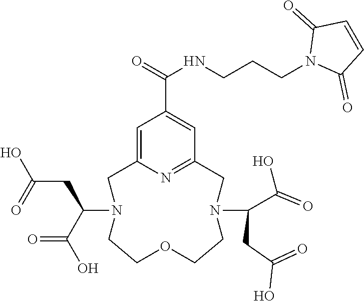 [SMILES: O=C(O)C[C@H](C(=O)O)N1CCOCCN([C@H](CC(=O)O)C(=O)O)Cc2cc(C(=O)NCCCN3C(=O)C=CC3=O)cc(n2)C1]

### Step 1: Tetra-tert-butyl 2,2′-(1⁴-(methoxycarbonyl)-6-oxa-3,9-diaza-1(2,6)-pyridinacyclodecaphane-3,9-diyl)(2R,2′R)-disuccinate

To a solution of intermediate F (0.2 g, 0.357 mmol) in CH₃CN (20 mL), DIPEA (0.311 mL, 1.783 mmol) and methyl 2,6-bis(bromomethyl)isonicotinate (0.115 g, 0.357 mmol), prepared according to the procedure from Bioorg. Med. Chem. Lett. 22 (2012) 2684-2688, page 2686, compound 9, were added. After stirring at 60° C. for 16 hours, the reaction mixture was treated with AcOEt, and washed with a saturated solution of NaHCO₃. The organic phase was dried over MgSO₄ and concentrated in vacuum to give 290 mg of crude material. The crude was purified by flash chromatography eluting with AcOEt/heptane afforded the title compound (150 mg, 0.204 mmol, 57.1% yield). LC-MS-12: Rt=1.16 mins; MS m/z [M+H]⁺ 722.8.

### Step 2: 3,9-Bis((R)-1,4-di-tert-butoxy-1,4-dioxobutan-2-yl)-6-oxa-3,9-diaza-1(2,6)-pyridinacyclodecaphane-1⁴-carboxylic acid

To a solution of tetra-tert-butyl 2,2′-(1⁴-(methoxycarbonyl)-6-oxa-3,9-diaza-1(2,6)-pyridinacyclodecaphane-3,9-diyl)(2R,2′R)-disuccinate (150 mg, 0.208 mmol) in MeOH (4 mL), NaOH (1M in H₂O) (0.208 mL, 0.208 mmol) was added. After stirring at 20° C. for 1 hour, the reaction mixture was treated with AcOEt, and washed with an NH₄Cl saturated solution. The organic phase was dried over MgSO₄ and concentrated in vacuum to give the title compound (150 mg, 0.201 mmol, 97% yield). LC-MS-12: Rt=1.18 mins; MS m/z [M+H]⁺ 708.5.

### Step 3: Tetra-tert-butyl 2,2′-(1⁴-((3-(2,5-dioxo-2,5-dihydro-1H-pyrrol-1-yl)propyl)carbamoyl)-6-oxa-3,9-diaza-1(2,6)-pyridinacyclodecaphane-3,9-diyl)(2R,2′R)-disuccinate

To a solution of 3,9-bis((R)-1,4-di-tert-butoxy-1,4-dioxobutan-2-yl)-6-oxa-3,9-diaza-1(2,6)-pyridinacyclodecaphane-14-carboxylic acid (50 mg, 0.071 mmol) in CH₂Cl₂ (3 mL) was added, HATU (34.9 mg, 0.092 mmol). After stirring 20 min at 20° C., a mixture of intermediate D (27.9 mg, 0.071 mmol) and TEA (0.022 ml, 0.155 mmol) in 2 mL CH₂Cl₂ was added dropwise. The reaction mixture was stirred for 1 hour at 20° C. After addition of CH₂Cl₂, the organic phase was separated, washed with a saturated solution of NaHCO₃, dried over MgSO₄ and concentrated under reduced pressure to give 80 mg of crude material. The crude was purified by preparative HPLC (RP-HPLC-3) afforded the title compound (15 mg, 0.017 mmol, 23.90% yield). LC-MS-12: Rt=1.18 mins; MS m/z [M+H]⁺ 845.3.

### Step 4: (2R,2′R)-2,2′-(1⁴-((3-(2,5-Dioxo-2,5-dihydro-1H-pyrrol-1-yl)propyl)carbamoyl)-6-oxa-3,9-diaza-1(2,6)-pyridinacyclodecaphane-3,9-diyl)disuccinic acid

A solution of tetra-tert-butyl 2,2′-(1⁴-((3-(2,5-dioxo-2,5-dihydro-1H-pyrrol-1-yl)propyl)carbamoyl)-6-oxa-3,9-diaza-1(2,6)-pyridinacyclodecaphane-3,9-diyl)(2R,2′R)-disuccinate (15 mg, 0.018 mmol) in CH₂Cl₂ (1 mL) was treated with TFA (1 mL). After stirring for 16 hours at 20° C., the reaction mixture was concentrated under reduced pressure to give 50 mg of crude material. The crude was purified by preparative HPLC (RP-HPLC-3) afforded the title compound (7 mg, 10.73 μmol, 60.4% yield). LC-MS-12: Rt=0.17 mins; MS m/z [M+H]⁺ 620; ¹HNMR (400 MHz, DMSO-d6) δ=12.31-13.48 (m, 4H), 9.07 (br s, 1H), 8.08 (br s, 2H), 7.02-7.05 (m, 2H), 4.40 (br s, 4H), 3.95 (dd, J=8.14, 6.05 Hz, 2H), 3.48 (br t, J=7.04 Hz, 3H), 3.27-3.30 (m, 7H), 2.99 (br s, 4H), 2.73 (br dd, J=16.23, 8.31 Hz, 2H), 2.61-2.65 (m, 2H), 1.77-1.83 (m, 2H).

### Example 7: (2R,2′R)-2,2′-(1⁴-(3-(2,5-Dioxo-2,5-dihydro-1H-pyrrol-1-yl)propoxy)-6-oxa-3,9-diaza-1(2,6)-pyridinacyclodecaphane-3,9-diyl)disuccinic acid

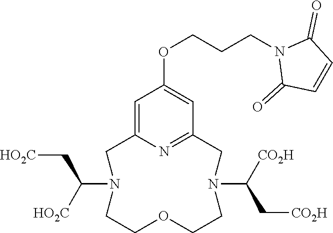 [SMILES: O=C(O)C[C@H](C(=O)O)N1CCOCCN([C@H](CC(=O)O)C(=O)O)Cc2cc(OCCCN3C(=O)C=CC3=O)cc(n2)C1]

### Step 1: Tetra-tert-butyl 2,2′-(1⁴-(3-((tert-butoxycarbonyl)amino)propoxy)-6-oxa-3,9-diaza-1(2,6)-pyridinacyclodecaphane-3,9-diyl)(2R,2′R)-disuccinate

To a solution of intermediate F (320 mg, 0.571 mmol) in anhydrous acetonitrile (10 mL) was added Na₂CO₃ (605 mg, 5.71 mmol) and the reaction mixture was refluxed for 1.5 hours. Intermediate G (250 mg, 0.571 mmol) was added in one portion and the reaction mixture was refluxed for 16 hours. Then the reaction mixture was cooled to RT, water was added and the resulting mixture was extracted with EtOAc. The combined organic layers were dried (Phase Separator) and concentrated under reduced pressure. The crude product was purified by flash chromatography eluting with CH₂Cl₂/MeOH/NH₄OH (90:10:1) afforded the title compound as a colorless oil (250 mg). LC-MS-8: Rt=1.48 mins; MS m/z [M+H]⁺ 838.0.

### Step 2: Tetra-tert-butyl 2,2′-(1⁴-(3-aminopropoxy)-6-oxa-3,9-diaza-1(2,6)-pyridinacyclodecaphane-3,9-diyl)(2R,2′R)-disuccinate

To a solution of tetra-tert-butyl 2,2′-(1⁴-(3-((tert-butoxycarbonyl)amino)propoxy)-6-oxa-3,9-diaza-1(2,6)-pyridinacyclodecaphane-3,9-diyl)(2R,2′R)-disuccinate (250 mg, 0.299 mmol) in CH₂Cl₂ (10 mL) was added at RT TFA (0.230 mL, 2.99 mmol). After stirring at RT for 4 hours, the reaction mixture was concentrated under reduced pressure. The residue was dissolved in EtOAc and the organic layer were washed with a saturated solution of NaHCO₃, brine, dried (Phase Separator) and concentrated under reduced pressure to afford the title compound (155 mg). It was used in the next step without further purification.

### Step 3: Tetra-tert-butyl 2,2′-(1⁴-(3-(2,5-dioxo-2,5-dihydro-1H-pyrrol-1-yl)propoxy)-6-oxa-3,9-diaza-1(2,6)-pyridinacyclodecaphane-3,9-diyl)(2R,2′R)-disuccinate

To a solution of tetra-tert-butyl 2,2′-(1⁴-(3-aminopropoxy)-6-oxa-3,9-diaza-1(2,6)-pyridinacyclodecaphane-3,9-diyl)(2R,2′R)-disuccinate (250 mg, 0.299 mmol) in CH₂Cl₂ (10 mL) was added at RT TFA (0.230 mL, 2.99 mmol). After stirring at RT for 4 hours, the reaction mixture was concentrated under reduced pressure and dried over MgSO₄. The residue was dissolved in EtOAc and the organic layer were washed with a saturated solution of NaHCO₃, brine, dried (Phase Separator) and concentrated under reduced pressure to afford the title compound as a yellow oil (155 mg).

### Step 4: (2R,2′R)-2,2′-(1⁴-(3-(2,5-Dioxo-2,5-dihydro-1H-pyrrol-1-yl)propoxy)-6-oxa-3,9-diaza-1(2,6)-pyridinacyclodecaphane-3,9-diyl)disuccinic acid

To a solution of tetra-tert-butyl 2,2′-(1⁴-(3-(2,5-dioxo-2,5-dihydro-1H-pyrrol-1-yl)propoxy)-6-oxa-3,9-diaza-1(2,6)-pyridinacyclodecaphane-3,9-diyl)(2R,2′R)-disuccinate (70 mg, 0.086 mmol) in CH₂Cl₂ (2 mL) was added at RT TFA (2 mL). After stirring at RT for 4 hours, the reaction mixture was concentrated under reduced pressure to afford the title compound as a yellow oil (60 mg). LC-MS-10: Rt=0.21 mins; MS m/z [M+H]⁺ 593.6; ¹HNMR (400 MHz, DMSO-d6) δ=7.34 (s, 2H), 7.03 (s, 2H), 4.41-4.12 (m, 6H), 3.91 (dd, J=8.2, 6.1 Hz, 2H), 3.58 (t, J=6.7 Hz, 4H), 2.95 (s, 4H), 2.70 (dd, J=16.2, 8.3 Hz, 2H), 2.60 (dd, J=16.3, 6.0 Hz, 2H), 2.03 (p, J=6.4 Hz, 2H).

### Example 8: 2,2′,2″-(1⁴-((4-(2,5-Dioxo-2,5-dihydro-1H-pyrrol-1-yl)butyl)(methyl)amino)-3,6,9-triaza-1(2,6)-pyridinacyclodecaphane-3,6,9-triyl)triacetic acid

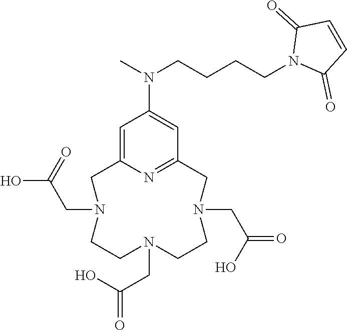 [SMILES: CN(CCCCN1C(=O)C=CC1=O)c1cc2nc(c1)CN(CC(=O)O)CCN(CC(=O)O)CCN(CC(=O)O)C2]

### Step 1: 2-(4-((2,6-Bis(hydroxymethyl)pyridin-4-yl)(methyl)amino)butyl)isoindoline-1,3-dione

To a solution of (4-chloropyridine-2,6-diyl)dimethanol (0.8 g, 4.61 mmol) prepared according to the procedure of Liebigs Ann. Chem. 1991, 987-998, page 992, compound 6, in n-butanol (12 mL) was added intermediate H (1.113 g, 4.15 mmol at RT into a 30 mL sealed tube. The suspension was heated to 165° C. using microwave radiation for 1 hour. After completion, the solvent was evaporated under reduced pressure and the residue was triturated with 70% ethylacetate in hexane and filtered. After drying, the title compound was isolated as a white solid (1.4 g, 82.23%). LC-MS-3: Rt=0.36 min, [M+H] 370.10; ¹H NMR (300 MHz, DMSO-d₆) δ=13.09 (s, 1H), 7.75-7.95 (m, 4H), 6.74-7.00 (m, 2H), 5.90-6.08 (m, 1H), 4.55-4.64 (m, 4H), 3.48-3.65 (m, 4H), 3.09-3.19 (m, 3H), 1.63 (br s, 4H).

### Step 2: 2-(4-((2,6-Bis(bromomethyl)pyridin-4-yl)(methyl)amino)butyl)isoindoline-1,3-dione

A solution of 2-(4-((2,6-bis(hydroxymethyl)pyridin-4-yl)(methyl)amino)butyl)isoindoline-1,3-dione (1.4 g, 3.789 mmol) in CHCl₃ (50 mL) was cooled to 0° C. PBr₃ (2.25 g, 8.34 mmol) was added dropwise over a period of 15 mins at 0° C. The reaction mixture was slowly warmed to RT and stirred for 16 hours at RT. The volatiles were evaporated and the residue was poured into crushed ice, quenched with sat. NaHCO₃ and extracted with CH₂Cl₂ twice. The combined organic phases were washed with brine, dried over anhydrous Na₂SO₄, filtered and concentrated under reduced pressure. The crude compound was purified by flash chromatography eluting with hexane and EtOAc afforded the title compound (1.87 g, 59%). LC-MS-3: Rt=0.33 min, m/z: 496.10 [M+H]; ¹HNMR (300 MHz, CDCl₃) δ=7.80-7.91 (m, 2H), 7.66-7.79 (m, 2H), 6.53 (s, 2H), 4.43 (s, 4H), 3.73 (t, J=1.0 Hz, 2H), 3.40 (t, J=1.0 Hz, 2H), 2.98 (s, 3H), 2.04 (s, 1H), 1.53-1.82 (m, 4H).

### Step 3: Tri-tert-butyl 2,2′,2″-(1⁴-((4-(1,3-dioxoisoindolin-2-yl)butyl)(methyl)amino)-3,6,9-triaza-1(2,6)-pyridinacyclodecaphane-3,6,9-triyl)triacetate

A stirred suspension of di-tert-butyl 2,2′-((((2-(tert-butoxy)-2-oxoethyl)azanediyl) bis(ethane-2,1-diyl))bis(azanediyl))diacetate (1.00 g, 2.244 mmol) and anhydrous Na₂CO₃ (1.188 g, 11.22 mmol) in anhydrous CH₃CN (1088.3 mL) was heated to reflux for 1.5 hours and cooled to RT. 2-(4-((2,6-Bis(bromomethyl)pyridin-4-yl)(methyl)amino)butyl)isoindoline-1,3-dione (1.112 g, 2.244 mmol) was added in one portion at RT and then heated to reflux for 16 hours. After completion, the reaction mixture was cooled to RT, filtered through short celite plug and concentrated under reduced pressure. The crude was treated with a saturated mono Na EDTA solution (14.75 mL, 58.82 w/v) in CH₂Cl₂ (44.11 mL, 176.47 w/v) and stirred at RT for 16 hours. The organic layer was separated, dried with anhydrous Na₂SO₄, filtered and concentrated under reduced pressure. The residue was absorbed onto isolute HMN and purified by flash chromatography (24 g, redisep silicagel) eluting with CH₂Cl₂/MeOH:aqNH₃ (90:9:1) afforded the title compound as a beige gum (620 mg, 35%). LC-MS-3: Rt=0.34 min, m/z: 779.60 [M+H]; ¹HNMR (CDCl₃, 300 MHz) δ=7.80-7.91 (m, 2H), 7.67-7.77 (m, 2H), 6.74 (s, 2H), 4.17 (s, 4H), 3.74 (t, J=6.7 Hz, 2H), 3.60 (t, J=1.0 Hz, 2H), 3.48 (s, 4H), 3.38 (br s, 2H), 3.20 (s, 3H), 2.98 (t, J=1.0 Hz, 4H), 2.84-2.93 (m, 4H), 1.75-1.84 (m, 2H), 1.57-1.75 (m, 2H), 1.46 (d, J=1.0 Hz, 27H).

### Step 4: Tri-tert-butyl 2,2′,2″-(1⁴-((4-aminobutyl)(methyl)amino)-3,6,9-triaza-1(2,6)-pyridinacyclodecaphane-3,6,9-triyl)triacetate

To a solution of tri-tert-butyl 2,2′,2″-(1⁴-((4-(1,3-dioxoisoindolin-2-yl)butyl)(methyl)amino)-3,6,9-triaza-1(2,6)-pyridinacyclodecaphane-3,6,9-triyl)triacetate (250 mg, 0.321 mmol) in CH₂Cl₂ (5349 μl) and MeOH (5349 μl) was added hydrazine hydrate (360 μl, 4.81 mmol). The solution was stirred at 20° C. for 16 hours. The precipitate was filtered off, washed with CH₂Cl₂ and the filtrate evaporated under reduced pressure.

The residue was partitioned between Na₂CO₃ 1M and EtOAc. The aqueous layer was extracted, separated and the aqueous layer extracted again with 1×EtOAc. The organic layers were combined, washed with water and brine. The organic extract was dried by passing through a phase separating cartridge and the volatiles were removed under reduced pressure to afford the title compound as a yellow oil (200 mg). LC-MS-12: Rt=0.80 mins; MS m/z [M+H]⁺ 649.6.

### Step 5: Tri-tert-butyl 2,2′,2″-(1⁴-((4-(2,5-dioxo-2,5-dihydro-1H-pyrrol-1-yl)butyl)(methyl)amino)-3,6,9-triaza-1(2,6)-pyridinacyclodecaphane-3,6,9-triyl)triacetate

To a solution of tri-tert-butyl 2,2′,2″-(1⁴-((4-aminobutyl)(methyl)amino)-3,6,9-triaza-1(2,6)-pyridinacyclodecaphane-3,6,9-triyl)triacetate (200 mg, 308 μmol) and Et₃N (85.9 μL, 616 μmol) in toluene (12 mL) was added maleic anhydride (33.2 mg, 339 μmol) at 0° C. The reaction mixture was stirred at 5-10° C. for 1 hour. The toluene was evaporated and the residue dried under reduced pressure to afford the title compound (265 mg) as a pale yellow resin. The crude was solved in 1,2-dichloroethane (1.2 mL) and under argon was added pentafluorophenol (85.1 mg, 462 μmol) and DIC (117 mg, 145 μL, 925 μmol). The resulting mixture was stirred at 20° C. for 20 hours. The reaction mixture was evaporated and the residue was partitioned between Na₂CO₃ 1M and EtOAc. The organic layer was separated and water layer extracted again with EtOAc (1×15 ml). The organic layers were combined, dried by passing through a phase separating cartridge and volatiles were removed under reduced pressure. The crude (240 mg) was purified by preparative HPLC (RP-HPLC-3) to afford the title compound as a white foam (45 mg). LC-MS-12: Rt=1.04 mins; MS m/z [M+H]⁺ 729.6.

### Step 6: 2,2′,2″-(1⁴-((4-(2,5-Dioxo-2,5-dihydro-1H-pyrrol-1-yl)butyl)(methyl)amino)-3,6,9-triaza-1(2,6)-pyridinacyclodecaphane-3,6,9-triyl)triacetic acid

To a solution of tri-tert-butyl 2,2′,2″-(1⁴-((4-(2,5-dioxo-2,5-dihydro-1H-pyrrol-1-yl)butyl)(methyl)amino)-3,6,9-triaza-1(2,6)-pyridinacyclodecaphane-3,6,9-triyl)triacetate (40 mg, 98% Wt, 1 Eq, 54 μmol) in water (44.4 μL) was added TFA (400 μL) at 0-5° C. The reaction mixture was stirred at RT for 17 hours. The solvent was evaporated, the residue solved in water (2 mL) and purified by preparative HPLC (RP-HPLC-3) to afford the title compound as a white powder (23 mg). LC-MS-12: Rt=0.15 mins; MS m/z [M+H]⁺ 561.3; ¹H NMR (400 MHz, DMSO) δ 7.05 (s, 1H), 6.99 (s, 2H), 6.95 (s, 1H), 4.23 (s, 2H), 4.14 (s, 4H), 3.69 (s, 4H), 3.55 (s, 2H), 3.43 (d, J=6.3 Hz, 2H), 3.21 (s, 3H), 3.13 (s, 3H), 2.93 (d, J=23.4 Hz, 4H), 1.54 (s, 4H).

### Example 9: (S)-2-(3,9-Bis(carboxymethyl)-1⁴-((4-(2,5-dioxo-2,5-dihydro-1H-pyrrol-1-yl)butyl)(methyl)amino)-3,6,9-triaza-1(2,6)-pyridinacyclodecaphane-6-yl)succinic acid

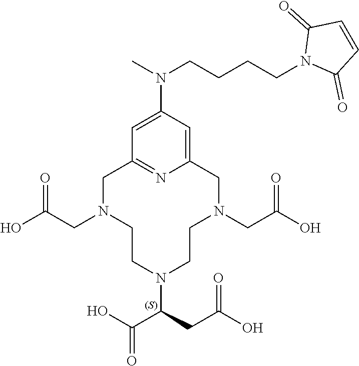

### Step 1: Di-tert-butyl (S)-2-(3,9-bis(2-(tert-butoxy)-2-oxoethyl)-1⁴-((4-(1,3-dioxoisoindolin-2-yl)butyl)(methyl)amino)-3,6,9-triaza-1(2,6)-pyridinacyclodecaphane-6-yl)succinate

To a stirred suspension of intermediate B (0.750 g, 1.340 mmol) in anhydrous CH₃CN, was added anhydrous Na₂CO₃ (0.709 g, 6.699 mmol) (544.115 mL). The resulting suspension was heated to reflux for 1.5 hours and then cooled to RT. 2-(4-((2,6-bis(bromomethyl)pyridin-4-yl)(methyl)amino)butyl)isoindoline-1,3-dione (example 8, step 2) (0.664 g, 1.340 mmol) was added in one portion at RT and then heated to reflux for 16 hours. After completion, the reaction mixture was cooled to RT and then filtered through a short celite plug and the volatiles were removed. The crude was treated with saturated mono NaEDTA solution (14.75 mL, 58.82 w/v) in CH₂Cl₂ (44.11 mL, 176.47 w/v) and stirred at RT for 16 hours. The organic layer was separated, dried with anhydrous Na₂SO₄, filtered and evaporated under reduced pressure. The residue was absorbed onto Isolute HMN and purified by flash chromatography (24 g, redisep silicagel) eluting using DCM/MeOH:aqNH₃ (90:9:1) afforded the title compound as a beige gum (400 mg, 36%). LC-MS-3: Rt=1.63 min, m/z: 893.80 [M+H]; ¹HNMR (CDCl₃, 300 MHz) δ=7.80-7.90 (m, 2H), 7.68-7.77 (m, 2H), 6.87 (s, 2H), 4.24 (s, 4H), 3.87-3.97 (m, 1H), 3.74 (t, J=1.0 Hz, 2H), 3.63 (t, J=1.0 Hz, 2H), 3.45-3.53 (m, 6H), 3.22 (s, 3H), 2.89 (br s, 4H), 2.61 (br s, 4H), 1.58-1.89 (m, 8H), 1.34-1.54 (m, 36H).

### Step 2: Di-tert-butyl (S)-2-(1⁴-((4-aminobutyl)(methyl)amino)-3,9-bis(2-(tert-butoxy)-2-oxoethyl)-3,6,9-triaza-1(2,6)-pyridinacyclodecaphane-6-yl)succinate

To a solution of di-tert-butyl (S)-2-(3,9-bis(2-(tert-butoxy)-2-oxoethyl)-1⁴-((4-(1,3-dioxoisoindolin-2-yl)butyl)(methyl)amino)-3,6,9-triaza-1(2,6)-pyridinacyclodecaphane-6-yl)succinate (250 mg, 0.277 mmol) in CH₂Cl₂ (4619 μl) and MeOH (4619 μl), hydrazine hydrate (311 μl, 4.16 mmol) was added. The solution was stirred at 20° C. for 1 hour. The precipitate was filtered off, washed with CH₂Cl₂ and the filtrate evaporated. The residue was partitioned between Na₂CO₃ 1M and EtOAc. The aqueous layer was extracted, separated and extracted again with 1×EtOAc. The organic layers were combined, washed with water and brine, dried by passing through a phase separating cartridge and the volatiles were removed under reduced pressure to afford the title compound (180 mg) as a yellow oil. LC-MS-12: Rt=0.9 mins; MS m/z [M+H]⁺ 763.8.

### Step 3: Di-tert-butyl (S)-2-(3,9-bis(2-(tert-butoxy)-2-oxoethyl)-1⁴-((4-(2,5-dioxo-2,5-dihydro-1H-pyrrol-1-yl)butyl)(methyl)amino)-3,6,9-triaza-1(2,6)-pyridinacyclodecaphane-6-yl)succinate

To a solution of di-tert-butyl (S)-2-(1⁴-((4-aminobutyl)(methyl)amino)-3,9-bis(2-(tert-butoxy)-2-oxoethyl)-3,6,9-triaza-1(2,6)-pyridinacyclodecaphane-6-yl)succinate (180 mg, 219 μmol) and Et₃N (61.2 μL, 439 μmol) in toluene (10 mL) was added maleic anhydride (24.9 mg, 241 μmol) at 0° C. The reaction mixture was stirred at 5-10° C. for 1 hour. The toluene was evaporated under vacuum (220 mg crude). The crude was solved in 1,2-dichloroethane (1 mL) and under argon were added pentafluorophenol (60.6 mg, 329 μmol) and DIC (103 μL, 658 μmol). The resulting mixture was stirred at 20° C. for 17 hours. The solvent was evaporated and the residue was partitioned between Na₂CO₃ 1M and EtOAc. The organic layer was separated and the aqueous water layer extracted again with 1×15 ml EtOAc. The organic layers were combined, dried by passing through a phase separating cartridge and the volatiles were removed under reduced pressure. The residue was purified by preparative HPLC to afford the title compound (45 mg) as a white solid. LC-MS-12: Rt=1.2 mins; MS m/z [M+H]⁺ 843.5.

### Step 4: (S)-2-(3,9-Bis(carboxymethyl)-1⁴-((4-(2,5-dioxo-2,5-dihydro-1H-pyrrol-1-yl)butyl)(methyl)amino)-3,6,9-triaza-1(2,6)-pyridinacyclodecaphane-6-yl)succinic acid

To a solution of di-tert-butyl (S)-2-(3,9-bis(2-(tert-butoxy)-2-oxoethyl)-1⁴-((4-(2,5-dioxo-2,5-dihydro-1H-pyrrol-1-yl)butyl)(methyl)amino)-3,6,9-triaza-1(2,6)-pyridinacyclodecaphane-6-yl)succinate (48 mg, 99% Wt, 1 Eq, 56 μmol) in water (44.4 μL) was added TFA (400 μL) at 0-5° C. The reaction mixture was stirred at RT for 17 hours. The reaction mixture was evaporated and the residue solved with 2 mL water and purified by preparative HPLC (RP-HPLC-3) to afford the title compound as a white powder (24 mg). LC-MS-12: Rt=0.16 mins; MS m/z [M+H]⁺ 619.3; ¹H NMR (400 MHz, DMSO) δ 6.99 (s, 2H), 6.94 (s, 2H), 4.54 (t, J=6.3 Hz, 1H), 4.20 (s, 4H), 3.81-3.65 (m, 4H), 3.55 (d, J=6.9 Hz, 2H), 3.43 (q, J=4.7 Hz, 2H), 3.12 (s, 8H), 2.97 (d, J=6.3 Hz, 5H), 1.54 (s, 4H).

### Example 10: (2R,2′R)-2,2′-(1⁴-(3-(2,5-dioxo-2,5-dihydro-1H-pyrrol-1-yl)propoxy)-3,7-diaza-1,5(2,6)-dipyridinacyclooctaphane-3,7-diyl)disuccinic acid

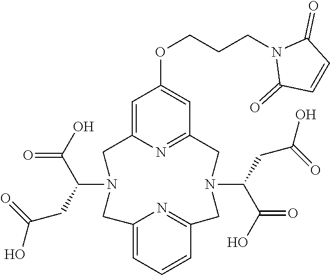 [SMILES: O=C(O)C[C@H](C(=O)O)N1Cc2cccc(n2)CN([C@H](CC(=O)O)C(=O)O)Cc2cc(OCCCN3C(=O)C=CC3=O)cc(n2)C1]

### Step 1: Tetra-tert-butyl 2,2′-(((4-(3-((tert-butoxycarbonyl)amino)propoxy)pyridine-2,6-diyl)bis(methylene))bis(azanediyl))(2R,2′R)-disuccinate

To a solution of di-tert-butyl D-aspartate (354 mg, 1.255 mmol) dissolved in 5 mL CH₃CN, DIPEA (0.498 mL, 2.85 mmol) was added and intermediate G (250 mg, 0.571 mmol) in 5 mL CH₃CN was added dropwise. After stirring at 60° C. for 2 days, the precipitate was filtered off and washed with CH₃CN. The filtrate was concentrated under reduced pressure to give 490 mg of crude material. The crude was purified by flash chromatography eluting using Heptane/AcOEt (+1% TEA) (40:60) afforded the title compound (125 mg, 0.161 mmol, 28.3% yield). LC-MS-12: Rt=1.33 mins; MS m/z [M+H]⁺ 767.3.

### Step 2: Tetra-tert-butyl 2,2′-(1⁴-(3-((tert-butoxycarbonyl)amino)propoxy)-3,7-diaza-1(2,6)-pyridina-5(1,3)-benzenacyclooctaphane-3,7-diyl)(2R,2′R)-disuccinate

To a solution of tetra-tert-butyl 2,2′-(((4-(3-((tert-butoxycarbonyl)amino)propoxy)pyridine-2,6-diyl)bis(methylene))bis(azanediyl))(2R,2′R)-disuccinate (125 mg, 0.163 mmol) in CH₃CN (5 mL), was added DIPEA (0.142 mL, 0.815 mmol) and 2,6-bis(bromomethyl)pyridine (43.2 mg, 0.163 mmol). After stirring 16 hours at 60° C., and 3 days at 20° C., the reaction mixture was treated with AcOEt, and washed with a saturated solution of NaHCO₃. The organic phase was separated, dried over MgSO₄ and concentrated under reduced pressure to give 140 mg of crude product. The crude was purified by flash chromatography eluting using AcOEt (+1% TEA)/Heptane (50:50) afforded the title compound (80 mg, 0.090 mmol, 55.3% yield). LC-MS-12: Rt=1.26 mins; MS m/z [M+H]⁺ 871.5.

### Step 3: Tetra-tert-butyl 2,2′-(1⁴-(3-aminopropoxy)-3,7-diaza-1(2,6)-pyridina-5(1,3)-benzenacyclooctaphane-3,7-diyl)(2R,2′R)-disuccinate

To a solution of tetra-tert-butyl 2,2′-(1⁴-(3-((tert-butoxycarbonyl)amino)propoxy)-3,7-diaza-1(2,6)-pyridina-5(1,3)-benzenacyclooctaphane-3,7-diyl)(2R,2′R)-disuccinate (80 mg, 0.092 mmol) in CH₂Cl₂ (3 mL), TFA (0.071 mL, 0.919 mmol) was added. After stirring at 20° C. for 16 hours, TFA (0.071 mL, 0.919 mmol) was added and the resulting mixture was stirred at 20° C. for 4 hours. The reaction mixture was concentrated under reduced pressure to give 124 mg of title compound (124 mg, 0.069 mmol, 75% yield) containing 25% of title compound without 1-tBu ester. LC-MS-12: Rt=0.87 mins; MS m/z [M+H]⁺ 770.3.

### Step 4: Tetra-tert-butyl 2,2′-(1⁴-(3-(2,5-dioxo-2,5-dihydro-1H-pyrrol-1-yl)propoxy)-3,7-diaza-1,5(2,6)-dipyridinacyclooctaphane-3,7-diyl)(2R,2′R)-disuccinate

To a solution of tetra-tert-butyl 2,2′-(1⁴-(3-aminopropoxy)-3,7-diaza-1(2,6)-pyridina-5(1,3)-benzenacyclooctaphane-3,7-diyl)(2R,2′R)-disuccinate (124 mg, 0.083 mmol) in toluene (3 mL), TEA (0.081 mL, 0.583 mmol) was added at 20° C., followed by dropwise addition of a solution of maleic anhydride (8.98 mg, 0.092 mmol) in toluene (0.5 mL). After stirring for 1 h, the reaction mixture was concentrated under reduced pressure. The residue was dissolved in CH₂Cl₂ (3 mL), and pentafluorophenol (22.99 mg, 0.125 mmol) and DIC (0.039 mL, 0.250 mmol) were added. After stirring for 16 hours at 30° C., the reaction mixture was concentrated under reduced pressure. The residue was dissolved in CH₂Cl₂ and washed with H₂O. The organic phase was dried over MgSO₄ and concentrated under reduced pressure to give 135 mg of crude material. The crude was purified by preparative HPLC (RP-HPLC-3) to provide the title compound (38 mg, 0.040 mmol, 48.3% yield) and di-tert-butyl (R)-2-(7-((R)-4-(tert-butoxy)-1,4-dioxo-1-(perfluorophenoxy)butan-2-yl)-14-(3-(2,5-dioxo-2,5-dihydro-1H-pyrrol-1-yl)propoxy)-3,7-diaza-1,5(2,6)-dipyridinacyclooctaphane-3-yl)succinate (28 mg, 0.026 mmol, 31.5% yield). LC-MS-12: Rt=1.13 mins; MS m/z [M+H]⁺ 851.3.

### Step 5: (2R,2′R)-2,2′-(1⁴-(3-(2,5-Dioxo-2,5-dihydro-1H-pyrrol-1-yl)propoxy)-3,7-diaza-1(2,6)-pyridina-5(1,3)-benzenacyclooctaphane-3,7-diyl)disuccinic acid

To a solution of tetra-tert-butyl 2,2′-(1⁴-(3-(2,5-dioxo-2,5-dihydro-1H-pyrrol-1-yl)propoxy)-3,7-diaza-1,5(2,6)-dipyridinacyclooctaphane-3,7-diyl)(2R,2′R)-disuccinate (38 mg, 0.045 mmol) in water (0.021 mL) was added TFA (0.4 mL). After stirring at 20° C. for 3 hours, the reaction mixture was concentrated under reduced pressure to give 34 mg of crude material. The crude was purified by preparative HPLC (RP-HPLC-3) afforded the title compound (16 mg, 0.025 mmol, 56.6% yield). LC-MS-13: Rt=0.61 mins; MS m/z [M+H]⁺ 626.4; ¹H NMR (400 MHz, DMSO-d6) δ 7.90 (br s, 1H), 7.38 (br s, 2H), 7.32 (br s, 2H), 7.08 (s, 2H), 4.34-4.57 (m, 8H), 4.31 (br s, 2H), 3.79-3.92 (m, 2H), 3.66 (t, J=6.68 Hz, 2H), 2.60-2.66 (m, 2H), 2.31 (br s, 2H), 2.09 (br t, J=6.20 Hz, 2H).

### Example 11: (S)-2-(9-((R)-1,2-Dicarboxyethyl)-1⁴-(3-(2,5-dioxo-2,5-dihydro-1H-pyrrol-1-yl)propoxy)-6-oxa-3,9-diaza-1(2,6)-pyridinacyclodecaphane-3-yl)succinic acid

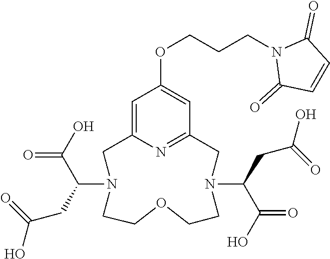 [SMILES: O=C(O)C[C@H](C(=O)O)N1CCOCCN([C@@H](CC(=O)O)C(=O)O)Cc2cc(OCCCN3C(=O)C=CC3=O)cc(n2)C1]

Following the procedure described for Example 7, (S)-2-(9-((R)-1,2-dicarboxyethyl)-1⁴-(3-(2,5-dioxo-2,5-dihydro-1H-pyrrol-1-yl)propoxy)-6-oxa-3,9-diaza-1(2,6)-pyridinacyclodecaphane-3-yl)succinic acid was prepared.

LC-MS-13: Rt=0.58 mins; MS m/z [M+H]⁺ 593.2; ¹H NMR (400 MHz, DMSO-d6) δ 7.33 (s, 2H), 7.00 (s, 2H), 4.27 (d, J=3.4 Hz, 6H), 3.89 (t, J=7.2 Hz, 2H), 3.58 (t, J=6.6 Hz, 2H), 3.43-3.30 (m, 4H), 3.02 (d, J=14.2 Hz, 2H), 2.94-2.84 (m, 2H), 2.70 (dd, J=16.4, 8.2 Hz, 2H), 2.55 (dd, J=16.4, 6.3 Hz, 2H), 2.03 (p, J=6.3 Hz, 2H).

### Example 12: (S)-2-(3,9-bis(carboxymethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinic acid

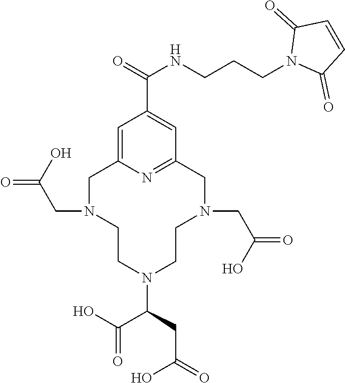 [SMILES: O=C(O)C[C@@H](C(=O)O)N1CCN(CC(=O)O)Cc2cc(C(=O)NCCCN3C(=O)C=CC3=O)cc(n2)CN(CC(=O)O)CC1]

### Step 1: Di-tert-butyl (S)-2-(3,9-bis(2-(tert-butoxy)-2-oxoethyl)-1⁴-(methoxycarbonyl)-3,6,9-triaza-1(2,6)-pyridinacyclodecaphane-6-yl)succinate

A suspension of di-tert-butyl N,N-bis(2-((2-(tert-butoxy)-2-oxoethyl)amino)ethyl)-L-aspartate (0.72 g, 1.29 mmol) and anhydrous Na₂CO₃ (0.41 g, 6.44 mmol) in dry CH₃CN (261.9 mL) was heated to reflux for 1.5 hours and then cooled to RT. Methyl 2,6-bis(bromomethyl)isonicotinate (1.44 g, 4.49 mmol), prepared according to the procedure from Bioorg. Med. Chem. Lett. 22 (2012) 2684-2688, page 2686, compound 9 (0.413 g, 1.287 mmol) was added in one portion and then heated to reflux for 24 h. After completion, the reaction mixture was cooled to RT and filtered through a short celite plug. The solvents were evaporated under reduced pressure to obtain a mixture of sodium and free complex of the title compound as beige solid (0.75 g, Crude). The residue was treated with saturated mono sodium EDTA solution in CH₂Cl₂ and stirred for 16 hours. The organic layer was separated, dried over anhydrous Na₂SO₄, filtered and evaporated under reduced pressure to obtain 650 mg of crude title compound as a free complex. LC-MS-1: Rt=1.467 min, m/z: 721.30 [M+H]; ¹H NMR (400 MHz, CDCl₃) δ=7.72 (s, 2H), 4.10 (t, J=1.0 Hz, 1H), 3.83-3.99 (m, 5H), 3.66-3.78 (m, 2H), 3.25-3.46 (m, 4H), 2.97 (t, J=1.0 Hz, 1H), 2.59-2.82 (m, 2H), 2.20-2.52 (m, 4H), 1.68-2.09 (m, 3H), 1.28-1.59 (m, 36H).

### Step 2: (S)-3,9-Bis(2-(tert-butoxy)-2-oxoethyl)-6-(1,4-di-tert-butoxy-1,4-dioxobutan-2-yl)-3,6,9-triaza-1(2,6)-pyridinacyclodecaphane-1⁴-carboxylic acid

To a solution of di-tert-butyl (S)-2-(3,9-bis(2-(tert-butoxy)-2-oxoethyl)-1⁴-(methoxycarbonyl)-3,6,9-triaza-1(2,6)-pyridinacyclodecaphane-6-yl)succinate (0.65 g, 0.98 mmol) in methanol (20 mL) and water (10 mL) was added anhydrous Na₂CO₃ (0.10 g, 0.98 mmol). After completion, the solvent was evaporated under reduced pressure, the aqueous layer was adjusted to pH 6 using 0.1 N HCl, and lyophilized. The residue was purified by preparative HPLC (RP-HPLC-1) eluting with 0.1% formic acid in water and MeCN to afford the title compound as a formate salt and white colour solid (315 mg, 41.8%). LC-MS-1: Rt=1.60 min, m/z: 707.20 [M+H]; ¹H NMR (400 MHz, CDCl₃) δ=7.87 (s, 2H), 4.26 (s, 4H), 4.04-4.15 (m, 2H), 3.45 (s, 4H), 2.94-3.20 (m, 8H), 2.66-2.86 (m, 1H), 1.39-1.46 (m, 36H).

### Step 3: (S)-2-(3,9-bis(carboxymethyl)-1⁴-((3-(2,5-dioxo-2,5-dihydro-1H-pyrrol-1-yl)propyl)carbamoyl)-3,6,9-triaza-1(2,6)-pyridinacyclodecaphane-6-yl)succinic acid

Following the procedure for Example 3, using (S)-3,9-Bis(2-(tert-butoxy)-2-oxoethyl)-6-(1,4-di-tert-butoxy-1,4-dioxobutan-2-yl)-3,6,9-triaza-1(2,6)-pyridinacyclodecaphane-1⁴-carboxylic acid and intermediate D, the title compound was prepared. LC-MS-12: Rt=0.13 mins; MS m/z [M+H]⁺ 619.3; ¹H NMR (400 MHz, DMSO) δ 8.88 (t, J=5.6 Hz, 1H), 7.86 (s, 2H), 6.94 (s, 2H), 4.85 (q, J=16.0 Hz, 4H), 4.33 (d, J=3.3 Hz, 4H), 3.98 (s, 1H), 3.55 (s, 4H), 3.51-3.40 (m, 3H), 3.28 (q, J=6.6 Hz, 2H), 3.06 (s, 4H), 2.89-2.78 (m, 1H), 2.68-2.60 (m, 1H), 1.78 (p, J=7.1 Hz, 2H).

### Example 13: (2R,2′R)-2,2′-(1⁴-(3-(2,5-dioxo-2,5-dihydro-1H-pyrrol-1-yl)propoxy)-6-(pyridin-2-ylmethyl)-3,6,9-triaza-1(2,6)-pyridinacyclodecaphane-3,9-diyl)disuccinic acid

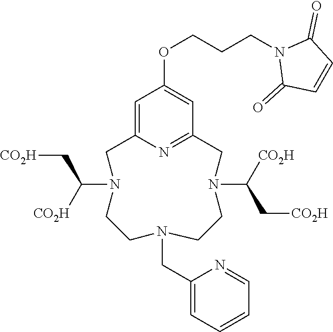 [SMILES: O=C(O)C[C@H](C(=O)O)N1CCN(Cc2ccccn2)CCN([C@H](CC(=O)O)C(=O)O)Cc2cc(OCCCN3C(=O)C=CC3=O)cc(n2)C1]

### Step 1: Tetra-tert-butyl 2,2′-((((pyridin-2-ylmethyl)azanediyl)bis(ethane-2,1-diyl))bis(((2-nitrophenyl)sulfonyl)azanediyl))(2R,2′R)-disuccinate

To a solution of 2,2′-((pyridin-2-ylmethyl)azanediyl)bis(ethan-1-ol) (200 mg, 1.02 mmol) in THF (20 mL) was added at RT under argon di-tert-butyl ((2-nitrophenyl)sulfonyl)-D-aspartate (965 mg, 2.24 mmol) and triphenylphosphine polymer-bound, 200-400 mesh particle size, 3.0 mmol/g loading (857 mg, 2.55 mmol). The reaction mixture was cooled to 0° C. and a solution of di-tert-butyl azodicarboxylate (587 mg, 2.55 mmol) in THF (4 mL) was added dropwise over 10 min. After shaking the reaction mixture for 2 days at RT, triphenylphosphine polymer-bound, 200-400 mesh particle size, 3.0 mmol/g loading (857 mg, 2.55 mmol) was added and the reaction mixture was cooled to 0° C. A solution of di-tert-butyl azodicarboxylate (587 mg, 2.55 mmol) in THF (4 mL) was added dropwise. After shaking the reaction mixture at RT for 16 hours, 2,2′-((pyridin-2-ylmethyl)azanediyl)bis(ethan-1-ol) (200 mg, 1.02 mmol) was added. After 16 hour, the reaction mixture was filtered through Celite and the filtrate was concentrated under reduced pressure to provide a brown oil. The crude product was purified twice by flash chromatography on silica gel eluting with (CH₂Cl₂/MeOH/NH₄OH, 80:20:0.4) afforded the title compound (3.6 g, 1.2 mmol, 35% purity, 24.0% yield). It was used in the next step without further purification. LC-MS-8: Rt=1.48 mins; MS m/z [M+H]⁺ 1021.8

### Step 2: Tetra-tert-butyl 2,2′-((((pyridin-2-ylmethyl)azanediyl)bis(ethane-2,1-diyl))bis(azanediyl))(2R,2′R)-disuccinate

To a solution of tetra-tert-butyl 2,2′-((((pyridin-2-ylmethyl)azanediyl)bis(ethane-2,1-diyl))bis(((2-nitrophenyl)sulfonyl)azanediyl))(2R,2′R)-disuccinate (3.6 g, 1.2 mmol) in DMF (33 mL) was added K₂CO₃ (3.1 g, 23 mmol) and dropwise thiophenol (1.9 g, 17 mmol). The reaction mixture was stirred at RT for 2 hours and concentrated under reduced pressure.

The residue was extracted with EtOAc/water, the combined organic layers were washed with a saturated aqueous solution of Na₂CO₃ and NaCl, dried (Phase Separator) and concentrated under reduced pressure. The crude product was purified by flash chromatography on silica gel eluting with CH₂Cl₂/MeOH/NH₄OH 95:5:1 afforded the title compound as a brown oil (1.85 g). The product was further purified using the method RP-HPLC-3 afforded the title compound as a yellow oil (940 mg, 52%).

LC-MS-8: Rt=1.13 mins; MS m/z [M+H]⁺ 651.8

### Step 3: Tetra-tert-butyl 2,2′-(1⁴-(3-((tert-butoxycarbonyl)amino)propoxy)-6-(pyridin-2-ylmethyl)-3,6,9-triaza-1(2,6)-pyridinacyclodecaphane-3,9-diyl)(2R,2′R)-disuccinate

To a solution of tetra-tert-butyl 2,2′-((((pyridin-2-ylmethyl)azanediyl)bis(ethane-2,1-diyl))bis(azanediyl))(2R,2′R)-disuccinate (297.1 mg, 456.5 μmol) in anhydrous CH₃CN (230.0 mL) were added Na₂CO₃ (483.8 mg, 4.56 mmol) and intermediate G (200.0 mg, 456.5 μmol) in one portion and the off white suspension was stirred at reflux for 16 hours. A second portion of intermediate G (200.0 mg, 1 Eq, 456.5 μmol) and the reaction mixture was stirred at 100° C. for 3 days. The reaction mixture was concentrated under reduced pressure, the residue was extracted with water/EtOAc, the combined organic layers were dried (Phase Separator) and concentrated under reduced pressure. The crude was purified by flash chromatography on silica gel eluting with (75/25) (CH₂Cl₂/CH₂Cl₂:MeOH:NH₄OH; 96:4:0.4) afforded the title compound (254 mg, 48%). LC-MS-12: Rt=1.33 mins; MS m/z [M+H]⁺ 927.8

### Step 4: (Z)-4-((3-((3,9-bis((R)-1,4-di-tert-butoxy-1,4-dioxobutan-2-yl)-6-(pyridin-2-ylmethyl)-3,6,9-triaza-1(2,6)-pyridinacyclodecaphane-1⁴-yl)oxy)propyl)amino)-4-oxobut-2-enoic acid

To a solution tetra-tert-butyl 2,2′-(1⁴-(3-((tert-butoxycarbonyl)amino)propoxy)-6-(pyridin-2-ylmethyl)-3,6,9-triaza-1(2,6)-pyridinacyclodecaphane-3,9-diyl)(2R,2′R)-disuccinate (130.0 mg, 140.2 μmol) in CH₂Cl₂ (5.0 mL) was added dropwise at 0° C. TFA (159.9 mg, 108.0 μL, 1.402 mmol). After stirring the reaction mixture at RT for 3 hours a second addition of TFA (159.9 mg, 108.0 μL, 1.402 mmol) was performed. After 2 hours at RT, TEA (212.8 mg, 293 μL, 15 Eq, 2.103 mmol) was added dropwise at 0° C. The reaction mixture was diluted with toluene (5.000 mL) and maleic anhydride (15.12 mg, 154.2 μmol) was added. After stirring the reaction mixture at 0° C. for 1 hour the reaction mixture was concentrated under reduced pressure. The crude product was purified using the method RP-HPLC-3 afforded the title compound (24 mg, 15%) as a white powder. LC-MS-8: Rt=1.07 mins; MS m/z [M+H]⁺ 926.5; ¹H NMR (400 MHz, DMSO) δ 13.20 (br dd, J=4.47, 2.64 Hz, 1H), 9.09 (t, J=5.85 Hz, 1H), 8.68 (d, J=4.98 Hz, 1H), 7.96 (br t, J=7.28 Hz, 1H), 7.77 (d, J=7.78 Hz, 1H), 7.48-7.51 (m, 1H), 6.87-7.02 (m, 2H), 6.41 (d, J=12.54 Hz, 1H), 6.25 (d, J=12.40 Hz, 1H), 4.47 (s, 1H), 4.41 (s, 1H), 4.05-4.25 (m, 4H), 3.93 (br s, 4H), 3.62-3.78 (m, 21H), 3.11-3.38 (m, 9H), 2.83-3.03 (m, 3H), 2.51 (dt, J=3.58, 1.77 Hz, 50H), 1.95 (br t, J=6.46 Hz, 2H), 1.48 (s, 9H), 1.37-1.44 (m, 4h), 1.36 (s, 8h), 1.30 (s, 9H), 1.24 (s, 9H).

### Step 5: Tetra-tert-butyl 2,2′-(1⁴-(3-(2,5-dioxo-2,5-dihydro-1H-pyrrol-1-yl)propoxy)-6-(pyridin-2-ylmethyl)-3,6,9-triaza-1(2,6)-pyridinacyclodecaphane-3,9-diyl)(2R,2′R)-disuccinate

To a solution of (Z)-4-((3-((3,9-bis((R)-1,4-di-tert-butoxy-1,4-dioxobutan-2-yl)-6-(pyridin-2-ylmethyl)-3,6,9-triaza-1(2,6)-pyridinacyclodecaphane-1⁴-yl)oxy)propyl)amino)-4-oxobut-2-enoic acid (24.00 mg, 25.94 μmol) in CH₂Cl₂ (4.0 mL), was added pentafluorophenol (7.187 mg, 38.91 μmol) and diisopropylcarbodiimide (9.822 mg, 12.1 μL, 77.83 μmol). After stirring at RT for 16 hours, the reaction mixture was concentrated under reduced pressure. The crude product was purified by flash chromatography on silica gel eluting with CH₂CL₂/MeOH/NH₄OH 90:10:1, provided the title compound as a colorless oil (19 mg, 65%). LC-MS-12: Rt=1.23 mins; MS m/z [M+H]⁺ 908.5.

### Step 6: (2R,2′R)-2,2′-(1⁴-(3-(2,5-dioxo-2,5-dihydro-1H-pyrrol-1-yl)propoxy)-6-(pyridin-2-ylmethyl)-3,6,9-triaza-1(2,6)-pyridinacyclodecaphane-3,9-diyl)disuccinic acid

A solution of tetra-tert-butyl 2,2′-(1⁴-(3-(2,5-dioxo-2,5-dihydro-1H-pyrrol-1-yl)propoxy)-6-(pyridin-2-ylmethyl)-3,6,9-triaza-1(2,6)-pyridinacyclodecaphane-3,9-diyl)(2R,2′R)-disuccinate (19.00 mg, 20.95 μmol) in water (33.33 μL) and TFA (300.0 μL) was stirred at RT. After 16 hours, 1 ml of water was added and the reaction mixture was filtered through a syringe filter and purified using the method RP-HPLC-3 to give 5 mg (20%) of title compound as a white powder. LC-MS-15: Rt=3.20 mins; MS m/z [M+H]⁺ 683.3. ¹H NMR (400 MHz, CD₃SOCD₃) δ 13.14-11.74 (m, 4H), 8.68 (d, J=5.0 Hz, 1H), 7.94 (dt, J=1.8, 7.7 Hz, 1H), 7.74 (d, J=7.9 Hz, 1H), 7.49 (dd, J=4.9, 7.0 Hz, 1H), 7.04 (s, 2H), 6.91 (br s, 1H), 6.81 (br s, 1H), 4.46 (d, J=15.1 Hz, 1H), 4.37 (d, J=15.1 Hz, 1H), 4.22-4.05 (m, 3H), 4.00 (br d, J=17.2 Hz, 1H), 3.91 (br s, 2H), 3.77 (br s, 1H), 3.74-3.67 (m, 1H), 3.57-3.56 (m, 2H), 3.53-3.46 (m, 1H), 3.33-3.11 (m, 6H), 2.95 (br d, J=14.4 Hz, 1H), 2.90-2.77 (m, 1H), 2.73-2.56 (m, 2H), 2.36-2.23 (m, 1H), 2.04-1.92 (m, 2H).

### Example 14: (2S,2′S,2″S)-2,2′,2″-(1⁴-(3-(2,5-dioxo-2,5-dihydro-1H-pyrrol-1-yl)propoxy)-3,6,9-triaza-1(2,6)-pyridinacyclodecaphane-3,6,9-triyl)tripropionic acid

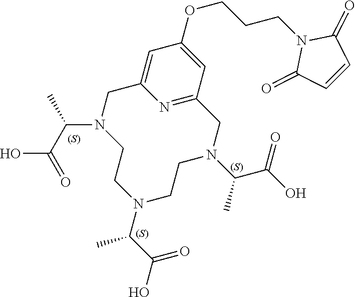

### Step 1: Tert-butyl (2-((tert-butyldimethylsilyl)oxy)ethyl)-L-alaninate (P1)

To a solution of tert-butyl-L-alaninate, hydrochloride (2000 mg, 11.01 mmol) in CH₂Cl₂ (60 mL), was added 2-((tert-butyldimethylsilyl)oxy)acetaldehyde (1.919 g, 11.01 mmol), and after 30 min sodium triacetoxyborohydride (3.5 g, 16.51 mmol) was added at RT. After stirring the solution at 20° C. for 16 hours, the reaction mixture was treated with CH₂Cl₂, and washed with a saturated solution of aqueous NaHCO₃. The organic phase was dried over MgSO₄ and concentrated under reduced pressure to give the title compound (3.17 g, 90% yield). LC-MS-12: Rt=0.95 min, MS m/z: 304.4 [M+H]⁺. ¹H NMR (400 MHz, DMSO-d6) b 3.51-3.62 (m, 2H), 3.11 (q, J=6.90 Hz, 1H), 2.53-2.61 (m, 1H), 2.41-2.46 (m, 1H), 1.38 (s, 9H), 1.08 (d, J=6.90 Hz, 3H), 0.83 (s, 9H), 0.00 (s, 6H).

### Step 2: Tert-butyl N-benzyl-N-(2-((tert-butyldimethylsilyl)oxy)ethyl)-L-alaninate

To a solution of tert-butyl (2-((tert-butyldimethylsilyl)oxy)ethyl)-L-alaninate (1.57 g, 5.17 mmol) in acetonitrile (40 mL), potassium carbonate (1.79 g, 12.9 mmol) and benzyl bromide (973 mg, 677 μL, 5.69 mmol) were added. After stirring the reaction mixture at 20° C. for 16 hours, AcOEt was added, the organic phase was separated and washed with an aqueous solution of saturated sodium bicarbonate. The organic phase was dried over MgSO₄ and concentrated under reduced pressure to give 2.15 g of crude material. The crude was purified by flash chromatography on silica gel eluting with Heptane/AcOEt+1% NEt₃ (90/10) to give the title compound (1.6 g, 78% yield). LC-MS-12: Rt=1.73 min, MS m/z: 394.4 [M+H]⁺. ¹H NMR (400 MHz, DMSO-d6) δ 7.31-7.45 (m, 4H), 7.26 (br d, J=6.68 Hz, 1H), 3.79-3.89 (m, 1H), 3.67-3.74 (m, 1H), 3.52 (td, J=6.55, 2.60 Hz, 2H), 3.40 (d, J=7.12 Hz, 1H), 2.68-2.77 (m, 1H), 2.58-2.67 (m, 1H), 1.47 (s, 9H), 1.18 (d, J=7.12 Hz, 3H), 0.85 (s, 9H) 0.00 (s, 6H).

### Step 3: Tert-butyl N-benzyl-N-(2-hydroxyethyl)-L-alaninate

To a solution of tert-butyl N-benzyl-N-(2-((tert-butyldimethylsilyl)oxy)ethyl)-L-alaninate (1600 mg, 4.065 mmol) in THF (20 mL) was added at 0° C. TBAF (5.314 g, 20.32 mL, 20.32 mmol). After stirring at 0° C. for 1 hour, the reaction mixture was concentrated under reduced pressure to give 8.7 g of crude material. The crude was purified by flash chromatography eluting with Heptane/AcOEt+0.1% NEt₃ (80/20) to give the title compound (525 mg, 46% yield). LC-MS-12: Rt=0.86 min, MS m/z: 280.4 [M+H]⁺. ¹H NMR (400 MHz, DMSO-d6) δ 7.32-7.40 (m, 4H), 7.27 (br d, J=6.60 Hz, 1H), 4.37 (t, J=5.43 Hz, 1H), 3.82-3.93 (m, 1H), 3.67-3.75 (m, 1H), 3.36 (s, 1H), 2.68-2.76 (m, 1H), 2.56-2.64 (m, 1H), 2.35 (t, J=7.01 Hz, 2H), 1.48 (s, 9H), 1.18 (d, J=7.12 Hz, 3H).

### Step 4: Tert-butyl N-benzyl-N-(2-chloroethyl)-L-alaninate

To a solution of tert-butyl N-benzyl-N-(2-hydroxyethyl)-L-alaninate (406 mg, 1.45 mmol) in CH₂Cl₂(16 mL), thionyl chloride (346 mg, 212 μL, 2.91 mmol) was added dropwise. After stirring at 40° C. for 1 hour, the reaction mixture was cooled to room temperature and neutralized carefully with an aqueous solution of sodium bicarbonate, followed by extraction with CH₂Cl₂. The organic phase was concentrated under reduced pressure to give the title compound (345 mg). It was used in the next step without further purification. LC-MS-12: Rt=1.59 min, MS m/z: 298.3 [M+H]⁺.

### Step 5: Di-tert-butyl 2,2′-(((((S)-1-(tert-butoxy)-1-oxopropan-2-yl)azanediyl)bis(ethane-2,1-diyl))bis(benzylazanediyl))(2S,2′S)-dipropionate

To a solution of tert-butyl N-benzyl-N-(2-chloroethyl)-L-alaninate (345 mg, 1.16 mmol) in CH₃CN (15 mL), tert-butyl L-alaninate hydrochloride (105 mg, 579 μmol), Na₂CO₃ (2.46 g, 23.2 mmol) and KI (577 mg, 3.48 mmol) were added. The solution was stirred at 60° C. for 16 hours, followed by 3 days at 20° C. The reaction mixture was treated with EtOAc, and washed with a saturated solution of aqueous sodium bicarbonate. The organic phase was dried over MgSO₄ and concentrated under reduced pressure. The crude material was purified by preparative HPLC (RP-HPLC-3) to give the title compound (230 mg, 29% yield). LC-MS-12: Rt=1.70 min, MS m/z: 668.6 [M+H]⁺. ¹H NMR (400 MHz, DMSO-d6) δ 7.34 (s, 10H), 4.08-4.18 (m, 1H), 3.83-3.89 (m, 2H), 3.75 (br d, J=13.86 Hz, 2H), 3.44-3.46 (m, 2H), 2.83-3.05 (m, 8H), 1.43-1.50 (m, 1H), 1.42 (s, 9H), 1.23 (br d, J=7.04 Hz, 6H).

### Step 6: Di-tert-butyl 2,2′-(((((S)-1-(tert-butoxy)-1-oxopropan-2-yl)azanediyl)bis(ethane-2,1-diyl))bis(azanediyl))(2S,2′S)-dipropionate

To a solution of di-tert-butyl 2,2′-(((((S)-1-(tert-butoxy)-1-oxopropan-2-yl)azanediyl)bis(ethane-2,1-diyl))bis(benzylazanediyl))(2S,2′S)-dipropionate (210 mg, 314 μmol) in EtOH (20 mL), Pd/C (33.5 mg, 10% Wt, 31.4 μmol) was added. The solution was stirred under H₂ pressure for 19 hours at 20° C. The reaction mixture was filtered and concentrated under reduced pressure to give the title compound (190 mg, 99% yield). LC-MS-12: Rt=0.75 min, MS m/z: 488.5 [M+H]*. ¹H NMR (400 MHz, DMSO-d6) δ 8.78-8.90 (m, 2H), 4.01-4.14 (m, 2H), 3.50 (br d, J=7.12 Hz, 1H), 3.04 (br s, 2H), 2.79-2.99 (m, 6H), 1.48 (s, 18H), 1.44 (s, 15H), 1.24 (d, J=7.12 Hz, 3H).

### Step 7: Tri-tert-butyl 2,2′,2″-(1⁴-(3-((tert-butoxycarbonyl)amino)propoxy)-3,6,9-triaza-1(2,6)-pyridinacyclodecaphane-3,6,9-triyl)(2S,2′S,2″S)-tripropionate

To a solution of di-tert-butyl 2,2′-(((((S)-1-(tert-butoxy)-1-oxopropan-2-yl)azanediyl)bis(ethane-2,1-diyl))bis(azanediyl))(2S,2′S)-dipropionate (190 mg, 80% Wt, 312 μmol) in CH₃CN (20 mL), DIPEA (201 mg, 271 μL, 1.56 mmol) and intermediate G (137 mg, 312 μmol) were added. After stirring the solution at 60° C. for 24 hours, the reaction mixture was treated with AcOEt, and washed with an aqueous solution of saturated sodium bicarbonate. The organic phase was dried over MgSO₄ and concentrated under reduced pressure to give 240 mg of crude material. The crude was purified by preparative HPLC (method RP-HPLC-3) to provide the title compound (130 mg, 52% yield). LC-MS-12: Rt=1.21 min, MS m/z: 764.4 [M+H]⁺. ¹H NMR (400 MHz, DMSO-d6) δ 1.20 (br d, J=6.97 Hz, 6H), 1.37 (br s, 30H), 1.45 (s, 9H), 1.80-1.86 (m, 2H), 3.06 (br d, J=5.94 Hz, 3H), 3.19-3.26 (m, 2H), 3.34 (br s, 2H), 3.72 (br d, J=7.04 Hz, 4H), 3.99 (br s, 4H), 4.11 (br t, J=5.39 Hz, 2H), 4.19-4.29 (m, 2H), 6.92 (br s, 1H) 6.96 (s, 2H).

### Step 8: Tri-tert-butyl 2,2′,2″-(1⁴-(3-aminopropoxy)-3,6,9-triaza-1(2,6)-pyridinacyclodecaphane-3,6,9-triyl)(2S,2′S,2″S)-tripropionate

To a solution of tri-tert-butyl 2,2′,2″-(1⁴-(3-((tert-butoxycarbonyl)amino)propoxy)-3,6,9-triaza-1(2,6)-pyridinacyclodecaphane-3,6,9-triyl)(2S,2′S,2″S)-tripropionate (150 mg, 196 μmol) in CH₂Cl₂ (10 mL), was added at 0° C. TFA (448 mg, 303 μL, 3.93 mmol). The solution was stirred at 0° C. for 1 hour, followed by 2 hours at 20° C. The reaction mixture was treated with CH₂Cl₂, and washed with an aqueous solution of saturated sodium bicarbonate. The organic phase was dried over MgSO₄ and concentrated under reduced pressure to give the title compound (97 mg, 67%). LC-MS-12: Rt=0.79 min, MS m/z: 664.5 [M+H]⁺. ¹H NMR (400 MHz, DMSO-d6) δ 6.75 (s, 2H), 4.03 (br t, J=6.35 Hz, 2H), 3.67-3.74 (m, 2H), 3.60 (br d, J=11.00 Hz, 2H), 3.31-3.33 (m, 3H), 2.99 (br dd, J=12.73, 6.64 Hz, 2H), 2.62-2.69 (m, 6H), 2.46 (br d, J=1.83 Hz, 2H), 1.74-1.77 (m, 2H), 1.38 (s, 22H), 1.26 (s, 9H), 1.14 (br s, 3H), 0.93 (br d, J=6.97 Hz, 3H).

### Step 9: Tri-tert-butyl 2,2′,2″-(14-(3-(2,5-dioxo-2,5-dihydro-1H-pyrrol-1-yl)propoxy)-3,6,9-triaza-1(2,6)-pyridinacyclodecaphane-3,6,9-triyl)(2S,2′S,2″S)-tripropionate

To a solution of tri-tert-butyl 2,2′,2″-(1⁴-(3-aminopropoxy)-3,6,9-triaza-1(2,6)-pyridinacyclodecaphane-3,6,9-triyl)(2S,2′S,2″S)-tripropionate, hydrochloride (97 mg, 0.12 mmol) dissolved in toluene (3 mL), NEt₃ (85 mg, 0.12 mL, 0.84 mmol) was added followed by a dropwise addition of a solution of maleic anhydride (13 mg, 9.1 μL, 0.13 mmol) in 0.5 mL of toluene. After stirring one hour, the reaction mixture was evaporated under reduced pressure. The intermediate was dissolved in CH₂Cl₂ (3 mL) and pentafluorophenol (22.99 mg, 0.125 mmol) and N,N′-diisopropylcarbodiimide (0.039 mL, 0.250 mmol) were added. After stirring the reaction mixture at 30° C. for 16 hours, the volatiles were evaporated. The residue was dissolved in CH₂Cl₂. The organic phase was washed with H₂O, dried over MgSO₄ and concentrated under reduced pressure to give 170 mg of crude material. The crude was purified preparative HPLC (RP-HPLC-3) to provide the title compound (54 mg, 47%). LC-MS-12: Rt=1.07 min, MS m/z: 744.5 [M+H]*. ¹H NMR (400 MHz, DMSO-d6) δ 7.03 (s, 2H), 6.92 (s, 2H), 4.28 (br d, J=6.97 Hz, 1H), 4.09 (br d, J=2.05 Hz, 2H), 4.00 (br s, 4H), 3.57 (br d, J=6.75 Hz, 4H), 3.34 (br d, J=8.66 Hz, 2H), 3.21-3.27 (m, 2H), 3.09 (br d, J=8.58 Hz, 2H), 2.97-3.04 (m, 2H), 1.95-2.00 (m, 2H), 1.45 (s, 9H), 1.40 (br s, 3H), 1.36 (s, 18H), 1.21 (br d, J=7.04 Hz, 6H).

### Step 10: (2S,2′S,2″S)-2,2′,2″-(1⁴-(3-(2,5-dioxo-2,5-dihydro-1H-pyrrol-1-yl)propoxy)-3,6,9-triaza-1(2,6)-pyridinacyclodecaphane-3,6,9-triyl)tripropionic acid

To a solution of tri-tert-butyl 2,2′,2″-(1⁴-(3-(2,5-dioxo-2,5-dihydro-1H-pyrrol-1-yl)propoxy)-3,6,9-triaza-1(2,6)-pyridinacyclodecaphane-3,6,9-triyl)(2S,2′S,2″S)-tripropionate (54 mg, 73 μmol) in water (0.03 mL), TFA (0.5 mL) was added. After stirring the solution at 20° C. for 3.5 hours, the volatiles were evaporated and the crude was purified by preparative HPLC (RP-HPLC-3) to provide the title compound (22 mg, 44%). LC-MS-13: Rt=0.57 min, MS m/z: 576.3 [M+H]*. ¹H NMR (400 MHz, DMSO-d6) δ 7.27 (br s, 2H), 7.06 (s, 1H), 4.52-4.67 (m, 2H), 4.43 (br d, J=6.82 Hz, 1H), 4.23-4.28 (m, 2H), 4.08-4.23 (m, 2H), 3.66 (t, J=6.53 Hz, 2H), 3.39 (br dd, J=5.87, 3.37 Hz, 2H), 2.84-3.16 (m, 2H), 2.03-2.14 (m, 2H), 1.53 (br d, J=6.53 Hz, 6H), 1.44 (br d, J=6.97 Hz, 3H).

### Example 15: (R)-2-(9-((R)-2-carboxy-1-(pyridin-2-yl)ethyl)-1⁴-((3-(2,5-dioxo-2,5-dihydro-1H-pyrrol-1-yl)propyl)carbamoyl)-6-oxa-3,9-diaza-1(2,6)-pyridinacyclodecaphane-3-yl)succinic acid

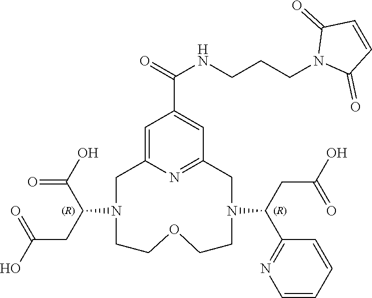

### Step 1: tert-butyl (R,Z)-3-((1-(4-methoxyphenyl)ethyl)amino)-3-(pyridin-2-yl)acrylate

To a solution of ethyl 3-oxo-3-(pyridin-2-yl)propanoate (4.13 g, 18.67 mmol) in toluene (160 mL), (R)-1-(4-methoxyphenyl)ethan-1-amine (0.783 g, 5.18 mmol) and AcOH (1.069 mL, 18.67 mmol) were added. The reaction mixture was stirred for 16 hours at 70° C. with Dean Stark trap under reduced pressure. Then AcOEt was added, the organic phase was separated and washed with an aqueous solution of saturated sodium bicarbonate. The organic phase was dried over MgSO₄ and concentrated under reduced pressure to give 1.54 g of crude material. The crude was purified by flash chromatography on silica gel eluting with Heptane/AcOEt containing 1% NEt₃ (70/30) to provide the title compound (1.7 g, 23.1% yield). LC-MS-12: Rt=1.27 min. m/z: 355.3 [M+H]; ¹H NMR (400 MHz, DMSO-d6) δ 8.61-8.69 (m, 2H), 7.78 (td, J=7.72, 1.58 Hz, 1H), 7.41 (dd, J=7.41, 4.92 Hz, 1H), 7.30 (d, J=7.78 Hz, 1H), 6.97 (d, J=8.66 Hz, 2H), 6.80 (d, J=8.66 Hz, 2H), 4.70 (dd, J=8.25, 7.15 Hz, 1H), 4.48 (s, 1H), 3.69 (s, 3H), 1.44 (s, 9H), 1.38 (d, J=6.75 Hz, 3H).

### Step 2: Tert-butyl (R)-3-(((R)-1-(4-methoxyphenyl)ethyl)amino)-3-(pyridin-2-yl)propanoate

To a solution tert-butyl (R,Z)-3-((1-(4-methoxyphenyl)ethyl)amino)-3-(pyridin-2-yl)acrylate (1700 mg, 4.80 mmol) dissolved in EtOH (25 mL), BF₃·OEt₂ (1.216 mL, 9.59 mmol) and Pd(OH)₂ (337 mg, 0.480 mmol) were added according to Tetrahedron Letters 43 (2002) 1977-1981. The reaction mixture was stirred for 90 min at 20° C. under atmospheric H₂ pressure. The reaction mixture was filtered and evaporated under reduced pressure. AcOEt was added and the organic phase was washed with an aqueous solution of saturated sodium bicarbonate. The organic phase was dried over MgSO₄ and concentrated under reduced pressure. The crude was purified flash chromatography on silica gel eluting with heptane/AcOEt (+1% NEt₃) (70/30) to give the title compound as major product (400 mg, 1.111 mmol, 23.16% yield). LC-MS-12: Rt=0.65 min, m/z: 357.3 [M+H]⁺; ¹H NMR (400 MHz, DMSO-d6) δ 8.53 (brd, J=4.18 Hz, 1H), 7.75 (td, J=7.59, 1.32 Hz, 1H), 7.21-7.35 (m, 2H), 7.14 (d, J=8.51 Hz, 2H), 6.86 (d, J=8.44 Hz, 2H), 3.74 (s, 3H), 3.73 (br s, 1H), 3.30 (br d, J=6.68 Hz, 1H), 2.62 (br d, J=6.75 Hz, 1H), 2.46 (br d, J=7.34 Hz, 2H), 1.29 (s, 9H), 1.12 (d, J=6.46 Hz, 3H).

### Step 3: Tert-butyl (R)-3-amino-3-(pyridin-2-yl)propanoate

To a solution of tert-butyl (R)-3-(((R)-1-(4-methoxyphenyl)ethyl)amino)-3-(pyridin-2-yl)propanoate (400 mg, 1.122 mmol) in MeOH/H₂O (5:1) (18 mL), ammonium cerium(IV) nitrate (2461 mg, 4.49 mmol) was added. After stirring for 16 hours at 20° C., the reaction mixture was treated with a saturated solution of aqueous sodium bicarbonate, and extracted four times with Et₂O. The organic phase was dried over MgSO₄ and concentrated under reduced pressure to give 360 mg of crude material. The crude was purified by flash chromatography on silica gel eluting with TBME/(TBME/MeOH 1/1) (80/20) to give the title compound (150 mg, 58.9% yield). LC-MS-12: Rt=0.31 min, m/z: 223.2 [M+H]⁺; ¹H NMR (400 MHz, DMSO-d6) δ 8.48 (br d, J=4.25 Hz, 1H), 7.74 (br t, J=7.12 Hz, 1H), 7.45 (br d, J=7.78 Hz, 1H), 7.18-7.28 (m, 1H), 4.17 (br t, J=6.82 Hz, 1H), 2.66 (br dd, J=14.86, 5.98 Hz, 1H), 1.33 (s, 9H), 2.47 (br s, 1H).

### Step 4: Tert-butyl (R)-3-((2-nitrophenyl)sulfonamido)-3-(pyridin-2-yl)propanoate

To a solution of tert-butyl (R)-3-amino-3-(pyridin-2-yl)propanoate (150 mg, 0.675 mmol) dissolved in 2.5 mL of THF, NaHCO₃ (283 mg, 3.37 mmol) followed by a solution of 2-nitrobenzenesulfonyl chloride (164 mg, 0.742 mmol) in THF (2.5 mL) were added dropwise. After stirring for 4 hours at 60° C., the reaction mixture was treated with AcOEt, and washed with a saturated solution of aqueous sodium bicarbonate. The organic phase was dried over MgSO₄ and concentrated under reduced pressure to give 400 mg of crude material. The crude was purified flash chromatography on silica gel eluting with AcOEt/Heptane (50/50) to give the title compound (210 mg, 76% yield). LC-MS-12: Rt=0.87 min, m/z: 407.2 [M]+; ¹H NMR (400 MHz, DMSO-d6) δ 8.73 (br s, 1H), 8.38 (d, J=4.25 Hz, 1H), 7.86 (dd, J=7.92, 0.81 Hz, 1H), 7.78 (dd, J=7.85, 1.10 Hz, 1H), 7.73 (td, J=7.70, 1.25 Hz, 1H), 7.57-7.68 (m, 2H), 7.26 (d, J=7.85 Hz, 1H), 7.16 (dd, J=6.75, 4.92 Hz, 1H), 4.86 (br t, J=7.26 Hz, 1H), 2.78-2.86 (m, 1H), 2.64-2.72 (m, 1H), 1.28 (s, 9H).

### Step 5: Di-tert-butyl ((2-nitrophenyl)sulfonyl)-D-aspartate

To a solution of di-tert-butyl D-aspartate (HCl) (6.0 g, 20.23 mmol) and NaHCO₃ (5.10 g, 60.7 mmol) in THF (144 mL) was added portion wise 2-nitrobenzenesulfonyl chloride (5.08 g, 22.25 mmol) at 5-10° C. After stirring overnight at RT, the THF was evaporated and the residue partitioned between an aqueous solution of Na₂CO₃ (1M) and EtOAc. The organic layer was separated and water layer extracted with EtOAc. The organic layers were combined, washed with water and brine, dried by passing through a phase separating cartridge and volatiles were removed under reduced pressure to provide 9.1 g of crude. It was used in the next step without further purification. LC-MS-10: Rt=1.10 min, m/z: 448.4 [M+H₂O]⁺.

### Step 6: Di-tert-butyl N-(2-(2-hydroxyethoxy)ethyl)-N-((2-nitrophenyl)sulfonyl)-D-aspartate

To a solution of diethylene glycol (3.97 ml, 41.4 mmol) in THF (188 mL) was added at RT under argon di-tert-butyl ((2-nitrophenyl)sulfonyl)-D-aspartate (9 g, 18.82 mmol) and triphenylphosphine (14.81 g, 56.4 mmol). The reaction mixture was cooled to 0° C. and di-tert-butyl azodicarboxylate (13.26 g, 56.4 mmol) was added portion wise over 10 min. After stirring for 3 hours at RT, the reaction mixture was partitioned between an aqueous solution of saturated sodium bicarbonate and EtOAc. The organic layer was separated and water layer extracted with EtOAc. The organic layers were combined, dried by passing through a phase separating cartridge and volatiles were removed under reduced pressure to provide 18.2 g of crude. The crude was dissolved in CH₂Cl₂ and purified twice by flash chromatography on silica gel eluting with heptane/EtOAc (30/70) providing 6.5 g of title compound. LC-MS-10: Rt=1.02 min, m/z: 536.5 [M+H₂O]*.

### Step 7: Di-tert-butyl N-(2-(2-((N—((R)-3-(tert-butoxy)-3-oxo-1-(pyridin-2-yl)propyl)-2-nitrophenyl)sulfonamido)ethoxy)ethyl)-N-((2-nitrophenyl)sulfonyl)-D-aspartate

To a solution of tert-butyl (R)-3-((2-nitrophenyl)sulfonamido)-3-(pyridin-2-yl)propanoate (210 mg, 0.515 mmol) and di-tert-butyl N-(2-(2-hydroxyethoxy)ethyl)-N-((2-nitrophenyl)sulfonyl)-D-aspartate (281 mg, 0.541 mmol) in THF (8 mL), triphenylphosphine (203 mg, 0.773 mmol) was added. The reaction mixture was cooled to 0° C., DTBAD (178 mg, 0.773 mmol) was added. After stirring for 16 hours at 20° C., the reaction mixture was treated with AcOEt, the organic phase was separated and washed with an aqueous solution of saturated sodium bicarbonate. The organic phase was dried over MgSO₄ and concentrated in vacuum to give 960 mg of crude material. The crude was purified flash chromatography on silica gel eluting with AcOEt/Heptane (50/50) to provide the title compound (365 mg, 77% yield). LC-MS-12: Rt=1.38 min, m/z: 909.4 [M+H]⁺; ¹H NMR (400 MHz, DMSO-d6) δ 8.48 (d, J=4.03 Hz, 1H), 8.06 (td, J=7.02, 1.14 Hz, 2H), 7.96-8.03 (m, 2H), 7.88-7.95 (m, 2H), 7.80-7.87 (m, 2H), 7.76 (td, J=7.72, 1.72 Hz, 1H), 7.32 (dd, J=7.15, 5.17 Hz, 1H), 7.24 (d, J=7.92 Hz, 1H), 5.38 (dd, J=10.31, 4.36 Hz, 1H), 4.79 (t, J=6.93 Hz, 1H), 4.10 (q, J=5.28 Hz, 1H), 4.03 (q, J=7.12 Hz, 1H), 3.46-3.56 (m, 1H), 3.37-3.44 (m, 3H), 3.24 (dt, J=9.89, 6.76 Hz, 1H), 3.06-3.15 (m, 1H) 2.98-3.05 (m, 1H), 2.87 (dd, J=16.51, 7.34 Hz, 1H), 2.63-2.69 (m, 1H), 2.53-2.58 (m, 1H), 1.39 (s, 9H), 1.25 (s, 9H), 1.21 (s, 9H).

### Step 8: Di-tert-butyl (2-(2-(((R)-3-(tert-butoxy)-3-oxo-1-(pyridin-2-yl)propyl)amino)ethoxy)ethyl)-D-aspartate

To a solution of di-tert-butyl N-(2-(2-((N—((R)-3-(tert-butoxy)-3-oxo-1-(pyridin-2-yl)propyl)-2-nitrophenyl)sulfonamido)ethoxy)ethyl)-N-((2-nitrophenyl)sulfonyl)-D-aspartate (365 mg, 0.402 mmol) in THF (5 mL), K₂CO₃ (278 mg, 2.010 mmol) was added followed by thiophenol (0.137 mL, 1.327 mmol). The solution was stirred at 50° C. for 16 hours. The reaction mixture was treated with AcOEt, the organic phase was separated and washed with 3 times with an aqueous solution of saturated sodium carbonate. The organic phase was dried over MgSO₄ and concentrated under reduced pressure to give 375 mg of crude material. The crude was purified by flash chromatography on silica gel eluting with heptane/AcOEt (+1% TEA) (90/10) to provide the title compound (135 mg, 61.2% yield). LC-MS-12: Rt=0.80 min, m/z: 538.4 [M+H]⁺; ¹H NMR (400 MHz, DMSO-d6) δ 8.51 (d, J=4.55 Hz, 1H), 7.68-7.82 (m, 1H), 7.41 (d, J=7.78 Hz, 1H), 7.25 (dd, J=7.01, 5.17 Hz, 1H), 4.01 (br t, J=7.12 Hz, 1H), 3.34-3.43 (m, 5H), 2.69-2.76 (m, 1H), 2.56 (br dd, J=7.08, 4.95 Hz, 4H), 2.37-2.44 (m, 1H), 2.20-2.32 (m, 1H), 2.06 (br d, J=3.45 Hz, 1H), 1.40 (s, 9H), 1.38 (s, 9H), 1.31 (s, 9H).

### Step 9: Di-tert-butyl (R)-2-(9-((R)-3-(tert-butoxy)-3-oxo-1-(pyridin-2-yl)propyl)-1⁴-(methoxycarbonyl)-6-oxa-3,9-diaza-1(2,6)-pyridinacyclodecaphane-3-yl)succinate

To a solution of di-tert-butyl (2-(2-(((R)-3-(tert-butoxy)-3-oxo-1-(pyridin-2-yl)propyl)amino)ethoxy)ethyl)-D-aspartate (135 mg, 0.251 mmol) in CH₃CN (12 mL), DIPEA (0.219 mL, 1.255 mmol) and methyl 2,6-bis(bromomethyl)isonicotinate, prepared according to the procedure from Biorg. Med. Chem. Lett. 22 (2012) 2684-2688, page 2686, compound 9 (81 mg, 0.251 mmol) were added. After stirring the reaction mixture at 60° C. for 16 hours, AcOEt was added, the organic phase was separated and washed with an aqueous solution of saturated sodium bicarbonate. The organic phase was dried over MgSO₄ and concentrated under reduced pressure to give 180 mg of crude material. The crude was purified by flash chromatography on silica gel eluting with AcOEt (+1% NEt₃)/heptane (50/50) to give the title compound (90 mg, 48.7% yield). LC-MS-12: Rt=1.01 min, m/z: 699.4 [M+H]⁺; ¹H NMR (400 MHz, DMSO-d6) δ 8.53 (br d, J=4.47 Hz, 1H) 7.72-7.79 (m, 1H) 7.62-7.67 (m, 2H) 7.57 (s, 1H) 7.26-7.32 (m, 1H) 4.39 (br t, J=7.41 Hz, 1H) 3.93-3.99 (m, 1H) 3.87-3.92 (m, 5H) 3.69-3.76 (m, 2H) 2.88-2.97 (m, 4H) 2.83 (br dd, J=13.79, 6.97 Hz, 4H) 2.59-2.67 (m, 3H) 1.46 (s, 9H 1.34 (s, 9H)) 1.33 (s, 9H).

### Step 10: 3-((R)-3-(tert-butoxy)-3-oxo-1-(pyridin-2-yl)propyl)-9-((R)-1,4-di-tert-butoxy-1,4-dioxobutan-2-yl)-6-oxa-3,9-diaza-1(2,6)-pyridinacyclodecaphane-1⁴-carboxylic acid

To a solution of di-tert-butyl (R)-2-(9-((R)-3-(tert-butoxy)-3-oxo-1-(pyridin-2-yl)propyl)-1⁴-(methoxycarbonyl)-6-oxa-3,9-diaza-1(2,6)-pyridinacyclodecaphane-3-yl)succinate (90 mg, 0.129 mmol) MeOH (5 mL), NaOH (1M in H₂O) (0.129 mL, 0.129 mmol) was added. The solution was stirred at 20° C. for 1 hour. The reaction mixture was treated with AcOEt, the organic phase was separated and washed with an aqueous solution of saturated ammonium chloride. The organic phase was dried over MgSO₄ and concentrated under reduced pressure to give the title compound (87 mg, 0.126 mmol, 98% yield). LC-MS-12: Rt=1.06 min, m/z: 685.4 [M+H]⁺. It was used in the next step without further purification.

### Step 11: Di-tert-butyl (R)-2-(9-((R)-3-(tert-butoxy)-3-oxo-1-(pyridin-2-yl)propyl)-1⁴-((3-(2,5-dioxo-2,5-dihydro-1H-pyrrol-1-yl)propyl)carbamoyl)-6-oxa-3,9-diaza-1(2,6)-pyridinacyclodecaphane-3-yl)succinate

To a solution of 3-((R)-3-(tert-butoxy)-3-oxo-1-(pyridin-2-yl)propyl)-9-((R)-1,4-di-tert-butoxy-1,4-dioxobutan-2-yl)-6-oxa-3,9-diaza-1(2,6)-pyridinacyclodecaphane-1⁴-carboxylic acid (87 mg, 0.127 mmol) in CH₂Cl₂ (3 mL), HATU (62.8 mg, 0.165 mmol) was added. After 20 min at 20° C., a mixture of intermediate D (50.2 mg, 0.127 mmol) and TEA (0.039 ml, 0.279 mmol) in CH₂Cl₂ (2 mL) was added dropwise. After stirring for 16 hours at 20° C. The reaction mixture was treated with AcOEt, the organic phase was separated and washed with an aqueous solution of saturated sodium bicarbonate. The organic phase was dried over MgSO₄ and concentrated under reduced pressure to give 157 mg of crude material. The crude was purified by preparative HPLC (method RP-HPLC-3) to give the title compound (42 mg, 39.9% yield). LC-MS-12: Rt=1.02 min, m/z: 821.4 [M+H]⁺; ¹H NMR (400 MHz, DMSO-d6) δ 9.09 (t, J=5.47 Hz, 1H), 8.53 (d, J=4.55 Hz, 1H), 8.08 (s, 1H), 8.03 (s, 1H), 7.77 (td, J=7.65, 1.65 Hz, 1H), 7.58 (d, J=7.78 Hz, 1H), 7.31 (dd, J=7.08, 5.10 Hz, 1H), 4.68 (br dd, J=18.71, 1.47 Hz, 1H), 4.43-4.56 (m, 2H), 4.17-4.35 (m, 2H), 3.94 (br dd, J=9.57, 4.29 Hz, 1H), 3.49 (br d, J=7.12 Hz, 4H), 3.27-3.33 (m, 4H), 3.12 (br dd, J=15.74, 10.82 Hz, 2H), 2.86-3.05 (m, 6H), 1.81 (quin, J=7.12 Hz, 2H), 1.42 (s, 9H), 1.41 (s, 9H), 1.22 (s, 9H).

### Step 12: (R)-2-(9-((R)-2-carboxy-1-(pyridin-2-yl)ethyl)-1⁴-((3-(2,5-dioxo-2,5-dihydro-1H-pyrrol-1-yl)propyl)carbamoyl)-6-oxa-3,9-diaza-1(2,6)-pyridinacyclodecaphane-3-yl)succinic acid

To a solution of di-tert-butyl (R)-2-(9-((R)-3-(tert-butoxy)-3-oxo-1-(pyridin-2-yl)propyl)-1⁴-((3-(2,5-dioxo-2,5-dihydro-1H-pyrrol-1-yl)propyl)carbamoyl)-6-oxa-3,9-diaza-1(2,6)-pyridinacyclodecaphane-3-yl)succinate (42 mg, 0.051 mmol) in water (0.026 mL), TFA (0.5 mL) was added. After stirring the solution at 20° C. for 3 hours, the reaction mixture was concentrated under reduced pressure to give 45 mg of cru, crude was purified by preparative HPLC (method RP-HPLC-3) to provide the title compound (24 mg, 68.8% yield). LC-MS-13: Rt=0.56 min, m/z: 653.4 [M+H]⁺; ¹H NMR (400 MHz, DMSO-d6) δ 9.11 (t, J=5.50 Hz, 1H), 8.86 (d, J=4.40 Hz, 1H), 8.29 (t, J=7.89 Hz, 1H), 8.12 (s, 1H), 8.04-8.08 (m, 2H), 7.78 (dd, J=7.15, 5.69 Hz, 1H), 7.05 (s, 2H), 5.07 (br dd, J=8.91, 5.54 Hz, 1H), 4.95 (br d, J=3.81 Hz, 1H), 4.81-4.90 (m, 1H), 4.72-4.80 (m, 1H), 4.40-4.55 (m, 2H), 3.49-3.58 (m, 4H), 3.40-3.48 (m, 2H), 3.32-3.40 (m, 4H), 3.21-3.31 (m, 4H), 2.94-3.12 (m, 2H), 1.87 (t, J=6.97 Hz, 2H).

### General Conjugation Protocols:

The reaction starts by decapping the introduced C-terminus GGC tag on the protein of interest (NY2547 or 7D12), with addition of 10 mol eq. of a 0.25M TCEP-HCl solution, under argon and at room temperature. The reduced compound is mixed with 6 mol eq. of a 10 mg/mL solution of maleimide-chelator prepared in DMSO. The reaction mixture is incubated either at 25° C. for 3-4 hours or at 4°-8° C. overnight. The reaction progression is checked overtime by LC-MS. Once fully converted to the conjugated compound, the reaction is stopped and the final mixture purified by AKTA on a HiLoad Superdex75 16/600 pg column, using as eluent 50 mM NaOAc pH 5.5 buffer and run flow=0.5 mL/min. The fractions corresponding to the final compound are collected, pooled together and concentrated by centrifugation on an Amicon Filter MWCO: 3 kDa. Final solution is analyzed by UV-Vis, analytical SEC on a SPX75 10/300 column and LC-MS to confirm final compound.

NY2547sequence:
EVQLVESGGGLVQAGGSLRLSCAASGITFSINAFGWHRQAPGKQR
DLVAAISSGGRTNYANSVKGRFTISRDNTKNTVYLQMNNLAPEDT
AIYYCAIFEDGRWKYWGQGTQVTVSSGC
7D12 sequence:
QVKLEESGGGSVQTGGSLRLTCAASGRTSRSYGMGWFRQAPGKER
EFVSGISWRGDSTGYADSVKGRFTISRDNAKNTVDLQMNSLKPED
TAIYYCAAAAGSAWYGTLYEYDYWGQGTQVTVSSALEHHHHHH

### Example C1: NY2547—Example 1 Conjugate

To NY2547 (4 mL, 0.44 μmol) in a 15 mL eppendorf, TCEP-HCl (18 mL, 4.44 μmol) was added. The reaction mixture was degassed with Argon, vortexed vigorously for 2 min and stirred at 25° C. for 30 minutes. To this mixture 6 mol eq. (149 μL) of example 1 were added and the reaction was left at 4° C. overnight. The LC-MS analysis indicated the presence of starting material, therefore 1 mole equivalent of example 1 was added and the reaction mixture placed at 23° C. for 2 hours. The reaction mixture was purified twice by AKTA on a HiLoad Superdex75 16/600 μg column using as eluent 50 mM NaOAc pH 5.5. Fractions D3-D6 were collected and concentrated on an Amicon Filter MWCO: 3 kDa.

anaSEC RT=5.948 min (analysis performed using SPX200 column)

LC-MS m/z [M+H+Fe]⁺ calcd.:, found: 13339.

### Example C2: 7D12—Example 2 Conjugate

The title compound was prepared according to the Example C1 protocol using the 7D12 protein.

anaSEC RT=6.098 min

LC-MS m/z [M+H]⁺ calcd: 15159, found: 15159.

### Example C3: 7D12—Example 3 Conjugate

The title compound was prepared according to the Example C1 protocol adding 2 equivalents of maleimide-chelator after stirring overnight at 12° C.

anaSEC RT=5.997 min

LC-MS m/z [M+H]⁺ calcd: 15203, found: 15203.

### Example C4: 7D12—Example 4 Conjugate

The title compound was prepared according to the Example C1 protocol.

anaSEC RT=6.120 min

LC-MS m/z [M+H]⁺ calcd: 15118, found: 15119.

### Example C5: 7D12—Example 5 Conjugate

The title compound was prepared according to the Example C1 protocol.

anaSEC RT=6.064 min

LC-MS m/z [M+H]⁺ calcd: 15176, found: 15177.

### Example C6: 7D12—Example 6 Conjugate

The title compound was prepared according to the Example C1 protocol adding 2 equivalents of maleimide-chelator after stirring overnight at 8° C.

anaSEC RT=5.931 min

LC-MS m/z [M+H]⁺ calcd: 15204, found: 15266 (+Zn).

### Example C7: 7D12—Example 7 Conjugate

The title compound was prepared according to the Example C1 protocol adding 3 equivalents of maleimide-chelator after stirring overnight at 8° C.

anaSEC RT=5.95 min

LC-MS m/z [M+H]⁺ calcd: 15177, found: 15178.

### Example CN2: NY2547—Example 2 Conjugate

The title compound was prepared according to the Example C1 procedure.

anaSEC RT=6.098 min

LC-MS m/z [M+H]⁺ calcd: 15160, found: 15159.

### Example CN3: NY2547—Example 3 Conjugate

The title compound was prepared according to the Example C1 protocol adding 2 equivalents of maleimide-chelator after stirring overnight at 12° C.

anaSEC RT=5.997 min

LC-MS m/z [M+H]⁺ calcd: 15203, found: 15203.

### Example CN4: NY2547—Example 4 Conjugate

The title compound was prepared according to the Example C1 protocol.

anaSEC RT=6.12 min

LC-MS m/z [M+H]⁺ calcd: 15119, found: 15119.

### Example CN5: NY2547—Example 5 Conjugate

The title compound was prepared according to the Example C1 protocol.

anaSEC RT=6.064 min

LC-MS m/z [M+H]⁺ calcd: 15177, found: 15177.

### Example CN6: NY2547—Example 6 Conjugate

The title compound was prepared according to the Example C1 protocol adding 6 equivalents of maleimide-chelator after stirring overnight at 8° C.

anaSEC RT=5.931 min

LC-MS m/z [M+H]⁺ calcd: 15206, found: 15206.

### Example CN7: NY2547—Example 7 Conjugate

The title compound was prepared according to the Example C1 protocol adding 3 equivalents of maleimide-chelator after stirring overnight at 8° C.

anaSEC RT=5.95 min

LC-MS m/z [M+H]⁺ calcd: 15178, found: 15177 Example CN8: NY2547—Example 8 Conjugate

The title compound was prepared according to the Example C1 protocol.

anaSEC RT=6.131 min

LC-MS m/z [M+H]⁺ calcd: 13315, found: 13315.

### Example CN9: NY2547—Example 9 Conjugate

The title compound was prepared according to the Example C1 protocol.

anaSEC RT=5.83 min

LC-MS m/z [M+H]⁺ calcd: 13373, found: 13426 (+Fe).

### Example CN10: NY2547—Example 10 Conjugate

The title compound was prepared according to the Example C1 protocol.

anaSEC RT=6.07 min

LC-MS m/z [M+H]⁺ calcd: 13380, found: 13380 (+Fe).

### Example CN11: NY2547—Example 11 Conjugate

The title compound was prepared according to the Example C1 protocol adding 6 equivalents of maleimide-chelator after stirring overnight at 8° C.

anaSEC RT=5.99 min.

LC-MS m/z [M+H]⁺ calcd: 13347, found: 13347.

### Example CN12: NY2547—Example 12 Conjugate

The title compound was prepared The title compound was prepared according to the Example C1 protocol adding 2 equivalents of maleimide-chelator after stirring overnight at 12° C.

anaSEC RT=5.98 min

LC-MS m/z [M+H]⁺ calcd: 13373, found: 13426 (+Fe).

### Example CN13: NY2547—Example 13 Conjugate

The title compound was prepared according to the Example C1 protocol adding 3 equivalents of maleimide-chelator after stirring overnight at 12° C.

anaSEC RT=5.77 min

LC-MS m/z [M+H]⁺ calcd: 13437, found: 13490 (+Fe).

### Example CN14: NY2547—Example 14 Conjugate

The title compound was prepared according to the Example C1 protocol adding 6 equivalents of maleimide-chelator after stirring overnight at 8° C.

anaSEC RT=6.046 min

LC-MS m/z [M+H]⁺ calcd: 13330, found: 13330.

### Example CN15: NY2547—Example 15 Conjugate

The title compound was prepared according to the Example C1 protocol adding 3 equivalents of maleimide-chelator after stirring overnight at 8° C.

anaSEC RT=6.067 min

LC-MS m/z [M+H]⁺ calcd: 13407, found: 13407.

Examples C2 to C7, CN4, CN5 and CN7 were labeled with a molar activity of 1 MBq/μg sdAB according to the protocol stated below:

Radiolabeling with [⁶⁸Ga]GaCl₃:

Elute the ⁶⁸Ge/⁶⁸Ga generator: Aqueous HCl solution (0.1 M, 5 mL) is passed through the generator, and the eluate is fractionated in 0.5 mL aliquots. Typically, fractions 2 and 3 (containing a total of ~800 MBq [⁶⁸Ga]GaCl₃) are collected. 50 μL sodium acetate (1 M, pH 4.0) is mixed with 50 μL [⁶⁸Ga]GaCl₃ eluate (approx. 30-50 MBq in 0.1 M HCl). This mixture is pre-heated to 45° C. for 5 min and 300 rpm. Add 50 μg sdAb in 50 mM sodium acetate (~25 μL from 2 μg/μL) to the mixture from point 2. Measure the activity. The estimated reaction pH is 3.5-3.8. The reaction is kept at 45° C. for 30 min under 300 rpm. After 30 min, the reaction is quenched by adding 1 μl of 50 μM DTPA solution. A sample is taken for radio-HPLC and radio-TLC analysis to determine the incorporation of the radionuclide. If the incorporation follows the criteria of ≥85% incorporation as stated, the product will be formulated in dPBS by a factor of 10. A sample of the final product is withdrawn for an EOS radio-TLC, radio-HPLC chromatogram to determinate radiochemically purity (RCP) and pH (3.5-3.8). The final product can be tested in the stability tests (Table 11).

TABLE 9
Radioisotope incorporation and radiochemical purity

| | [⁶⁸Ga] gallium ion | HPLC purity of final |
|---|---|---|
| Compound | incorporation | product, EOS (%) |
| Example C2 | 100 | 96 |
| Example C3 | 100 | 94 |
| Example C4 | 100 | 97 |
| Example C5 | 95 | 92 |
| Example C6 | 95 | 92 |
| Example C7 | 100 | 96 |
| Example CN4 | 100 | 100 |
| Example CN5 | 100 | 95 |
| Example CN7 | 100 | 100 |

Radiolabeling with [¹⁷⁷Lu]LuCl₃:

50 μL sodium acetate (0.25 M, pH 5.5) is mixed with 75-100 MBq [¹⁷⁷Lu]LuCl₃ solution (in 0.05 M HCl, ~20 MBq/μl). This mixture is pre-heated to 45° C. for 5 min and 300 rpm. Add 50 μg sdAb in 50 mM sodium acetate (~50 μL from 2 μg/μL) to the pre-heated mixture. Measure the activity. The estimated reaction pH is 5.0-5.5. The reaction is kept at 45° C. for 30 min under 300 rpm. After the 30 min, the reaction is quenched by adding 1 μl of 50 μM DTPA solution. A sample is taken for radio-HPLC and radio-TLC to determine the incorporation of the radionuclide. If the incorporation follows the criteria of ≥85% incorporation, the product will be formulated in dPBS by a factor of 10. A sample of the final product will be withdrawn for an EOS radio-TLC and radio-HPLC chromatogram to determinate radiochemical purity (RCP) and for pH (5.0-5.5). The final product can be tested in the stability tests (Table 11).

TABLE 10
Radioisotope incorporation and radiochemical purity

| | [¹⁷⁷Lu] lutetium ion | HPLC purity of final |
|---|---|---|
| Compound | incorporation | product (%) |
| Example C2 | 100 | 96 |
| Example C3 | 100 | 94 |
| Example C4 | 100 | 97 |
| Example C5 | 95 | 92 |
| Example C6 | 95 | 92 |
| Example C7 | 100 | 96 |
| Example CN4 | 100 | 93 |
| Example CN5 | 100 | 91 |
| Example CN7 | 86 | 100 |

### In Vitro Mouse and Human Serum Stabilities:

Serum stability analysis was performed in human and mouse serum, and the procedure is as follows: 50 μl of each of the labeled compounds is mixed with 449 μl mouse serum and 1 μl 50 μM DTPA in an HPLC vial. This vial is kept at 37° C. and analyzed by SEC-radio-HPLC (100 μl injection) at the following intervals: [⁶⁸Ga]Ga-sdAb-conjugates: 0, 4 hours; [¹⁷⁷Lu]Lu-sdAb-conjugates: 0, 24 hours (Table 11). Stability data are corrected for 100% purity at t=0.

TABLE 11
Stability in mouse/human serum

| Mouse/ | ⁶⁸Ga-conjugates | ¹⁷⁷Lu-conjugates |
|---|---|---|
| human serum | stability at 4 hours | stability at 24 hours |
| Example C2 | 91/87 | 78/76 |
| Example C3 | 100/100 | 84/80 |
| Example C4 | 87/93 | 86/84 |
| Example C5 | 98/100 | 87/84 |
| Example C6 | 77/83 | 90/89 |
| Example C7 | 87/93 | 86/84 |
| Example CN4 | 98/100 | 100/72 |
| Example CN5 | 100/100 | 100/93 |
| Example CN7 | 100/100 | 80/88 |

Examples C1, CN8 to CN15 were evaluated according to the protocol stated below:

Radiolabeling with [⁶⁸Ga]GaCl₃ and Human Serum Stability:

25 μg of NY2547-chelator were placed in a 1.5 mL microtube and MilliQ water (if necessary, to bring the final radiolabelling solution volume up to 60-62 μL) and Somakit gallium buffer ( 1/10 of the volume of Ga-68 to adjust the pH at 3.2-3.5) were added. Finally, 22 MBq of [⁶⁸Ga]GaCl₃ (formulated in 0.1 M HCl) were added and, after vortexing for 5 seconds, the microtube was incubated in a heating block at 45° C. for 15 min. After 15 min, the radiolabeling solution was diluted 4-fold using PBS and then 1 μL of DTPA 7.7 mM per 30 μL of diluted solution (e.g. 15 μL radiolabeling solution+45 μL PBS+2 μL DTPA 7.7 mM) was added.

20 μL of the diluted radiolabeling solution were injected to determine the radiolabeling efficiency using SE radio-HPLC chromatography.

In parallel, 7 μL of DTPA 7.7 mM were added to a 200 μL aliquot of human serum.
180 μL of the obtained serum solution were added to 20 μL of the non-diluted radiolabelling solution. The final solution was incubated for 4 hours at 37° C. in the HPLC autosampler. Stability was assessed injecting from 50 to 80 μL of this serum solution after 4 hours. Experiments were done in duplicate or in triplicate. Stability data are corrected for 100% purity at t=0 (Table 12).

TABLE 12
Radiochemical purity and stability in human serum

| ⁶⁸Ga | HPLC purity of | Stability at |
|---|---|---|
| conjugates | final product (%) | 4 hours |
| Example C1 | 98 | 97 |
| Example CN8 | 30 | Not tested |
| Example CN9 | 96 | 97 |
| Example CN10 | 88 | 99 |
| Example CN11 | 99 | 96 |
| Example CN12 | 98 | 99 |
| Example CN13 | 99 | 97 |
| Example CN14 | 98 | 98 |
| Example CN15 | 93 | <5% |

Radiolabeling with [¹⁷⁷Lu]LuCl₃ and human serum stability:

30 μg of NY2547-chelator were placed in a 1.5 mL microtube and MilliQ water (to have a final radiolabelling solution volume of 30 μL) and sodium acetate 47 mg/mL buffer (approximately half of the volume of Lu-177 to adjust the pH at 5-5.5) were added. Finally, 85-110 MBq of [¹⁷⁷Lu]LuCl₃ (formulated in 0.05 M HCl) were added and, after vortexing for 5 seconds, the microtube was incubated in a heating block at 45° C. for 10 minutes. NY2547-chelator/Lu ratio is ~1.8. After 10 min, the radiolabeling solution was diluted 10-fold using PBS and 1 μL of DTPA was added.

7.7 mM per 30 μL of diluted solution (e.g. 3 μL radiolabeling solution+27 μL PBS+1 μL DTPA 7.7 mM). 15 μL of the diluted radiolabeling solution were injected to determine the radiolabeling efficiency using SE radio-HPLC chromatography.

20 μL of the diluted radiolabeling solution were added to 180 μL of human serum and incubated for 24 hours at 37° C. in the HPLC autosampler. Stability was assessed by injecting 50 μL of these serum solution at t 0 and t 24 hours. Experiments were done in duplicate. Stability data are corrected for 100% purity at t=0 (Table 13).

TABLE 13
Radiochemical purity and stability in human serum

| ¹⁷⁷Lu | HPLC purity of | Stability at 24 |
|---|---|---|
| conjugates | final product (%) | hours (%) |
| Example C1 | 98 | 63 |
| Example CN8 | 94 | 78 |
| Example CN9 | 96 | 65 |
| Example CN10 | 92 | 82 |
| Example CN11 | 98 | 80 |
| Example CN12 | 98 | 86 |
| Example CN13 | 97 | 78 |
| Example CN14 | 97 | 77 |
| Example CN15 | 98 | 83 |

### Example 16: (2R,2′R)-2,2′-(1³-(3-(2,5-dioxo-2,5-dihydro-1H-pyrrol-1-yl)propoxy)-3,7-diaza-1,5(2,6)-dipyridinacyclooctaphane-3,7-diyl)disuccinic acid

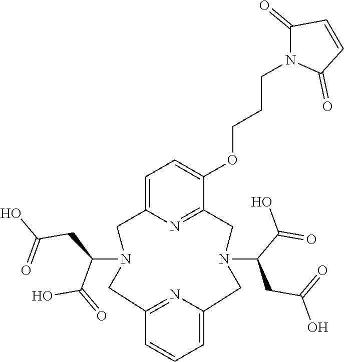 [SMILES: O=C(O)C[C@H](C(=O)O)N1Cc2cccc(n2)CN([C@H](CC(=O)O)C(=O)O)Cc2nc(ccc2OCCCN2C(=O)C=CC2=O)C1]

### Step 1: 2-(3-((2,6-bis(hydroxymethyl)pyridin-3-yl)oxy)propyl)isoindoline-1,3-dione

To a suspension of (3-hydroxypyridine-2,6-diyl)dimethanol hydrochloride (200 mg, 1.04 mmol) and K₂CO₃ (721.3 mg, 5.22 mmol) in DMF (4.0 mL) was added 2-(3-bromopropyl)isoindoline-1,3-dione (419.8 mg, 1.57 mmol). After stirring at 80° C. for 4 hours, the reaction mixture was cooled to RT and extracted with EtOAc. The combined organic layers were washed with water, dried (Phase Separator) and concentrated under reduced pressure. The crude product was purified by flash chromatography on silica gel and eluted with CH₂Cl₂/MeOH/NH₄OH 95:5:0.5 to afford 143 mg as a pale powder. LC-MS-15: Rt=0.46 min; m/z [M+H]⁺ 343. ¹H NMR (400 MHz, DMSO) δ 7.93-7.74 (m, 4H), 7.40-7.22 (m, 2H), 5.28 (t, J=5.8 Hz, 1H), 4.71 (t, J=5.6 Hz, 1H), 4.47 (dd, J=16.5, 5.7 Hz, 4H), 4.04 (t, J=5.8 Hz, 2H), 3.78 (t, J=6.7 Hz, 2H), 2.07 (q, J=6.3 Hz, 2H).

### Step 2: 2-(3-((2,6-bis(bromomethyl)pyridin-3-yl)oxy)propyl)isoindoline-1,3-dione

To a suspension of 2-(3-((2,6-bis(hydroxymethyl)pyridin-3-yl)oxy)propyl)isoindoline-1,3-dione (140 mg, 408.9 μmol) in acetonitrile (4 mL) was added at 0° C., CBr₄ (474.7 mg, 1.43 mmol) and triphenylphosphine (375.4 mg, 1.4 mmol). After stirring the reaction mixture at RT for 6 hours, the reaction mixture was filtered (celite) and the filtrate was concentrated under reduced pressure. The residue was purified by flash chromatography on silica gel and was eluted with cyclohexane/EtOAc (+1% TEA) from 100:0 to 0:100. LC-MS-15: Rt=1.10 min; m/z [M+H]⁺ 468.9. ¹H NMR (400 MHz, DMSO) δ 7.84 (dtd, J=8.9, 6.1, 3.4 Hz, 4H), 7.62-7.40 (m, 2H), 4.66 (s, 2H), 4.58 (s, 2H), 4.15 (t, J=5.8 Hz, 2H), 3.82 (t, J=6.7 Hz, 2H), 2.11 (p, J=6.3 Hz, 2H).

### Step 3: tetra-tert-butyl 2,2′-((pyridine-2,6-diylbis(methylene))bis(azanediyl))(2R,2′R)-disuccinate

To a solution of di-tert-butyl D-aspartate hydrochloride (5.3 g, 18.87 mmol) in CH₃CN (30 mL) was added DIPEA (2.9 g, 3.94 mL, 22.65 mmol), followed by the dropwise addition of a solution of 2,6-bis(bromomethyl)pyridine (1.0 g, 3.7 mmol) in CH₃CN (30.0 mL) at 0° C. After stirring at 20° C. for 16 hours, the temperature was raised to 60° C. for 2 hours. The reaction mixture was concentrated under reduced pressure. The residue was purified by flash chromatography on silica gel and eluted with cyclohexane/EtOAc (+1% Et₃N) from 100:0 to 0:100 to afford 4.2 g as a yellow oil. A second purification was performed using the method RP-HPLC-6 to afford 980 mg as pure material. LC-MS-15: Rt=1.25 min; m/z [M+H]⁺ 594.4. ¹H NMR (400 MHz, DMSO) δ 7.69 (t, J=7.7 Hz, 1H), 7.26 (d, J=7.7 Hz, 2H), 3.84 (d, J=14.6 Hz, 2H), 3.70 (d, J=14.6 Hz, 2H), 3.41 (t, J=6.5 Hz, 2H), 2.07 (s, 1H), 1.39 (d, J=7.5 Hz, 40H).

### Step 4: tetra-tert-butyl 2,2′-(1³-(3-(1,3-dioxoisoindolin-2-yl)propoxy)-3,7-diaza-1,5(2,6)-dipyridinacyclooctaphane-3,7-diyl)(2R,2′R)-disuccinate

To a solution of tetra-tert-butyl 2,2′-((pyridine-2,6-diylbis(methylene))bis(azanediyl))(2R,2′R)-disuccinate (122.9 mg, 149.5 μmol) in CH₃CN (4.0 mL) was added DIPEA (96.6 mg, 130 μL, 747.6 μmol) followed by 2-(3-((2,6-bis(bromomethyl)pyridin-3-yl)oxy)propyl)isoindoline-1,3-dione (70.00 mg, 149.5 μmol). After stirring for 16 hours at 40° C., the precipitate was filtered off and the filter cake was washed with CH₃CN. The filtrate was concentrated under reduced pressure. The residue was purified by flash chromatography on silica gel and eluted with cyclohexane/EtOAc (+1% TEA) from 100:0 to 0:100 to afford the title compound (130 mg) as a yellow oil. LC-MS-15: Rt=1.19 min; m/z [M+H]⁺ 901.5. ¹H NMR (400 MHz, DMSO) δ 7.95-7.75 (m, 3H), 7.26 (d, J=7.7 Hz, 1H), 7.11 (t, J=7.6 Hz, 1H), 6.80 (d, J=7.6 Hz, 1H), 6.78-6.63 (m, 2H), 4.03 (q, J=7.1 Hz, 2H), 3.97-3.56 (m, 4H), 2.85 (ddd, J=31.1, 16.1, 8.2 Hz, 1H), 2.71 (dd, J=16.1, 8.3 Hz, 2H), 2.05 (s, 1H), 1.99 (s, 4H), 1.58-1.42 (m, 21H), 1.42-1.32 (m, 17H), 1.17 (t, J=7.1 Hz, 4H).

### Step 5: tetra-tert-butyl 2,2′-(1³-(3-aminopropoxy)-3,7-diaza-1,5(2,6)-dipyridinacyclooctaphane-3,7-diyl)(2R,2′R)-disuccinate

To a solution of tetra-tert-butyl 2,2′-(1³-(3-(1,3-dioxoisoindolin-2-yl)propoxy)-3,7-diaza-1,5(2,6)-dipyridinacyclooctaphane-3,7-diyl)(2R,2′R)-disuccinate (350.0 mg, 388.9 μmol) in CH₂Cl₂ (4.0 mL) and MeOH (4.0 mL) was added hydrazine and water (286.0 μL, 5.8 mmol). After stirring overnight at RT, the reaction mixture was washed with a saturated solution of sodium bicarbonate, the combined organic layers were dried (Phase Separator) and concentrated under reduced pressure. The residue was purified by flash chromatography on silica gel and was eluted with CH₂Cl₂/MeOH/NH₄OH 50:50:1 to afford 275 mg of title compound as a colorless oil. LC-MS-15: Rt=0.82 min; m/z [M+H]⁺ 770.5. ¹H NMR (400 MHz, DMSO) δ 7.12 (t, J=7.7 Hz, 1H), 6.88-6.62 (m, 5H), 4.05-3.65 (m, 9H), 3.01-2.64 (m, 6H), 1.85-1.65 (m, 2H), 1.52 (s, 20H), 1.47 (s, 8H), 1.43 (s, 9H), 1.05 (t, J=6.9 Hz, 3H).

### Step 6: tetra-tert-butyl 2,2′-(1³-(3-(2,5-dioxo-2,5-dihydro-1H-pyrrol-1-yl)propoxy)-3,7-diaza-1,5(2,6)-dipyridinacyclooctaphane-3,7-diyl)(2R,2′R)-disuccinate

To a solution of tetra-tert-butyl 2,2′-(1³-(3-aminopropoxy)-3,7-diaza-1,5(2,6)-dipyridinacyclooctaphane-3,7-diyl)(2R,2′R)-disuccinate (270 mg, 350.7 μmol) in toluene (4.0 mL) was added at RT under argon TEA (70.97 mg, 97.7 μL, 701.3 μmol) and at 0° C. dropwise a solution of furan-2,5-dione (41.26 mg, 420.8 μmol) in toluene (4.0 mL). After stirring the reaction mixture at RT for 1 hour, the reaction mixture was concentrated under reduced pressure. The residue was dissolved in CH₂Cl₂ (4.0 mL), pentafluorophenol (97.15 mg, 1.5 Eq, 526.0 μmol) and diisopropylcarbodiimide (132.8 mg, 163 μL, 3.0 Eq, 1.05 mmol) were added. After stirring at RT for 16 hours, the reaction mixture was concentrated under reduced pressure and the residue was purified using the method RP-HPLC-6 to afford 175 mg as a colorless oil. LC-MS-15: Rt=1.11 min; m/z [M+H]⁺ 850.6. ¹H NMR (400 MHz, DMSO) δ 8.21 (t, J=7.8 Hz, 1H), 7.79-7.58 (m, 4H), 7.49 (d, J=8.4 Hz, 1H), 7.00 (s, 2H), 4.39 (dd, J=47.2, 15.4 Hz, 3H), 4.13 (dt, J=28.3, 6.5 Hz, 3H), 3.79-3.58 (m, 4H), 2.13-1.93 (m, 2H), 1.44 (d, J=3.4 Hz, 21H), 1.27 (d, J=3.2 Hz, 22H).

### Step 7: (2R,2′R)-2,2′-(1³-(3-(2,5-dioxo-2,5-dihydro-1H-pyrrol-1-yl)propoxy)-3,7-diaza-1,5(2,6)-dipyridinacyclooctaphane-3,7-diyl)disuccinic acid

To a solution of tetra-tert-butyl 2,2′-(1³-(3-(2,5-dioxo-2,5-dihydro-1H-pyrrol-1-yl)propoxy)-3,7-diaza-1,5(2,6)-dipyridinacyclooctaphane-3,7-diyl)(2R,2′R)-disuccinate (175.0 mg, 205.9 μmol) in water (277.8 μL) was added TFA (2.5 mL). After stirring at RT for 16 hours, a second addition of TFA (2.5 mL) and water (277.8 μL) was performed and the reaction mixture was stirred at RT for 2 hours. Water was added (1 mL) and the resulting solution was purified using the RP-HPLC-6 conditions to afford 121 mg as white powder. LC-MS-13: Rt=0.70 min; m/z [M+H]⁺ 626.3. ¹H NMR (400 MHz, DMSO) δ 8.19 (s, 1H), 7.66 (s, 3H), 7.43 (s, 1H), 7.01 (s, 2H), 4.84-3.98 (m, 6H), 3.96-3.23 (m, 10H), 2.30 (d, J=19.8 Hz, 2H), 2.01 (q, J=6.3 Hz, 2H).

### Example 17: (((1S)-5-((2R)-2-amino-3-((2,5-dioxo-1-(4-(3,6,9-tris(carboxymethyl)-3,6,9-triaza-1(2,6)-pyridinacyclodecaphane-4-yl)butyl)pyrrolidin-3-yl)thio)propanamido)-1-carboxypentyl)carbamoyl)-L-glutamic acid

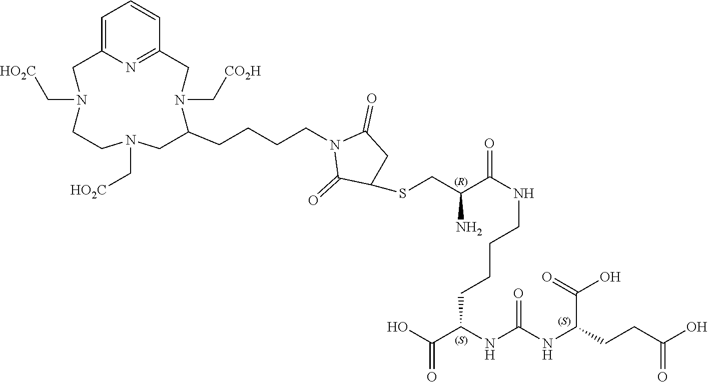

### Step 1: tri-tert-butyl (9S,13S)-3,11-dioxo-1-phenyl-2-oxa-4,10,12-triazapentadecane-9,13,15-tricarboxylate

A solution of di-tert-butyl L-glutamate hydrochloride (500 mg, 1.69 mmol) and DIPEA (dried over molecular sieve) (720.9 mg, 972 μL, 5.58 mmol) in CH₂Cl₂ (20.0 mL) was cooled under argon to −78° C. using a dry ice/acetone bath. Triphosgene (175.5 mg, 0.35 Eq, 591.6 μmol) dissolved in CH₂Cl₂ (5.0 mL) was added dropwise to the reaction. After the addition was complete, the reaction was allowed to warm to room temperature and stirred for 30 minutes. H-Lys(Z)-OtBu, hydrochloride (504.2 mg, 1.35 mmol) was then added to the reaction mixture, followed by DIPEA (dried over molecular sieve) (174.8 mg, 236 μL, 1.35 mmol). After stirring overnight at RT, the reaction mixture was extracted with CH₂Cl₂, the organic layers washed with H₂O (3×50 mL), dried using a phase separator and concentrated under reduced pressure. The crude product was purified by flash chromatography on silica gel and eluted with cyclohexane/EtOAc 3:2 to afford the title compound as a yellow oil (960 mg, 87%). LC-MS-15: Rt=1.48 min; m/z [M+H]⁺ 622.5. ¹H NMR (400 MHz, DMSO) δ 7.44-7.16 (m, 6H), 6.28 (dd, J=15.8, 8.3 Hz, 2H), 4.99 (s, 2H), 4.15-3.86 (m, 6H), 2.97 (q, J=6.6 Hz, 2H), 2.31-2.08 (m, 2H), 1.99 (s, 5H), 1.86 (dq, J=11.7, 5.9 Hz, 1H), 1.75-1.48 (m, 1H), 1.39 (d, J=2.9 Hz, 30H), 1.34-1.23 (m, 2H), 1.17 (t, J=7.1 Hz, 6H).

### Step 2: di-tert-butyl (((S)-6-amino-1-(tert-butoxy)-1-oxohexan-2-yl)carbamoyl)-L-glutamate

A solution of tri-tert-butyl (9S,13S)-3,11-dioxo-1-phenyl-2-oxa-4,10,12-triazapentadecane-9,13,15-tricarboxylate (960.0 mg, 1.54 mmol) in MeOH (20.0 mL) was evacuated and back-filled with argon 3 times. Pd/C 10% (164.3 mg, 10% Wt, 0.1 Eq, 154.4 μmol) was added and the flask was evacuated and back-filled with argon 3 times, then evacuated and back-filled with hydrogen 3 times. The reaction mixture was stirred vigorously at RT for 16 hours under H₂ atmosphere at a pressure of 1 bar. The reaction mixture was filtered through celite and the filtrate was concentrated under reduced pressure. The residue (820 mg, pale oil) was purified by flash chromatography on silica gel, eluted with CH₂Cl₂/MeOH/NH₄OH 90:10:1 to afford 575 mg as a colorless oil. LC-MS-15: Rt=1.08 min; m/z [M+H]⁺ 488. ¹H NMR (400 MHz, DMSO) δ 6.28 (dd, J=16.9, 8.3 Hz, 2H), 4.36 (s, 1H), 3.99 (dtd, J=25.7, 8.3, 5.2 Hz, 2H), 3.44 (q, J=7.0 Hz, 2H), 2.35-2.08 (m, 2H), 1.86 (dp, J=13.6, 6.1 Hz, 1H), 1.73-1.45 (m, 2H), 1.45-1.35 (m, 30H), 1.34-1.17 (m, 2H), 1.05 (t, J=6.9 Hz, 3H).

### Step 3: tri-tert-butyl (6R,13S,17S)-2,2-dimethyl-4,7,15-trioxo-6-((tritylthio)methyl)-3-oxa-5,8,14,16-tetraazanonadecane-13,17,19-tricarboxylate

To a solution of di-tert-butyl (((S)-6-amino-1-(tert-butoxy)-1-oxohexan-2-yl)carbamoyl)-L-glutamate (100.0 mg, 205.1 μmol) in THF (2.0 mL) was added under argon 2,5-dioxopyrrolidin-1-yl N-(tert-butoxycarbonyl)-S-trityl-L-cysteinate (138.0 mg, 246.1 μmol) and NEt₃ (26.98 mg, 37.2 μL, 266.6 μmol). After stirring overnight at RT, the reaction mixture was concentrated under reduced pressure and the residue was extracted with EtOAc. The combined organic layers were washed with a saturated solution of sodium bicarbonate and brine, dried (Phase Separator) and concentrated under reduced pressure. The crude product was purified by flash chromatography on silica gel and eluted with cyclohexane/EtOAc 3:2 to provide 185 mg as a colorless oil (spontaneous crystallization). LC-MS-15: Rt=1.68 min; m/z [M+H]⁺ 933.9. ¹H NMR (400 MHz, DMSO) δ 7.76 (t, J=5.7 Hz, 1H), 7.43-7.16 (m, 18H), 6.84 (d, J=8.5 Hz, 1H), 6.25 (dd, J=12.2, 8.3 Hz, 2H), 3.92 (dd, J=8.9, 6.1 Hz, 2H), 2.97 (ddd, J=39.8, 13.2, 6.5 Hz, 2H), 2.36-2.12 (m, 5H), 1.86 (td, J=13.2, 6.6 Hz, 1H), 1.74-1.42 (m, 2H), 1.42-1.29 (m, 38H), 1.28-1.20 (m, 1H).

### Step 4: di-tert-butyl (((S)-1-(tert-butoxy)-6-((R)-2-((tert-butoxycarbonyl)amino)-3-mercaptopropanamido)-1-oxohexan-2-yl)carbamoyl)-L-glutamate

To a solution of tri-tert-butyl (6R,13S,17S)-2,2-dimethyl-4,7,15-trioxo-6-((tritylthio)methyl)-3-oxa-5,8,14,16-tetraazanonadecane-13,17,19-tricarboxylate (180.0 mg, 80% purity, 154.3 μmol)) in CH₂Cl₂ (1.5 mL) was added at 0° C. triethylsilane (21.53 mg, 29.6 μL, 185.2 μmol) followed by TFA (62.50 μL). After stirring the reaction mixture at 0° C. for 10 min, the reaction mixture was extracted with CH₂Cl₂. The combined organic layers were washed with a saturated solution of sodium bicarbonate, dried (Phase Separator) and concentrated under reduced pressure. The crude product was purified by flash chromatography and eluted with cyclohexane/EtOAc from 100:0 to 0:100, to provide the title compound (100 mg) as a colorless oil (spontaneous crystallization). LC-MS-15: Rt=1.45 min; m/z [M+H]⁺ 691.3. ¹H NMR (400 MHz, DMSO) δ 7.89 (t, J=5.6 Hz, 2H), 6.87 (d, J=8.2 Hz, 2H), 6.26 (dd, J=10.7, 8.3 Hz, 5H), 4.14-3.81 (m, 10H), 3.03 (ddt, J=18.9, 13.0, 6.4 Hz, 5H), 2.82-2.56 (m, 4H), 2.33-2.11 (m, 7H), 1.99 (s, 5H), 1.86 (dq, J=13.3, 6.7 Hz, 2H), 1.75-1.45 (m, 3H), 1.41 (s, 3H), 1.30-1.00 (m, 11H).

### Step 6: di-tert-butyl (((2S)-1-(tert-butoxy)-6-((2R)-2-((tert-butoxycarbonyl)amino)-3-((2,5-dioxo-1-(4-(3,6,9-tris(2-(tert-butoxy)-2-oxoethyl)-3,6,9-triaza-1(2,6)-pyridinacyclodecaphane-4-yl)butyl)pyrrolidin-3-yl)thio)propanamido)-1-oxohexan-2-yl)carbamoyl)-L-glutamate

To a solution of example 1 (30.0 mg, 42.9 μmol) and Et₃N (21.69 mg, 29.9 μL, 214.3 μmol) was added di-tert-butyl (((S)-1-(tert-butoxy)-6-((R)-2-((tert-butoxycarbonyl)amino)-3-mercaptopropanamido)-1-oxohexan-2-yl)carbamoyl)-L-glutamate (35.5 mg, 51.4 μmol). After stirring at RT for 16 hours, the reaction mixture was concentrated under reduced pressure and the residue purified using RP-HPLC-4 conditions to afford the title compound (28 mg) as a white powder. LC-MS-16: Rt=6.86 min; m/z [M+H]⁺ 1391.8. ¹H NMR (400 MHz, DMSO) δ 8.08-7.84 (m, 1H), 7.77 (t, J=7.7 Hz, 1H), 7.24 (dd, J=16.6, 7.7 Hz, 1H), 7.00 (d, J=8.6 Hz, 1H), 6.27 (t, J=8.8 Hz, 2H), 4.38-3.88 (m, 6H), 3.88-3.35 (m, 34H), 3.33-2.84 (m, 2H), 2.86-2.59 (m, 1H), 2.37-2.11 (m, 3H), 1.86 (dq, J=13.9, 7.0 Hz, 1H), 1.73-1.56 (m, 1H), 1.53-1.18 (m, 51H), 1.12 (s, 5H).

### Step 7: (((1S)-5-((2R)-2-amino-3-((2,5-dioxo-1-(4-(3,6,9-tris(carboxymethyl)-3,6,9-triaza-1(2,6)-pyridinacyclodecaphane-4-yl)butyl)pyrrolidin-3-yl)thio)propanamido)-1-carboxypentyl)carbamoyl)-L-glutamic acid

To a solution of di-tert-butyl (((2S)-1-(tert-butoxy)-6-((2R)-2-((tert-butoxycarbonyl)amino)-3-((2,5-dioxo-1-(4-(3,6,9-tris(2-(tert-butoxy)-2-oxoethyl)-3,6,9-triaza-1(2,6)-pyridinacyclodecaphane-4-yl)butyl)pyrrolidin-3-yl)thio)propanamido)-1-oxohexan-2-yl)carbamoyl)-L-glutamate (14.0 mg, 10.07 μmol) in water (22.2 μL), TFA (200.0 μL) was added. After stirring for 16 hours at RT, water was added (1 mL) and the reaction mixture was purified following RP-HPLC-5 conditions to afford the title compound (28 mg) as a white powder. LC-MS-15: Rt=0.54 min; m/z [M+H]⁺ 954.5. ¹H NMR (400 MHz, DMSO) δ 12.52 (s, 12H), 8.58 (dd, J=45.7, 9.4 Hz, 2H), 8.31 (s, 6H), 7.72 (d, J=7.9 Hz, 2H), 7.20 (dd, J=15.4, 7.7 Hz, 3H), 6.32 (t, J=9.2 Hz, 4H), 4.43-3.85 (m, 6H), 2.41-2.14 (m, 4H), 2.08-1.85 (m, 2H), 1.71 (td, J=15.1, 8.4 Hz, 3H), 1.59-1.18 (m, 14H).

Radiolabeling and Buffer Stability of Example 17 with ¹⁷⁷LuCl₃

To a mixture of sodium acetate in water (35.0 μL, 1.5 M), ascorbic acid in H₂O (35.00 μL, 0.04 M), ethanol (58.5 μL) and acetic acid (1.5 μL) was added [¹⁷⁷Lu]LuCl₃ (250 ml, 0.04 M HCl, 58.2 MBq). A solution of (((1S)-5-((2R)-2-amino-3-((2,5-dioxo-1-(4-(3,6,9-tris(carboxymethyl)-3,6,9-triaza-1(2,6)-pyridinacyclodecaphane-4-yl)butyl)pyrrolidin-3-yl)thio)propanamido)-1-carboxypentyl)carbamoyl)-L-glutamic acid (7.9 μL) of a 0.2 mM in water was added. After shaking (450 rpm) at 95° C. for 15 min, the reaction mixture was left cooling down for few minutes. The solution was diluted with 385 μl of PBS to keep the final formulation with EtOH below 9%. The radiochemical purity and stability were assessed using HPLC-1 conditions: Waters; column: phenomenex Luna; particle size 2.5 μm; size: 4.6×100 mm; flow rate: 1 mL/min; mobile phase A: H2O+0.1% TFA and B: CH3CN; gradient: 10 to 80% solvent B in 9 min.

RCP at t=0, 93% and stability t=4 hours, 94%; t=8 hours, 95%; t=24 hours, 94%.

Radiolabeling and Buffer Stability of Example 17 with ⁶⁸GaCl₃

A solution of sodium acetate trihydrate in water 1.5 M (740.0 μL), acetic acid (260.0 μL) and (((1S)-5-((2R)-2-amino-3-((2,5-dioxo-1-(4-(3,6,9-tris(carboxymethyl)-3,6,9-triaza-1(2,6)-pyridinacyclodecaphane-4-yl)butyl)pyrrolidin-3-yl)thio)propanamido)-1-carboxypentyl)carbamoyl)-L-glutamic acid (2 mM; 1.907 mg/mL; 2 nmol in 1 ml) were added to a 8 mL glass V-vial. This vial was sealed, the vacuum needle and the ⁶⁸Ga/HCl 0.1M needle were connected to the vial. The gallium generator was eluted to the reaction vial (approximately 260 MBq [⁶⁸Ga]GaCl₃ in HCl 0.1M, 1.1 mL). The vacuum needle and the ⁶⁸Ga/HCl 0.1M needle were disconnected from the reaction vial. After shaking (600 rpm) at 45° C. for 30 min, the reaction mixture was left cooling down for few minutes. The radiochemical purity and stability were assessed using HPLC-1 conditions: Waters; column: Phenomenex Luna; particle size 2.5 μm; size: 4.6×100 mm; flow rate: 1 mL/min; mobile phase A: H2O+0.1% TFA and B: CH3CN; gradient: 10 to 80% solvent B in 9 min. RCP at t=0, 75% and stability t=4 hours, 74%.

1. A compound of formula (I) 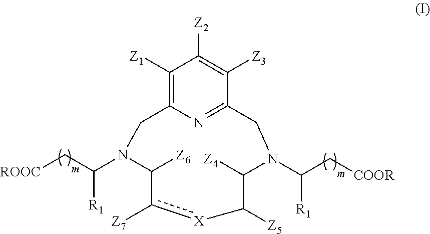
or a pharmaceutically acceptable salt thereof,
wherein [symbol]
is a single bond or a double bond,
when [symbol] is a single bond, X is —O— or 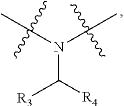
when [symbol] is a double bond, X is ═N— and Z₅ and Z₇ form, together with the N atom, a heteroaryl having 5 or 6 ring atoms, otherwise, Z₅ and Z₇ are each independently selected from the group consisting of H, an albumin-binder and —X₁-L₁-X₂-L₂-A;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms, or any combination thereof;
Z₁, Z₂, Z₃, Z₄, and Z₆ are each independently selected from the group consisting of H, an albumin-binder and —X₁-L₁-X₂-L₂-A, provided that at least one of Z₁, Z₂, Z₃, Z₄, Z₅, Z₆, and Z₇ is the group —X₁-L₁-X₂-L₂-A;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—, —CH₂—O—, and —NR′C(═O)NR′—, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents selected from: an albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂ is selected from the group consisting of: 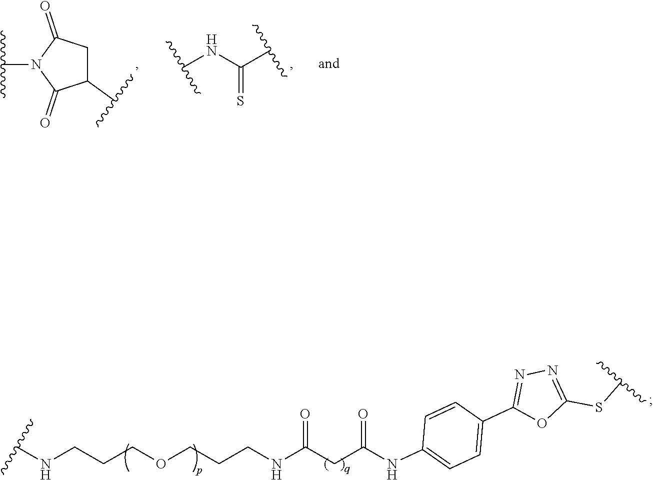
L₂ is a bond or a linker comprising one or more amino acids, one or more N-substituted amino acids, optionally substituted polyether, optionally substituted C₁-C₁₂ alkylene, optionally substituted C₂-C₁₀ alkenylene, optionally substituted arylene having 6 to 10 ring atoms, optionally substituted C₃-C₆ cycloalkylene, optionally substituted heterocycloalkylene having 5 to 10 ring atoms, optionally substituted heteroarylene having 5 to 10 ring atoms, or any combination thereof, wherein the alkylene and alkenylene optionally comprise one or more heteroatom or chemical groups selected from —O—, —S—, —C(═O)—, —NR″—, —C(═O)NR″—, —NR″—C(═O)—, —NR″—C(═O)—NR′″—, —NR″—C(═O)—O—, —O—C(═O)NR″—; and
each m is independently 0 or 1;
n is 1, 2, 3, 4, 5, or 6;
p is 1, 2, 3, 4, 5, or 6;
q is 1, 2, 3, 4, 5, or 5; and
A is a target binding moiety.

2. The compound according to claim 1, or a pharmaceutically acceptable salt thereof, wherein R is H.

3. The compound according to claim 1 or 2, or a pharmaceutically acceptable salt thereof, wherein each R₁ is independently selected from the group consisting of H, C₁-C₆ alkyl, C(═O)OR₂, (C₁-C₆ alkyl)-C(═O)OR₂, and heteroaryl having 5 to 10 ring atoms.

4. The compound according to any one of claims 1 to 3, or a pharmaceutically acceptable salt thereof, wherein each R₁ is independently selected from the group consisting of H, CH₃, C(═O)OH, CH₂C(═O)OH and pyridyl.

5. The compound according to any one of claims 1 to 4, or a pharmaceutically acceptable salt thereof, wherein R₃ is selected from the group consisting of H, C₁-C₃ alkyl, and C(═O)OR.

6. The compound according to any one of claims 1 to 5, or a pharmaceutically acceptable salt thereof, wherein R₃ is selected from the group consisting of H, CH₃ and C(═O)OH.

7. The compound according to any one of claims 1 to 6, or a pharmaceutically acceptable salt thereof, wherein R₄ is selected from the group consisting of H, C(═O)OR, (C₁-C₆ alkyl)-C(═O)OR and heteroaryl having 5 to 10 ring atoms.

8. The compound according to any one of claims 1 to 7, or a pharmaceutically acceptable salt thereof, wherein R₄ is selected from the group consisting of H, C(═O)OH, CH₂C(═O)OH and pyridyl.

9. The compound according to any one of claims 1 to 8, or a pharmaceutically acceptable salt thereof, wherein only one of Z₁, Z₂, Z₃, Z₄, Z₅, Z₆, and Z₇ is —X₁-L₁-X₂-L₂-A, and the other Z groups are H.

10. The compound according to any one of claims 1 to 9, or a pharmaceutically acceptable salt thereof, wherein X₁ is selected from the group consisting of —O—, —N(CH₃)— and —C(═O)NH— or absent.

11. The compound according to any one of claims 1 to 10, or a pharmaceutically acceptable salt thereof, wherein L₁ is C₁-C₅ alkylene.

12. The compound according to any one of claims 1 to 11, or a pharmaceutically acceptable salt thereof, wherein X₂ is

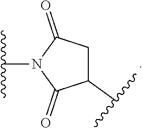 [SMILES: *C1CC(=O)N(*)C1=O]

13. The compound according to any one of claims 1 to 12, or a pharmaceutically acceptable salt thereof, wherein A is a target binding moiety comprising a peptide, polypeptide, protein, peptidomimetic, aptamer, DARPin, antisense oligonucleotide, siNA, small molecule, microparticle or nanoparticle.

14. The compound according to any one of claims 1 to 13, or a pharmaceutically acceptable salt thereof, wherein A is a target binding moiety comprising a peptide, polypeptide or protein.

15. The compound of any one of claims 1 to 14, or a pharmaceutically acceptable salt thereof, wherein A is a target binding moiety comprising an antibody, a VhH antibody, a nanobody, a single domain antibody, a protein comprising an antigen-binding region of an antibody, or a fusion protein.

16. The compound according to any one of claims 1 to 15, wherein A is a target binding moiety comprising nimotuzumab, trastuzumab, sacituzumab, ramucirumab, cetuximab, enolituzumab, tusamitamab, amivantamab, or datopotamab.

17. The compound according to any one of claims 1 to 16, of formula (Id) 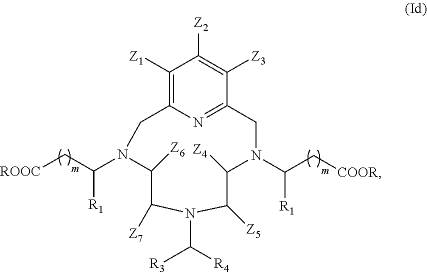
or a pharmaceutically acceptable salt thereof.

18. The compound according to any one of claims 1 to 16, of formula (Ie) 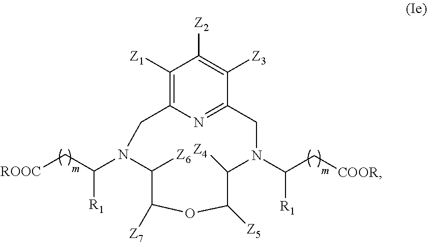
or a pharmaceutically acceptable salt thereof.

19. The compound according to any one of claims 1 to 16, of formula (If) 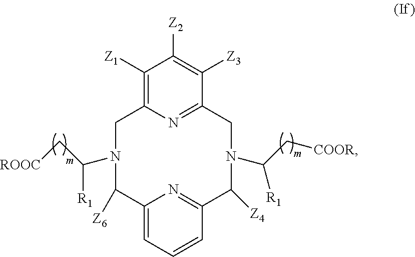
or a pharmaceutically acceptable salt thereof.

20. The compound according to any one of claims 1 to 16, which is selected from 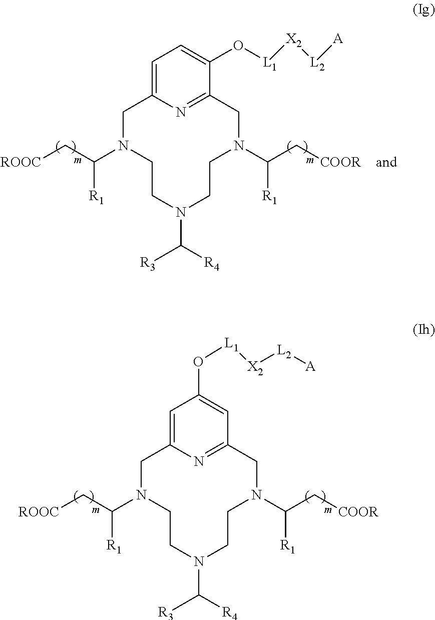
or a pharmaceutically acceptable salt thereof.

21. The compound according to any one of claims 1 to 16, which is selected from 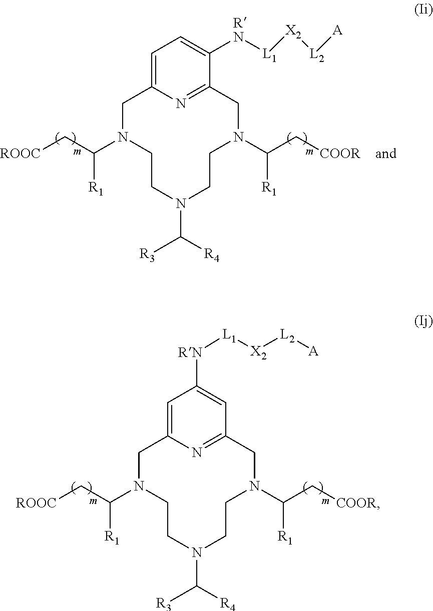
or a pharmaceutically acceptable salt thereof.

22. The compound according to any one of claims 1 to 16, which is selected from 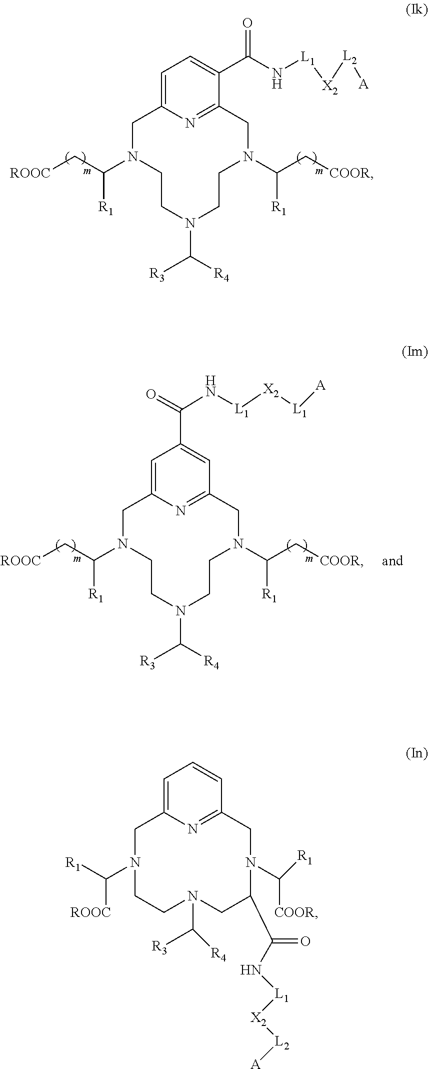
or a pharmaceutically acceptable salt thereof.

23. The compound according to any one of claims 1 to 16, which is selected from 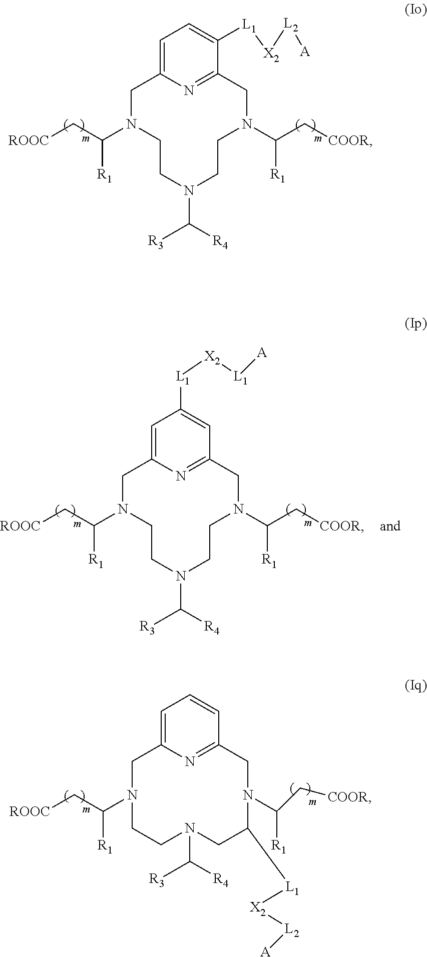
or a pharmaceutically acceptable salt thereof.

24. The compound according to any one of claims 1 to 16, which is selected from 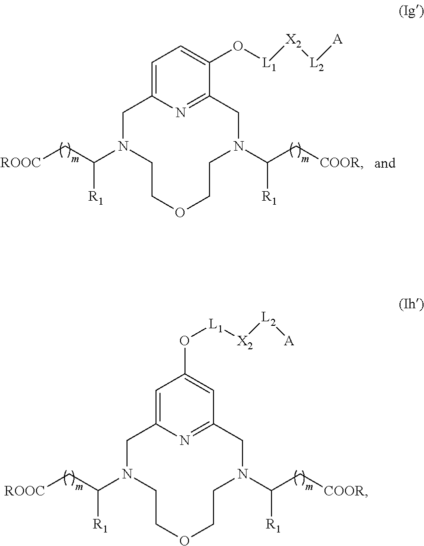
or a pharmaceutically acceptable salt thereof.

25. The compound according to any one of claims 1 to 16, which is selected from 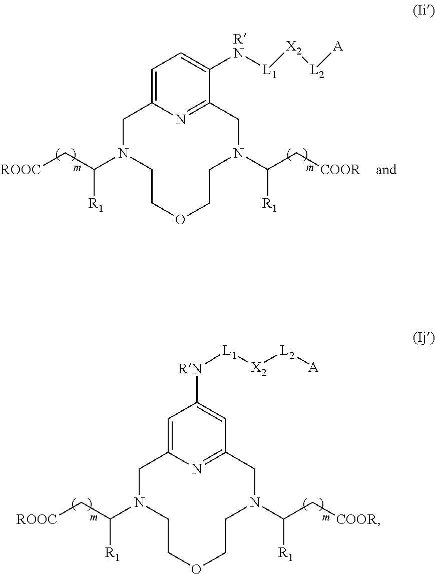
or a pharmaceutically acceptable salt thereof.

26. The compound according to any one of claims 1 to 16, which is selected from 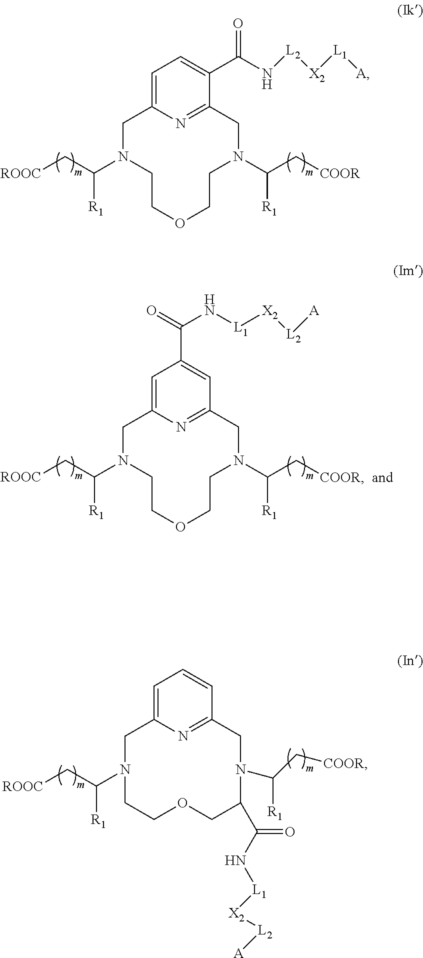
or a pharmaceutically acceptable salt thereof.

27. The compound according to any one of claims 1 to 16, which is selected from 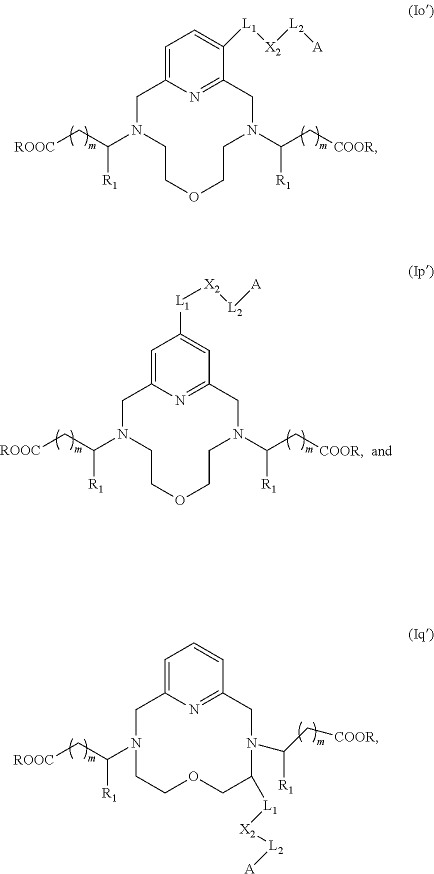
or a pharmaceutically acceptable salt thereof.

28. The compound according to any one of claims 1 to 16, which is selected from 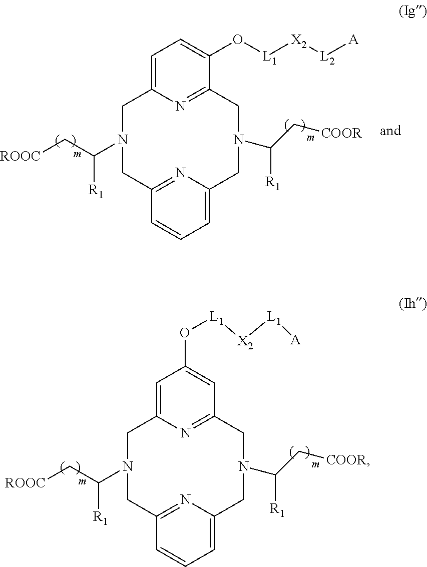
or a pharmaceutically acceptable salt thereof.

29. The compound according to any one of claims 1 to 16, which is selected from 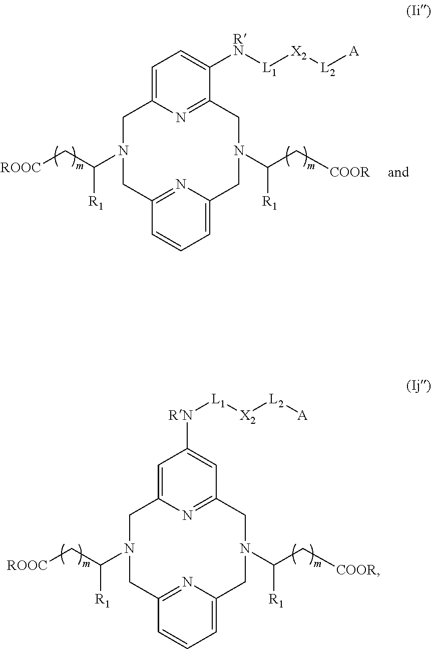
or a pharmaceutically acceptable salt thereof.

30. The compound according to any one of claims 1 to 16, which is selected from 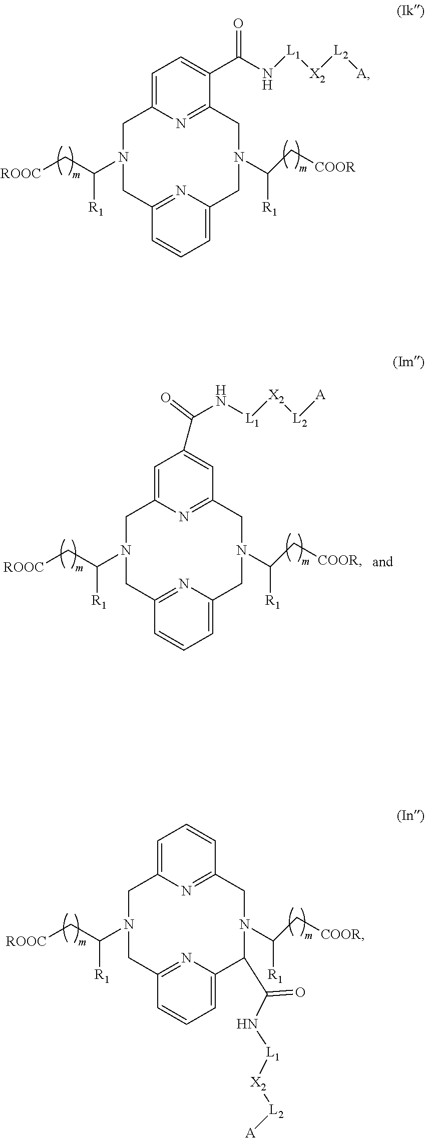
or a pharmaceutically acceptable salt thereof.

31. The compound according to any one of claims 1 to 16, which is selected from 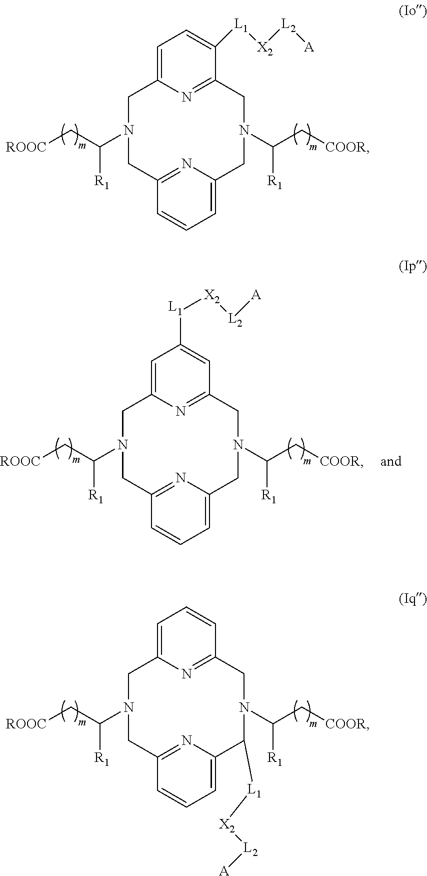
or a pharmaceutically acceptable salt thereof.

32. The compound according to any one of claims 1 to 16, which is selected from 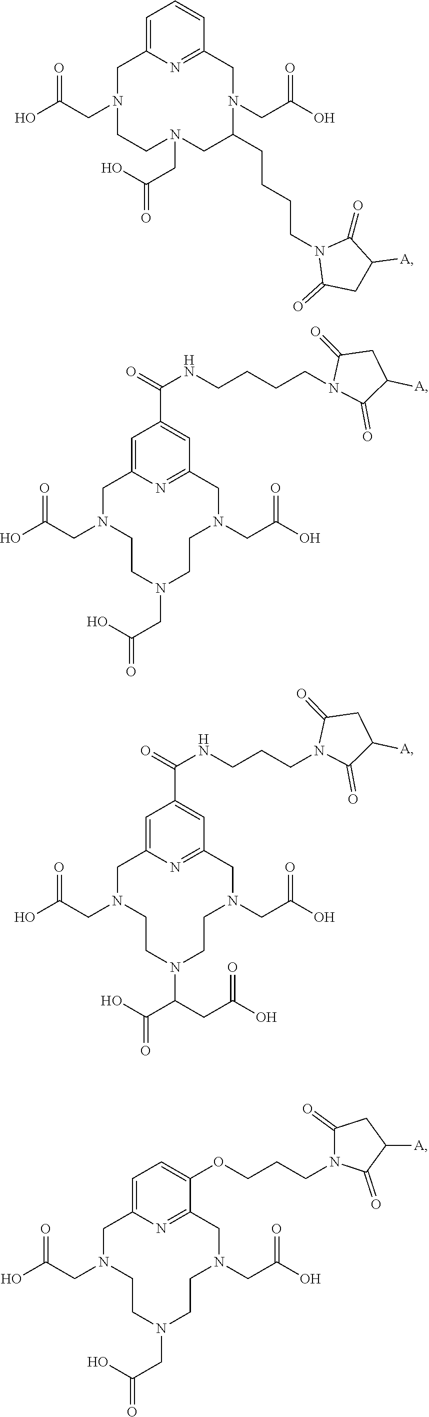 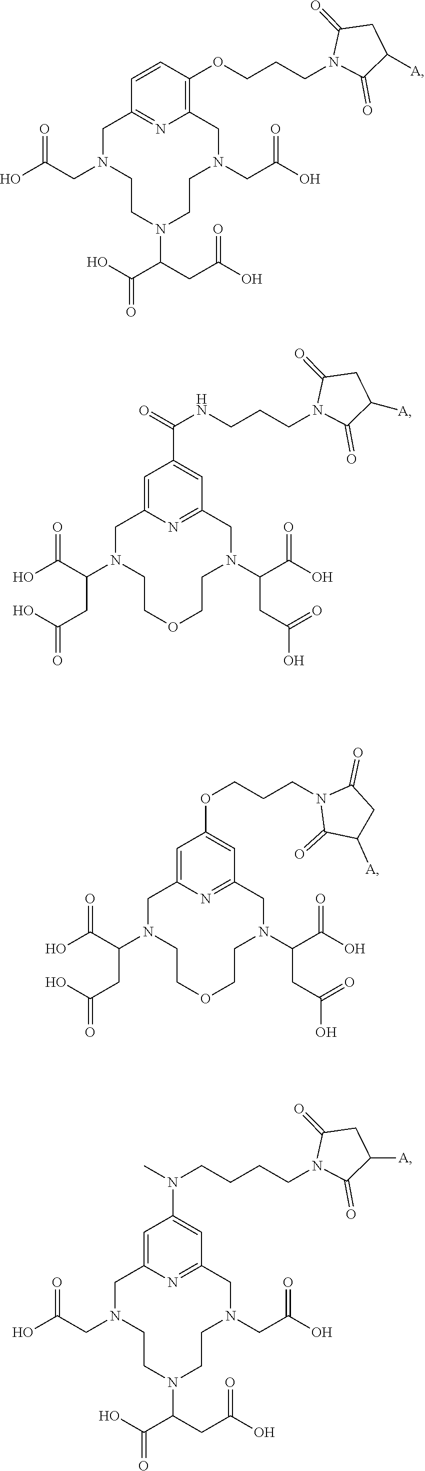 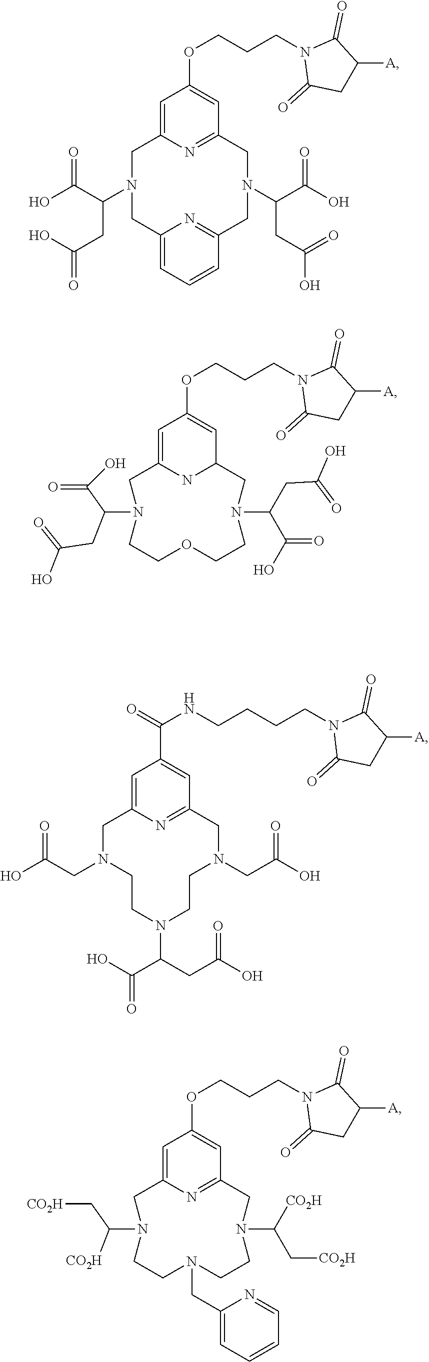 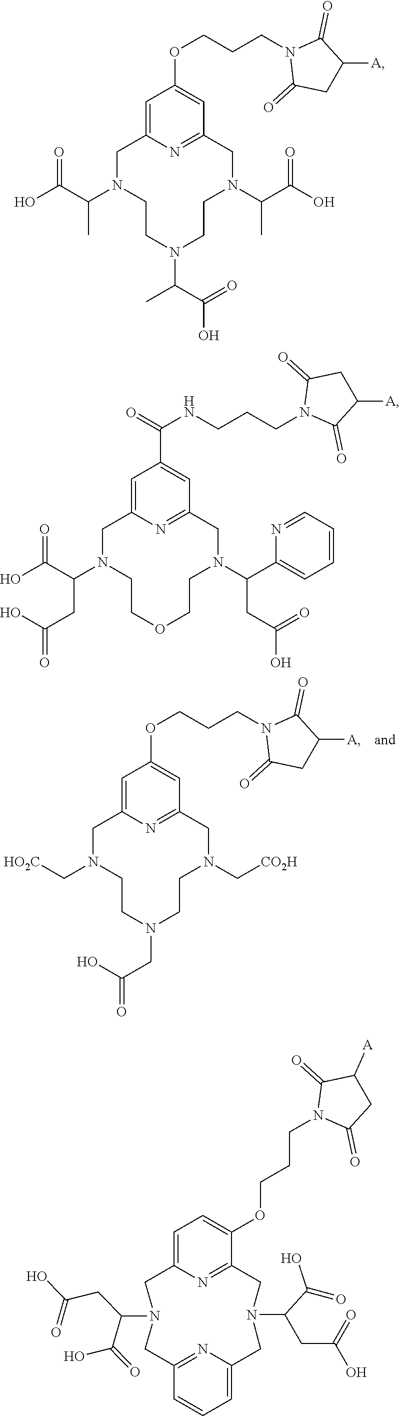
or a pharmaceutically acceptable salt thereof.

33. The compound according to claim 32, which is selected from 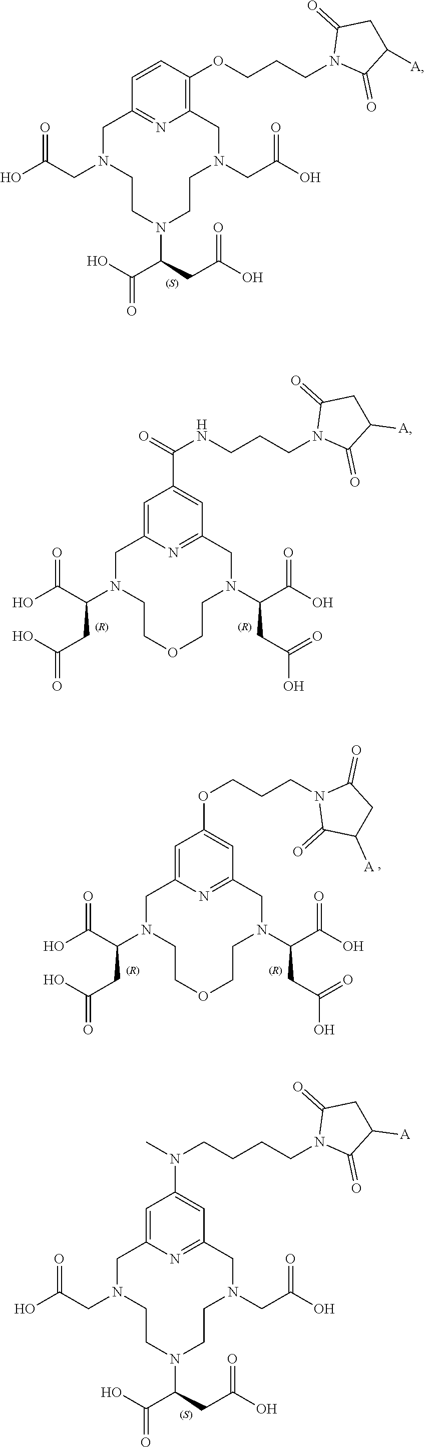 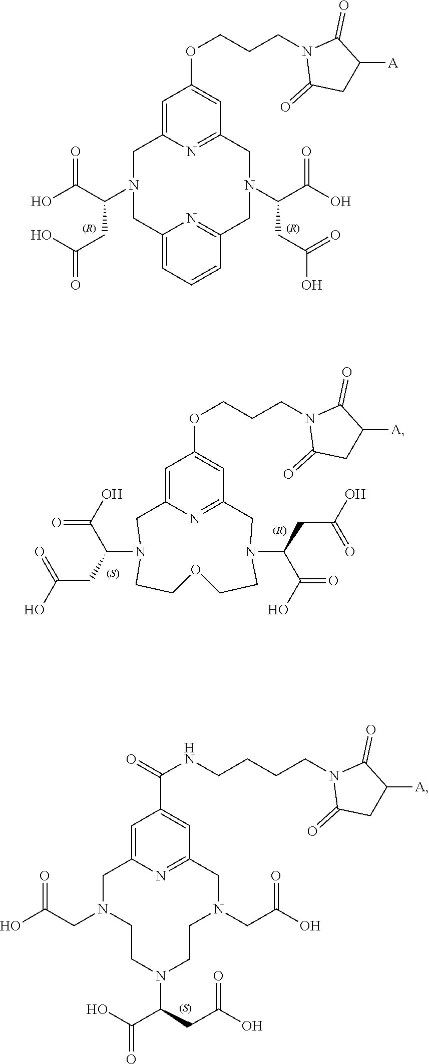 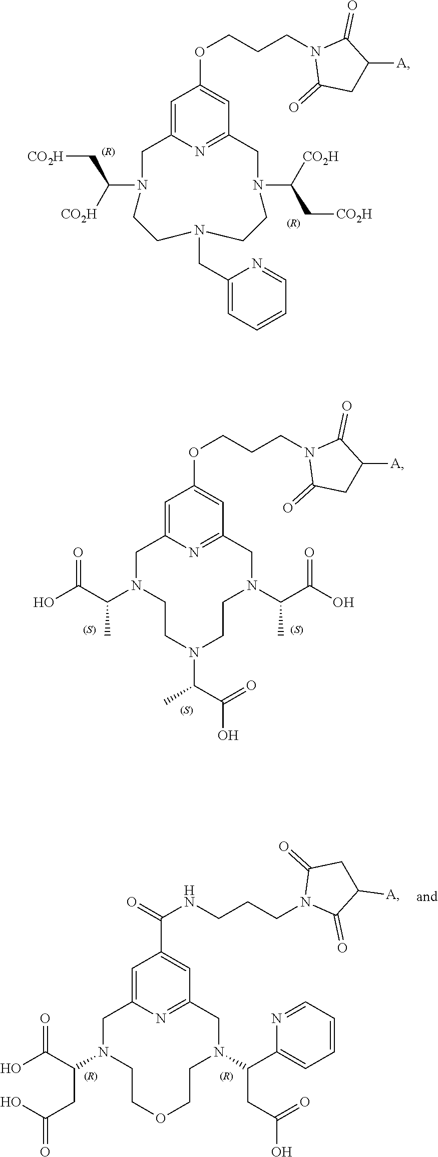 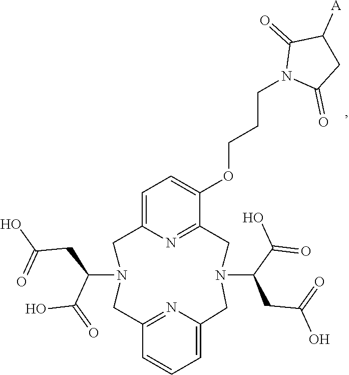
or a pharmaceutically acceptable salt thereof,
wherein A is a target-binding moiety as defined in any one of claims 13-16.

34. The compound according to any one of claims 1 to 33, or a pharmaceutically acceptable salt thereof, which is further complexed with a radionuclide.

35. The compound according to claim 34, wherein the radionuclide is selected from the group consisting of ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, Al¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

36. A compound of formula (II) 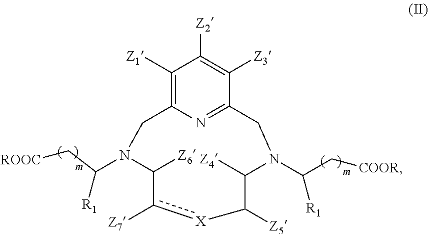
or a pharmaceutically acceptable salt thereof,
wherein [symbol]
is a single bond or a double bond, wherein
when [symbol] is a single bond, X is —O— or 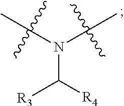
when [symbol] is a double bond, X is ═N— and Z₅, and Z₇′ form, together with the N atom, a heteroaryl having 5 or 6 ring atoms, otherwise, Z₅′ and Z₇′ are each independently selected from the group consisting of H, an albumin-binder and —X₁-L₁-X₂′;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is independently selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl and heteroaryl having 5 to 10 ring atoms, or any combination thereof;
Z₁′, Z₂′, Z₃′, Z₄′, and Z₆′ are each independently from the group consisting of H, an albumin-binder H or a group —X₁-L₁-X₂′, provided that at least one of Z₁′, Z₂′, Z₃′, Z₄, Z₅′, Zr, of Z₇′ is a group —X₁-L₁-X₂′;
X₁ present or absent wherein when present:
X₁ is selected from the group consisting of —O—, —NR′—, —C(═O)NR′, —NR′C(═O)—, —OC(═O)—, —C(═O)O—, —OC(═O)NR′—, —NR′C(═O)O—; —CH₂—O—; and —NR′C(═O)NR′, with the proviso that when X₁ is NR′, R₄ is not H;
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene-, heteroarylene having 5 to 10 ring atoms, one or more natural or non-natural amino acids, and any combination thereof, said alkylene, heteroalkylene, cycloalkylene, heterocycloalkylene, heteroarylene, and amino acid are optionally substituted with one or more substituents preferably selected from: albumin-binder, C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from a group consisting of H, C₁-C₆ alkyl, C₁-C₆ heteroalkyl, C₃-C₆ cycloalkyl, C₃-C₈ heterocycle, 3 to 10 membered heterocycloalkyl, C₆-C₁₀ aryl and heteroaryl having 5 to 10 ring atoms;
X₂′ is a group selected from the group consisting of: 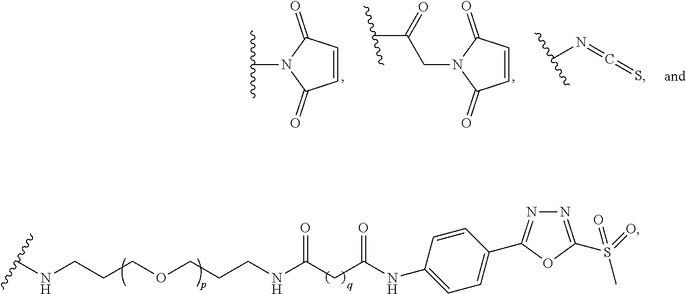
each m is independently 0 or 1;
n is 1, 2, 3, 4, 5, or 6;
p is 1, 2, 3, 4, 5, or 6; and
q is 1, 2, 3, 4, 5, or 5.

37. The compound according to claim 36, or a pharmaceutically acceptable salt thereof, wherein R is H.

38. The compound according to claim 36 or 37, or a pharmaceutically acceptable salt thereof, wherein each R₁ is independently selected from the group consisting of H, C₁-C₆ alkyl, C(═O)OR₂, (C₁-C₆ alkyl)-C(═O)OR₂ and heteroaryl having 5 to 10 ring atoms.

39. The compound according to any one of claims 36 to 38, or a pharmaceutically acceptable salt thereof, wherein each R₁ is independently selected from the group consisting of H, CH₃, C(═O)OH, CH₂C(═O)OH and pyridyl.

40. The compound according to any one of claims 36 to 39, or a pharmaceutically acceptable salt thereof, wherein R₃ is selected from the group consisting of H, C₁-C₃ alkyl and C(═O)OR.

41. The compound according to any one of claims 36 to 40, or a pharmaceutically acceptable salt thereof, wherein R₃ is selected from the group consisting of H, CH₃ and C(═O)OH.

42. The compound according to any one of claims 36 to 41, or a pharmaceutically acceptable salt thereof, wherein R₄ is selected from the group consisting of H, C(═O)OR, (C₁-C₆ alkyl)-C(═O)OR₂ and heteroaryl having 5 to 10 ring atoms.

43. The compound according to any one of claims 36 to 42, or a pharmaceutically acceptable salt thereof, wherein R₄ is selected from the group consisting of H, C(═O)OH, CH₂C(═O)OH and pyridyl.

44. The compound according to any one of claims 36 to 43, or a pharmaceutically acceptable salt thereof, wherein only one of Z₁′, Z₂′, Z₃′, Z₄′, Z₅′, Z₆′, and Z₇′ is —X₁-L₁-X₂′, and the other Z groups are H.

45. The compound according to any one of claims 36 to 44, or a pharmaceutically acceptable salt thereof, wherein X₁ is selected from the group consisting of —O—, —N(CH₃)— and —C(═O)NH—.

46. The compound according to any one of claims 36 to 45, or a pharmaceutically acceptable salt thereof, wherein L₁ is C₁-C₅ alkylene.

47. The compound according to any one of claims 36 to 46, or a pharmaceutically acceptable salt thereof, wherein X₂′ is

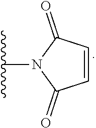 [SMILES: *N1C(=O)C=CC1=O]

48. The compound according to any one of claims 36 to 47, which is selected from 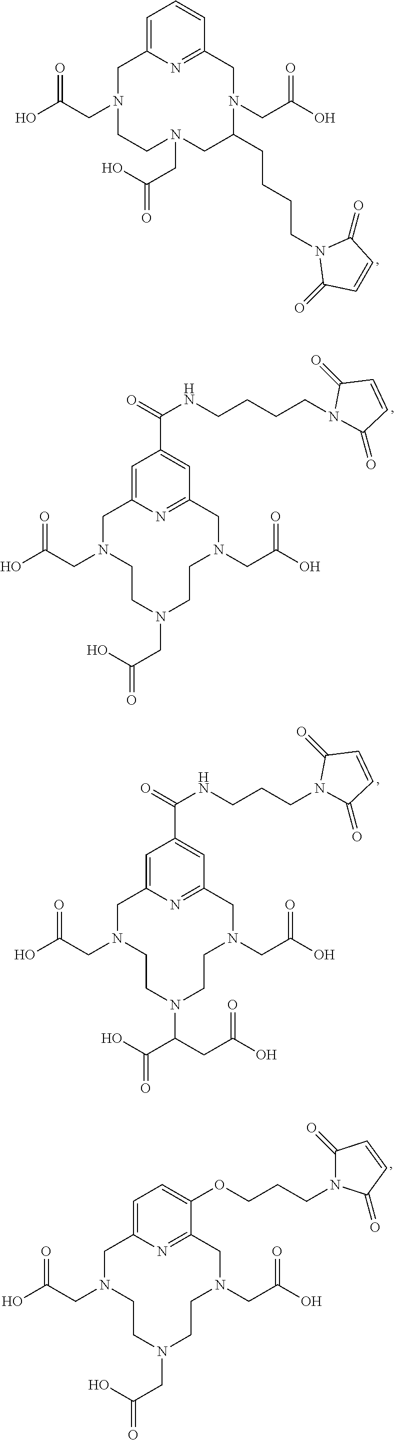 [SMILES: O=C(O)CN1CCN(CC(=O)O)CC(CCCCN2C(=O)C=CC2=O)N(CC(=O)O)Cc2cccc(n2)C1 ; O=C(O)CN1CCN(CC(=O)O)Cc2cc(C(=O)NCCCCN3C(=O)C=CC3=O)cc(n2)CN(CC(=O)O)CC1 ; O=C(O)CC(C(=O)O)N1CCN(CC(=O)O)Cc2cc(C(=O)NCCCN3C(=O)C=CC3=O)cc(n2)CN(CC(=O)O)CC1 ; O=C(O)CN1CCN(CC(=O)O)Cc2ccc(OCCCN3C(=O)C=CC3=O)c(n2)CN(CC(=O)O)CC1] 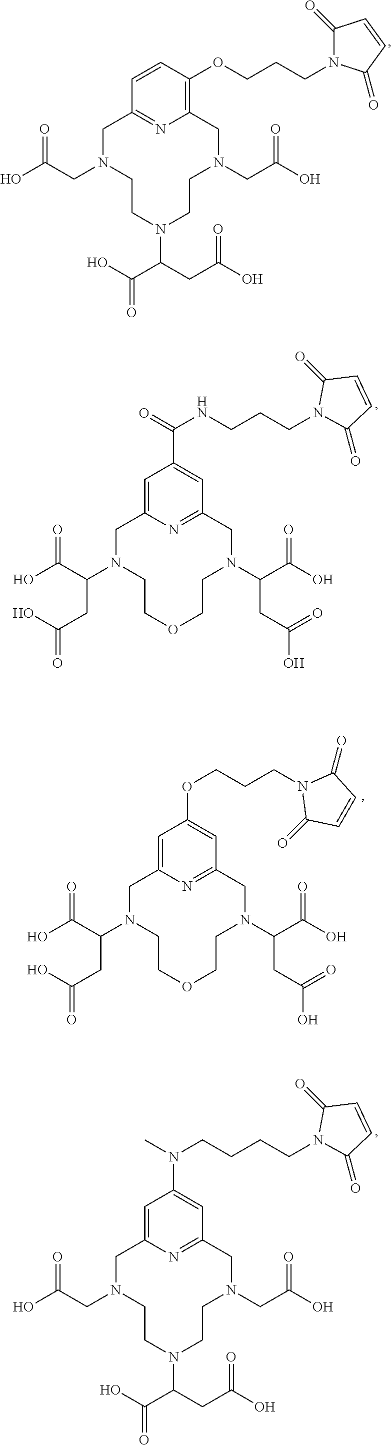 [SMILES: O=C(O)CC(C(=O)O)N1CCN(CC(=O)O)Cc2ccc(OCCCN3C(=O)C=CC3=O)c(n2)CN(CC(=O)O)CC1 ; O=C(O)CC(C(=O)O)N1CCOCCN(C(CC(=O)O)C(=O)O)Cc2cc(C(=O)NCCCN3C(=O)C=CC3=O)cc(n2)C1 ; O=C(O)CC(C(=O)O)N1CCOCCN(C(CC(=O)O)C(=O)O)Cc2cc(OCCCN3C(=O)C=CC3=O)cc(n2)C1 ; CN(CCCCN1C(=O)C=CC1=O)c1cc2nc(c1)CN(CC(=O)O)CCN(C(CC(=O)O)C(=O)O)CCN(CC(=O)O)C2] 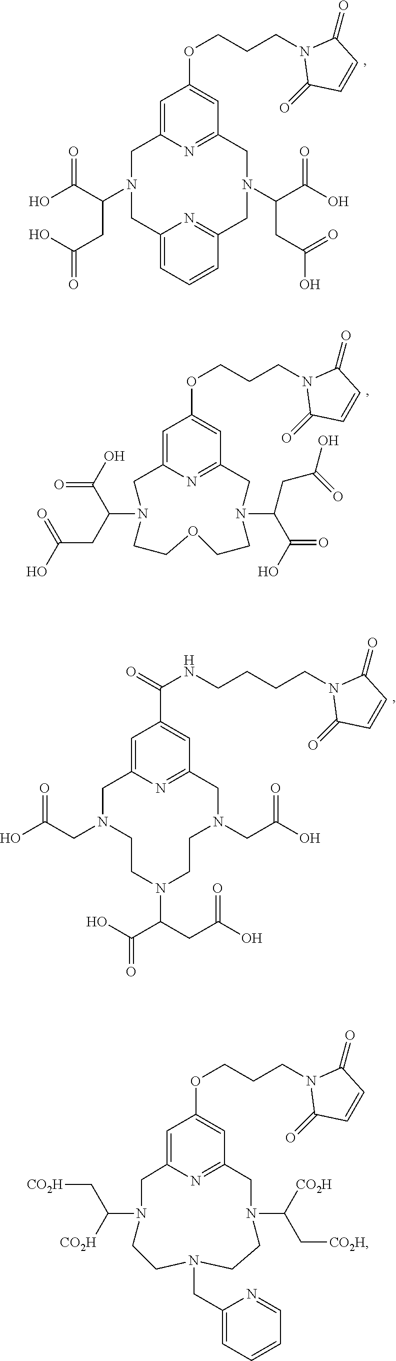 [SMILES: O=C(O)CC(C(=O)O)N1Cc2cccc(n2)CN(C(CC(=O)O)C(=O)O)Cc2cc(OCCCN3C(=O)C=CC3=O)cc(n2)C1 ; O=C(O)CC(C(=O)O)N1CCOCCN(C(CC(=O)O)C(=O)O)Cc2cc(OCCCN3C(=O)C=CC3=O)cc(n2)C1 ; O=C(O)CC(C(=O)O)N1CCN(CC(=O)O)Cc2cc(C(=O)NCCCCN3C(=O)C=CC3=O)cc(n2)CN(CC(=O)O)CC1 ; O=C(O)CC(C(=O)O)N1CCN(Cc2ccccn2)CCN(C(CC(=O)O)C(=O)O)Cc2cc(OCCCN3C(=O)C=CC3=O)cc(n2)C1] 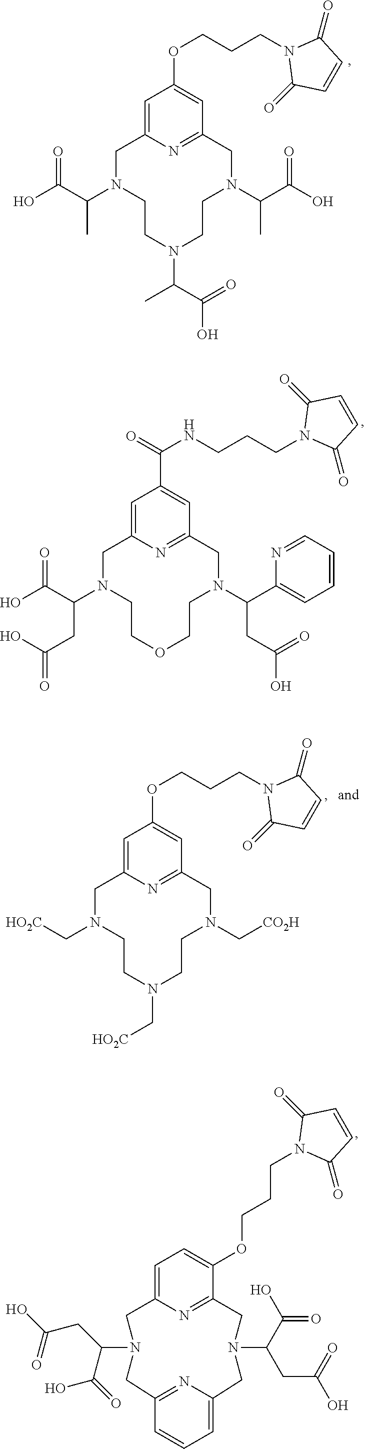 [SMILES: CC(C(=O)O)N1CCN(C(C)C(=O)O)Cc2cc(OCCCN3C(=O)C=CC3=O)cc(n2)CN(C(C)C(=O)O)CC1 ; O=C(O)CC(C(=O)O)N1CCOCCN(C(CC(=O)O)c2ccccn2)Cc2cc(C(=O)NCCCN3C(=O)C=CC3=O)cc(n2)C1 ; O=C(O)CN1CCN(CC(=O)O)Cc2cc(OCCCN3C(=O)C=CC3=O)cc(n2)CN(CC(=O)O)CC1 ; O=C(O)CC(C(=O)O)N1Cc2cccc(n2)CN(C(CC(=O)O)C(=O)O)Cc2nc(ccc2OCCCN2C(=O)C=CC2=O)C1]
or a pharmaceutically acceptable salt thereof.

49. The compound according to claim 48, which is selected from 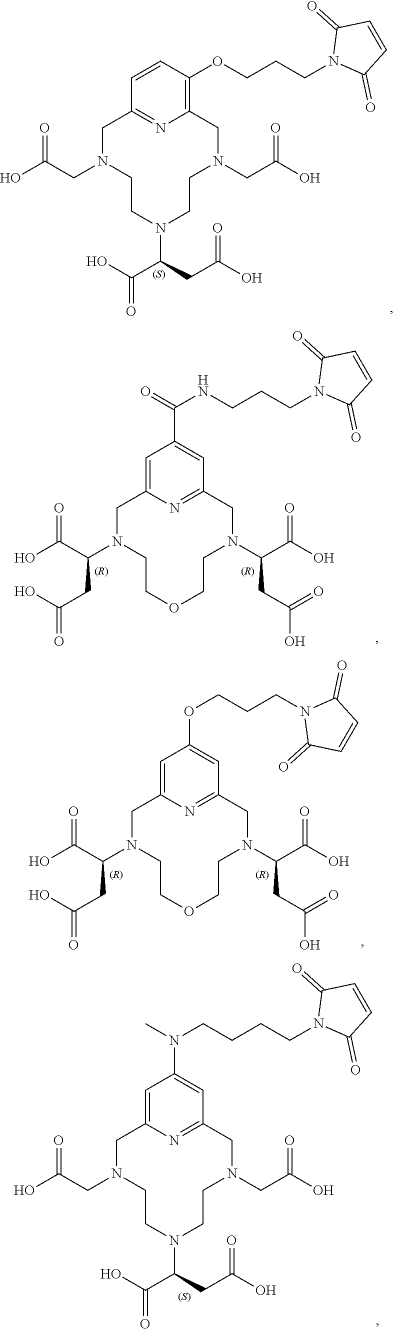 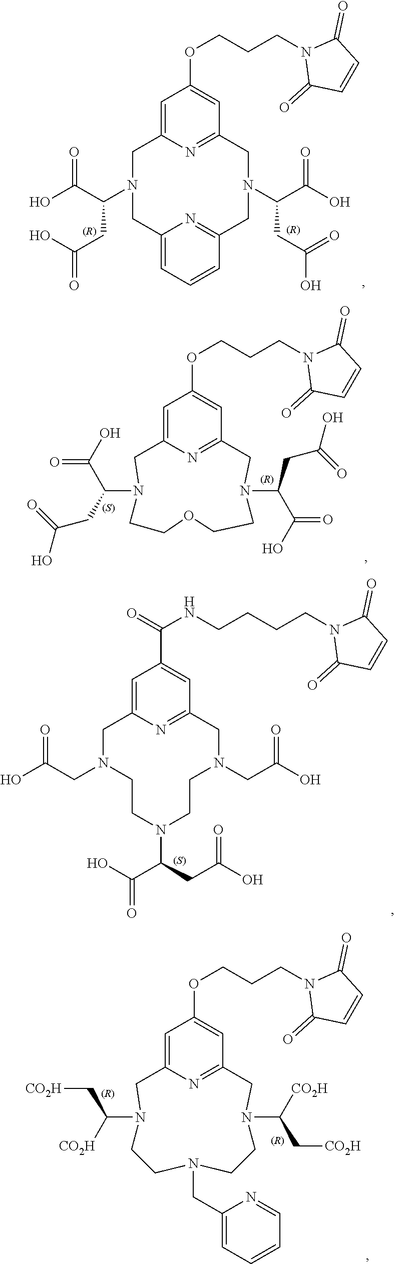 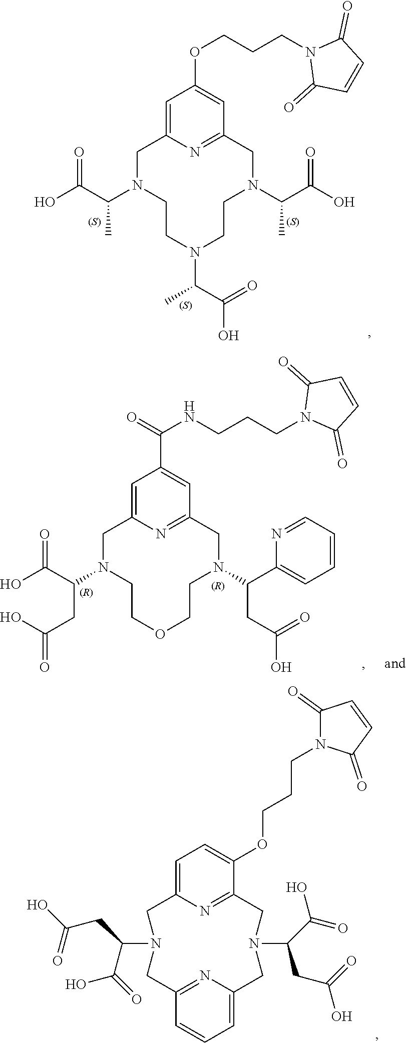
or a pharmaceutically acceptable salt thereof.

50. The compound according to any one of claims 36 to 49, or a pharmaceutically acceptable salt thereof, which is further complexed with a radionuclide.

51. The compound according to claim 50, wherein the radionuclide is selected from the group consisting of ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, Al¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

52. The compound according to any one of claims 1 to 33 and 36 to 49, or a pharmaceutically acceptable salt, for use as a medicament.

53. The compound according to claim 52, or a pharmaceutically acceptable salt, wherein the medicament is for treating cancer, and the compound is complexed with a radionuclide selected from the group consisting of ¹⁶⁹Er, ⁶⁴Cu, ⁶⁷Cu, ¹⁸⁶Re, ¹⁸⁸Re, ⁹⁰Y, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ¹⁴²Pr, ¹⁴³Pr, ¹⁹⁸Au, ¹⁹⁹Au, ¹⁴⁹Tb, ⁴⁷Sc, ²²⁵Ac, ²¹²Pb, ²¹¹At, ²²³Ra, ²²⁷Th, ¹³¹I, ⁷⁶As, ¹¹¹Ag, ¹⁶⁵Er and ²²⁷Ac.

54. The compound according to claim 52, or a pharmaceutically acceptable salt, wherein the medicament is for diagnosing cancer disorders, and the compound is complexed with a radionuclide selected from the group consisting of ¹¹¹In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁷⁷Lu, ¹⁶⁹Yb, ¹⁷²Tm, ^{117m}Sn, ¹²³I, ¹²⁴I, ¹²⁵I, ¹⁸F, Al¹⁸F, ¹⁵²Tb, ¹⁵⁵Tb, ⁴⁴Sc, ⁴³Sc, ⁸²Rb, ⁸⁹Zr and ⁶¹Cu.

55. A method for radiolabelling the compound according to any one of claims 1 to 33 and 36 to 49, or a pharmaceutically acceptable salt, said method comprising reacting said compound with a radionuclide to form a complex of said compound and radionuclide, and recovering the complex; thereby obtaining a radiolabelled compound.

56. The method according to claim 55, or a pharmaceutically acceptable salt, wherein said radionuclide is selected from the group consisting of ¹¹¹In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹³Bi, ¹⁴²Pr, ¹⁴³Pr, ¹⁹⁸Au, ¹⁹⁹Au, ¹²³I, ¹²⁴I, ¹²⁵I, ¹⁸F, ¹⁴⁹Tb, ¹⁵²Tb, ¹⁵⁵Tb, ⁴⁷Sc, ⁴⁴Sc, ⁴³Sc, ²²⁵Ac, ²¹²Pb, ²¹¹At, ²²³Ra, ²²⁷Th, ¹³¹I, ⁸²Rb, ⁷⁶As, ⁸⁹Zr, ¹¹¹Ag, ¹⁶⁵Er, ²²⁷Ac, and ⁶¹Cu.

57. A pharmaceutical composition comprising the compound according to any one of claims 1 to 56, or a pharmaceutically acceptable salt thereof, and at least one pharmaceutically acceptable carrier

58. A method of treating cancer in a subject in need thereof, comprising administering to the subject a therapeutically efficient amount of the compound of any one of claims 1 to 53, or a pharmaceutically acceptable salt thereof, or the pharmaceutical composition according to claim 57.

59. A method of imaging cancer in a subject, comprising administering to the subject the compound of any one of claims 1 to 52 and 54, or a pharmaceutically acceptable salt thereof, or the pharmaceutical composition according to claim 57.

60. A method for synthesizing a compound of formula (I) as described in claim 1, or a pharmaceutically acceptable salt thereof, said method comprising (a) reacting a target-binding compound L₂-A, wherein L₂ is an optional linker and A is a target-binding moiety, with a compound of formula (II): 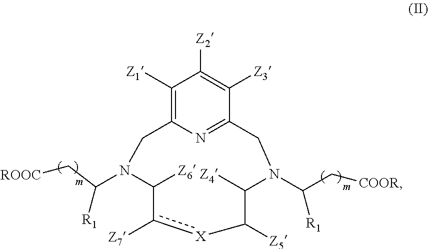
wherein [symbol]
is a single bond or a double bond,
when [symbol] is a single bond, X is —O— or 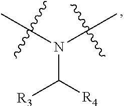
when [symbol] is a double bond, X is ═N— and Z₅′ and Z₇′ form, together with the N atom, a heteroaryl having 5 or 6 ring atoms, otherwise, Z₅′ and Z₇′ are each independently selected from the group consisting of H, albumin binder and —X₁-L₁-X₂′
each R is independently selected from the group consisting of H, and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁₀ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R₂ is independently selected from the group consisting of H and C₁-C₆ alkyl
R₃ is selected from the group consisting of H C₁-C₆ alkyl and C(═O)OR,
R₄ is selected from the group consisting of H, C(═O)OR, (C₁-C₆ alkyl)-C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, heteroaryl having 5 to 10 ring atoms, or any combination thereof;
Z₁′, Z₂′, Z₃′, Z₄′, and Z₆′ are each independently selected from the group consisting of H, an albumin-binder and —X₁-L₁-X₂′;
X₁ is present or absent wherein when present:
X₁ is selected from the group consisting of —O—, —NR′—, —C(═O)NR′—, —NR′C(═O)—, —OC(═O)—, —C(═O)O—, —OC(═O)NR′—, —NR′C(═O)O—; —CH₂—O—; and —NR′C(═O)NR′—, with the proviso that when X₁ is NR′, R₄ is not H, preferably X₁ is selected from the group consisting of —O—, —N(CH₃)—, —C(═O)NH—.
L₁ is a linker selected from the group consisting of C₁-C₅ alkylene, (—CH₂CH₂O)ₙ, C₁-C₆ heteroalkylene, C₃-C₆ cycloalkylene, —C₃-C₈ heterocycloalkylene, heteroarylene having 5 to 10 ring atoms, one or more natural or non-natural amino acids, and any combination thereof, said alkylene, heteroalkylene, cycloalkylene, heterocyclo alkylene, heteroarylene, and amino acid are optionally substituted with one or more substituents preferably selected from: C₁-C₆ alkyl, —OR′, ═O, ═NR′, ═N—OR′, —NR′R″, —SR′, -halogen, —SiR′R″R′″, —OC(═O)R′, (C₁-C₆ alkyl)-C(═O)OR′, —C(═O)R′, —C(═O)OR′, (C₁-C₆ alkyl)-C(═O)OR′—C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
R′, R″, R′″ and R″″ are each independently selected from the group consisting of H, C₁-C₆ alkyl, C₁-C₆ heteroalkyl, C₃-C₆ cycloalkyl, C₃-C₈ heterocycle, C₆-C₁₀ aryl, and heteroaryl having 5 to 10 ring atoms;
X₂′ is selected from the group consisting of: 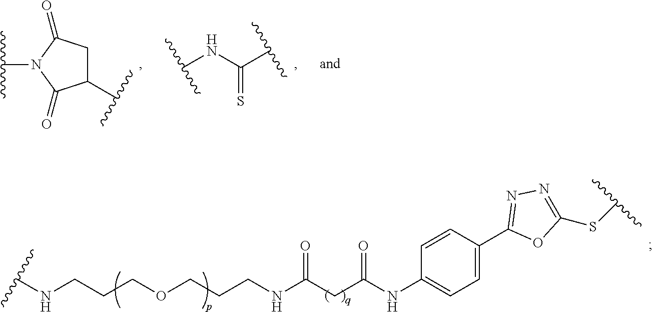
each m is independently 0 or 1;
n is 1, 2, 3, 4, 5, or 6;
p is 1, 2, 3, 4, 5, or 6;
q is 1, 2, 3, 4, 5, or 5; and
b) recovering the compound of formula (I).

61. A compound capable of complexing ⁶⁸Ga and/or ¹⁷⁷Lu at a temperature of ≤60° C., to achieve 85% complexation of the compound in a composition, said compound comprising formula (C) 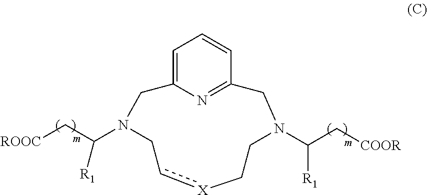
or a pharmaceutically acceptable salt thereof
wherein [symbol]
is a single bond or a double bond,
when [symbol] is a single bond, X is —O— or 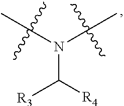
when [symbol] is a double bond, X is ═N—
each m is 0 to 5;
each R is independently selected from the group consisting of H and C₁-C₆ alkyl;
each R₁ is independently selected from the group consisting of H, C(═O)OR₂, (C₁-C₆ alkyl)-C(═O)OR₂, C₁-C₆ alkyl, C₁-C₆ heteroalkyl, C₃-C₆ cycloalkyl, C₆-C₁ aryl, heteroaryl having 5 to 10 ring atoms, and any combination thereof, wherein said, alkyl, heteroalkyl, cycloalkyl, aryl and heteroaryl are optionally substituted with one or more substituents independently selected from: —OR′, ═O, ═NR′, ═N—OR′, —NR′R″, —SR′, -halogen, —SiR′R″R′″, —OC(═O)R′, —C(═O)R′, —C(═O)OR′, —C(═O)NR′R″, —OC(═O)NR′R″, —NR″C(═O)R′, —NR′—C(═O)NR″R′″, —NR″C(═O)OR′, —NR′—C(NR″R′″)═NR″″, —S(═O)R′, —S(═O)₂R′, —S(═O)₂NR′R″, —NRS(═O)₂R′, —CN and —NO₂;
each R₂ is selected from the group consisting of H and C₁-C₆ alkyl;
R₃ is selected from the group consisting of H, C₁-C₆ alkyl, and C(═O)OR;
R₄ is selected from the group consisting of H, C(═O)OR, (C₁-C₆ alkyl)-C(═O)OR, C₁-C₆ alkyl, C₁-C₆ heteroalkyl, C₃-C₆ cycloalkyl, C₃-C₈ heterocycle, C₆-C₁₀ aryl, heteroaryl having 5 to 10 ring atoms, or any combination thereof, and
the compound of formula (C) being optionally substituted.
